SEQUENCE LISTING (38 sequences)

SEQ ID NO: 1 - DNA, 1965 bp, Brassica napus
  CDS (1)..(1965)
atggcggcggcaacatcgtcttctccgatctccttaaccgctaaaccttcttccaaatcccctctacccatttccagattctcccttcccttctccttaaccccacagaaagactcctcccgtctccaccgtcctctcgccatctccgccgttctcaactcacccgtcaatgtcgcacctccttcccctgaaaaaaccgacaagaacaagactttcgtctcccgctacgctcccgacgagccccgcaagggtgctgatatcctcgtcgaagccctcgagcgtcaaggcgtcgaaaccgtctttgcttatcccggaggtgcttccatggagatccaccaagccttgactcgctcctccaccatccgtaacgtccttccccgtcacgaacaaggaggagtcttcgccgccgagggttacgctcgttcctccggcaaaccgggaatctgcatagccacttcgggtcccggagctaccaacctcgtcagcgggttagcagacgcgatgcttgacagtgttcctcttgtcgccattacaggacaggtccctcgccggatgatcggtactgacgccttccaagagacaccaatcgttgaggtaacgaggtctattacgaaacataactatttggtgatggatgttgatgacatacctaggatcgttcaagaagctttctttctagctacttccggtagacccggaccggttttggttgatgttcctaaggatattcagcagcagcttgcgattcctaactgggatcaacctatgcgcttacctggctacatgtctaggttgcctcagcctccggaagtttctcagttaggtcagatcgttaggttgatctcggagtctaagaggcctgttttgtacgttggtggtggaagcttgaactcgagtgaagaactggggagatttgtcgagcttactgggatccccgttgcgagtactttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggctttcgctagcagggctaaaattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttccctttgagcttcaaaacgtttggagaagccattcctccgcagtacgcgattcagatcctcgacgagctaaccgaagggaaggcaattatcagtactggtgttggacagcatcagatgtgggcggcgcagttttacaagtacaggaagccgagacagtggctgtcgtcatcaggcctcggagctatgggttttggacttcctgctgcgattggagcgtctgtggcgaaccctgatgcgattgttgtggatattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacaccaggaccatacctgttggatgtgatatgtccgcaccaagaacatgtgttaccgatgatcccaagtggtggcactttcaaagatgtaataacagaaggggatggtcgcactaagtac

SEQ ID NO: 2 - protein, 655 aa, Brassica napus
MAAATSSSPISLTAKPSSKSPLPISRFSLPFSLTPQKDSSRLHRPLAISAVLNSPVNVAPPSPEKTDKNKTFVSRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQILDELTEGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPSGGTFKDVITEGDGRTKY

SEQ ID NO: 3 - DNA, 1956 bp, Brassica napus
  CDS (1)..(1956)
atggcggcggcaacatcgtcttctccgatctccttaaccgctaaaccttcttccaaatcccctctacccatttccagattctcccttcccttctccttaaccccacagaaaccctcctcccgtctccaccgtcctctcgccatctccgccgttctcaactcacccgtcaatgtcgcacctgaaaaaaccgacaagatcaagactttcatctcccgctacgctcccgacgagccccgcaagggtgctgatatcctcgtggaagccctcgagcgtcaaggcgtcgaaaccgtcttcgcttatcccggaggtgcctccatggagatccaccaagccttgactcgctcctccaccatccgtaacgtcctcccccgtcacgaacaaggaggagtcttcgccgccgagggttacgctcgttcctccggcaaaccgggaatctgcatagccacttcgggtcccggagctaccaacctcgtcagcgggttagccgacgcgatgcttgacagtgttcctctcgtcgccatcacaggacaggtccctcgccggatgatcggtactgacgccttccaagagacgccaatcgttgaggtaacgaggtctattacgaaacataactatctggtgatggatgttgatgacatacctaggatcgttcaagaagcattctttctagctacttccggtagacccggaccggttttggttgatgttcctaaggatattcagcagcagcttgcgattcctaactgggatcaacctatgcgcttgcctggctacatgtctaggctgcctcagccaccggaagtttctcagttaggccagatcgttaggttgatctcggagtctaagaggcctgttttgtacgttggtggtggaagcttgaactcgagtgaagaactggggagatttgtcgagcttactgggatccctgttgcgagtacgttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggcgtttgcgagcagggctaagattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttcccgttgagcttcaaaacgtttggagaagccattcctccgcagtacgcgattcaggtcctagacgagctaacccaagggaaggcaattatcagtactggtgttggacagcatcagatgtgggcggcgcagttttacaagtacaggaagccgaggcagtggctgtcgtcctcaggactcggagctatgggtttcggacttcctgctgcgattggagcgtctgtggcgaaccctgatgcgattgttgtggacattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacacctggaccgtacctgttggatgtcatctgtccgcaccaagaacatgtgttaccgatgatcccaagtggtggcactttcaaagatgtaataaccgaaggggatggtcgcactaagtac

SEQ ID NO: 4 - protein, 652 aa, Brassica napus
MAAATSSSPISLTAKPSSKSPLPISRFSLPFSLTPQKPSSRLHRPLAISAVLNSPVNVAPEKTDKIKTFISRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQVLDELTQGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPSGGTFKDVITEGDGRTKY

SEQ ID NO: 5 - DNA, 1956 bp, Brassica juncea
  CDS (1)..(1956)
atggcggcggcaacatcgtcttctccgatctccttaaccgctaaaccttcttccaaatcccctctacccatttccagattctcccttcccttctccttaaccccacagaaaccctcctcccgtctccaccgtcctctcgccatctccgccgttctcaactcacccgtcaatgtcgcacctgaaaaaaccgacaagatcaagactttcatctcccgctacgctcccgacgagccccgcaagggtgctgatatcctcgtggaagccctcgagcgtcaaggcgtcgaaaccgtcttcgcttatcccggaggtgcctccatggagatccaccaagccttgactcgctcctccaccatccgtaacgtcctcccccgtcacgaacaaggaggagtcttcgccgccgagggttacgctcgttcctccggcaaaccgggaatctgcattgccacttcgggtcccggagctaccaacctcgtcagcgggttagccgacgcgatgcttgacagtgttcctctcgtcgccattacaggacaggtccctcgccggatgatcggtactgacgccttccaagagacgccaatcgttgaggtaacgaggtctattacgaaacataactatctggtgatggatgttgatgacatacctaggatcgttcaagaagctttctttctagctacttccggtagacccggaccggttttggttgacgttcctaaggatattcagcagcagcttgcgattcctaactgggatcaacctatgcgcttgcctggctacatgtctaggctgcctcagccaccggaagtttctcagttaggtcagatcgttaggttgatctcggagtctaagaggcctgttttgtacgttggtggtggaagcttgaactcgagtgaagaactggggagatttgtcgagcttactgggatccctgttgcgagtacgttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggcgtttgcgagcagggctaagattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttcccgttgagcttcaaaacgtttggagaagccattcctccgcagtacgcgattcaggtcctagacgagctaacccaagggaaggcaattatcagtactggtgttggacagcatcagatgtgggcggcgcagttttacaagtacaggaagccgaggcagtggctgtcgtcctcaggactcggagctatgggtttcggacttcctgctgcgattggagcgtctgtggcgaaccctgatgcgattgttgtggacattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacacctggaccgtacctgttggatgtcatctgtccgcaccaagaacatgtgttaccgatgatcccaagtggtggcactttcaaagatgtaataaccgaaggggatggtcgcactaagtac

SEQ ID NO: 6 - protein, 652 aa, Brassica juncea; identical to SEQ ID NO: 4

SEQ ID NO: 7 - DNA, 1965 bp, Brassica juncea
  CDS (1)..(1965)
atggcggcggcaacatcgtcttctccaatctccttcaccgctaaaccttcttccaaatcccttttacccatttccagattctcccttcccttctccttaatcccgcagaaaccctcctcccttcgccacagtcctctctccatctcagccgttctcaacacacccgtcaatgtcgcacctccttcccctgaaaaaattgaaaagaacaagactttcatctcccgctacgctcccgacgagccccgcaagggcgccgatatcctcgtcgaagccctcgagcgtcaaggcgtcgaaaccgtcttcgcttacccgggaggtgcttccatggagatccaccaagccttaactcgatcctctaccatccgtaacgtcctcccccgtcacgaacaaggaggagtctttgccgccgagggttacgctcgttcctctggtaaaccgggaatctgcatagccacgtcaggtcccggagccaccaacctcgttagcggtttagccgacgcgatgctcgacagtgtccctctcgtcgctattacaggacaggtccctcgtcggatgattggtactgacgcgttccaggagacgccaatcgttgaggtaacgaggtctattacgaaacataactatctggtcatggatgttgatgacatacctaggatcgtgcaagaggctttctttctagctacttccggtagacccggaccggttttagttgatgttcctaaggatattcagcagcagcttgcgattcctaactgggatcagcctatgcgcttacctggttacatgtctaggctgcctcagcctccggaagtttctcagttagggcagatcgttaggttgatctctgaatctaagaggcctgttttgtatgttggtggtggaagcttgaactcgagtgatgaactggggaggtttgtggagcttactgggatccctgtcgcgagtactttgatggggcttggttcttatccttgtaacgatgagttgtctctgcagatgcttggtatgcacgggactgtgtacgctaattacgctgtggagcatagtgatttgttgctggcgtttggtgttaggtttgatgaccgtgtcactggaaagctcgaggcttttgcgagcagggctaagattgtgcacattgacattgattctgctgagattgggaagaacaagacgcctcatgtgtctgtgtgtggtgatgttaagctggctttgcaagggatgaacaaggttcttgagaaccgagcagaggagctcaagcttgacttcggagtttggaggagtgaattgagcgagcagaaacaaaagttcccgttgagttttaaaacgtttggagaagctattcctccacagtacgcgattcaggtcctcgacgagctaaccgatgggaaggcaatcatcagtactggtgttgggcaacatcagatgtgggcggcgcagttttacaagtacaggaagccgaggcagtggttgtcatcatcaggccttggagctatgggttttggacttcctgctgccattggagcgtctgtggcgaaccctgatgcgattgttgtggacattgacggtgacggaagcttcatcatgaatgttcaagagctggccacaatccgtgtagagaatcttcctgtgaaggtactcttgttaaacaaccagcatcttggcatggttatgcaatgggaagatcggttctacaaagctaacagagctcacacttatctcggggatccggcaaaggagaacgagatcttcccaaacatgctgcagtttgcaggagcctgtgggattccagctgcgagggtgacgaagaaagaagaactccgagatgctattcagacaatgctggatacaccaggaccatacctgttggatgtgatctgtccgcaccaagagcatgtgttaccgatgatcccaagtggtggtactttcaaagatgtcataacagaaggggatggtcgcactaagtac

SEQ ID NO: 8 - protein, 655 aa, Brassica juncea
MAAATSSSPISFTAKPSSKSLLPISRFSLPFSLIPQKPSSLRHSPLSISAVLNTPVNVAPPSPEKIEKNKTFISRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSDELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQVLDELTDGKAIISTGVGQHQMWAAQFYKYRKPRQWLSSSGLGAMGFGLPAAIGASVANPDAIVVDIDGDGSFIMNVQELATIRVENLPVKVLLLNNQHLGMVMQWEDRFYKANRAHTYLGDPAKENEIFPNMLQFAGACGIPAARVTKKEELRDAIQTMLDTPGPYLLDVICPHQEHVLPMIPSGGTFKDVITEGDGRTKY

SEQ ID NO: 9 - DNA, 2010 bp, Arabidopsis thaliana
  CDS (1)..(2010)
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtgacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatggcaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatac

SEQ ID NO: 10 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWQDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 11 - DNA, 1980 bp, Gossypium hirsutum
  CDS (1)..(1980)
atggcggctgccacttcgaactccgctttgcctaagctttctacgttaacttcgtccttcaaatcttccatacccatttccaaatccagccttcccttctccacaacccctcaaaagcccaccccttaccgctccttcgacgtttcctgctctctttctcatgcaagctccaaccccagatccgctgccgcatccgttacccaaaaaactgctcctccccattatttcatttccaggtatgcggatgatgagccccggaaaggcgctgatatcctggtggaagccctggaacgtgaaggggtcaaggatgtgtttgcctacccaggtggagcttcaatggagatccaccaggctttaacccgctcaaaaatcatccgaaatgtccttccgcgacacgagcaaggtggggtctttgccgccgagggttacgcgcgctcctctggcatttccggcgtttgcattgcgacgtctggccctggggcaaccaacttggtgagtggtctcgctgatgcaatgctcgatagtatccctctcgtggcgatcactggtcaagtccctcgtcggatgatcggtaccgatgctttccaggaaactccaattgttgaggtaacaaggtctattacgaagcataattatcttgttcttgatgtggatgatattcctaggattgttagtgaggctttctttttagcttcatcgggcaggccgggacctgttctgattgatgttcctaaggatatacaacagcaacttgctgttcctaaatggaaccattctcttagattgccagggtatttgtctaggttgcctaaggctcccgctgaggctcatctcgaacagatcgtgagattggtttctgagtctaagaagcctgttttatatgttggtggtgggtgtttgaactctagtgaggagttgaagaggtttgttgagcttacagggatacccgttgcaagtactttgatgggtcttggagcctttccgatttcggatgagttgtcgttacaaatgcttgggatgcacggaactgtgtatgccaattatgctgttgataagagtgatttgttgcttgcttttggagtgagatttgatgatagggtgacaggaaaacttgaggcttttgccagccgggcaaagattgtgcatatcgatatcgactctgctgagattgggaagaacaagcagcctcatatgtcagtgtgttccgatgtgaaattggcattgcaggggataaataagatattggagaccacgggagctaagctgaatcttgattattcggaatggaggcaggagttaaacgagcagaagctgaaattccctttgagttacaagacctttggtgaagctattccacctcaatatgcaattcaggttcttgatgaattaactggcgggaatgcaattataagtaccggtgttggccagcatcaaatgtgggctgctcaattttacaagtataagaagcctcgtcaatggttaacgtctgggggattgggtgctatgggatttggattgcctgctgctattggagctgctgttgcaaacccggaggcagttgttgtagacatcgacggtgatggaagttttatcatgaatgtgcaagagttggcgactatgcgtgtggaaaatcttccggttaagatattattgttgaataatcagcatttgggtatggttgttcaatgggaggaccggttttacaaggcaaacagggctcatacatacttgggagacccatccaacgagtcggaaatattcccaaatatgttgaaatttgctgaagcatgcgggataccagctgcccgggtgacgaagaaagaagatctcaaagcagcaattcagaaaatgttggacactcctggaccttacttgttggatgtgattgtcccacatcaagaacatgtcctgcctatgatccccagtggaggcgctttcaaagatgtgatcacagagggtgatggaagaacacaatattga

SEQ ID NO: 12 - protein, 659 aa, Gossypium hirsutum
MAAATSNSALPKLSTLTSSFKSSIPISKSSLPFSTTPQKPTPYRSFDVSCSLSHASSNPRSAAASVTQKTAPPHYFISRYADDEPRKGADILVEALEREGVKDVFAYPGGASMEIHQALTRSKIIRNVLPRHEQGGVFAAEGYARSSGISGVCIATSGPGATNLVSGLADAMLDSIPLVAITGQVPRRMIGTDAFQETPIVEVTRSITKHNYLVLDVDDIPRIVSEAFFLASSGRPGPVLIDVPKDIQQQLAVPKWNHSLRLPGYLSRLPKAPAEAHLEQIVRLVSESKKPVLYVGGGCLNSSEELKRFVELTGIPVASTLMGLGAFPISDELSLQMLGMHGTVYANYAVDKSDLLLAFGVRFDDRVTGKLEAFASRAKIVHIDIDSAEIGKNKQPHMSVCSDVKLALQGINKILETTGAKLNLDYSEWRQELNEQKLKFPLSYKTFGEAIPPQYAIQVLDELTGGNAIISTGVGQHQMWAAQFYKYKKPRQWLTSGGLGAMGFGLPAAIGAAVANPEAVVVDIDGDGSFIMNVQELATMRVENLPVKILLLNNQHLGMVVQWEDRFYKANRAHTYLGDPSNESEIFPNMLKFAEACGIPAARVTKKEDLKAAIQKMLDTPGPYLLDVIVPHQEHVLPMIPSGGAFKDVITEGDGRTQY

SEQ ID NO: 13 - DNA, 1980 bp, Gossypium hirsutum
  CDS (1)..(1980)
atggcggctgccactgcgaactccgctttgcctaagctttctacgttaacttcctccttcaaatcttccatacccatttccaaatccagccttcccttctccacaacccctcaaaagcccaccccttaccgctccttcgacgtttcctgctctctttctcatgcaagctctaaccccagatccgccgccacatccgttaccccaaaaaatgctcctccccatgatttcatttccaggtatgcggatgatgagccccggaaaggcgctgatatcctggtggaagccctggttcgtgaaggggtcaaggatgtgtttgcctacccaggtggagcttcaatggagatccaccaggctttaacccgctcaaaaatcatccgaaatgtccttccgcgacacgagcaaggtggggtctttgccgccgagggctacgcgcgctcctctggcattcccggcgtttgcattgcgacgtctggccctggggcaaccaacttggtgagtggtctcgctgatgcaatgctcgatagtatccctctcgtggcgatcactggtcaagtccctcgtcggatgatcggtaccgatgctttccaggaaactccaattgttgaggtaacaaggtctattacgaagcataattatcttgttcttgatgtggatgatattcctaggattgttagtgaggctttctttttagcttcctcgggcaggccgggacctgttctgattgatgttcctaaggatatacaacagcaacttgctgttcctaaatggaaccattctcttagattgccagggtatttgtctaggttgcctaaggctcccggtgaggctcatctcgaacagattgttagattggtttctgagtctaagaagcctgttttatatgttggtggtgggtgtttgaactctagtgaggagttgaagaggtttgttgagcttacagggatacctgttgcaagtactttgatgggtcttggagcctttccgatttcggatgacttgtcgttacaaatgcttgggatgcacggaactgtgtatgccaattatgctgttgataagagtgatttgttgcttgcttttggagtgagatttgatgatagggtgacgggaaaacttgaggcttttgccagccgggcaaagattgtgcatatcgatatcgattctgccgagattgggaagaacaagcagcctcatgtgtcagtgtgttccgatgtgaaattggcattgcaggggataaataagatattggagaccaaggtagcaaagctgaatcttgattattcggaatggaggcaggagttaaacgagcagaagctgaaattccctttgagttacaagacctttggtgaagctattccacctcaatatgcaattcaggttcttgatgaattaactggcgggaatgcaattataagtactggtgttggtcagcatcaaatgtgggctgctcaattttacaagtataagaagcctcgtcaatggttaacatctgggggattgggtgctatgggatttggattgcctgctgctattggagctgccgttgcaaacccagaggcagtcgttgtagacatcgatggtgatggaagttttatcatgaacgtgcaagagttggcgactatccgtgtggaaaatcttccggttaagatattattgttgaataatcagcatttgggtatggttgttcaatgggaggaccggttttacaaggcaaacagggctcatacatacttgggagacccatccaacgagtcggaaatattcccaaatatgttgaaatttgctgaagcatgcgggataccagctgcccgggtgacaaagaaagaagatctaaaagcagcaatgcagaaaatgttggacactcctggaccttacttgttggatgtgattgtcccacatcaagaacatgtcctgcctatgatccccagtggaggggctttcaaagatgtgatcacagagggtgatggaagaacacaatattga

SEQ ID NO: 14 - protein, 659 aa, Gossypium hirsutum
MAAATANSALPKLSTLTSSFKSSIPISKSSLPFSTTPQKPTPYRSFDVSCSLSHASSNPRSAATSVTPKNAPPHDFISRYADDEPRKGADILVEALVREGVKDVFAYPGGASMEIHQALTRSKIIRNVLPRHEQGGVFAAEGYARSSGIPGVCIATSGPGATNLVSGLADAMLDSIPLVAITGQVPRRMIGTDAFQETPIVEVTRSITKHNYLVLDVDDIPRIVSEAFFLASSGRPGPVLIDVPKDIQQQLAVPKWNHSLRLPGYLSRLPKAPGEAHLEQIVRLVSESKKPVLYVGGGCLNSSEELKRFVELTGIPVASTLMGLGAFPISDDLSLQMLGMHGTVYANYAVDKSDLLLAFGVRFDDRVTGKLEAFASRAKIVHIDIDSAEIGKNKQPHVSVCSDVKLALQGINKILETKVAKLNLDYSEWRQELNEQKLKFPLSYKTFGEAIPPQYAIQVLDELTGGNAIISTGVGQHQMWAAQFYKYKKPRQWLTSGGLGAMGFGLPAAIGAAVANPEAVVVDIDGDGSFIMNVQELATIRVENLPVKILLLNNQHLGMVVQWEDRFYKANRAHTYLGDPSNESEIFPNMLKFAEACGIPAARVTKKEDLKAAMQKMLDTPGPYLLDVIVPHQEHVLPMIPSGGAFKDVITEGDGRTQY

SEQ ID NO: 15 - DNA, 1938 bp, Glycine max
  CDS (1)..(1938)
atggcggctataactgccccaaaagctgcgttcagcgtcctcccttcatcatcccattccccgaatccctttgtgcgtttcgcaatcccatacagcccccatcactcccagcgccgttctctcagaatctccagcgccctatccgatgcaaccacgaaatcctccaccgccgccgctgaagctttcgcctcccgattcggcctcgatgagccccgcaagggcgccgacatcctcgtggaggcgctggagcggcagggcgtgacggacgtcttcgcctaccccggaggcgcctccatggagatccaccaggcgctcactcgctcatcctccatccgcaacgtcctccctcgccacgaacagggcggcgtcttcgccgccgagggctacgcccgctcttccggcctccccggcgtctgcatcgccacctccggccccggcgccaccaacctcgtctccggcctcgccgacgccttgcttgacagcgtccccctcgtcgccatcaccggccaggtcccccgccgcatgatcggcacagacgccttccaagaaacccccatcgtcgaggtaacacgttccatcactaagcataactatctcgttctcgatgttgatgacattcctagaatcgttaatgaagcgtttttcttagccacttcgggtagacctggccctgtgttaattgatatacctaaagatattcagcaacagcttgcgattcctaattgggatcaaccaattaggttacctggttacacgtctaggttgcctaagtctcctaatgagaaacatttggagcttattgtgaggttggttatggaatctaagaaaccggttttgtatgttggtggtggttgtttgaattctagtgaggaattgcggcgttttgtcgagctcaccggtgttcctgttgctagcactttgatgggtcttggtgcttatcccattgctgatgataattctcttcagatgcttgggatgcatgggactgtttatgctaattatgctgtggatagggctgatcttttgcttgcgtttggggtgaggtttgatgaccgtgtcaccgggaagcttgaggcttttgcgagccgggctaagattgttcacattgatattgattcagctgagattgggaagaacaagcagccgcatgtgtcggtttgtgcggatttgaagttggcattgaaggggattaatcgcgtgttagagagcagaggggttgcagggaagcttgattttagaggttggagggaagaactgaatgagcagaagcgaaggtttcctttgagttataagacttttgagaaagagatttctcctcagtatgctatacaggttcttgacgagttaaccaatggggaagctattgtgagtaccggagttggacagcaccagatgtgggctgctcagttttacaagtacaagaggcctaggcagtggttaacatccggtggtcttggtgctatggggtttggattgccggctgccattggtgcggctgtggctaatcccggtgcggttgtggttgacattgatggtgatgggagttttatgatgaatgttcaggagctggccaccatcaaggtggagaagctccctgttaagatattgttgttgaataatcaacacttgggtatggttgttcagtgggaggatcggttctataagtctaacagggctcacacatatctgggagacccctctaatgagaatgccatataccccaatatgttgaagtttgctgatgcttgtgggataccggcagctcgtgtgaccaagaaagaagaccttagagcggcaattcagaaaatgctggagacccctggcccctatcttcttgatgtcattgtaccccatcaagagcatgtcttgcccatgattcctagcaatggaaccttccaggacgtgataactgagggtgatggcaggacaagttactga

SEQ ID NO: 16 - protein, 645 aa, Glycine max
MAAITAPKAAFSVLPSSSHSPNPFVRFAIPYSPHHSQRRSLRISSALSDATTKSSTAAAEAFASRFGLDEPRKGADILVEALERQGVTDVFAYPGGASMEIHQALTRSSSIRNVLPRHEQGGVFAAEGYARSSGLPGVCIATSGPGATNLVSGLADALLDSVPLVAITGQVPRRMIGTDAFQETPIVEVTRSITKHNYLVLDVDDIPRIVNEAFFLATSGRPGPVLIDIPKDIQQQLAIPNWDQPIRLPGYTSRLPKSPNEKHLELIVRLVMESKKPVLYVGGGCLNSSEELRRFVELTGVPVASTLMGLGAYPIADDNSLQMLGMHGTVYANYAVDRADLLLAFGVRFDDRVTGKLEAFASRAKIVHIDIDSAEIGKNKQPHVSVCADLKLALKGINRVLESRGVAGKLDFRGWREELNEQKRRFPLSYKTFEKEISPQYAIQVLDELTNGEAIVSTGVGQHQMWAAQFYKYKRPRQWLTSGGLGAMGFGLPAAIGAAVANPGAVVVDIDGDGSFMMNVQELATIKVEKLPVKILLLNNQHLGMVVQWEDRFYKSNRAHTYLGDPSNENAIYPNMLKFADACGIPAARVTKKEDLRAAIQKMLETPGPYLLDVIVPHQEHVLPMIPSNGTFQDVITEGDGRTSY

SEQ ID NO: 17 - DNA, 1962 bp, Glycine max
  CDS (1)..(1962)
atggcggcaaccactgccccaaaacctgcgttcaccgccctcccttcttcatcatcatcatcttcccagaagccctttctgcgtctcgccctccaatttccctccctcccaaacagttcctatcactcccaacgcccttctctcaaaatctccagcgccctctccgatgcaaccgcgaaaaccaccaccgccgccgccgccgaagattttgtctcccgattcggcctggaggagccccgcaagggcgccgacatcctcgtggaggcgctggagcggcagggcgtgacggacgtgttcgcctaccccggtggtgcttccatggagatccaccaggcactcacccgctccgcctccatccgcaacgtcctcccccgccacgagcagggtggcgtcttcgccgccgagggctacgcccgttcctccggcatccccggcgtctgcatcgccacctccggccccggcgccaccaacctcgtcagcggcctcgccgacgccatgctcgacagcgtccccctcgtcgccatcaccggccaggtcccccgccgcatgatcggcaccgacgccttccaagaaacccccatcgtcgaggtaacgcgttccgtcacaaagcataactatctcgttctagatgttgatgatattcctagaatcgttaatgaagcgtttttcttagccacttcgggtagacctggccctgtgttaattgatatacccaaagatattcagcaacagtttgcgattcctaattgggatcaaccaattaggttacctggttacatgtctaggttgcctaagtctcccaatgagaatcatttggagcttattgtgaggttggttatggaatctaagaaacctgttttgtatgttggtggtggttgtttgaattctagtgaagaattgcggcgttttgtcgagctcactggtgttcctgttgctagtactttgatgggtcttggtgcttatcccattgctgatgagaattctcttcagatgcttgggatgcacgggactgtttatgctaattatgctgtggacaaggctgatattttgcttgcgtttggggtgaggtttgatgaccgtgtcacggggaagctcgaggcttttgcgagccgggctaagatagttcacattgatattgattcggcagagattggaaagaacaagcagccgcatgtttcggtttgcgctgatttgaagctggcgttgaaggggattaatcacatgttggagagcagaggggttggggggaagcttgattttagaggttggagggaagagctgaatgagcagaagcgaaggtttcctttgagctataagacttttgaggatgagatttctcctcagtatgctatacaggttcttgatgagttgactaatggggatgctattgtgagtactggagttggacagcaccagatgtgggctgctcagttttacaagtacaagaggcctaggcagtggttaacatccggtggtcttggtgctatggggtttggattgccggctgccatcggtgcggctgtggctaatccgggtgcggttgtggttgacattgatggtgatgggagttttataatgaatgttcaggagctggccactatcaaggtagagaagctcccggttaagatattgttgttgaataatcaacacttgggtatggttgttcagtgggaggatcggttctataagtctaacagggctcacacgtatctgggagacccctctaatgagaatgccatatttcctgatatgttgaagtttgctgatgcttgcgggataccggcagctcgtgtgaccaagaaagaagaccttagagcggcaattcagaaaatgttggacacccctggcccctatcttcttgatgtcattgtaccccatcaagagcatgtcttgcccatgattcctagcaatggaaccttccaggacgtgataactgagggtgatggcaggacaagttactga

SEQ ID NO: 18 - protein, 653 aa, Glycine max
MAATTAPKPAFTALPSSSSSSSQKPFLRLALQFPSLPNSSYHSQRPSLKISSALSDATAKTTTAAAAEDFVSRFGLEEPRKGADILVEALERQGVTDVFAYPGGASMEIHQALTRSASIRNVLPRHEQGGVFAAEGYARSSGIPGVCIATSGPGATNLVSGLADAMLDSVPLVAITGQVPRRMIGTDAFQETPIVEVTRSVTKHNYLVLDVDDIPRIVNEAFFLATSGRPGPVLIDIPKDIQQQFAIPNWDQPIRLPGYMSRLPKSPNENHLELIVRLVMESKKPVLYVGGGCLNSSEELRRFVELTGVPVASTLMGLGAYPIADENSLQMLGMHGTVYANYAVDKADILLAFGVRFDDRVTGKLEAFASRAKIVHIDIDSAEIGKNKQPHVSVCADLKLALKGINHMLESRGVGGKLDFRGWREELNEQKRRFPLSYKTFEDEISPQYAIQVLDELTNGDAIVSTGVGQHQMWAAQFYKYKRPRQWLTSGGLGAMGFGLPAAIGAAVANPGAVVVDIDGDGSFIMNVQELATIKVEKLPVKILLLNNQHLGMVVQWEDRFYKSNRAHTYLGDPSNENAIFPDMLKFADACGIPAARVTKKEDLRAAIQKMLDTPGPYLLDVIVPHQEHVLPMIPSNGTFQDVITEGDGRTSY

SEQ ID NO: 19 - DNA, 1971 bp, Glycine max
  CDS (1)..(1971)
atggcggccacagcttccagaaccaccccattctcttctccctcttcctcttcacaccccaccttccccaaacgcattgctagatccaccctccctctctctcatcaaaccttcattaaacccaaccacgcgctcaaaatcaaatgttccatctccaaacccccctcgccgccggccaccaccaccactgcagcgccggcaacatcagagcccttcgtgtcgcggttcgcctccgctgaaccgcgcaagggctcggacatccttgtggaggcccttgagcggctgggcgtgactaatgtcttcgcctaccccggcggcgcgtcgatggagatccaccaggctctcacgcgctcctccaccatccgcaacgtcctccctcgccacgagcagggcggcatcttcgccgccgagggctacgcgcgctcctctggcctccccggcgtctgcatcgccacctccggccccggcgccaccaacctcgtcagcggcctcgccgacgccttaatggacagcgtccccgtcgtcgccatcaccggccaggtcccccggcgcatgattggcaccgacgccttccaagaaacccccatcgtcgaagtcaccagatccatcaccaagcacaactacctcatcctcgacgtcgacgacattccccgcatcgtcgccgaagccttcttcgtcgccacctccggccgccccggcccggtcctcatcgacatccccaaagacgtccaacagcaactcgccgtgcctaattgggacgagcccatcaacctccccgggtacctcgccaggcttcccaggccccccaccgaggcccaattagagcacattgtcagactcataaccgaggcccaaaagcccgtcctctacgtcggaggtggcagtttgaattccagtgatgaattgagacgctttgtggaactgactggtattcccgttgctagcactttaatgggccttggaacttatcctattggggatgaatattcccttcaaatgttgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgctgagattggaaagaataagcaggcgcatgtgtcggtttgcgcggatttgaagctggccttgcaggggattaatatgattttggagaaaaaaagagtggggggtggtaagcttgatcttggaggctggagggaagagattaatgtgcagaaacacaagtttccattgggttacaaaacattccaggacgccatttccccgcagcatgctattgaggttcttgatgagttgacaaatggggatgctattgttagtactggggttgggcagcatcaaatgtgggctgcacagttttacaagtacaagagaccgaggcagtggttgacctcggggggtcttggagccatgggttttggattgcctgcggctattggcgctgctgttgctaaccccggcgctgttgtggttgacattgatggagatggtagtttcatcatgaatgttcaagaattggcaactataagagtggagaatcttcctgttaagatattgttgttgaataatcagcatttgggcatggtggttcagtgggaggatcggttctacaagtccaatagagctcacacctatcttggagacccgtctagcgagaatgagatattcccaaacatgctcaagtttgctgacgcttgtgggataccggcagcgcgtgtgacgaagaaggaagagcttagagcagaaattcagaaaatgttggacacccctggcccctaccttcttgatgtcattgtgccacatcaggagcatgtgttgccgatgattcccagtaatggatccttcaaggatgtgataacggagggagacggtagaacgaggtactga

SEQ ID NO: 20 - protein, 656 aa, Glycine max
MAATASRTTPFSSPSSSSHPTFPKRIARSTLPLSHQTFIKPNHALKIKCSISKPPSPPATTTTAAPATSEPFVSRFASAEPRKGSDILVEALERLGVTNVFAYPGGASMEIHQALTRSSTIRNVLPRHEQGGIFAAEGYARSSGLPGVCIATSGPGATNLVSGLADALMDSVPVVAITGQVPRRMIGTDAFQETPIVEVTRSITKHNYLILDVDDIPRIVAEAFFVATSGRPGPVLIDIPKDVQQQLAVPNWDEPINLPGYLARLPRPPTEAQLEHIVRLITEAQKPVLYVGGGSLNSSDELRRFVELTGIPVASTLMGLGTYPIGDEYSLQMLGMHGTVYANYAVDNSDLLLAFGVRFDDRVTGKLEAFASRAKIVHIDIDSAEIGKNKQAHVSVCADLKLALQGINMILEKKRVGGGKLDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQWEDRFYKSNRAHTYLGDPSSENEIFPNMLKFADACGIPAARVTKKEELRAEIQKMLDTPGPYLLDVIVPHQEHVLPMIPSNGSFKDVITEGDGRTRY

SEQ ID NO: 21 - DNA, 1902 bp, Glycine max
  CDS (1)..(1902)
atggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactga

SEQ ID NO: 22 - protein, 633 aa, Glycine max
MAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQWEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 23 - DNA, 1995 bp, Nicotiana tabacum
  CDS (1)..(1995)
atggcggcggctgcggcggctccatctccctctttctccaaaaccctatcgtcctcctcctccaaatcctccaccctcctccctagatccaccttccctttcccccaccacccccacaaaaccaccccaccacccctccacctcacccccacccacattcacagccaacgccgtcgtttcaccatctccaatgtcatttccactacccaaaaagtttccgagacccaaaaagccgaaactttcgtttcccgttttgcccctgacgaacccagaaagggttccgacgttctcgtggaggccctcgaaagagaaggggttacggacgtttttgcgtacccaggcggcgcttccatggagattcaccaagctttgacgcgctcaagcatcatccgcaacgtgctaccacgtcacgagcagggtggtgtcttcgccgctgagggttacgcacgcgccaccggcttccccggcgtttgcattgccacctccggccctggcgccaccaatctcgtcagtggcctcgcggacgccctactggatagcgtccccattgttgctataaccggtcaagtgccacgtaggatgatcggtactgatgcttttcaggaaactccgattgttgaggtaactagatcgattaccaagcataattatctcgttatggacgtagaggatattcctagggttgtacgtgaggcttttttccttgcgagatcgggccggcctggccctgttttgattgatgtacctaaggatattcagcaacaattggtgatacctgactgggatcagccaatgaggttgcctggttacatgtctaggttacctaaattgcccaatgagatgcttttagaacaaattgttaggcttatttctgagtcaaagaagcctgttttgtatgtggggggtgggtgttcgcaatcgagtgaggagttgagacgattcgtggagctcaccggtatccccgtggcaagtactttgatgggtcttggagcttttccaactggggatgagctttccctttcaatgttgggtatgcatggtactgtttatgctaattatgctgtggacagtagtgatttattgctcgcatttggggtgaggtttgatgatagagttactggaaagttagaagcttttgctagccgagcgaaaattgttcacattgatattgattcagctgagattggaaagaacaagcagcctcatgtttccatttgtgcggatatcaagttggcgttacagggtttgaattcgatattggagagtaaggaaggtaaactgaagttggatttttctgcttggaggcaggagttgacggtgcagaaagtgaagtacccgttgaattttaaaacttttggtgatgctattcctccgcaatatgctatccaggttctagatgagttaactaatgggagtgctattataagtaccggtgttgggcagcaccagatgtgggctgctcaatattataagtacagaaagccacgccaatggttgacatctggtggattaggagcgatgggatttggtttgcccgctgctattggtgcggctgttggaagacctgatgaagttgtggttgacattgatggtgatggcagtttcatcatgaatgtgcaggagctagcaactattaaggtggagaatctcccagttaagattatgttactgaataatcaacacttgggaatggtggttcaatgggaggatcggttctataaggctaacagagcacacacatacctggggaatccttctaatgaggcggagatctttcctaatatgttgaaatttgcagaggcttgtggcgtacctgctgcgagagtgacacacagggatgatcttagagcggctattcaaaagatgttagacactcctgggccatacttgttggatgtgattgtacctcatcaggaacatgttctacctatgattcccagtggcggggctttcaaagatgtgatcacagagggtgacgggagaagttcctattga

SEQ ID NO: 24 - protein, 664 aa, Nicotiana tabacum
MAAAAAAPSPSFSKTLSSSSSKSSTLLPRSTFPFPHHPHKTTPPPLHLTPTHIHSQRRRFTISNVISTTQKVSETQKAETFVSRFAPDEPRKGSDVLVEALEREGVTDVFAYPGGASMEIHQALTRSSIIRNVLPRHEQGGVFAAEGYARATGFPGVCIATSGPGATNLVSGLADALLDSVPIVAITGQVPRRMIGTDAFQETPIVEVTRSITKHNYLVMDVEDIPRVVREAFFLARSGRPGPVLIDVPKDIQQQLVIPDWDQPMRLPGYMSRLPKLPNEMLLEQIVRLISESKKPVLYVGGGCSQSSEELRRFVELTGIPVASTLMGLGAFPTGDELSLSMLGMHGTVYANYAVDSSDLLLAFGVRFDDRVTGKLEAFASRAKIVHIDIDSAEIGKNKQPHVSICADIKLALQGLNSILESKEGKLKLDFSAWRQELTVQKVKYPLNFKTFGDAIPPQYAIQVLDELTNGSAIISTGVGQHQMWAAQYYKYRKPRQWLTSGGLGAMGFGLPAAIGAAVGRPDEVVVDIDGDGSFIMNVQELATIKVENLPVKIMLLNNQHLGMVVQWEDRFYKANRAHTYLGNPSNEAEIFPNMLKFAEACGVPAARVTHRDDLRAAIQKMLDTPGPYLLDVIVPHQEHVLPMIPSGGAFKDVITEGDGRSSY

SEQ ID NO: 25 - DNA, 2004 bp, Nicotiana tabacum
  CDS (1)..(2004)
atggcggcggcggctccatctccctcttcttccgctttctccaaaaccctatcgccttcctcctccacatcctccaccctcctccctagatcaaccttccctttcccccaccacccccacaagaccaccccaccacccctccacctcacccacactcacattcacattcacagccaacgccgtcgtttcaccatatccaatgtcatttccactaaccaaaaagtttcccagaccgaaaaaaccgaaactttcgtttcccgttttgctcctgacgaacccagaaagggttccgacgttctcgtggaggctctcgaaagagaaggggttacggacgtctttgcgtacccaggtggcgcttccatggagattcaccaagctttgacccgttcaagcatcatccgcaacgtgctgccacgtcacgagcagggcggtgtcttcgccgctgagggttacgcacgcgccaccggatttcccggcgtttgcattgccacctctggccccggcgccaccaatctcgtcagcggcctcgctgacgcgctactggatagcgtccccattgttgctataacaggtcaagtgccacgtaggatgataggtactgatgcttttcaggaaactcctattgttgaggtaactagatcgattaccaagcataattatctcgttatggacgtagaggatattcctagggttgtacgtgaagcttttttcctcgcgagatcgggccggcctggccctattttgattgatgtacctaaggatattcagcaacaattggtgatacctgactgggatcagccaatgaggttacctggttacatgtctaggttacctaaattgcccaatgagatgcttttagaacaaattgttaggcttatttctgagtcaaagaagcctgttttgtatgtggggggtgggtgttcgcaatcgagtgaggacttgagacgattcgtggagctcacgggtatccccgtggcaagtactttgatgggtcttggagcttttccaactggggatgagctttccctttcaatgttgggtatgcatggtactgtttatgctaattatgctgtggacagtagtgatttgttgctcgcatttggggtgaggtttgatgatagagttactggaaagttagaagcttttgctagccgagcaaaaattgttcacattgatattgattcagctgagattggaaagaacaagcagcctcatgtttccatttgtgcagatatcaagttggcgttacagggtttgaattcgatactggagagtaaggaaggtaaactgaagttggatttttctgcttggaggcaggagttgacggagcagaaagtgaagcacccattgaactttaaaacttttggtgatgcaattcctccgcaatatgctatccaggttctagatgagttaactaatgggaatgctattataagtactggtgtggggcaacaccagatgtgggctgctcaatactataagtacagaaagccacgccaatggttgacatctggtggattaggagcaatgggatttggtttgcccgctgctattggtgcggctgttggaagaccggatgaagttgtggttgacattgatggtgatggcagtttcatcatgaatgtgcaggagcttgcaacaattaaggtggagaatctcccagttaagattatgttactgaataatcaacacttgggaatggtggttcaatgggaggatcggttctataaggctaacagagcacacacatacctggggaatccttctaatgaggcggagatctttcctaatatgctgaaatttgcagaggcttgtggcgtacctgctgcaagagtgacacatagggatgatcttagagctgccattcagaagatgttagacactcctgggccatacttgttggatgtgattgtacctcatcaggaacatgttttacctatgattcccagtggcggagctttcaaagatgtgatcacagagggtgacgggagaagttcctattga

SEQ ID NO: 26 - protein, 667 aa, Nicotiana tabacum
MAAAAPSPSSSAFSKTLSPSSSTSSTLLPRSTFPFPHHPHKTTPPPLHLTHTHIHIHSQRRRFTISNVISTNQKVSQTEKTETFVSRFAPDEPRKGSDVLVEALEREGVTDVFAYPGGASMEIHQALTRSSIIRNVLPRHEQGGVFAAEGYARATGFPGVCIATSGPGATNLVSGLADALLDSVPIVAITGQVPRRMIGTDAFQETPIVEVTRSITKHNYLVMDVEDIPRVVREAFFLARSGRPGPILIDVPKDIQQQLVIPDWDQPMRLPGYMSRLPKLPNEMLLEQIVRLISESKKPVLYVGGGCSQSSEDLRRFVELTGIPVASTLMGLGAFPTGDELSLSMLGMHGTVYANYAVDSSDLLLAFGVRFDDRVTGKLEAFASRAKIVHIDIDSAEIGKNKQPHVSICADIKLALQGLNSILESKEGKLKLDFSAWRQELTEQKVKHPLNFKTFGDAIPPQYAIQVLDELTNGNAIISTGVGQHQMWAAQYYKYRKPRQWLTSGGLGAMGFGLPAAIGAAVGRPDEVVVDIDGDGSFIMNVQELATIKVENLPVKIMLLNNQHLGMVVQWEDRFYKANRAHTYLGNPSNEAEIFPNMLKFAEACGVPAARVTHRDDLRAAIQKMLDTPGPYLLDVIVPHQEHVLPMIPSGGAFKDVITEGDGRSSY

SEQ ID NO: 27 - DNA, 1980 bp, Solanum tuberosum
  CDS (1)..(1980)
atggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgccgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctaggttacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaatgggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagtggcggtgctttcaaagatgtgatcacagagggtgatgggagacgttcatattga

SEQ ID NO: 28 - protein, 659 aa, Solanum tuberosum
MAAAASPSPCFSKTLPPSSSKSSTILPRSTFPFHNHPQKASPLHLTHTHHHRRGFAVSNVVISTTTHNDVSEPETFVSRFAPDEPRKGCDVLVEALEREGVTDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYMSRLPKLPNEMLLEQIIRLISESKKPVLYVGGGCLQSSEELRRFVELTGIPVASTLMGLGAFPTGDELSLQMLGMHGTVYANYAVDGSDLLLAFGVRFDDRVTGKLEAFASRAKIVHIDIDSAEIGKNKQPHVSICADIKLALQGLNSILEGKEGKLKLDFSAWRQELTEQKVKYPLSFKTFGEAIPPQYAIQVLDELTNGNAIISTGVGQHQMWAAQYYKYKKPHQWLTSGGLGAMGFGLPAAIGAAVGRPGEIVVDIDGDGSFIMNVQELATIKVENLPVKIMLLNNQHLGMVVQWEDRFYKANRAHTYLGDPANEEEIFPNMLKFAEACGVPAARVSHRDDLRAAIQKMLDTPGPYLLDVIVPHQEHVLPMIPSGGAFKDVITEGDGRRSY

SEQ ID NO: 29 - DNA, 1974 bp, Solanum tuberosum
  CDS (1)..(1974)
atggcggctgcatctccatctccttgtttttccaaaaacctacctccatcttcatcaaaatcttccatccttcttcccaaatctacctttactttccacaatcaccccaaaaatacctcaccccttcaccttacccacacccaacatcatagccgtttcactgtctcaaatgtcatcctatcaaccacgacccataacgacgtttctgaacccgaaatcttcgtttcacgtttcgcccctgacgaacccagaaagggttgtgatgttcttgtggaggcacttgaaagggaaggggttaaggatgtatttgcatacccaggaggtgcttccatggagattcatcaggctttgacacgttccaatattattcgtaatgtgctgccacgtcatgaacagggtggtgtgtttgctgcagagggttacgcacgggccactgggttccctggtgtttgcattgctacatctggtccgggagctacgaatcttgttagcggtcttgcggatgctttgttggatagtattccgattgttgctattacgggtcaagtgccgaggaggatgattggtactgatgcgtttcaggaaactcctattgttgaggtaacgagatccattacgaagcataattatcttgttatggatgtagaggatattcctagggttgttcgtgaagcgttttttctagcgaaatcgggacggcctggaccggttctgattgatgttcctaaggatattcagcaacaattggtgatacctaattgggatcagccaatgaggttgcctggttacatctctaggttgcctaaattgcctaatgagatgcttttggaacaaattgttaggctgatttcggagtcgaagaagcctgttttgtatgtgggtggtgggtgtacacaatcgagtgaggagctgagacgatttgtggagcttacgggtattcctgtggcgagtactttgatgggtcttggaacttttccatgtggggatgagctttctcttcaaatgttgggtatgcatgggactgtgtatgctaattatgcggtggatagtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggaggcaggagttaacggagcagaaggtgaagtacccattgaattataagacttttggtgaagccatccctccacaatatgctattcaggttcttgatgagttaactaacggaaatgccattattagtactggtgtggggcaacaccaaatgtgggctgcccaatactataagtacaaaaagccacgccaatggttgacatctggtggattaggagcaatgggatttggtttgcctgctgctataggtgcggctgttggaagaccgggtgagattgtggttgacattgatggtgacgggagttttatcatgaatgtgcaggagttagcaacaattaaggtggagaatctcccagttaagattatgttactgaataatcaacacttgggaatggtggttcagtgggaggatcgattctataaggctaacagagcacacacttacttgggtaatcctgctaatgaggaagaaatcttccctaatatgctgaaatttgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccagtggtggcgctttcaaagatgtgattacggagggtgatgggagacgttcctattga

SEQ ID NO: 30 - protein, 657 aa, Solanum tuberosum
MAAASPSPCFSKNLPPSSSKSSILLPKSTFTFHNHPKNTSPLHLTHTQHHSRFTVSNVILSTTTHNDVSEPEIFVSRFAPDEPRKGCDVLVEALEREGVKDVFAYPGGASMEIHQALTRSNIIRNVLPRHEQGGVFAAEGYARATGFPGVCIATSGPGATNLVSGLADALLDSIPIVAITGQVPRRMIGTDAFQETPIVEVTRSITKHNYLVMDVEDIPRVVREAFFLAKSGRPGPVLIDVPKDIQQQLVIPNWDQPMRLPGYISRLPKLPNEMLLEQIVRLISESKKPVLYVGGGCTQSSEELRRFVELTGIPVASTLMGLGTFPCGDELSLQMLGMHGTVYANYAVDSSDLLLAFGVRFDDRVTGKLEAFASRAKIVHIDIDSAEIGKNKQPHVSICADIKLALQGLNSILEGKEGKLKLDFSAWRQELTEQKVKYPLNYKTFGEAIPPQYAIQVLDELTNGNAIISTGVGQHQMWAAQYYKYKKPRQWLTSGGLGAMGFGLPAAIGAAVGRPGEIVVDIDGDGSFIMNVQELATIKVENLPVKIMLLNNQHLGMVVQWEDRFYKANRAHTYLGNPANEEEIFPNMLKFAEACGVPAARVSHRDDLRAAIQKMLDTPGPYLLDVIVPHQEHVLPMIPSGGAFKDVITEGDGRRSY

SEQ ID NO: 31 - DNA, 1971 bp, Solanum tuberosum
  CDS (1)..(1971)
atggcggcatcttctccaaatccttcctcttccactttcctcaacaatccttttacatcatcttcttattattcctctaagcttctttccagaaccttctttccttttccacacactccaacaattattcctcataaatctcttaccatcaccatcgacgtttccacaaccccaaatggtgctccagtaccattaatggagacgacattcacttctcgattcctccccgatgaaccccgaaaaggatgtgacatcttggttgaagcccttgagcgtgaaggcgttaacaatgtatttgcttatccaggcggtactactctggaaattcaccaggctcttacccgttctccaaccataaggaatatattgccacgtcatgaacagggtggcgttttcgcggctgaaggttatgcacgcgccaccgggttccctggtgtttgtatggctacctctggccctggagctactaatcttattagtggtcttgctgatgctatggctgatagtattccaattgttgctattacaggtcaagttcctcgtagaatgattggaacagacgcatttcaagaaattccaattattgaggtaactagatcaattaccaagcataattatctagtcatggacgttcacgatattcccaggattgtccgagaggcgtttttcctagctctatcaggacgacctggaccggttttaattgatgttcctaaagatgttcaacaacaaatggatattcctaattgggatcagcccatgaagttgccaggttatatgtctagactcccgttaccaccaaaaaagacgctattggaacaaattgttagattattttccgagtcaaagaagcctgttttgtatgttggtggagggtgcgtacaatccagcaatgagttaagacgttttgttcagctcacaggtattcctgtggcgagtactttgatgggactaggagcgtttccaacaggggatgagctttcgcttcaaatgctagggatgcatggtactgtttattcaaattacgcggtggatagtagtgatttgcttctagcatttggagtgaggtttgatgatcgtgtcacgggtaaattagaaacatttgcaagcagagcaaaaattgttcacattgatatcgactccaaagagattgggaaaaacaagaagcctcatgtatctatttgtactgatattaagctggcgttacaggggttgaatttgatattaatggagagggaaaatacaagaaacgcgttgaagctgaatttctcgccttggaggaaagagttaacagagcaaaaattgaagtatcctttgaaatacaagttttatggtggatctattcctccacaatatgcaattgaagtccttgatgagttaactaatggaaatgctattataacaagtggtgtagggcaacaccaaatgtggtctgctcaatactataagtacaaaaatccaatgcagtgtttgacatctggtggattcggagcgatggggtttggattacctgctgcaataggagcagcaatagcaagaccagatgcaattgttgtggacatcgatggggatggtagtttcatgatgaatgtgcaagagttagcaacagttagagtcgagaatcttcctgttaagatgatgattttaaataatcaacatttgggaatggcgacacagtgggaggataggttctataaggcgaatagagcagattcttatctcggaaatccttctaacaaggcacggattttccctaacatgttgaaattcgcggaggcttgtgatatacctgctgcccgagtgattaataataatgatgttagagatgctatacgaaagatgttagatactcctgggccttacttgttggatgtgattgtgtctcatcaggagcatgttttgcctatgattcccagtgatggggcttttaaggatgttatcacggagggcgatgggagatgttcctactga

SEQ ID NO: 32 - protein, 656 aa, Solanum tuberosum
MAASSPNPSSSTFLNNPFTSSSYYSSKLLSRTFFPFPHTPTIIPHKSLTITIDVSTTPNGAPVPLMETTFTSRFLPDEPRKGCDILVEALEREGVNNVFAYPGGTTLEIHQALTRSPTIRNILPRHEQGGVFAAEGYARATGFPGVCMATSGPGATNLISGLADAMADSIPIVAITGQVPRRMIGTDAFQEIPIIEVTRSITKHNYLVMDVHDIPRIVREAFFLALSGRPGPVLIDVPKDVQQQMDIPNWDQPMKLPGYMSRLPLPPKKTLLEQIVRLFSESKKPVLYVGGGCVQSSNELRRFVQLTGIPVASTLMGLGAFPTGDELSLQMLGMHGTVYSNYAVDSSDLLLAFGVRFDDRVTGKLETFASRAKIVHIDIDSKEIGKNKKPHVSICTDIKLALQGLNLILMERENTRNALKLNFSPWRKELTEQKLKYPLKYKFYGGSIPPQYAIEVLDELTNGNAIITSGVGQHQMWSAQYYKYKNPMQCLTSGGFGAMGFGLPAAIGAAIARPDAIVVDIDGDGSFMMNVQELATVRVENLPVKMMILNNQHLGMATQWEDRFYKANRADSYLGNPSNKARIFPNMLKFAEACDIPAARVINNNDVRDAIRKMLDTPGPYLLDVIVSHQEHVLPMIPSDGAFKDVITEGDGRCSY

SEQ ID NO: 33 - DNA, 1797 bp, Triticum aestivum
gccgcgtcccccgccgccacctccgtcgcgcctcccgccaccgcgctccggccgtggggcccctccgagccccgcaagggcgccgacatcctcgtcgaggcgctggagcgctgcggcatcgtcgacgtcttcgcctaccctggcggcgcgtccatggagatccaccaggcgctgacgcgctcgccagtcatcaccaaccacctcttccgccacgagcagggggaggcgttcgcggcgtccgggtacgcccgcgcgtccggccgcgtcggcgtctgcgtcgccacctccggcccgggggccaccaacctcgtctccgcgctcgccgacgctctcctcgactccatccccatggtcgccatcacgggccaggtcccccgccgcatgatcggcacggatgcgttccaggagacgcccatcgtggaggtcacgcgctccatcaccaagcacaactacctggtccttgacgtggaggatatcccccgcgtcatccaggaagccttcttcctcgcatcctctggccgcccggggccggtgctggttgatatccccaaggacatccagcagcagatggctgtgcctgtctgggacacgccgatgagtttgccagggtacatcgcccgcctgcccaagccaccatctactgaatcgcttgagcaggtcctgcgtctggttggcgagtcacggcgcccaattctgtatgttggtggtggctgcgctgcatctggtgaggagttgcgccgctttgttgagctcactgggattccagttacaactactcttatgggccttggcaacttccccagtgacgacccactgtctctgcgcatgctggggatgcatggcactgtgtatgcaaattatgcagtagataaggctgacctgttgcttgcatttggtgtgcggtttgatgatcgtgtgaccgggaaaatcgaggcttttgcaagcaggtccaagattgtgcacattgacattgacccagctgagattggcaagaacaagcagccacatgtctccatttgtgcagatgttaagcttgctttacaggggttgaatgctctattaaatgggagcaaagcacaacagggtctggattttggtccatggcacaaggagttggatcagcagaagagggagtttcctctaggattcaagacttttggtgaggccatcccgccgcaatatgctatccaggtactggatgagctgacaaaaggggaggcgatcattgccaccggtgttgggcagcatcagatgtgggcggctcagtattacacttacaagcggccacggcagtggctgtcttcatccggtttgggtgcaatgggatttgggttgccagctgcagctggcgctgctgtggccaacccaggtgttacagttgttgacattgatggggatggtagtttcctcatgaacattcaggagttggcgttgatccgtattgagaacctcccagtgaaggtgatgatattgaacaaccagcatctgggaatggtggtgcagtgggaggataggttttacaaggccaaccgggcgcacacataccttggcaacccagaaaatgagagtgagatatatccagattttgtgacgattgctaaaggattcaacgttccggcagttcgtgtgacgaagaagagcgaagtcactgcagcaatcaagaagatgcttgagaccccagggccatacttgttggatatcattgtcccgcatcaggagcacgtgctgcctatgatcccaagcggtggtgcttttaaggacatgatcatggagggtgatggcaggacctcgtactga

SEQ ID NO: 34 - protein, 598 aa, Triticum aestivum
AASPAATSVAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNALLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 35 - DNA, 1797 bp, Triticum aestivum
gccgcgtcccccgccgccacctccgccgcgcctcccgcaaccgcgctccggccctggggcccgtccgagccccgcaagggcgccgacatcctcgtcgaggcgctcgagcgctgcggcatcgtcgacgtcttcgcctaccccggcggcgcctccatggagatccaccaggcgctgacgcgctcgcccgtcatcaccaaccacctcttccgccacgagcagggggaggcgttcgcggcgtccggctacgcccgcgcgtccggccgcgtcggcgtctgcgtcgccacctccggcccgggggccaccaacctcgtctccgcgctcgccgacgccctcctcgactccatccccatggtcgccatcacgggccaggtcccccgccgcatgatcggcacggacgcgttccaggagacgcccatagtggaggtcacgcgctccatcaccaagcacaactacctggtccttgacgtggaggatatcccccgcgtcatccaggaagccttcttccttgcatcctctggccgcccggggccggtgctagttgatatccccaaggacatccagcagcagatggctgtgcccgtctgggacactccaatgagtttgccagggtacatcgcccgcctgcccaagccaccatctactgaatcgcttgagcaggtcctgcgtctggttggcgagtcacggcgcccaattctgtatgttggtggtggctgcgctgcgtctggcgaggagttgcgccgctttgttgagcttactgggattccagttacaactactctgatgggccttggcaacttccccagcgacgacccactgtctctgcgcatgcttgggatgcatggcactgtgtatgcaaattatgcagtagataaggctgacctgttgctcgcatttggtgtgcggtttgatgatcgtgtgactgggaaaatcgaggcttttgcaagcaggtccaagattgtgcacattgacattgacccagctgagattggcaagaacaagcagccacatgtctccatttgtgcagatgttaagcttgctttacaggggttgaatgatctattaaatgggagcaaagcacaacagggtctggattttggtccatggcacaaggagttggatcagcagaagagggagtttcctctaggattcaagacttttggcgaggccatcccgccgcaatatgctatccaggtactggatgagctgacaaaaggggaggcgatcattgccactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggtgatggtagtttcctcatgaacattcaggagttggcgttgatccgcattgagaacctcccagtgaaggtgatgatattgaacaaccagcatctgggaatggtggtgcagtgggaggataggttttacaaggccaatcgggcgcacacataccttggcaacccagaaaatgagagtgagatatatccagattttgtgacgattgctaaaggattcaacgttccagcagttcgagtgacgaagaagagcgaagtcactgcagcaatcaagaagatgcttgagaccccagggccatacttgttggatatcatagtcccgcatcaggagcacgtgctgcctatgatcccaagcggtggtgctttcaaggacatgatcatggagggtgatggcaggacctcgtactga

SEQ ID NO: 36 - protein, 598 aa, Triticum aestivum
AASPAATSAAPPATALRPWGPSEPRKGADILVEALERCGIVDVFAYPGGASMEIHQALTRSPVITNHLFRHEQGEAFAASGYARASGRVGVCVATSGPGATNLVSALADALLDSIPMVAITGQVPRRMIGTDAFQETPIVEVTRSITKHNYLVLDVEDIPRVIQEAFFLASSGRPGPVLVDIPKDIQQQMAVPVWDTPMSLPGYIARLPKPPSTESLEQVLRLVGESRRPILYVGGGCAASGEELRRFVELTGIPVTTTLMGLGNFPSDDPLSLRMLGMHGTVYANYAVDKADLLLAFGVRFDDRVTGKIEAFASRSKIVHIDIDPAEIGKNKQPHVSICADVKLALQGLNDLLNGSKAQQGLDFGPWHKELDQQKREFPLGFKTFGEAIPPQYAIQVLDELTKGEAIIATGVGQHQMWAAQYYTYKRPRQWLSSSGLGAMGFGLPAAAGAAVANPGVTVVDIDGDGSFLMNIQELALIRIENLPVKVMILNNQHLGMVVQWEDRFYKANRAHTYLGNPENESEIYPDFVTIAKGFNVPAVRVTKKSEVTAAIKKMLETPGPYLLDIIVPHQEHVLPMIPSGGAFKDMIMEGDGRTSY

SEQ ID NO: 37 - DNA, 455 bp, Saccharum officinarum
ctggtctgggggcaatgggatttggtttgccggctgctgctggcgctgctgtggccaacccaggtgtcactgttgttgacatcgacggagatggtagcttcctcatgaacattcaggagctagctatgatccgaattgagaacctcccagtgaaggtctttgcgctaaacaaccagcacctggggatggtggtgcagtgggaggacaggttctataaggccaacagagcacacacatacttgggaaacccagagaatgaaagtgagatatatccagatttcgtgacaattgccaaagggttcaacattccagcagtccgtgtgacaaagaagagcgaagtccatgcagcaatcaagaagatgcttgagactccagggccgtacctcttggatataatcgtcccgcaccaggagcatgtgttgcctatgatcccgagtggtggagctttcaaggac

SEQ ID NO: 38 - protein, 151 aa, Saccharum officinarum
GLGAMGFGLPAAAGAAVANPGVTVVDIDGDGSFLMNIQELAMIRIENLPVKVFALNNQHLGMVVQWEDRFYKANRAHTYLGNPENESEIYPDFVTIAKGFNIPAVRVTKKSEVHAAIKKMLETPGPYLLDIIVPHQEHVLPMIPSGGAFKD

(19) United States
(12) Patent Application Publication
(10) Pub. No.: US 2019/0136254 A1
(43) Pub. Date: May 9, 2019

(54) ALS Inhibitor Herbicide Tolerant Mutant Plants

(71) Applicant: BASF SE, Ludwigshafen am Rhein (DE)

(72) Inventors: Rene Ruiter, Heusden (Destelbergen) (BE); Rudiger Hain, Frankfurt (DE); Gerhard Johann, Burscheid (DE); Bernd Laber, Idstein (DE)

(21) Appl. No.: 16/247,658

(22) Filed: Jan. 15, 2019

Related U.S. Application Data
(63) Continuation of application No. 14/913,020, filed on Feb. 19, 2016, now abandoned.

(30) Foreign Application Priority Data
Aug. 21, 2013 (EP) 13181128.3
Dec. 10, 2013 (EP) 13196378.7

(51) Int. Cl. C12N 15/82; A01N 37/30; C12N 9/10

(52) U.S. Cl. CPC C12N 15/8274; C12Y 202/01006; C12N 9/1022; A01N 37/30; C12N 15/8278

(57) ABSTRACT

The present invention relates to an ALS inhibitor herbicide tolerant polyploid plants, such as B. napus or B. juncea plants, progeny and parts thereof comprising mutations in all acetolactase genes.

### CROSS-REFERENCE TO PRIOR APPLICATIONS

This application is a continuation of U.S. patent application Ser. No. 14/913,020, filed Feb. 19, 2016, the entire contents of which is hereby incorporated herein by reference. U.S. patent application Ser. No. 14/913,020 is a National Stage Application of International Application No. PCT/EP2014/067716, filed Aug. 20, 2014, the entire contents of which is hereby incorporated herein by reference. This application also claims priority under 35 U.S.C. § 119 to European Application No. 13181128.3, filed Aug. 21, 2013, and European Application No. 13196378.7, filed Dec. 10, 2013, the entire contents of each of which are hereby incorporated herein by reference.

### REFERENCE TO SEQUENCE LISTING SUBMITTED ELECTRONICALLY

The official copy of the sequence listing is submitted electronically via EFS-Web as an ASCII formatted sequence listing with a file named “BCS13-2013-WO1.txt,” created on Aug. 19, 2013, and having a size of 257 kilobytes and is filed concurrently with the specification. The sequence listing contained in this ASCII formatted document is part of the specification and is herein incorporated by reference in its entirety.

### FIELD OF THE INVENTION

This invention relates to herbicide-resistant polyploid plants, such as Brassica napus or Brassica juncea plants, seed of such plants, parts thereof, progeny thereof as well as a method for their manufacture, and methods using such plants, and to crop protection by using ALS (acetolactate synthase; also known as AHAS (acetohydroxyacid synthase; EC 2.2.1.6; formerly EC 4.1.3.18)) inhibitor herbicides against unwanted vegetation in areas of growing such herbicide-resistant plants.

### BACKGROUND OF THE INVENTION

Since more than 40 years, herbicides are the preferred tools to control weeds in B. napus. The products used for this purpose, namely Metazachlor, Dimethachlor, Quinmerac, Clomazone, Metolachlor, Napropamide, Clopyralid, Propyzamide, Propaquizafop, Fluazifop and others allow suppressing weeds in B. napus fields without damaging the crop. Nevertheless, under adverse environmental conditions the efficacy of these products leaves room for improvements, especially if noxious weeds like Geranium dissectum, Centaurea cyanus, Sinapis arvensis and/or Alopecurus myosuroides germinate over an extended period of time.

Acetohydroxyacid synthase (AHAS), also known as “acetolactate synthase” (ALS [EC 2.2.1.6; formerly EC 4.1.3.18]) is the first enzyme that catalyzes the biochemical synthesis of the branched chain amino acids valine, leucine and isoleucine (Singh (1999) “Biosynthesis of valine, leucine and isoleucine,” in Plant Amino Acid, Singh, B. K., ed., Marcel Dekker Inc. New York, N.Y., pp. 227-247).

The ALS/AHAS enzyme is present in bacteria, fungi, and plants and from various organisms protein isolates have been obtained and their corresponding amino acid/nucleic acid sequences as well as their biochemical characteristics have been determined/characterized (see, e.g., Umbarger et al., Annu. Rev. Biochem. (1978), 47, 533-606; Chiman et al., Biochim. Biophys. Acta (1998), 1385, 401-419; Duggleby and Pang, J. Biochem. Mol. Biol. (2000), 33, 1-36; Duggleby: Structure and Properties of Acetohydroxyacid Synthase in Thiamine: Catalytic Mechanisms in Normal and Disease States, Vol 11, Marcel Dekker, New York, 2004, 251-274).

ALS is the target of five structurally diverse herbicide families belonging to the class of ALS inhibitor herbicides, like (a) sulfonylurea herbicides (Beyer E. M et al. (1988), Sulfonylureas in Herbicides: Chemistry, Degradation, and Mode of Action; Marcel Dekker, New York, 1988, 117-189), (b) sulfonylaminocarbonyltriazolinone herbicides (Pontzen, R., Pflanz.-Nachrichten Bayer, 2002, 55, 37-52), (c) imidazolinone herbicides (Shaner, D. L., et al., Plant Physiol., 1984, 76, 545-546; Shaner, D. L., and O'Connor, S. L. (Eds.) The Imidazolinone Herbicides, CRC Press, Boca Raton, Fla., 1991), (d) triazolopyrimidine herbicides (Kleschick, W. A. et al., Agric. Food Chem., 1992, 40, 1083-1085), and (e) pyrimidinyl(thio)benzoate herbicides (Shimizu, T. J., Pestic. Sci., 1997, 22, 245-256; Shimizu, T. et al., Acetolactate Syntehase Inhibitors in Herbicide Classes in Development, Boger, P., Wakabayashi, K., Hirai, K., (Eds.), Springer Verlag, Berlin, 2002, 1-41).

Inhibitors of the ALS interrupt the biosynthesis of valine, leucine and isoleucine in plants. The consequence is an immediate depletion of the respective amino acid pools causing a stop of protein biosynthesis leading to a cessation of plant growth and eventually the plant dies, or—at least—is damaged.

ALS inhibitor herbicides such as imidazolinone and sulfonylurea herbicides are widely used in modem agriculture due to their effectiveness at moderate application rates and relative non-toxicity in animals. By inhibiting ALS activity, these families of herbicides prevent further growth and development of susceptible plants including many weed species.

Various mutants in ALS in various plants have been described that confer resistance to one or more ALS inhibitor herbicides. Plants conferring mutant ALS alleles show different levels of tolerance to ALS inhibitor herbicides, depending on the chemical structure of the ALS inhibitor herbicide and the site of the point mutation(s) in the ALS gene and the hereby encoded ALS protein.

Several mutants (naturally occurring in weeds but also artificially induced in crops by either mutation or transgenic approaches) of the ALS conferring tolerance to one or more chemicals defined under the above given ALS inhbitor herbicide classes/groups are known at various parts of the enzyme (i.e. in the α-, β-, and γ-domain of the ALS) are known and have been identified in various organisms, including plants (U.S. Pat. No. 5,378,82; Duggleby, R. G. et al., (2008), Plant Physiol. and Biochem., pp 309-324; Siyuan, T. et al. (2005), Pest Management Sci., 61, pp 246-257; Jung, S. (2004) Biochem J., pp 53-61; Kolkman, J. M. (2004), Theor. Appl. Genet., 109, pp 1147-1159; Duggleby, R. G. et al (2003), Eur. J. Biochem., 270, pp 1295-2904; Pang, S. S., et al. (2003), J. Biol. Chem., pp 7639-7644); Yadav, N. et al., (1986), Proc. Natl. Acad. Sci., 83, pp 4418-4422), Jander G. et al. (2003), Plant Physiol., 131, pp. 139-146); Tranel, P. J., and Wright, T. R. (2002), Weed Science, 50, pp 700-712); Chang, A. K., and Duggleby, R. G. (1998), Biochem J., 333, pp. 765-777).

Among the artificially obtained various mutants, it has already been described that these are tolerant against various classes of ALS inhibitor herbicides, such as against certain sulfonylureas or representative compounds of the class of imidazolinones.

EP-A-0360750 describes the production of ALS inhibitor herbicide tolerant plants by producing an increased amount of the targeted ALS inside the plant. Such plants show an increased tolerance against certain sulfonyureas, like chlorsulfuron, sulfometuron-methyl, and triasulfuron.

U.S. Pat. No. 5,198,599 describes sulfonylurea and imidazolinone tolerant plants that have been obtained via a selection process and which show a tolerance against chlorsulfuron, bensulfuron, chlorimuron, thifensulfuron and sulfometuron.

WO09/046334 describes mutated acetohydroxyacid synthase (AHAS) nucleic acids and the proteins encoded by the mutated nucleic acids, as well as canola plants, cells, and seeds comprising the mutated genes, whereby the plants display increased tolerance to imidazolinones and sulfonylureas.

WO09/031031 discloses herbicide-resistant Brassica plants and novel polynucleotide sequences that encode wild-type and imidazolinone-resistant Brassica acetohydroxyacid synthase large subunit proteins, seeds, and methods using such plants.

U.S. patent application Ser. No. 09/001,3424 describes improved imidazolinone herbicide resistant Brassica lines, including Brassica juncea, methods for generation of such lines, and methods for selection of such lines, as well as Brassica AHAS genes and sequences and a gene allele bearing a point mutation that gives rise to imidazolinone herbicide resistance.

WO08/124495 discloses nucleic acids encoding mutants of the acetohydroxyacid synthase (AHAS) large subunit comprising at least two mutations, for example double and triple mutants, which are useful for producing transgenic or non-transgenic plants with improved levels of tolerance to AHAS-inhibiting herbicides. The invention also provides expression vectors, cells, plants comprising the polynucleotides encoding the AHAS large subunit double and triple mutants, plants comprising two or more AHAS large subunit single mutant polypeptides, and methods for making and using the same.

WO 2010/037061 describes transgenic and non-transgenic plants with improved tolerance to AHAS-inhibiting herbicides such as an oilseed rape which is tolerant towards one specific class of ALS inhibitors, the Imidazolinone herbicides.

WO2011/114232 describes herbicide-tolerant winter-type Brassica plants which express an AHAS enzyme that is tolerant to the action of one or more AHAS enzyme inhibitors.

Tan et al. (Pest. Manag. Sci (2005), 61: 246-257) inter alia refers to imidazolinone-tolerant oilseed rape.

WO2012/049268 and WO2012/049266 describe an ALS inhibitor herbicide tolerant Beta vulgaris plant comprising a mutation of an endogenous ALS gene encoding an ALS polypeptide containing a leucine instead of a tryptophan at a position 569 of the ALS polypeptide. WO2010/014007 describes a sunflower plant comprising an AHASL1 gene encoding an AHASL1 protein with a tryptophan to leucine substitution at a position corresponding to Trp574 of the Arabidopsis protein. WO2007/149069 describes herbicide tolerant Coreopsis tinctoria comprising an AHAS allele with both a P197L and a W574L mutation.

In order to provide plants with an increased tolerance to even high concentrations of ALS inhibitor herbicides and to mixtures of herbicidal compounds that may be required for sufficient weed control, additional ALS-inhibiting herbicide-resistant breeding lines and varieties of crop plants, as well as methods and compositions for the production and use of ALS inhibiting herbicide-resistant breeding lines and varieties, are needed.

Thus, the technical problem is to comply with this need.

The present invention addresses this need and thus provides as a solution to the technical problem of obtaining ALS inhibitor herbicide tolerant polyploid plants, such as Brassica napus plants and Brassica juncea plants and parts thereof according to the present invention.

By applying various breeding methods, high yielding commercial varieties highly competitive in all specific markets with the add-on of a robust ALS inhibitor herbicide tolerance can be developed subsequently by using the originally obtained mutant plants.

### SUMMARY OF THE INVENTION

In one aspect, the present invention provides a polyploid ALS inhibitor herbicide tolerant plant or parts thereof, such as an allotetraploid Brassica plant or parts thereof, comprising at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine, such as plants or parts thereof wherein all ALS genes encode an ALS polypeptide comprising at a position corresponding to position 574 of SEQ ID NO: 10 instead of the naturally encoded amino acid tryptophan the amino acid leucine, and comprising at a position corresponding to position 197 of SEQ ID NO: 10 instead of the naturally encoded amino acid proline the amino acid serine. In another embodiment, said polyploid ALS inhibitor herbicide tolerant plant or parts thereof is selected from the group consisting of:
a. Brassica napus comprising an ALS I gene encoding an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 556 of SEQ ID NO: 4 instead of the naturally encoded amino acid tryptophan the amino acid leucine;
b. Brassica napus comprising an ALS I gene encoding an ALS I polypeptide comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine;
c. Brassica napus comprising an ALS I gene encoding an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine;
d. Brassica juncea compring an ALS-A gene encoding an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 559 of SEQ ID NO: 8 instead of the naturally encoded amino acid tryptophan the amino acid leucine;
e. Brassica juncea compring an ALS-A gene encoding an ALS-A polypeptide comprising at a position corresponding to position 556 of SEQ ID NO: 6 instead of the naturally encoded amino acid tryptophan the amino acid leucine, and an ALS-B gene encoding an ALS-B polypeptide polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine; and
f. Brassica juncea compring an ALS-A gene encoding an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine.

In another embodiment, said B. napus plants or parts thereof comprise an ALS I gene encoding an ALS I polypeptide which is at least 90% identical to SEQ ID NO: 2 of which the proline at position 182 is substituted with a serine, and of which the tryptophan at position 559 is substituted with a leucine, and an ALS III gene encoding an ALS III polypeptide which is at least 90% identical to SEQ ID NO: 4 of which the tryptophan at position 556 is substituted with a leucine, such as an ALS I gene encoding an ALS I polypeptide which is identical to SEQ ID NO: 2 of which the proline at position 182 is substituted with a serine, and of which the tryptophan at position 559 is substituted with a leucine, and an ALS III gene encoding an ALS III polypeptide which is identical to SEQ ID NO: 4 of which the tryptophan at position 556 is substituted with a leucine, or such as an ALS I gene comprising the nucleotide sequence corresponding to SEQ ID NO: 1 of which the C at position 544 is substituted with T and of which the G at position 1676 is substituted with T, and an ALS III gene comprising the nucleotide sequence corresponding to SEQ ID NO: 3 of which the G at position 1667 is substituted with T, or said B. napus plants or parts thereof comprise an ALS I gene encoding an ALS I polypeptide which is at least 90% identical to SEQ ID NO: 2 of which the tryptophan at position 559 is substituted with a leucine, and an ALS III gene encoding an ALS III polypeptide which is at least 90% identical to SEQ ID NO: 4 of which the proline at position 179 is substituted with a serine and of which the tryptophan at position 556 is substituted with a leucine, such as an ALS I gene encoding an ALS I polypeptide which is identical to SEQ ID NO: 2 of which the tryptophan at position 559 is substituted with a leucine, and an ALS III gene encoding an ALS III polypeptide which is identical to SEQ ID NO: 4 of which the proline at position 179 is substituted with a serine, and of which the tryptophan at position 556 is substituted with a leucine, or such as an ALS I gene comprising the nucleotide sequence corresponding to SEQ ID NO: 1 of which the G at position 1676 is substituted with T, and an ALS III gene comprising the nucleotide sequence corresponding to SEQ ID NO: 3 of which the C at position 535 is substituted with T and of which the G at position 1667 is substituted with T, or said B. napus plants or parts thereof comprise an ALS I gene encoding an ALS I polypeptide which is at least 90% identical to SEQ ID NO: 2 of which the proline at position 182 is substituted with a serine, and of which the tryptophan at position 559 is substituted with a leucine, and an ALS III gene encoding an ALS III polypeptide which is at least 90% identical to SEQ ID NO: 4 of which the proline at position 179 is substituted with a serine and of which the tryptophan at position 556 is substituted with a leucine, such as an ALS I gene encoding an ALS I polypeptide which is identical to SEQ ID NO: 2 of which the proline at position 182 is substituted with a serine, and of which the tryptophan at position 559 is substituted with a leucine, and an ALS III gene encoding an ALS III polypeptide which is identical to SEQ ID NO: 4 of which the proline at position 179 is substituted with a serine and of which the tryptophan at position 556 is substituted with a leucine, or such as an ALS I gene comprising the nucleotide sequence corresponding to SEQ ID NO: 1 of which the C at position 544 is substituted with T and of which the G at position 1676 is substituted with T, and an ALS III gene comprising the nucleotide sequence corresponding to SEQ ID NO: 3 of which the C at position 535 is substituted with T and of which the G at position 1667 is substituted with T, or said B. juncea plants or parts thereof comprise an ALS-A gene encoding an ALS-A polypeptide which is at least 90% identical to SEQ ID NO: 6 of which the proline at position 179 is substituted with a serine and of which the tryptophan at position 556 is substituted with a leucine, and an ALS-B gene encoding an ALS-B polypeptide which is at least 90% identical to SEQ ID NO: 8 of which the tryptophan at position 559 is substituted with a leucine, or such as an ALS-A gene encoding an ALS-A polypeptide which is identical to SEQ ID NO: 6 of which the proline at position 179 is substituted with a serine and of which the tryptophan at position 556 is substituted with a leucine, and an ALS-B gene encoding an ALS-B polypeptide which is identical to SEQ ID NO: 8 of which the tryptophan at position 559 is substituted with a leucine, or such as an ALS-A gene comprising the nucleotide sequence corresponding to SEQ ID NO: 5 of which the C at position 535 is substituted with T and of which the G at position 1667 is substituted with T, and an ALS-B gene comprising the nucleotide sequence corresponding to SEQ ID NO: 7 of which the G at position 1676 is substituted with T, or said B. juncea plants or parts thereof comprise an ALS-A gene encoding an ALS-A polypeptide which is at least 90% identical to SEQ ID NO: 6 of which the tryptophan at position 556 is substituted with a leucine, and an ALS-B gene encoding an ALS-B polypeptide which is at least 90% identical to SEQ ID NO: 8 of which the proline at position 182 is substituted with a serine, and of which the tryptophan at position 559 is substituted with a leucine, or such as an ALS-A gene encoding an ALS-A polypeptide which is identical to SEQ ID NO: 6 of which the tryptophan at position 556 is substituted with a leucine, and an ALS-B gene encoding an ALS-B polypeptide which is identical to SEQ ID NO: 8 of which the proline at position 182 is substituted with a serine, and of which the tryptophan at position 559 is substituted with a leucine, or such as an ALS-A gene comprising the nucleotide sequence corresponding to SEQ ID NO: 5 of which the G at position 1667 is substituted with T, and an ALS-B gene comprising the nucleotide sequence corresponding to SEQ ID NO: 7 of which the C at position 544 is substituted with T and of which the G at position 1676 is substituted with T, or said B. juncea plants or parts thereof comprise an ALS-A gene encoding an ALS-A polypeptide which is at least 90% identical to SEQ ID NO: 6 of which the proline at position 179 is substituted with a serine and of which the tryptophan at position 556 is substituted with a leucine, and an ALS-B gene encoding an ALS-B polypeptide which is at least 90% identical to SEQ ID NO: 8 of which the proline at position 182 is substituted with a serine, and of which the tryptophan at position 559 is substituted with a leucine, such as an ALS-A gene encoding an ALS-A polypeptide which is identical to SEQ ID NO: 6 of which the proline at position 179 is substituted with a serine and of which the tryptophan at position 556 is substituted with a leucine, and an ALS-B gene encoding an ALS-B polypeptide which is identical to SEQ ID NO: 8 of which the proline at position 182 is substituted with a serine, and of which the tryptophan at position 559 is substituted with a leucine, or such as an ALS-A gene comprising the nucleotide sequence corresponding to SEQ ID NO: 5 of which the C at position 535 is substituted with T and of which the G at position 1667 is substituted with T, and an ALS-B gene comprising the nucleotide sequence corresponding to SEQ ID NO: 7 of which the C at position 544 is substituted with T and of which the G at position 1676 is substituted with T.

Another embodiment refers to a B. napus plant or parts thereof according to the invention which is obtainable from seeds deposited at NCIMB under accession number NCIMB 42145, NCIMB 42235, and/or NCIMB 42260, or which is obtainable from seeds deposited at NCIMB under accession number NCIMB 42145, NCIMB 42235, NCIMB 42153, and/or NCIMB 42259, or which is obtainable from seeds deposited at NCIMB under accession number NCIMB 42153, NCIMB 42259 and/or NCIMB 42260, whereas yet another embodiment refers to a B. napus plant or parts thereof according to the invention, reference seeds of said plant having been deposited at NCIMB under accession number NCIMB 42145, NCIMB 42235 and NCIMB 42260, or under accession number NCIMB 42145, NCIMB 42235, NCIMB 42153, and NCIMB 42259, or under accession number NCIMB 42153, NCIMB 42259, and NCIMB 42260.

In yet another embodiment, the Brassica plants or parts thereof are Brassica napus winter oilseed rape.

Yet another embodiment refers to a plant or parts thereof according to the present invention, which are tolerant to one or more ALS-inhibitor herbicides belonging to the group consisting of sulfonylurea herbicides, sulfonylaminocarbonyltriazolinone herbicides, imidazolinone herbicides, triazolopyrimidine herbicides, and pyrimidinyl(thio)benzoate herbicides.

Yet another embodiment refers to a plant or parts thereof according to the present invention, characterized in that said plant or parts thereof is homozygous for said ALS genes encoding an ALS polypeptide comprising at a position corresponding to position 574 of SEQ ID NO: 10 instead of the naturally encoded amino acid tryptophan the amino acid leucine, and is homozygous for said ALS genes encoding an ALS polypeptide comprising at a position corresponding to position 197 of SEQ ID NO: 10 instead of the naturally encoded amino acid proline the amino acid serine and comprising at a position corresponding to position 574 of SEQ ID NO: 10 instead of the naturally encoded amino acid tryptophan the amino acid leucine.

Yet another embodiment refers to parts of plant according to the present invention, wherein the parts are organs, tissues, cells or seeds.

Another aspect refers to food, feed, or an industrial product obtainable from a plant according to the invention. Yet another aspect refers to food, feed, or an industrial product obtainable from a plant according to the invention, wherein the food or feed is oil, meal, grain, starch, flour or protein, or the industrial product is biofuel, fiber, industrial chemicals, a pharmaceutical or a nutraceutical.

Yet another aspect refers to progeny of a plant according to the present invention obtained by further breeding with said plant according to the present invention, wherein wherein all ALS genes of said progeny encode an ALS polypeptide comprising at a position corresponding to position 574 of SEQ ID NO: 10 instead of the naturally encoded amino acid tryptophan the amino acid leucine and wherein at least one of the ALS genes encodes an ALS polypeptide which further comprises at a position corresponding to position 197 of SEQ ID NO: 10 instead of the naturally encoded amino acid proline the amino acid serine.

Yet another aspect refers to a method of producing a hybrid seed, comprising crossing a parent plant according to the present invention with a second parent plant.

Yet another aspect refers to a hybrid plant produced from crossing a parent plant according to the present invention with a second parent plant and harvesting a resultant hybrid seed and growing said seed, wherein all ALS genes of said hybrid plant encode an ALS polypeptide comprising at a position corresponding to position 574 of SEQ ID NO: 10 instead of the naturally encoded amino acid tryptophan the amino acid leucine and wherein at least one of the ALS genes encodes an ALS polypeptide which further comprises at a position corresponding to position 197 of SEQ ID NO: 10 instead of the naturally encoded amino acid proline the amino acid serine.

Another embodiment of the invention refers to a method for producing food, feed, or an industrial product, such as oil, meal, grain, starch, flour, protein, biofuel, fiber, industrial chemicals, a pharmaceutical or a nutraceutical, comprising obtaining the plant according to the present invention or a part thereof, and preparing the food, feed, or industrial product from the plant or part thereof.

A further embodiment refers to a method to increase the tolerance to ALS inhibitor herbicide(s) of polyploid plants, said method comprising introducing at least two ALS genes, wherein all ALS genes encode an ALS polypeptide comprising at a position corresponding to position 574 of SEQ ID NO: 10 instead of the naturally encoded amino acid tryptophan the amino acid leucine, and wherein one ALS gene encodes an ALS polypeptide which further comprises at a position corresponding to position 197 of SEQ ID NO: 10 instead of the naturally encoded amino acid proline the amino acid serine.

A further aspect of the present invention refers to the use of one or more ALS inhibitor herbicide(s) for controlling unwanted vegetation in a growing area, such as a Brassica growing area, or such as B. napus growing area which plants, such as Brassica plants, or such as B. napus plants, comprise at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine.

One embodiment refers to the use according to the invention, wherein the ALS inhibitor herbicide(s) belong(s) to:

the group of the (sulfon)amides (group (A)) consisting of:
the subgroup (A1) of the sulfonylureas, consisting of: amidosulfuron [CAS RN 120923-37-7] (=A1-1); azimsulfuron [CAS RN 120162-55-2] (=A1-2); bensulfuron-methyl [CAS RN 83055-99-6] (=A1-3); chlorimuron-ethyl [CAS RN 90982-32-4] (=A1-4); chlorsulfuron [CAS RN 64902-72-3] (=A1-5); cinosulfuron [CAS RN 94593-91-6] (=A1-6); cyclosulfamuron [CAS RN 136849-15-5] (=A1-7); ethametsulfuron-methyl [CAS RN 97780-06-8] (=A1-8); ethoxysulfuron [CAS RN 126801-58-9] (=A1-9); flazasulfuron [CAS RN 104040-78-0] (=A1-10); flucetosulfuron [CAS RN 412928-75-7] (=A1-11); flupyrsulfuron-methyl-sodium [CAS RN 144740-54-5] (=A1-12); foramsulfuron [CAS RN 173159-57-4] (=A1-13); halosulfuron-methyl [CAS RN 100784-20-1] (=A1-14); imazosulfuron [CAS RN 122548-33-8] (=A1-15); iodosulfuron-methyl-sodium [CAS RN 144550-36-7] (=A1-16); mesosulfuron-methyl [CAS RN 208465-21-8] (=A1-17); metsulfuron-methyl [CAS RN 74223-64-6] (=A1-18); monosulfuron [CAS RN 155860-63-2] (=A1-19); nicosulfuron [CAS RN 111991-09-4] (=A1-20); orthosulfamuron [CAS RN 213464-77-8] (=A1-21); oxasulfuron [CAS RN 144651-06-9] (=A1-22); primisulfuron-methyl [CAS RN 86209-51-0] (=A1-23); prosulfuron [CAS RN 94125-34-5] (=A1-24); pyrazosulfuron-ethyl [CAS RN 93697-74-6] (=A1-25); rimsulfuron [CAS RN 122931-48-0] (=A1-26); sulfometuron-methyl [CAS RN 74222-97-2] (=A1-27); sulfosulfuron [CAS RN 141776-32-1](=A1-28); thifensulfuron-methyl [CAS RN 79277-27-3] (=A1-29); triasulfuron [CAS RN 82097-50-5](=A1-30); tribenuron-methyl [CAS RN 101200-48-0] (=A1-31); trifloxysulfuron [CAS RN 145099-21-4]-(sodium) (=A1-32); triflusulfuron-methyl [CAS RN 126535-15-7] (=A1-33); tritosulfuron [CAS RN 142469-14-5] (=A1-34); NC-330 [CAS RN 104770-29-8] (=A1-35); NC-620 [CAS RN 868680-84-6](=A1-36); TH-547 [CAS RN 570415-88-2] (=A1-37); monosulfuron-methyl [CAS RN 175076-90-1](=A1-38); metazosulfuron [CAS RN 868680-84-6] (=A1-39); methiopyrsulfuron [CAS RN 441050-97-1] (=A1-40); iofensulfuron-sodium [CAS RN 1144097-30-2] (=A1-41); propyrisulfuron [CAS RN 570415-88-2] (=A1-42);
the subgroup of the sulfonylaminocarbonyltriazolinones (subgroup ((A2)), consisting of: flucarbazone-sodium [CAS RN 181274-17-9] (=A2-1); propoxycarbazone-sodium [CAS RN 181274-15-7] (=A2-2); thiencarbazone-methyl [CAS RN 317815-83-1] (=A2-3);
the subgroup of the triazolopyrimidines (subgroup (A2)), consisting of: cloransulam-methyl [147150-35-4] (=A3-1); diclosulam [CAS RN 145701-21-9] (=A3-2); florasulam [CAS RN 145701-23-1] (=A3-3); flumetsulam [CAS RN 98967-40-9] (=A3-4); metosulam [CAS RN 139528-85-1] (=A3-5); penoxsulam [CAS RN 219714-96-2] (=A3-6); pyroxsulam [CAS RN 422556-08-9] (=A3-7);
the subgroup of the sulfonanilides (subgroup (A4)), consisting of: compounds or salts thereof, and racemates and enantiomers thereof, from the group described by the general formula (I):

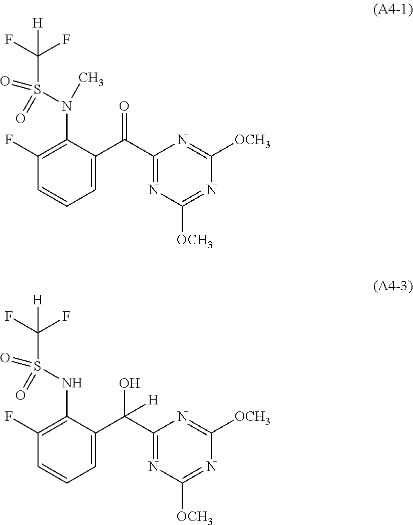

in which
R¹ is halogen, preferably fluorine or chlorine,
R² is hydrogen and R³ is hydroxyl or
R² and R³ together with the carbon atom to which they are attached are a carbonyl group C═O and
R⁴ is hydrogen or methyl;
and more especially compounds of the below given chemical structure (A4-1) to (A4-8)

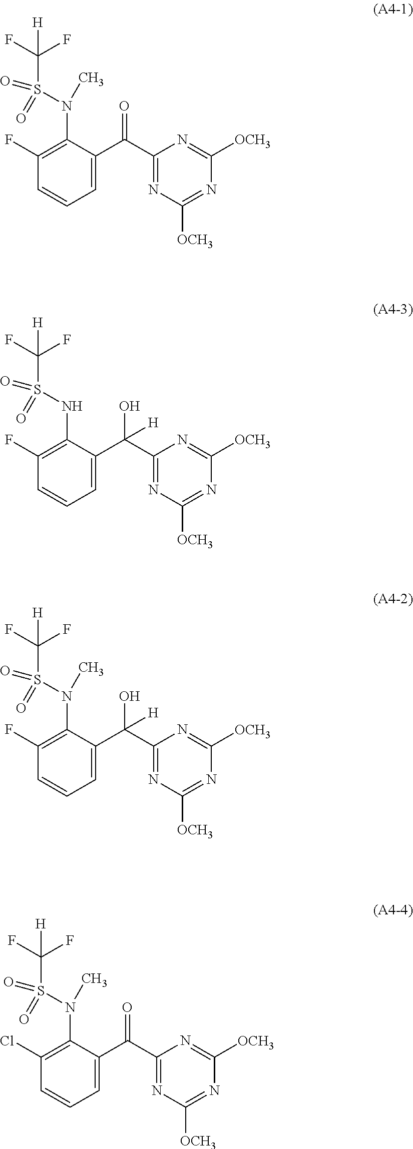 [SMILES: COc1nc(OC)nc(C(=O)c2cccc(F)c2N(C)S(=O)(=O)C(F)F)n1 ; COc1nc(OC)nc(C(O)c2cccc(F)c2NS(=O)(=O)C(F)F)n1 ; COc1nc(OC)nc(C(O)c2cccc(F)c2N(C)S(=O)(=O)C(F)F)n1 ; COc1nc(OC)nc(C(=O)c2cccc(Cl)c2N(C)S(=O)(=O)C(F)F)n1]

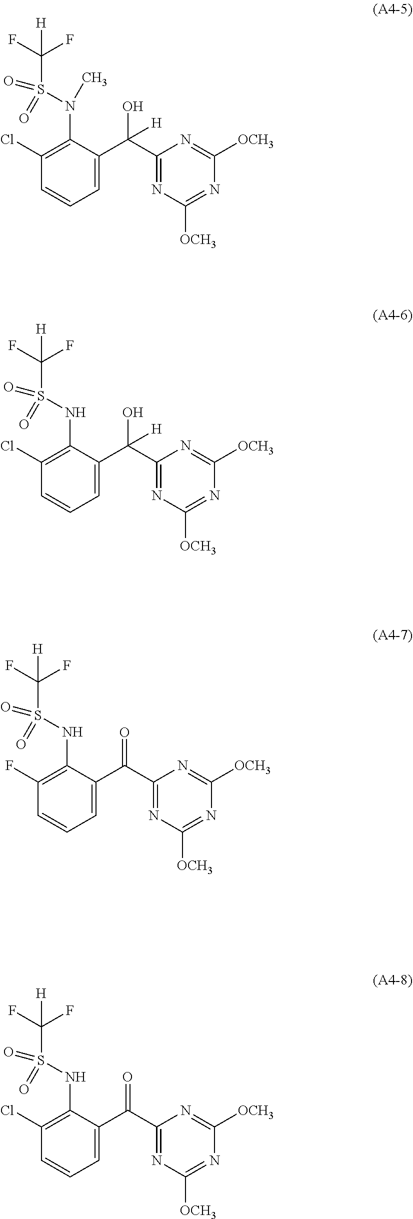 [SMILES: COc1nc(OC)nc(C(O)c2cccc(Cl)c2N(C)S(=O)(=O)C(F)F)n1 ; COc1nc(OC)nc(C(O)c2cccc(Cl)c2NS(=O)(=O)C(F)F)n1 ; COc1nc(OC)nc(C(=O)c2cccc(F)c2NS(=O)(=O)C(F)F)n1 ; COc1nc(OC)nc(C(=O)c2cccc(Cl)c2NS(=O)(=O)C(F)F)n1]

the group of the imidazolinones (group (B)), consisting of:
imazamethabenzmethyl [CAS RN 81405-85-8] (=B1-1); imazamox [CAS RN 114311-32-9] (=B1-2); imazapic [CAS RN 104098-48-8] (=B1-3); imazapyr [CAS RN 81334-34-1] (=B1-4); imazaquin [CAS RN 81335-37-7] (=B1-5); imazethapyr [CAS RN 81335-77-5] (=B1-6); SYP-298 [CAS RN 557064-77-4] (=B1-7); and SYP-300 [CAS RN 374718-10-2] (=B1-8);
the group of the pyrimidinyl(thio)benzoates (group (C)), consisting of:
the subgroup of the pyrimidinyloxybenzoeacids (subgroup (C1)) consisting of: bispyribac-sodium [CAS RN 125401-92-5] (=C1-1); pyribenzoxim [CAS RN 168088-61-7] (=C1-2); pyriminobac-methyl [CAS RN 136191-64-5] (=C1-3); pyribambenz-isopropyl [CAS RN 420138-41-6] (=C1-4); and pyribambenz-propyl [CAS RN 420138-40-5] (=C1-5);
the subgroup of the pyrimidinylthiobenzoeacids (subgroup (C2)), consisting of: pyriftalid [CAS RN 135186-78-6] (=C2-1); and pyrithiobac-sodium [CAS RN 123343-16-8] (=C2-2).

Another embodiment refers to the use according to the invention, wherein the ALS inhibitor herbicide(s) belong(s) to the group consisting of: amidosulfuron [CAS RN 120923-37-7] (=A1-1); chlorimuron-ethyl [CAS RN 90982-32-4] (=A1-4); chlorsulfuron [CAS RN 64902-72-3] (=A1-5); ethametsulfuron-methyl [CAS RN 97780-06-8] (=A1-8); ethoxysulfuron [CAS RN 126801-58-9] (=A1-9); flupyrsulfuron-methyl-sodium [CAS RN 144740-54-5] (=A1-12); foramsulfuron [CAS RN 173159-57-4] (=A1-13); iodosulfuron-methyl-sodium [CAS RN 144550-36-7] (=A1-16);

mesosulfuron-methyl [CAS RN 208465-21-8] (=A1-17); metsulfuron-methyl [CAS RN 74223-64-6] (=A1-18); monosulfuron [CAS RN 155860-63-2] (=A1-19); nicosulfuron [CAS RN 111991-09-4] (=A1-20); rimsulfuron [CAS RN 122931-48-0] (=A1-26); sulfosulfuron [CAS RN 141776-32-1] (=A1-28); thifensulfuron-methyl [CAS RN 79277-27-3] (=A1-29); tribenuron-methyl [CAS RN 101200-48-0] (=A1-31); triflusulfuron-methyl [CAS RN 126535-15-7] (=A1-33); iofensulfuron-sodium [CAS RN 1144097-30-2] (=A1-41); flucarbazone-sodium [CAS RN 181274-17-9] (=A2-1); propoxycarbazone-sodium [CAS RN 181274-15-7] (=A2-2); thiencarbazone-methyl [CAS RN 317815-83-1] (=A2-3); florasulam [CAS RN 145701-23-1] (=A3-3); metosulam [CAS RN 139528-85-1] (=A3-5); pyroxsulam [CAS RN 422556-08-9] (=A3-7); (A4-1); (A4-2); (A4-3); imazamox [CAS RN 114311-32-9] (=B1-2); and bispyribac-sodium [CAS RN 125401-92-5] (=C1-1).

Another embodiment refers to the use according to the present invention, wherein the ALS inhibitor herbicide(s) belong(s) to the group consisting of: amidosulfuron [CAS RN 120923-37-7] (=A1-1); foramsulfuron [CAS RN 173159-57-4] (=A1-13); iofensulfuron-sodium [CAS RN 1144097-30-2] (=A1-41); thiencarbazone-methyl [CAS RN 317815-83-1] (=A2-3); imazamox [CAS RN 114311-32-9] (=B1-2); and bispyribac-sodium [CAS RN 125401-92-5] (=C1-1).

Yet another embodiment refers to the use according to the present invention, wherein the Brassica plants are selected from the group consisting of:
a. B. napus plants comprising an ALS I B. napus gene encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and wherein an ALS III B. napus gene encodes an ALS III polypeptide comprising at a position corresponding to position 556 of SEQ ID NO: 4 instead of the naturally encoded amino acid tryptophan the amino acid leucine; and
b. B. napus plants comprising an ALS I B. napus gene encoding an ALS I polypeptide comprising at a position corresponding to position 559 of SEQ ID NO: 2 instead of the naturally encoded amino acid tryptophan the amino acid leucine, and wherein an ALS III B. napus gene encodes an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine.

Yet another embodiment refers to the use according to the present invention, wherein the ALS inhibitor herbicide(s) are used in combination with non-ALS inhibitor herbicides (i.e. herbicides showing a mode of action that is different to the inhibition of the ALS enzyme [acetohydroxyacid synthase; EC 2.2.1.6] (group B herbicides according to the HRAC classification on mode of action), and wherein the non ALS inhibitor herbicide(s) is/are selected form the group consisting of: acetochlor (=D1), carbetamide (=D56), fenoxaprop-P-ethyl (=D164), fluazifop-P-butyl (=D174), haloxyfop-P-methyl (=D222), metolachlor (=D275), dimethenamid (=D132), napropamide (=D290), pethoxamid (=D317), propaquizafop (=D341), propisochlor (=D344), propyzamide (=D345), quinmerac (=D363), propachlor (D 427), clomazone (=D83), clopyralid (=D86), dimethachlor (=D130), metazachlor (=D265), picloram (=D321), and quizalofop-P-ethyl (=D368).

Yet another embodiment refers to the use according to the present invention, wherein the ALS inhibitor herbicide(s) are used in combination with non-ALS inhibitor herbicide(s) is/are selected form the group consisting of: clomazone (=D83), clopyralid (=D86), dimethachlor (=D130), metazachlor (=D265), picloram (=D321), and quizalofop-P-ethyl (=D368).

Another aspect of the present invention refers to a method for controlling unwanted vegetation in plant growing areas, such as Brassica growing areas, or such as B. napus growing areas, by applying one or more ALS inhibitor herbicide(s) alone or in combination with one or more herbicide(s) that do(es) not belong to the class of ALS inhibitor herbicides for weed control in growing areas, such as Brassica growing areas, or such as B. napus growing areas, which plants, such as Brassica plants, or such as B. napus plants comprise at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine.

One embodiment refers to a method according to the present invention for controlling unwanted vegetation, and wherein the ALS inhibitor herbicide(s) are taken from the groups as defined in [39].

One embodiment refers to a method according to the present invention, and wherein the ALS inhibitor herbicide(s) are taken from the groups as defined in [40].

One embodiment refers to a method according to the present invention, and wherein the non ALS inhibitor herbicide(s) are taken from the group as defined in [43].

One embodiment refers to a method according to the present invention, and wherein the non ALS inhibitor herbicide(s) are taken from the group as defined in [44].

### BRIEF DESCRIPTION OF THE DRAWINGS

FIG. 1: Alignment of SEQ ID NOs: 9 (AtALS), 1 (BnALS I), 3 (BnALS III), 5 (BjALS-A), 7 (BjALS-B). The codon encoding the Proline at a position corresponding to position 197 of SEQ ID NO: 10, and the codon encoding the Tryptophan at a position corresponding to position 574 of SEQ ID NO: 10 are indicated with bold capitals on gray background.

FIG. 2: Alignment of SEQ ID NOs: 10 (AtALS protein), 2 (BnALS I protein), 4 (BnALS III protein), 6 (BjALS-A protein), 8 (BjALS-B protein). The Proline (P) at a position corresponding to position 197 of SEQ ID NO: 10 and the Tryptophan (W) at a position corresponding to position 574 of SEQ ID NO: 10 are indicated with bold underlined capitals on gray background.

FIG. 3: Alignment of SEQ ID NOs: 10, 12, 14, 16, 18, 20, 22, 24, 26, 28, 30, 32, 34, 36, and 38. The Proline (P) at a position corresponding to position 197 of SEQ ID NO: 10 and the Tryptophan (W) at a position corresponding to position 574 of SEQ ID NO: 10 is indicated with bold underlined capitals on a gray background. The P at a position corresponding to position 197 of SEQ ID NO: 10 is absent from SEQ ID NO: 22, but this might be due to an error in the annotation of the gene, in which the sequence encoding the amino acids corresponding to amino acids 196-213 of SEQ ID NO: 10 are annotated as an intron (see EnsemblPlants number GLYMA04G37270.1). The P at a position corresponding to position 197 of SEQ ID NO: 10 is absent from SEQ ID NO: 38 as this concerns only a partial sequence of the Saccharum officinarum ALS gene (see GenBank number EU243998.1).

### DETAILED DESCRIPTION

### General Definitions

It must be noted that as used herein, the terms “a”, “an”, and “the”, include singular and plural references unless the context clearly indicates otherwise, i.e., such terms may refer to “one”, “one or more” or “at least one”. Thus, for example, reference to “a reagent” includes one or more of such different reagents and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

### Plant

When used herein the term “polyploid plant” refers to a plant containing more than two paired sets of chromosomes. A polyploid plant can be an autopolyploid plant, which contains multiple chromosome sets from a single species. A polyploid plant can further be an allopolyploid plant, which contains multiple chromosome sets derived from different species, such as an allotetraploid plant, which contains four sets of chromosomes derived from two different species. Such polyploid plants can be, for example, triploid plants, comprising three sets of chromosomes, or can be tetraploid plants, comprising four sets of chromosomes, or can be pentaploid plants, comprising five sets of chromosomes, or can be hexaploid plants, comprising six sets of chromosomes, or can be octaploid plants, comprising eight sets of chromosomes, or can be decaploid plants, comprising ten sets of chromosomes, or can be dodecaploid plants, comprising twelve sets of chromosomes. Examples of polyploid plants include Brasssica napus, Brassica juncea, Brassica carinata, wheat, cotton (Gossypium hirsutum), potato, alfalfa, sugar cane, soybeans, leek, tobacco, peanut, kinnow, pelargonium, chrysanthemum, triticale, oat, kiwifruit, strawberry, dahlia, pansies, oca, tulips, lilies, daylilies, apple, banana, citrus, coffee and watermelon.

When used herein the term “Brassica napus” is abbreviated as “B. napus”. Furthermore, the term “oilseed rape” is used herein. Said three terms are interchangeably used and should be understood to fully comprise the cultivated forms of B. napus, e.g., as defined in Tang et al, Plant Breeding, Volume 116, Issue 5, pages 471-474, October 1997 and Jesske et al., Tagung der Vereinigung der Pflanzenzfüchter und Saatgutkaufleute Österreichs, 2009, 171-172, ISBN: 978-3-902559-37-1). Similarly, for example, the term “Brassica juncea” is abbreviated as “B. juncea”, and the term “Arabidopsis thaliana” is abbreviated as “A. thaliana”. Both terms are interchangeably used herein.

When used herein “winter-type Brassica plant” can be winter-type Brassica juncea, or winter-type Brassica napus. Winter-type Brassica napus as used herein is also referred to as winter oilseed rape (WOSR). The term ‘winter-type’ refers to plant species that require cold treatment, or vernalization, before it will flower. In nature such plant species are mainly biennal species. In the first year the biennal plant grows vegetative (leafs, stems, roots) as rozet, and after a cold period between first and second year (winter season) the plant will elongate and start to flower in the second year. Winter oilseed rape is planted right after the harvest, typically from September to November in the Northern Hemisphere, sprouting before freezing occurs, then becomes dormant until the soil warms in the spring and is ready to be harvested in summer.

The term “wild-type” as used herein refers to a plant, a nucleic acid molecule or protein that can be found in nature as distinct from being artificially produced or mutated by man. Thus, in one embodiment, a “wild type” plant does not produce or comprise ALS proteins with an amino acid different from tryptophan 574 (the numbers behind the amino acids indicate the positions corresponding to these positions of SEQ ID NO: 10, which is the ALS protein as derived from A. thaliana).

In one embodiment, a “wild-type” B. napus plant refers to a B. napus plant having at least one ALS nucleic acid sequence containing at least 60%, or 70%, or 80%, or 90%, or 95%, or 97%, or 98%, or 99% sequence identity, or is identical to SEQ ID NO: 1 and at least one ALS nucleic acid sequence containing at least 60%, or 70%, or 80%, or 90%, or 95%, or 97%, or 98%, or 99% sequence identity, or is identical to SEQ ID NO: 3, provided that said plant does not carry an ALS I gene carrying a mutation in the Trp574 codon yielding an amino acid different from Trp or an ALS I carrying a mutation in the P197 codon yielding an amino acid different from Pro and a mutation in the Trp574 codon yielding an amino acid different from Trp, and does not carry an ALS III gene carrying a mutation in the Trp574 codon yielding an amino acid different from Trp or an ALS III gene carrying a mutation in the P197 codon yielding an amino acid different from Pro and a mutation in the Trp574 codon yielding an amino acid different from Trp, wherein the amino acid position referred to is the position in the reference A. thaliana sequence (SEQ ID NO: 10). The use of the term “wild-type” is not intended to necessarily imply that a plant, plant tissue, plant cell, or other host cell lacks recombinant DNA in its genome, and/or does not possess herbicide resistant characteristics that are different from those disclosed herein.

In one embodiment, a “wild-type” B. juncea plant refers to a B. juncea plant having at least one ALS nucleic acid sequence containing at least 60%, or 70%, or 80%, or 90%, or 95%, or 97%, or 98%, or 99% sequence identity, or is identical to SEQ ID NO: 5 and at least one ALS nucleic acid sequence containing at least 60%, or 70%, or 80%, or 90%, or 95%, or 97%, or 98%, or 99% sequence identity, or is identical to SEQ ID NO: 7, provided that said plant does not carry an ALS-A gene carrying a mutation in the Trp574 codon yielding an amino acid different from Trp or an ALS-A gene carrying a mutation in the P197 codon yielding an amino acid different from Pro and a mutation in the Trp574 codon yielding an amino acid different from Trp, and does not carry an ALS-B gene carrying a mutation in the Trp574 codon yielding an amino acid different from Trp or an ALS-B gene carrying a mutation in the P197 codon yielding an amino acid different from Pro and a mutation in the Trp574 codon yielding an amino acid different from Trp, wherein the amino acid position referred to is the position in the reference A. thaliana sequence (SEQ ID NO: 10). The use of the term “wild-type” is not intended to necessarily imply that a plant, plant tissue, plant cell, or other host cell lacks recombinant DNA in its genome, and/or does not possess herbicide resistant characteristics that are different from those disclosed herein.

Due to the fact that the plants of the present invention which are herbicide resistant were generated by “random evolution”, i.e., methods preferably leading to fertile plants having two point mutation as described herein in more detail without exogenous genetic manipulation, they are non-transgenic as far as the ALS gene in its endogenous gene locus is concerned.

Mutant ALS alleles according to the invention can also be provided to plant cells as transgene. Accordingly, plants may contain a mutant ALS gene according to the invention as transgene.

Moreover, the plants of the present invention and their offspring are fertile and thus useful for breeding purposes in order to generate varieties conferring agronomically useful levels of tolerance to ALS inhibitor herbicides, thus allowing innovative weed control measures plant growing areas.

The term “Brassica plant” as used herein refers to the genus of plants in the mustard family (Brassicaceae). The members of the genus may be collectively known either as cabbages, or as mustards. The genus “Brassica” encompasses, e.g., B. carinata, B. elongata, B. fruticulosa, B. juncea, B. napus, B. narinosa, B. nigra, B. oleracea, B. perviridis, B. rapa, B. rupestris, B. septiceps, and B. tournefortii. The skilled person will understand that the term not only encompasses B. napus but also other hybrids which have at least one parent plant of the genus “Brassica”.

As used herein unless clearly indicated otherwise, the term “plant” intends to mean a plant at any developmental stage. Moreover, the term also encompasses “parts of a plant”. The term “plant” encompasses a plant as described herein, or progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

Parts of (a) plant(s) may be attached to or separate from a whole intact plant. Such parts of a plant include, but are not limited to, cells of a plant, tissues or organs, seeds, severed parts such as roots, leaves, flowers, pollen, etc.

The obtained plants according to the invention can be used in a conventional breeding scheme to produce more plants with the same characteristics or to introduce the ALS alleles according to the invention in other varieties of the same or related plant species, or in hybrid plants. The obtained plants can further be used for creating propagating material. Plants according to the invention can further be used to produce gametes, seeds (including crushed seeds and seed cakes), seed oil, embryos, either zygotic or somatic, progeny or hybrids of plants obtained by methods of the invention.

“Creating propagating material”, as used herein, relates to any means know in the art to produce further plants, plant parts or seeds and includes inter alia vegetative reproduction methods (e.g. air or ground layering, division, (bud) grafting, micropropagation, striking or cutting), sexual reproduction (crossing with another plant) and asexual reproduction (e.g. apomixis, somatic hybridization).

In one embodiment, a plant of the invention comprises at least two ALS genes wherein all ALS genes encode an ALS polypeptide wherein Trp at a position corresponding to position 574 of SEQ ID NO: 10 is substituted by Leu, and wherein at least one of the ALS genes encodes an ALS polypeptide which further comprises at a position corresponding to position 197 of SEQ ID NO: 10 instead of the naturally encoded amino acid proline the amino acid serine. An ALS gene encoding an ALS polypeptide wherein Trp at a position corresponding to position 574 of SEQ ID NO: 10 is substituted by Leu may, or may not, further comprise at a position corresponding to position 197 of SEQ ID NO: 10 instead of the naturally encoded amino acid proline the amino acid serine. In one embodiment, the plant according to the invention comprises at least two ALS genes, wherein all ALS genes encode an ALS polypeptide wherein Trp at a position corresponding to position 574 of SEQ ID NO: 10 is substituted by Leu, and wherein one of the ALS genes encodes an ALS polypeptide which further comprises at a position corresponding to position 197 of SEQ ID NO: 10 instead of the naturally encoded amino acid proline the amino acid serine. In another embodiment, said plant comprises at least two ALS genes, wherein all ALS genes encode an ALS polypeptide comprising at a position corresponding to position 574 of SEQ ID NO: 10 instead of the naturally encoded amino acid tryptophan the amino acid leucine, and comprising at a position corresponding to position 197 of SEQ ID NO: 10 instead of the naturally encoded amino acid proline the amino acid serine.

In one embodiment, a B. napus plant of the invention comprises an ALS I protein wherein Pro at a position corresponding to position 182 of SEQ ID NO: 2 is substituted by Ser, and wherein Trp at a position corresponding to position 559 of SEQ ID NO: 2 is substituted by Leu and an ALS III protein wherein Trp at a position corresponding to position 556 of SEQ ID NO: 4 is substituted by Leu, or a B. napus plant of the invention comprises an ALS I protein wherein Trp at a position corresponding to position 559 of SEQ ID NO: 2 is substituted by Leu and an ALS III protein wherein Pro at a position corresponding to position 179 of SEQ ID NO: 4 is substituted by Ser, and wherein Trp at a position corresponding to position 556 of SEQ ID NO: 4 is substituted by Leu, or a B. napus of the invention comprises an ALS I protein wherein Pro at a position corresponding to position 182 of SEQ ID NO: 2 is substituted by Ser, and wherein Trp at a position corresponding to position 559 of SEQ ID NO: 2 is substituted by Leu and an ALS III protein wherein Pro at a position corresponding to position 179 of SEQ ID NO: 4 is substituted by Ser, and wherein Trp at a position corresponding to position 556 of SEQ ID NO: 4 is substituted by Leu.

In a further embodiment, a B. napus plant of the invention comprises an ALS I protein wherein Pro at a position corresponding to position 182 of SEQ ID NO: 2 is substituted by Ser, and wherein Trp at a position corresponding to position 559 of SEQ ID NO: 2 is substituted by Leu and an ALS III protein wherein Trp at a position corresponding to position 556 of SEQ ID NO: 4 is substituted by Leu, and does neither comprise a wild type ALS I protein nor a wild type ALS III protein, or a B. napus plant of the invention comprises an ALS I protein wherein Trp at a position corresponding to position 559 of SEQ ID NO: 2 is substituted by Leu and an ALS III protein wherein Pro at a position corresponding to position 179 of SEQ ID NO: 4 is substituted by Ser, and wherein Trp at a position corresponding to position 556 of SEQ ID NO: 4 is substituted by Leu and does neither comprise a wild type ALS I protein nor a wild type ALS III protein, or a B. napus plant of the invention comprises an ALS I protein wherein Pro at a position corresponding to position 182 of SEQ ID NO: 2 is substituted by Ser, and wherein Trp at a position corresponding to position 559 of SEQ ID NO: 2 is substituted by Leu and an ALS III protein wherein Pro at a position corresponding to position 179 of SEQ ID NO: 4 is substituted by Ser, and wherein Trp at a position corresponding to position 556 of SEQ ID NO: 4 is substituted by Leu and does neither comprise a wild type ALS I protein nor a wild type ALS III protein.

In one embodiment, a B. napus plant of the invention comprises an ALS I gene of SEQ ID NO: 1 of which the C at position 544 is substituted with T and of which the G at position 1676 is substituted with T and an ALS III gene of SEQ ID NO: 3 of which the G at position 1667 is substituted with T, or a B. napus plant of the invention comprises an ALS I gene of SEQ ID NO: 1 of which the G at position 1676 is substituted with T, and an ALS III gene of SEQ ID NO: 3 of which the C at position 535 is substituted with T and of which the G at position 1667 is substituted with T, or a B. napus plant of the invention comprises an ALS I gene of SEQ ID NO: 1 of which the C at position 544 is substituted with T and of which the G at position 1676 is substituted with T and an ALS III gene of SEQ ID NO: 3 of which the C at position 535 is substituted with T and of which the G at position 1667 is substituted with T.

In one embodiment, a B. juncea plant of the invention comprises an ALS-A protein wherein Pro at a position corresponding to position 179 of SEQ ID NO: 6 is substituted by Ser, and wherein Trp at a position corresponding to position 556 of SEQ ID NO: 6 is substituted by Leu and an ALS-B protein wherein Trp at a position corresponding to position 559 of SEQ ID NO: 8 is substituted by Leu, or a B. juncea plant of the invention comprises an ALS-A protein wherein Trp at a position corresponding to position 556 of SEQ ID NO: 6 is substituted by Leu and an ALS-B protein wherein Pro at a position corresponding to position 182 of SEQ ID NO: 8 is substituted by Ser, and wherein Trp at a position corresponding to position 559 of SEQ ID NO: 8 is substituted by Leu, or a B. juncea plant of the invention comprises an ALS-A protein wherein Pro at a position corresponding to position 179 of SEQ ID NO: 6 is substituted by Ser, and wherein Trp at a position corresponding to position 556 of SEQ ID NO: 6 is substituted by Leu and an ALS-B protein wherein Pro at a position corresponding to position 182 of SEQ ID NO: 8 is substituted by Ser, and wherein Trp at a position corresponding to position 559 of SEQ ID NO: 8 is substituted by Leu.

In a further embodiment, a B. juncea plant of the invention comprises an ALS-A protein wherein Pro at a position corresponding to position 179 of SEQ ID NO: 6 is substituted by Ser, and wherein Trp at a position corresponding to position 556 of SEQ ID NO: 6 is substituted by Leu and an ALS-B protein wherein Trp at a position corresponding to position 559 of SEQ ID NO: 8 is substituted by Leu, and does neither comprise a wild type ALS-A protein nor a wild type ALS-B protein, or a B. juncea plant of the invention comprises an ALS-A protein wherein Trp at a position corresponding to position 556 of SEQ ID NO: 6 is substituted by Leu and an ALS-B protein wherein Pro at a position corresponding to position 182 of SEQ ID NO: 8 is substituted by Ser, and wherein Trp at a position corresponding to position 559 of SEQ ID NO: 8 is substituted by Leu and does neither comprise a wild type ALS-A protein nor a wild type ALS-B protein, or a B. juncea plant of the invention comprises an ALS-A protein wherein Pro at a position corresponding to position 179 of SEQ ID NO: 6 is substituted by Ser, and wherein Trp at a position corresponding to position 556 of SEQ ID NO: 6 is substituted by Leu and an ALS-B protein wherein Pro at a position corresponding to position 182 of SEQ ID NO: 8 is substituted by Ser, and wherein Trp at a position corresponding to position 559 of SEQ ID NO: 8 is substituted by Leu and does neither comprise a wild type ALS-A protein nor a wild type ALS-B protein.

In one embodiment, a B. juncea plant of the invention comprises an ALS-A gene of SEQ ID NO: 5 of which the C at position 535 is substituted with T and of which the G at position 1667 is substituted with T and an ALS-B gene of SEQ ID NO: 7 of which the G at position 1676 is substituted with T, or a B. juncea plant of the invention comprises an ALS-A gene of SEQ ID NO: 5 of which the G at position 1667 is substituted with T and an ALS-B gene of SEQ ID NO: 7 of which the C at position 544 is substituted with T and of which the G at position 1676 is substituted with T, or a B. juncea plant of the invention comprises an ALS-A gene of SEQ ID NO: 5 of which the C at position 535 is substituted with T and of which the G at position 1667 is substituted with T and an ALS-B gene of SEQ ID NO: 7 of which the C at position 544 is substituted with T and of which the G at position 1676 is substituted with T.

In one embodiment, a plant in accordance with the present invention is obtainable from or derivable from or can be obtained from or derived from seeds deposited with the NCIMB, Ferguson Building, Craibstone Estate, Bucksburn, Aberdeen, AB 21 9YA UK, under the Budapest Treaty on May 20, 2013, under accession number NCIMB 42145, from seeds deposited with the NCIMB, Ferguson Building, Craibstone Estate, Bucksburn, Aberdeen, AB 21 9YA UK, under the Budapest Treaty on May 8, 2014, under accession number NCIMB 42235, from seeds deposited with the NCIMB, Ferguson Building, Craibstone Estate, Bucksburn, Aberdeen, AB 21 9YA UK, under the Budapest Treaty on Jul. 5, 2013, under accession number NCIMB 42153, from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42259, and/or from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42260.

It will be clear that a B. napus plant comprising an ALS I gene encoding an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 556 of SEQ ID NO: 4 instead of the naturally encoded amino acid tryptophan the amino acid is obtainable from or derivable from or can be obtained from or derived from seeds deposited with the NCIMB under Number 42145 and Number 42260, or from seeds deposited with the NCIMB under Number 42235 and Number 42260, and that a B. napus plant comprising an ALS I gene encoding an ALS I polypeptide comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine is obtainable from or derivable from or can be obtained from or derived from seeds deposited with the NCIMB under Number 42145 and Number 42153, or from seeds deposited with the NCIMB under Number 42235 and Number 42153, or from seeds deposited with the NCIMB under Number 42145 and Number 42259, or from seeds deposited with the NCIMB under Number 42235 and Number 42259, and that a B. napus plant comprising an ALS I gene encoding an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine is obtainable from or derivable from or can be obtained from or derived from seeds deposited with the NCIMB under Number 42153 and Number 42260, or from seeds deposited with the NCIMB under Number 42259 and Number 42260.

In one embodiment, said plant obtainable from or derivable from or can be obtained from or derived from seeds deposited with the NCIMB under Number 42145, from seeds deposited with NCIMB under number 42235, from seeds deposited with the NCIMB under Number 42153 and/or from seeds deposited with the NCIMB under Number 42259 is a plant directly grown or regenerated from one of said deposited seeds or a plant comprising both mutant alleles described herein, i.e., an ALS I allele coding for an ALS I protein having a mutation at a position corresponding to position 559 of SEQ ID NO:2 as described herein and an ALS III allele coding for an ALS III protein having a mutation at a position corresponding to position 179 and at a position corresponding to position 556 of SEQ ID NO: 4 as described herein. In another embodiment, said plant obtainable from or derivable from or can be obtained from or derived from seeds deposited with the NCIMB under Number 42145, from seeds deposited with NCIMB under number 42235, and/or from seeds deposited with the NCIMB under Number 42260 is a plant directly grown or regenerated from one of said deposited seeds or a plant comprising both mutant alleles described herein, i.e., an ALS I allele coding for an ALS I protein having a mutation at a position corresponding to position 182 and at a position corresponding to position 559 of SEQ ID NO:2 as described herein and an ALS III allele coding for an ALS III protein having a mutation at a position corresponding to position 556 of SEQ ID NO: 4 as described herein. In one embodiment, said plant obtainable from or derivable from or can be obtained from or derived from seeds deposited with the NCIMB under Number 42153, from seeds deposited with the NCIMB under Number 42259, and/or from seeds deposited with the NCIMB under Number 42260 is a plant directly grown or regenerated from one of said deposited seeds or a plant comprising both mutant alleles described herein, i.e., an ALS I allele coding for an ALS I protein having a mutation at a position corresponding to position 182 and at a position corresponding to position 559 of SEQ ID NO:2 as described herein and an ALS III allele coding for an ALS III protein having a mutation at a position corresponding to position 179 and at a position corresponding to position 556 of SEQ ID NO: 4 as described herein.

In one embodiment, such a plant obtainable from or derivable from or can be obtained from or derived from seeds deposited with the NCIMB under Number 42145, 42235, 42153, and/or 42259 encompasses also a first, second, third, fourth or higher generation progeny of a plant directly grown or regenerated from said deposited seed or a first, second, third, fourth or higher generation progeny of a plant having at least one ALS I allele decoding for an ALS I protein having a mutation at a position corresponding to position 559 of SEQ ID NO:2 as described herein and at least one ALS III allele decoding for an ALS III protein having a mutation at a position corresponding to position 179 and at a position corresponding to position 556 of SEQ ID NO: 4 as described herein. In one embodiment, such a plant is homozygous regarding its ALS I and ALS III alleles. In another embodiment, a plant obtainable from or derivable from or can be obtained from or derived from seeds deposited with the NCIMB under Number 42145, 42235 and/or 42260 encompasses also a first, second, third, fourth or higher generation progeny of a plant directly grown or regenerated from said deposited seed or a first, second, third, fourth or higher generation progeny of a plant having at least one ALS I allele decoding for an ALS I protein having a mutation at a position corresponding to position 182 and at a position corresponding to position 559 of SEQ ID NO:2 as described herein and at least one ALS III allele decoding for an ALS III protein having a mutation at a position corresponding to position 556 of SEQ ID NO: 4 as described herein. In one embodiment, such a plant is homozygous regarding its ALS I and ALS III alleles. In another embodiment, a plant obtainable from or derivable from or can be obtained from or derived from seeds deposited with the NCIMB under Number 42153, 42259 and/or 42260 encompasses also a first, second, third, fourth or higher generation progeny of a plant directly grown or regenerated from said deposited seed or a first, second, third, fourth or higher generation progeny of a plant having at least one ALS I allele decoding for an ALS I protein having a mutation at a position corresponding to position 182 and at a position corresponding to position 559 of SEQ ID NO:2 as described herein and at least one ALS III allele decoding for an ALS III protein having a mutation at a position corresponding to position 179 and at a position corresponding to position 556 of SEQ ID NO: 4 as described herein. In one embodiment, such a plant is homozygous regarding its ALS I and ALS III alleles.

In a further embodiment, a plant in accordance with the present invention is provided which comprises an ALS I allele coding for an ALS I protein having a mutation at a position corresponding to position 559 of SEQ ID NO:2 and an ALS III allele coding for an ALS III protein having a mutation at a position corresponding to position 179 and at a position corresponding to position 556 of SEQ ID NO: 4 as present in reference seeds deposited with the NCIMB, Ferguson Building, Craibstone Estate, Bucksburn, Aberdeen, AB 21 9YA UK, under the Budapest Treaty on May 20, 2013, under accession number NCIMB 42145, on May 8, 2014 under accession number NCIMB 42235, on Jul. 5, 2013 under accession number NCIMB 42153, and on Jul. 1, 2014 under accession number NCIMB 42259. In yet a further embodiment, a plant in accordance with the present invention is provided which comprises an ALS I allele coding for an ALS I protein having a mutation at a position corresponding to position 182 and at a position corresponding to position 559 of SEQ ID NO:2 and an ALS III allele coding for an ALS III protein having a mutation at a position corresponding to position 556 of SEQ ID NO: 4 as present in reference seeds deposited with the NCIMB, Ferguson Building, Craibstone Estate, Bucksburn, Aberdeen, AB 21 9YA UK, under the Budapest Treaty on May 20, 2013, under accession number NCIMB 42145, on May 8, 2014, under accession number NCIMB 42235, and on Jul. 1, 2014, under accession number NCIMB 42260. In another embodiment, a plant in accordance with the present invention is provided which comprises an ALS I allele coding for an ALS I protein having a mutation at a position corresponding to position 182 and at a position corresponding to position 559 of SEQ ID NO:2 and an ALS III allele coding for an ALS III protein having a mutation at a position corresponding to position 179 and at a position corresponding to position 556 of SEQ ID NO: 4 as present in reference seeds deposited with the NCIMB, Ferguson Building, Craibstone Estate, Bucksburn, Aberdeen, AB 21 9YA UK, under the Budapest Treaty on Jul. 5, 2013, under accession number NCIMB 42153, on Jul. 1, 2014, under accession number NCIMB 42259, and on Jul. 1, 2014, under accession number NCIMB 42260.

Moreover, also plant cells are obtainable from or are derivable from or are obtained from or are derived from said deposited seeds; or plant cells are obtainable from or are derivable from or are obtained from or are derived from plants which were grown from said deposited seeds.

Accordingly, one embodiment of the present invention is also directed to reference seeds comprising the mutant alleles described herein having been deposited under Number NCIMB 42145, NCIMB 42235, NCIMB 42153, NCIMB 42259 or NCIMB 42260.

One embodiment of the present invention refers to progeny of a polyploid ALS inhibitor herbicide tolerant plant or parts thereof comprising at least two ALS genes, wherein all ALS genes encode an ALS polypeptide comprising at a position corresponding to position 574 of SEQ ID NO: 10 instead of the naturally encoded amino acid tryptophan the amino acid leucine and wherein at least one of the ALS genes encodes an ALS polypeptide which further comprises at a position corresponding to position 197 of SEQ ID NO: 10 instead of the naturally encoded amino acid proline the amino acid serine, such as an ALS inhibitor herbicide tolerant B. napus plant or parts thereof comprising an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and an ALS III polypeptide comprising at a position corresponding to position 556 of SEQ ID NO: 4 instead of the naturally encoded amino acid tryptophan the amino acid leucine, or such as an ALS inhibitor herbicide tolerant B. napus plant or parts thereof comprising an ALS I gene encoding an ALS I polypeptide comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine, or such as an ALS inhibitor herbicide tolerant B. napus plant or parts thereof comprising an ALS I gene encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine, or such as an ALS inhibitor herbicide tolerant B. juncea plant or parts thereof comprising an ALS-A gene encoding an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 559 of SEQ ID NO: 8 instead of the naturally encoded amino acid tryptophan the amino acid leucine, or such as an ALS inhibitor herbicide tolerant B. juncea plant or parts thereof comprising an ALS-A gene encoding an ALS-A polypeptide comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine, or such as an ALS inhibitor herbicide tolerant B. juncea plant or parts thereof comprising an ALS-A gene encoding an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine.

“Progeny” as used herein refers to plants derived from a polyploid ALS inhibitor herbicide tolerant plant comprising at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine, such as a Brassica juncea plant or parts thereof comprising an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B polypeptide comprising at a position corresponding to position 559 of SEQ ID NO: 8 instead of the naturally encoded amino acid tryptophan the amino acid leucine, or such as a Brassica juncea plant or parts thereof comprising an ALS-A polypeptide comprising at a position corresponding to position 556 of SEQ ID NO: 6 instead of the naturally encoded amino acid tryptophan the amino acid leucine, and an ALS-B polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine, or such as a Brassica juncea plant or parts thereof comprising an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine, or such as a Brassica napus plant or parts thereof comprising an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III polypeptide comprising at a position corresponding to position 556 of SEQ ID NO: 4 instead of the naturally encoded amino acid tryptophan the amino acid leucine, e.g., a plant obtainable from or derivable from or obtained from or derived from seeds deposited with the NCIMB, Ferguson Building, Craibstone Estate, Bucksburn, Aberdeen, AB 21 9YA UK, under the Budapest Treaty on May 20, 2013, under accession number NCIMB 42145, from seeds deposited with the NCIMB, Ferguson Building, Craibstone Estate, Bucksburn, Aberdeen, AB 21 9YA UK, under the Budapest Treaty on 8 May 2014, under accession number NCIMB 42235, and/or from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42260, or such as a Brassica napus plant or parts thereof comprising an ALS I polypeptide comprising at a position corresponding to position 559 of SEQ ID NO: 2 instead of the naturally encoded amino acid tryptophan the amino acid leucine, and an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine, e.g., a plant obtainable from or derivable from or obtained from or derived from seeds deposited with the NCIMB, Ferguson Building, Craibstone Estate, Bucksburn, Aberdeen, AB 21 9YA UK, under the Budapest Treaty on May 20, 2013, under accession number NCIMB 42145, from seeds deposited with the NCIMB, Ferguson Building, Craibstone Estate, Bucksburn, Aberdeen, AB 21 9YA UK, under the Budapest Treaty on 8 May 2014, under accession number NCIMB 42235, from seeds deposited with the NCIMB, Ferguson Building, Craibstone Estate, Bucksburn, Aberdeen, AB 21 9YA UK, under the Budapest Treaty on Jul. 5, 2013, under accession number NCIMB 42153, and/or from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42259, or such as a Brassica napus plant or parts thereof comprising an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine, e.g., a plant obtainable from or derivable from or obtained from or derived from seeds deposited with the NCIMB, Ferguson Building, Craibstone Estate, Bucksburn, Aberdeen, AB 21 9YA UK, under the Budapest Treaty on Jul. 5, 2013, under accession number NCIMB 42153, from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42259, and/or from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42260. Progeny may be derived by regeneration of cell or tissue culture or parts of a plant in accordance with the present invention or selfing of a plant in accordance with the present invention or by growing seeds of a plant in accordance with the present invention. In further embodiments, progeny may also encompass plants derived from crossing of at least a plant in accordance with the present invention with another B. napus or Brassica plant, backcrossing, inserting of a locus into a plant or further mutation(s). In one embodiment, a progeny is, e.g., a first generation plant such as a hybrid plant (F1) of a crossing of a plant according to the present invention with another plant, such as B. napus, B. juncea or Brassica plant, or a progeny is regenerated from a plant part of a plant according to the present invention or is the result of self pollination. In another embodiment, a progeny is, e.g., a first, second, third, fourth, fifth, or sixth or higher generation plant derived from, derivable from, obtained from or obtainable from a plant, such as a Brassica plant or a B. napus plant or a B. juncea plant in accordance with the present invention.

Provided herein is an Essentially Derived Variety having at least an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and an ALS III polypeptide comprising at a position corresponding to position 556 of SEQ ID NO: 4 instead of the naturally encoded amino acid tryptophan the amino acid leucine, or having at least an ALS I polypeptide comprising at a position corresponding to position 559 of SEQ ID NO: 2 instead of the naturally encoded amino acid tryptophan the amino acid leucine, and an ALS III polypeptide comprising comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or having at least an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and an ALS III polypeptide comprising comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine.

An “Essentially Derived Variety” (EDV) shall be deemed to be essentially derived from another variety, “the initial variety”, under the following circumstances and in the case that the Initial Variety is a plant which is derived from seeds deposited with the NCIMB, Ferguson Building, Craibstone Estate, Bucksburn, Aberdeen, AB 21 9YA UK, under the Budapest Treaty on May 20, 2013, under accession number NCIMB 42145, from seeds deposited with the NCIMB, Ferguson Building, Craibstone Estate, Bucksburn, Aberdeen, AB 21 9YA UK, under the Budapest Treaty on May 8, 2014, under accession number NCIMB 42235, from seeds deposited with the NCIMB, Ferguson Building, Craibstone Estate, Bucksburn, Aberdeen, AB 21 9YA UK, under the Budapest Treaty on Jul. 5, 2013, under accession number NCIMB 42153, from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42259, and/or from seeds deposited with the NCIMB, Ferguson Building, Craibstone Estate, Bucksburn, Aberdeen, AB 21 9YA UK, under the Budapest Treaty on Jul. 1, 2014, under accession number NCIMB 42260: (i) it is predominantly derived from the initial variety, or from a variety that is itself predominantly derived from the initial variety, while retaining the expression of the essential characteristics that result from the genotype or combination of genotypes of the initial variety, comprising an ALS I polypeptide comprising at a position corresponding to position 559 of SEQ ID NO: 2 instead of the naturally encoded amino acid tryptophan the amino acid leucine, and an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or comprising an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and an ALS III polypeptide comprising at a position corresponding to position 556 of SEQ ID NO: 4 instead of the naturally encoded amino acid tryptophan the amino acid leucine, or or comprising an ALS I polypeptide comprising at a position corresponding to position 182 of SEQ ID NO: 2 instead of the naturally encoded amino acie proline the amino acid serine and at a position corresponding to position 559 of SEQ ID NO: 2 instead of the naturally encoded amino acid tryptophan the amino acid leucine, and an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine; (ii) it is clearly distinguishable from the initial variety (e.g., by its phenotype or genotype); and (iii) except for the differences which result from the act of derivation, it conforms to the initial variety in the expression of the essential characteristics that result from the genotype or combination of genotypes of the initial variety. Thus, an EDV may be obtained for example by the selection of a natural or induced mutant, or of a somaclonal variant, the selection of a variant individual from plants of the initial variety, backcrossing, or transformation by genetic engineering.

“Plant line” is for example a breeding line which can be used to develop one or more varieties. One embodiment of the present invention refers to a polyploid ALS inhibitor herbicide tolerant plant line comprising at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine, such as a B. napus plant line comprising an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and an ALS III polypeptide comprising at a position corresponding to position 556 of SEQ ID NO: 4 instead of the naturally encoded amino acid tryptophan the amino acid leucine, or such as a B. napus plant line comprising an ALS I polypeptide comprising at a position corresponding to position 559 of SEQ ID NO: 2 instead of the naturally encoded amino acid tryptophan the amino acid leucine, and an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or such as a B. napus plant line comprising an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or such as a B. juncea plant line comprising an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine, and an ALS-B polypeptide comprising at a position corresponding to position 559 of SEQ ID NO: 8 instead of the naturally encoded amino acid tryptophan the amino acid leucine, or such as a B. juncea plant line comprising an ALS-A polypeptide comprising at a position corresponding to position 556 of SEQ ID NO: 6 instead of the naturally encoded amino acid tryptophan the amino acid leucine, and an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine, or such as a B. juncea plant line comprising an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine, and an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine.

A “variety” is used herein in conformity with the UPOV convention and refers to a plant grouping within a single botanical taxon of the lowest known rank, which grouping can be defined by the expression of the characteristics resulting from a given genotype or combination of genotypes, can be distinguished from any other plant grouping by the expression of at least one of the said characteristics and is considered as a unit with regard to its suitability for being propagated unchanged (stable).

“Hybrid” refers to the seeds harvested from crossing one plant line or variety with another plant line or variety.

“F₁ Hybrid” refers to the first generation progeny of the cross of two genetically divergent plants. In one embodiment, such a F₁ Hybrid is homozygous in the essential feature, i.e., said F₁ Hybrid being a hybrid of a polyploid plant comprising at least two ALS genes, wherein all ALS genes encode an ALS polypeptide comprising at a position corresponding to position 574 of SEQ ID NO: 10 instead of the naturally encoded amino acid tryptophan the amino acid leucine and wherein at least one of the ALS genes encodes an ALS polypeptide which further comprises at a position corresponding to position 197 of SEQ ID NO: 10 instead of the naturally encoded amino acid proline the amino acid serine, such as an F₁ B. napus hybrid comprising ALS I alleles encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine and comprising ALS III alleles encoding an ALS III polypeptide comprising at a position corresponding to position 556 of SEQ ID NO: 4 instead of the naturally encoded amino acid tryptophan the amino acid leucine, or such as an F1 B. napus hybrid comprising ALS I alleles encoding an ALS I polypeptide comprising at a position corresponding to position 559 of SEQ ID NO: 2 instead of the naturally encoded amino acid tryptophan the amino acid leucine and comprising ALS III alleles encoding an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or such as an F1 B. napus hybrid comprising ALS I alleles encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine and comprising ALS III alleles encoding an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or such as an F1 B. juncea hybrid comprising ALS-A alleles encoding an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine and comprising ALS-B alleles encoding an ALS-B polypeptide comprising at a position corresponding to position 559 of SEQ ID NO: 8 instead of the naturally encoded amino acid tryptophan the amino acid leucine, or such as an F1 B. juncea hybrid comprising ALS-A alleles encoding an ALS-A polypeptide comprising at a position corresponding to position 556 of SEQ ID NO: 6 instead of the naturally encoded amino acid tryptophan the amino acid leucine and comprising ALS-B alleles encoding an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine, or such as an F1 B. juncea hybrid comprising ALS-A alleles encoding an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine and comprising ALS-B alleles encoding an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine.

“Crossing” refers to the mating of two parent plants.

“Backcrossing” refers to a process in which a breeder repeatedly crosses hybrid progeny, for example a first generation hybrid (F₁), back to one of the parents of the hybrid progeny. Backcrossing can be used to introduce one or more single locus conversions from one genetic background into another.

“Cross-pollination” refers to fertilization by the union of two gametes from different plants.

“Regeneration” refers to the development of a plant from tissue culture.

“Selfing” refers to self-pollination of a plant, i.e., the transfer of pollen from the anther to the stigma of the same plant.

Single Locus Converted (Conversion) Plant: Plants which are developed by a plant breeding technique called backcrossing, wherein essentially all of the desired morphological and physiological characteristics of a oilseed rape variety are recovered in addition to the characteristics of the single locus transferred into the variety via the backcrossing technique and/or by genetic transformation.

Plants of the present invention can be identified using any genotypic analysis method. Genotypic evaluation of the plants includes using techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Allele-specific PCR (AS-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as “Microsatellites”. Additional compositions and methods for analyzing the genotype of the plants provided herein include those methods disclosed in U.S. Publication No. 2004/0171027, U.S. Publication No. 2005/02080506, and U.S. Publication No. 2005/0283858.

### Sequences/Position

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used.

Generally, the skilled person knows, because of his common general knowledge and the context when the terms ALS, ALSL, AHAS or AHASL are used herein as to whether the nucleotide sequence or nucleic acid, or the amino acid sequence or polypeptide, respectively, is meant. The terms acetohydroxyacid synthase, AHAS, acetolactate synthase and ALS are used as interchangeably throughout this text.

The term B. napus “ALS” or “AHAS” gene refers to B. napus nucleotide sequences which are at least 60, 70, 80, 90, 95, 97, 98, 99% or 100% identical to the B. napus ALS nucleotide sequence of SEQ ID NO: 1 or 3.

The term “ALS I” or “AHAS I” gene refers to a B. napus ALS gene present on the C genome, wherein the sequence of said gene is at least 60, 70, 80, 90, 95, 97, 98, 99% or 100% identical to the nucleotide sequence of SEQ ID NO: 1.

The term “ALS III” or “ALS III” gene refers to a B. napus ALS gene present on the A genome, wherein the sequence of said gene is at least 60, 70, 80, 90, 95, 97, 98, 99% or 100% identical to the nucleotide sequence of SEQ ID NO: 3.

The term B. napus “ALS” or “AHAS” polypeptide refers to amino acid sequences which are at least 90, 95, 97, 98, 99% or 100% identical to the ALS amino acid sequence of SEQ ID NO: 2 or 4. Said X % identical amino acid sequences retain the activity of ALS as described herein, more preferably the ALS polypeptide is tolerant to ALS inhibitor herbicides as described herein. However, such “ALS” or “AHAS” polypeptides still show ALS enzymatic activity at a level of at least 20%, 30%, 40%, 50%, 60%, 70%, 80%, 90% compared to the level of the ALS enzymatic activity of an protein having the SEQ ID NO: 2 (when referring to an ALS I protein) or 4 (when referring to an ALS III protein).

The term “ALS I” or “AHAS I” protein refers to the protein encoded by the ALS I gene, wherein said ALS I protein contains at least 90, 95, 97, 98, 99 or 100% sequence identity to the ALS amino acid sequence of SEQ ID NO: 2.

The term “ALS III” or “AHAS III” protein refers to the protein encoded by the ALS III gene, wherein said ALS III protein contains at least 90, 95, 97, 98, 99% or 100% sequence identity to the ALS amino acid sequence of SEQ ID NO: 4.

The term B. juncea “ALS” or “AHAS” gene refers to B. juncea nucleotide sequences which are at least 60, 70, 80, 90, 95, 97, 98, 99% or 100% identical to the B. juncea ALS nucleotide sequence of SEQ ID NO: 5 or 7.

The term “ALS-A” or “AHAS-A” gene refers to a B. juncea ALS gene present on the A genome, wherein the sequence of said gene is at least 60, 70, 80, 90, 95, 97, 98, 99% or 100% identical to the nucleotide sequence of SEQ ID NO: 3.

The term “ALS-B” or “ALS-B” gene refers to a B. juncea ALS gene present on the B genome, wherein the sequence of said gene is at least 60, 70, 80, 90, 95, 97, 98, 99% or 100% identical to the nucleotide sequence of SEQ ID NO: 5.

The term B. juncea “ALS” or “AHAS” polypeptide refers to amino acid sequences which are at least 90, 95, 97, 98, 99% or 100% identical to the ALS amino acid sequence of SEQ ID NO: 6 or 8. Said X % identical amino acid sequences retain the activity of ALS as described herein, more preferably the ALS polypeptide is tolerant to ALS inhibitor herbicides as described herein. However, such “ALS” or “AHAS” polypeptides still show ALS enzymatic activity at a level of at least 20%, 30%, 40%, 50%, 60%, 70%, 80%, 90% compared to the level of the ALS enzymatic activity of an protein having the SEQ ID NO: 6 (when referring to an ALS-A protein) or 8 (when referring to an ALS-B protein).

The term “ALS-A” or “AHAS-A” protein refers to the protein encoded by the ALS-A gene, wherein said ALS-A protein contains at least 90, 95, 97, 98, 99 or 100% sequence identity to the ALS amino acid sequence of SEQ ID NO: 6.

The term “ALS-B” or “AHAS-B” protein refers to the protein encoded by the ALS-B gene, wherein said ALS-B protein contains at least 90, 95, 97, 98, 99% or 100% sequence identity to the ALS amino acid sequence of SEQ ID NO: 8.

It is well known to the skilled person that the genomes of three allotetraploid, or amphidiploid Brassica species B. napus, B. juncea and B. carinata are derived from three ancestral genomes, denoted by the letters AA (derived from B. rapa); BB (derived from B. nigra), and CC (derived from B. oleracea). B. napus contains an A genome and a C genome; B. juncea contains an A genome and a B genome, and B. carinata contains a B genome and a C genome. The ALS gene on a given genome is therefore essentially similar when present in different species. Thus, the ALS gene from the A genome (ALS III from B. napus or ALS-A from B. juncea) is therefore essentially similar in B. napus, B. juncea and B. rapa. The ALS gene from the B genome (ALS-B from B. juncea) is essentially similar in B. juncea, B. carinata, and B. nigra. The ALS gene from the C genome (ALS I from B. napus) is essentially similar in B. napus, B. carinata and B. oleracea. Also provided are therefore B. napus plants comprising ALS genes essentially similar to ALS-A and to ALS I, B. juncea plants comprising ALS genes essentially similar to ALS III and ALS-B, and B. carinata plants comprising ALS genes essentially similar to ALS-B and ALS I.

Essentially similar as used herein refers to having at least 90, 95, 97, 98, 99% or 100% sequence identity to the sequence referred to.

The term “position” when used in accordance with the present invention means the position of either an amino acid within an amino acid sequence depicted herein or the position of a nucleotide within a nucleotide sequence depicted herein. The term “corresponding” as used herein also includes that a position is not only determined by the number of the preceding nucleotides/amino acids.

The position of a given nucleotide in accordance with the present invention which may be substituted may vary due to deletions or additional nucleotides elsewhere in the ALS 5′-untranslated region (UTR) including the promoter and/or any other regulatory sequences or gene (including exons and introns). Similarly, the position of a given amino acid in accordance with the present invention which may be substituted may vary due to deletion or addition of amino acids elsewhere in the ALS polypeptide.

Thus, under a “corresponding position” or “a position corresponding to position” in accordance with the present invention it is to be understood that nucleotides/amino acids may differ in the indicated number but may still have similar neighbouring nucleotides/amino acids. Said nucleotides/amino acids which may be exchanged, deleted or added are also comprised by the term “corresponding position”.

In order to determine whether a nucleotide residue or amino acid residue in a given ALS nucleotide/amino acid sequence corresponds to a certain position in the nucleotide sequence of SEQ ID NO: 1, 3, 5, 7 or 9, respectively, or their corresponding amino acid sequences of SEQ ID NO: 2, 4, 6, 8 or 10, respectively, the skilled person can use means and methods well-known in the art, e.g., alignments, either manually or by using computer programs such as BLAST (Altschul et al. (1990), Journal of Molecular Biology, 215, 403-410), which stands for Basic Local Alignment Search Tool or ClustalW (Thompson et al. (1994), Nucleic Acid Res., 22, 4673-4680) or any other suitable program which is suitable to generate sequence alignments.

SEQ ID NO: 1 is the nucleotide sequence encoding a B. napus wild type ALS I, whereas SEQ ID NO: 2 is the B. napus amino acid sequence derived from SEQ ID NO: 1. Accordingly, the codon at position 544-546 of the nucleotide sequence of SEQ ID NO: 1 encodes the amino acid at position 182 of SEQ ID NO: 2 (this position, again, corresponds to position 197 of SEQ ID NO: 10), whereas the codon at position 1675-1677 of the nucleotide sequence of SEQ ID NO: 1 encodes the amino acid at position 559 of SEQ ID NO: 2 (this position, again, corresponds to position 574 of SEQ ID NO: 10). In other words, the amino acid proline (“Pro” (three letter code) or “P” (one letter code)) of SEQ ID NO: 2 is encoded by the codon at positions 544-546 of the nucleotide sequence of SEQ ID NO: 1, and the amino acid tryptophan (“Trp” (three letter code) or “W” (one letter code)) of SEQ ID NO: 2 is encoded by the codon at positions 1675-1677 of the nucleotide sequence of SEQ ID NO: 1.

SEQ ID NO: 3 is the nucleotide sequence encoding a B. napus wild type ALS III, whereas SEQ ID NO: 4 is the B. napus amino acid sequence derived from SEQ ID NO: 3. Accordingly, the codon at position 535-537 of the nucleotide sequence of SEQ ID NO: 3 encodes the amino acid at position 179 of SEQ ID NO: 4 (this position, again, corresponds to position 197 of SEQ ID NO: 10), whereas the codon at position 1666-1668 of the nucleotide sequence of SEQ ID NO: 3 encodes the amino acid at position 556 of SEQ ID NO: 4 (this position, again, corresponds to position 574 of SEQ ID NO: 10). In other words, the amino acid proline (“Pro” (three letter code) or “P” (one letter code)) of SEQ ID NO: 4 is encoded by the codon at positions 535-537 of the nucleotide sequence of SEQ ID NO: 3, and the amino acid tryptophan (“Trp” (three letter code) or “W” (one letter code)) of SEQ ID NO: 4 is encoded by the codon at positions 1666-1668 of the nucleotide sequence of SEQ ID NO: 3.

SEQ ID NO: 5 is the nucleotide sequence encoding a B. juncea wild type ALS-A, whereas SEQ ID NO: 6 is the B. juncea amino acid sequence derived from SEQ ID NO: 5. Accordingly, the codon at position 535-537 of the nucleotide sequence of SEQ ID NO: 5 encodes the amino acid at position 179 of SEQ ID NO: 6 (this position, again, corresponds to position 197 of SEQ ID NO: 10), whereas the codon at position 1666-1668 of the nucleotide sequence of SEQ ID NO: 5 encodes the amino acid at position 556 of SEQ ID NO: 6 (this position, again, corresponds to position 574 of SEQ ID NO: 10). In other words, the amino acid proline (“Pro” (three letter code) or “P” (one letter code)) of SEQ ID NO: 6 is encoded by the codon at positions 535-537 of the nucleotide sequence of SEQ ID NO: 5, and the amino acid tryptophan (“Trp” (three letter code) or “W” (one letter code)) of SEQ ID NO: 6 is encoded by the codon at positions 1666-1668 of the nucleotide sequence of SEQ ID NO: 5.

SEQ ID NO: 7 is the nucleotide sequence encoding a B. juncea wild type ALS-B, whereas SEQ ID NO: 8 is the B. juncea amino acid sequence derived from SEQ ID NO: 7. Accordingly, the codon at position 544-546 of the nucleotide sequence of SEQ ID NO: 7 encodes the amino acid at position 182 of SEQ ID NO: 8 (this position, again, corresponds to position 197 of SEQ ID NO: 10), whereas the codon at position 1675-1677 of the nucleotide sequence of SEQ ID NO: 7 encodes the amino acid at position 559 of SEQ ID NO: 8 (this position, again, corresponds to position 574 of SEQ ID NO: 10). In other words, the amino acid proline (“Pro” (three letter code) or “P” (one letter code)) of SEQ ID NO: 8 is encoded by the codon at positions 544-546 of the nucleotide sequence of SEQ ID NO: 7, the amino acid tryptophan (“Trp” (three letter code) or “W” (one letter code)) of SEQ ID NO: 8 is encoded by the codon at positions 1675-1677 of the nucleotide sequence of SEQ ID NO: 7.

In the alternative to determine whether a nucleotide residue or amino acid residue in a given ALS nucleotide/amino acid sequence corresponds to a certain position in the nucleotide sequence of SEQ ID NO: 1, 3, 5, or 7, respectively, the nucleotide sequence encoding A. thaliana wild type ALS shown in SEQ ID NO: 9 can be used. SEQ ID NO: 10 is the A. thaliana amino acid sequence derived from SEQ ID NO: 9.

The codon at position 589-591 of the nucleotide sequence of SEQ ID NO: 9 encodes the amino acid at position 197 of SEQ ID NO: 10, whereby position 197 of SEQ ID NO: 10 corresponds to position 182 of SEQ ID NOs: 2 and 8 and corresponds to position 179 of SEQ ID NOs: 4 and 6, and the codon at position 1720-1722 of the nucleotide sequence of SEQ ID NO: 9 encodes the amino acid at position 574 of SEQ ID NO: 10, whereby position 574 of SEQ ID NO: 10 corresponds to position 559 of SEQ ID NOs: 2 and 8 and corresponds to position 556 of SEQ ID NOs: 4 and 6.

If the A. thaliana wild type ALS nucleotide sequence shown in SEQ ID NO: 9 is used as reference sequence (as it is done in most of the relevant literature and, therefore, is used to enable an easier comparison to such known sequences), the codon encoding a serine instead of a proline at position 182 of SEQ ID NO: 2 and SEQ ID NO: 8 is at a position 544-546 of SEQ ID NO: 1 and SEQ ID NO: 7, respectively, which corresponds to position 589-591 of SEQ ID NO: 9, the codon encoding a serine instead of a proline at a position 179 of SEQ ID NO: 4 and SEQ ID NO: 6 is at a position 535-537 of SEQ ID NO: 3 and SEQ ID NO: 5, respectively, which corresponds to position 589-591 of SEQ ID NO: 9. If the A. thaliana wild type ALS nucleotide sequence shown in SEQ ID NO: 9 is used as reference sequence, the codon encoding a leucine instead of a tryptophan at position 559 of SEQ ID NO: 2 and SEQ ID NO: 8 is at a position 1675-1677 of SEQ ID NO: 1 and SEQ ID NO: 7, respectively, which corresponds to position 1720-1722 of SEQ ID NO: 9, the codon encoding a leucine instead of a tryptophan at a position 556 of SEQ ID NO: 4 and SEQ ID NO: 6 is at a position 1666-1668 of SEQ ID NO: 3 and SEQ ID NO: 5, respectively, which corresponds to position 1720-1722 of SEQ ID NO: 9.

However, SEQ ID NO: 1 is preferred as the reference nucleotide sequence for mutated ALS I protein encoding sequences, and SEQ ID NO: 2 is preferred as the reference amino acid sequence for mutated sequences in all of the subsequent disclosures.

Similarly, SEQ ID NO: 3 is preferred as the reference nucleotide sequence for mutated ALS III protein encoding sequences and SEQ ID NO: 4 is preferred as the reference amino acid sequence for mutated sequences in all of the subsequent disclosures.

Similarly, SEQ ID NO: 5 is preferred as the reference nucleotide sequence for mutated ALS-A protein encoding sequences, and SEQ ID NO: 6 is preferred as the reference amino acid sequence for mutated sequences in all of the subsequent disclosures.

Similarly, SEQ ID NO: 7 is preferred as the reference nucleotide sequence for mutated ALS-B protein encoding sequences and SEQ ID NO: 8 is preferred as the reference amino acid sequence for mutated sequences in all of the subsequent disclosures.

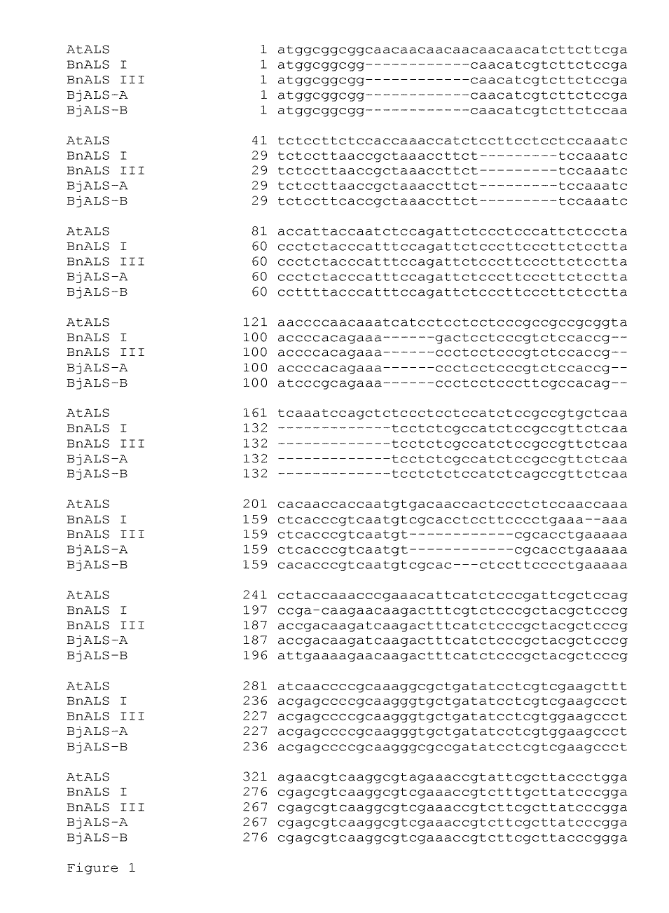

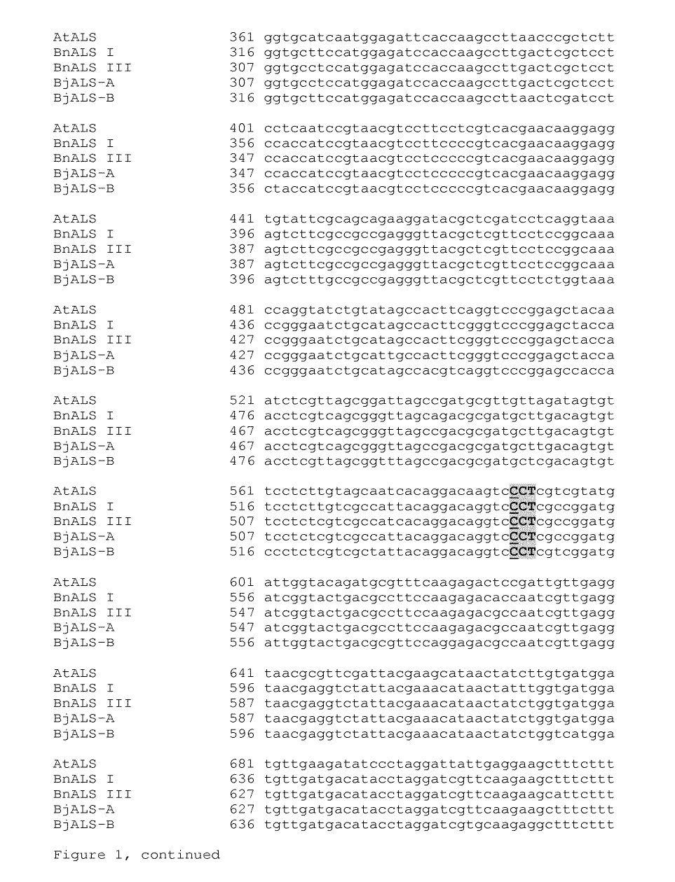

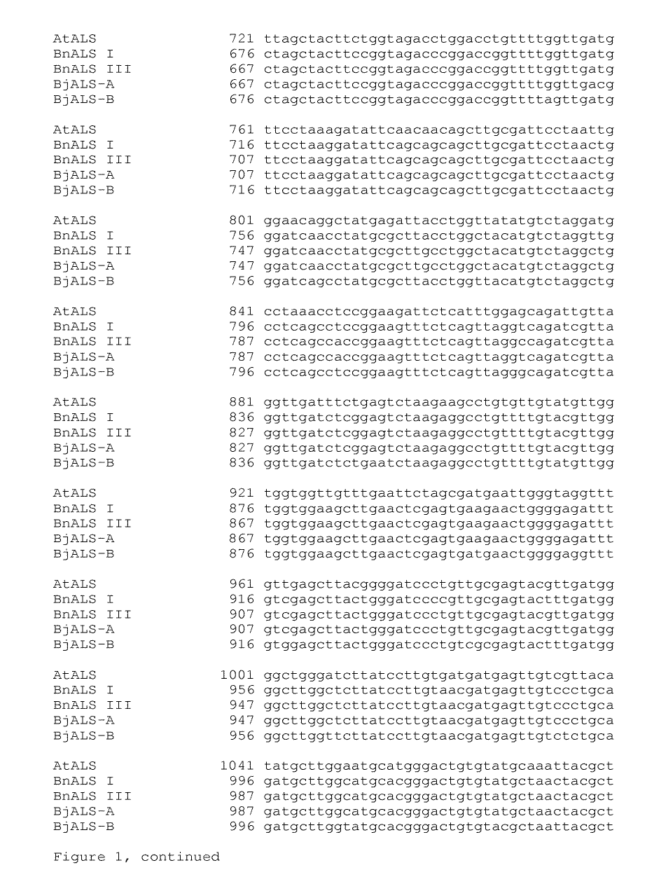

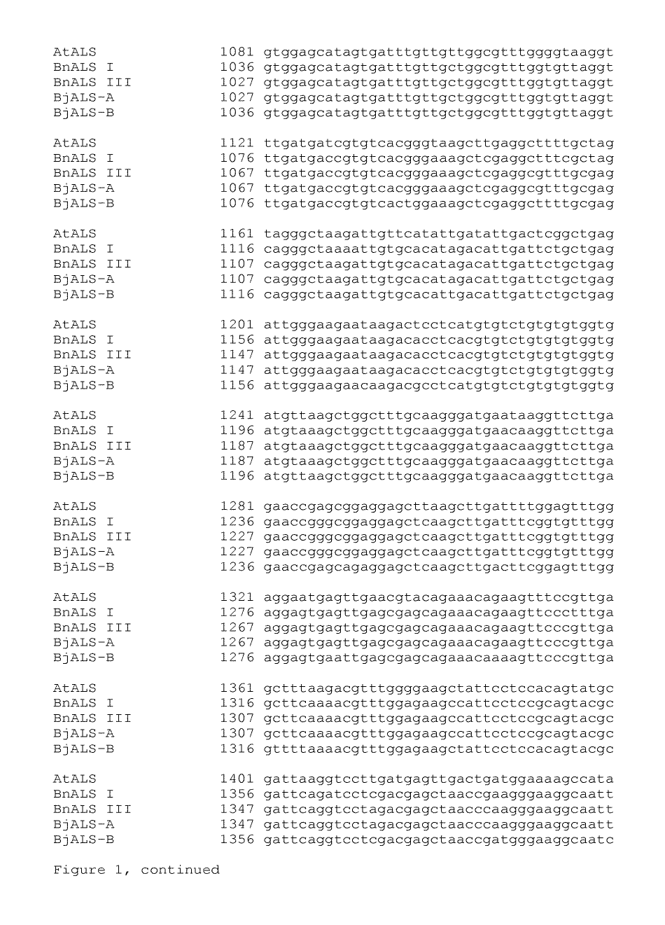

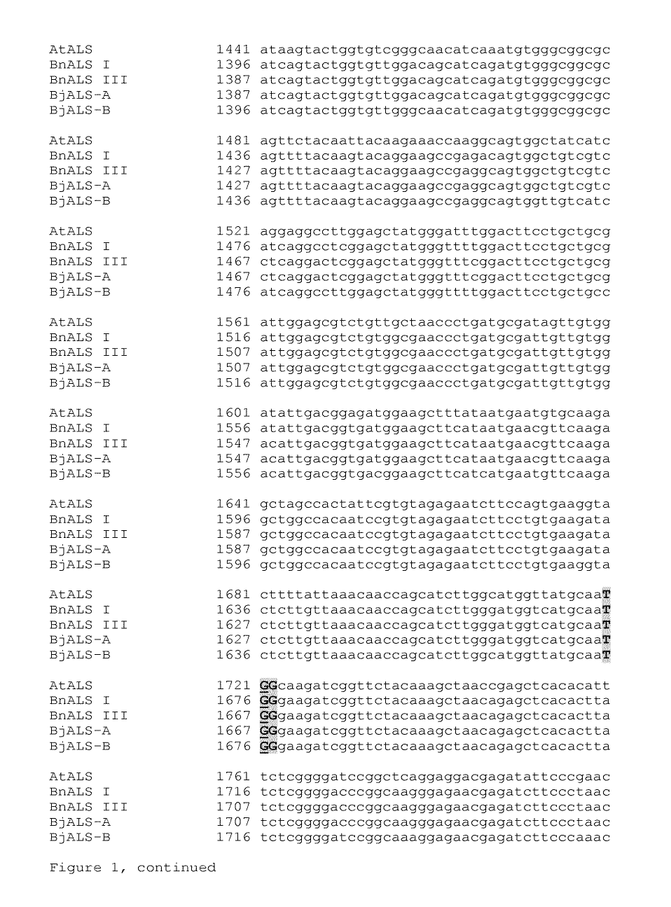

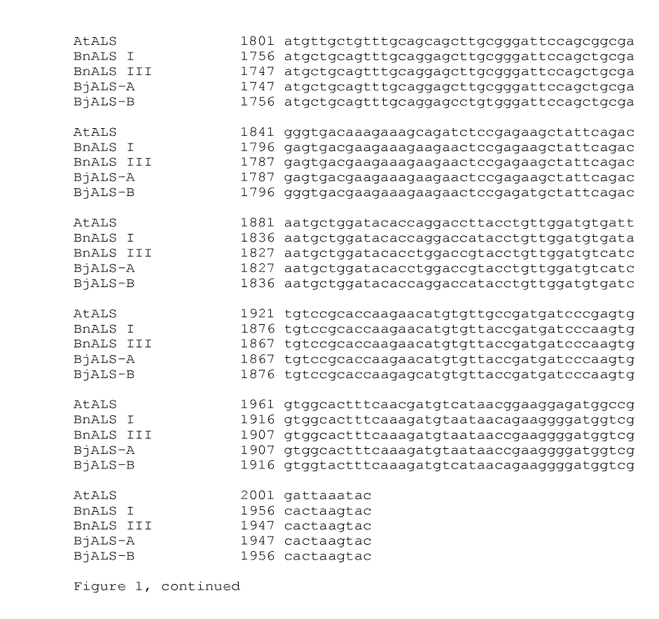

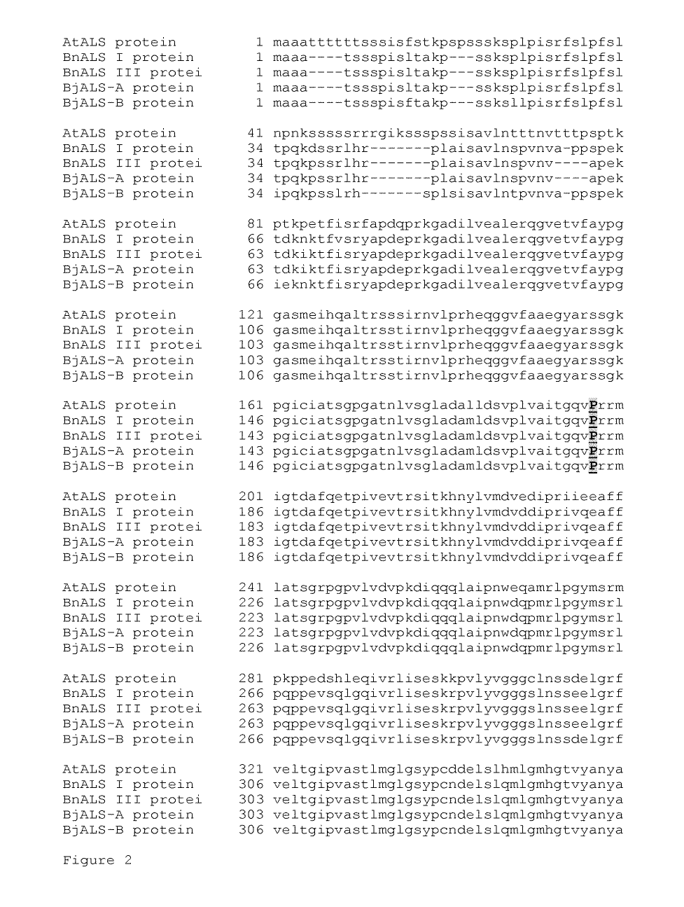

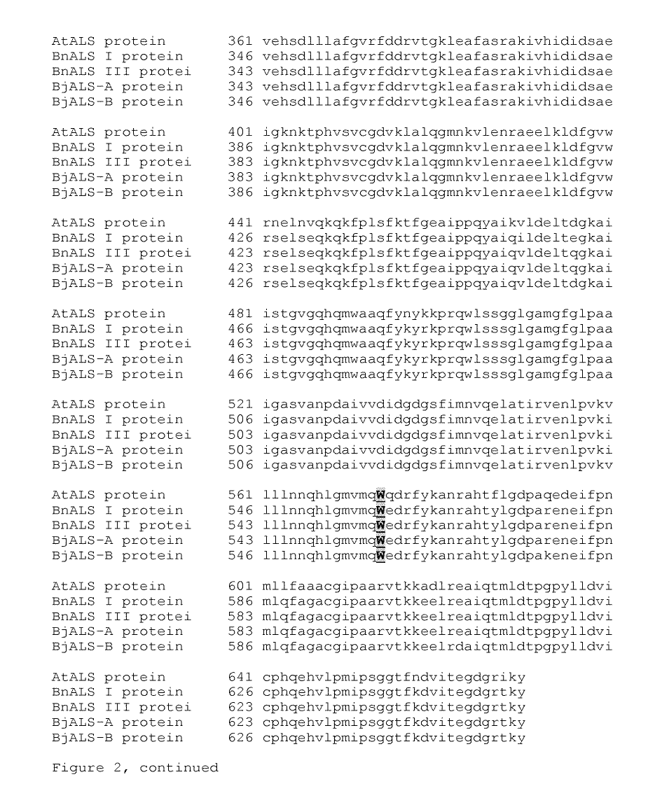

Thus, in any event, the equivalent position can still be determined through alignment with a reference sequence, such as SEQ ID NO: 1, 3, 5 or 7 (nucleotide sequence) or SEQ ID NO: 2, 4, 6 or 8 (amino acid sequence). Alignments of the various sequences listed above are given in FIGS. 1 and 2.

In view of the difference between the wild-type ALS genes (such as the B. napus ALS I and III gene, and the B. juncea ALS-A and ALS-B gene) and the mutated ALS genes comprised by a plant of the present invention or progeny thereof, the ALS genes (or polynucleotides or nucleotide sequences) comprised by a plant of the present invention or progeny thereof may also be regarded as a “mutant ALS gene”, “mutant ALS allele”, “mutant ALS polynucleotide” or the like. Thus, throughout the specification, the terms “mutant allele”, “mutant ALS allele”, “mutant ALS gene” or “mutant ALS polynucleotide” are used interchangeably.

Unless indicated otherwise herein, these terms refer to a nucleotide sequence encoding an ALS protein that comprises a codon at a position which corresponds to position 1720-1722 of the Arabidopsis ALS gene of SEQ ID NO: 9, and said codon encodes a leucine instead of a tryptophan and, optionally, that further comprises a codon at a position which corresponds to position 589-591 of the Arabidopsis ALS gene of SEQ ID NO: 9, and said codon encodes a serine instead of a proline, such as a nucleotide sequence encoding an ALS I protein that comprises a codon at a position which corresponds to position 1675-1677 of SEQ ID NO: 1 and said codon encodes a leucine instead of a tryptophan and, optionally, further comprises comprises a codon at a position which corresponds to position 544-546 of SEQ ID NO: 1 and said codon encodes a serine instead of a proline; to a nucleotide sequence encoding for an ALS III protein that comprises a codon at a position which corresponds to position 1666-1668 of SEQ ID NO: 3 and said codon of said second nucleotide sequence encodes a leucine instead of a tryptophan and, optionally, further comprises comprises a codon at a position which corresponds to position 535-537 of SEQ ID NO: 3 and said codon encodes a serine instead of a proline; to a nucleotide sequence encoding for an ALS-A protein that comprises a codon at a position which corresponds to position 1666-1668 of SEQ ID NO: 5 and said codon of said second nucleotide sequence encodes a leucine instead of a tryptophan and, optionally, further comprises comprises a codon at a position which corresponds to position 535-537 of SEQ ID NO: 5 and said codon encodes a serine instead of a proline; and to a nucleotide sequence encoding an ALS-B protein that comprises a codon at a position which corresponds to position 1675-1677 of SEQ ID NO: 7 and said codon encodes a leucine instead of a tryptophan and, optionally, further comprises comprises a codon at a position which corresponds to position 544-546 of SEQ ID NO: 7 and said codon encodes a serine instead of a proline.

The term “P197S mutation” refers to a mutation in the codon corresponding to nt 589-591 in A. thaliana (SEQ ID NO 9) leading to a substitution of the amino acid proline by a serine.

The term “P197S mutation” in ALS I refers to a mutation in the codon corresponding to nt 589-591 in A. thaliana (SEQ ID NO 9) or in the codon corresponding to nt 544-546 of B. napus ALS I (SEQ ID NO: 1) leading to a substitution of the amino acid proline by a serine.

The term “P197S mutation” in ALS III refers to a mutation in the codon corresponding to nt 589-591 in A. thaliana (SEQ ID NO 9) or in the codon corresponding to nt 535-537 of B. napus ALS III (SEQ ID NO: 3) leading to a substitution of the amino acid proline by a serine.

The term “P197S mutation” in ALS-A refers to a mutation in the codon corresponding to nt 589-591 in A. thaliana (SEQ ID NO 9) or in the codon corresponding to nt 535-537 of B. juncea ALS-A (SEQ ID NO: 5) leading to a substitution of the amino acid proline by a serine.

The term “P197S mutation” in ALS-B refers to a mutation in the codon corresponding to nt 589-591 in A. thaliana (SEQ ID NO 9) or in the codon corresponding to nt 544-546 of B. juncea ALS-A (SEQ ID NO: 7) leading to a substitution of the amino acid proline by a serine.

The term “W574L mutation” refers to a mutation in the codon corresponding to nt 1720-1722 in A. thaliana (SEQ ID NO 9) leading to a substitution of the amino acid tryptophan by a leucine.

The term “W574L mutation” in ALS I refers to a mutation in the codon corresponding to nt 1720-1722 in A. thaliana (SEQ ID NO 9) or in the codon corresponding to nt 1675-1677 of B. napus ALS I (SEQ ID NO: 1) leading to a substitution of the amino acid tryptophan by a leucine.

The term “W574L mutation” in ALS III refers to a mutation in the codon corresponding to nt 1720-1722 in A. thaliana (SEQ ID NO 9) or in the codon corresponding to nt 1666-1668 of B. napus ALS III (SEQ ID NO: 3) leading to a substitution of the amino acid tryptophan by a leucine.

The term “W574L mutation” in ALS-A refers to a mutation in the codon corresponding to nt 1720-1722 in A. thaliana (SEQ ID NO 9) or in the codon corresponding to nt 1666-1668 of B. juncea ALS-A (SEQ ID NO: 5) leading to a substitution of the amino acid tryptophan by a leucine.

The term “W574L mutation” in ALS-B refers to a mutation in the codon corresponding to nt 1720-1722 in A. thaliana (SEQ ID NO 9) or in the codon corresponding to nt 1675-1677 of B. juncea ALS-A (SEQ ID NO: 7) leading to a substitution of the amino acid tryptophan by a leucine.

The terms “nucleotide sequence(s)”, “polynucleotide(s)”, “nucleic acid sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

### Homology/Identity

In order to determine whether a nucleic acid sequence has a certain degree of identity to the nucleotide sequences of the present invention, the skilled person can use means and methods well-known in the art, e.g., alignments, either manually or by using computer programs such as those mentioned further down below in connection with the definition of the term “hybridization” and degrees of homology.

For the purpose of this invention, the “sequence identity” or “sequence homology” (the terms are used interchangeably herein) of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

The term “ALS” or “AHAS” gene also includes nucleotide sequences which are at least 60, 70, 80, 90, 95, 97, 98, 99% or 100% identical to the ALS nucleotide sequences as described herein, wherein these 60, 70, 80, 90, 95, 97, 98, 99, or 100% identical nucleotide sequences comprise at a position corresponding to position 1720-1722 of the nucleotide sequence of SEQ ID NO: 9 a codon encoding Leu instead of Trp (at a position corresponding to position 574 of SEQ ID NO: 10) and, optionally, comprise at a position corresponding to position 589-591 of the nucleotide sequence of SEQ ID NO: 9 a codon encoding Ser instead of Pro (at a position corresponding to position 197 of SEQ ID NO: 10).

The term B. napus “ALS” or “AHAS” gene also includes B. napus nucleotide sequences which are at least 60, 70, 80, 90, 95, 97, 98, 99% or 100% identical to the B. napus ALS nucleotide sequence of SEQ ID NO: 1 or 3, wherein these 60, 70, 80, 90, 95, 97, 98, 99, or 100% identical nucleotide sequences comprise at a position corresponding to position 1675-1677 of the nucleotide sequence of SEQ ID NO: 1 a codon encoding Leu instead of Trp (at position 559 of SEQ ID NO: 2) or at a position corresponding to position 1666-1668 of the nucleotide sequence of SEQ ID NO: 3 a codon encoding Leu instead of Trp (at position 556 of SEQ ID NO: 4) and, optionally, comprise at a position corresponding to position 544-546 of the nucleotide sequence of SEQ ID NO: 1 a codon encoding Ser instead of Pro (at position 182 of SEQ ID NO: 2) or at a position corresponding to position 535-537 of the nucleotide sequence of SEQ ID NO: 3 a codon encoding Ser instead of Pro (at position 179 of SEQ ID NO: 4).

The term B. juncea “ALS” or “AHAS” gene also includes B. juncea nucleotide sequences which are at least 60, 70, 80, 90, 95, 97, 98, 99% or 100% identical to the B. juncea ALS nucleotide sequence of SEQ ID NO: 5 or 7, wherein these 60, 70, 80, 90, 95, 97, 98, 99, or 100% identical nucleotide sequences comprise at a position corresponding to position 1666-1668 of the nucleotide sequence of SEQ ID NO: 5 a codon encoding Leu instead of Trp (at position 556 of SEQ ID NO: 6) or at a position corresponding to position 1675-1677 of the nucleotide sequence of SEQ ID NO: 7 a codon encoding Leu instead of Trp (at position 559 of SEQ ID NO: 8) and, optionally, comprise at a position corresponding to position 535-537 of the nucleotide sequence of SEQ ID NO: 5 a codon encoding Ser instead of Pro (at position 179 of SEQ ID NO: 6) or at a position corresponding to position 544-546 of the nucleotide sequence of SEQ ID NO: 7 a codon encoding Ser instead of Pro (at position 182 of SEQ ID NO: 8).

Likewise, these at least 60, 70, 80, 90, 95, 97, 98, 99, or 100% identical nucleotide sequences include sequences encoding an ALS polypeptide comprising at a position corresponding to position 197 of SEQ ID NO: 10 Ser instead of Pro and at a position corresponding to position 574 of SEQ ID NO: 10 Leu instead of Trp, or at a position corresponding to position 182 of SEQ ID NO: 2 or of SEQ ID NO: 8 Ser instead of Pro and at a position corresponding to position 559 of SEQ ID NO: 2 or of SEQ ID NO: 8 Leu instead of Trp, or at a position corresponding to position 179 of SEQ ID NO: 4 or of SEQ ID NO: 6 Ser instead of Pro and at a position corresponding to position 556 of SEQ ID NO: 4 or of SEQ ID NO: 6 Leu instead of Trp, and include sequences encoding an ALS polypeptide comprising at a position corresponding to position 574 of SEQ ID NO: 10 Leu instead of Trp, or at a position corresponding to position 559 of SEQ ID NO: 2 or of SEQ ID NO: 8 Leu instead of Trp, or at a position corresponding to position 556 of SEQ ID NO: 4 or of SEQ ID NO: 6 Leu instead of Trp. Of course, these nucleotide sequences encode for ALS proteins which retain the activity as described herein, more preferably the thus-encoded ALS polypeptide is tolerant to one or more ALS inhibitor herbicides as described herein. Said term also includes allelic variants and homologs encoding an ALS polypeptide which is preferably tolerant to one or more ALS inhibitor herbicides as described herein.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature. The polypeptide (or protein) that are preferably meant herein have an amino acid sequence that comprises the mutated ALS polypeptides, such as B. napus ALS I and III polypeptides (or ALS I and III proteins) of SEQ ID NO: 2 and 4, of which the tryptophan at a position corresponding to position 574 of SEQ ID NO: 10 is substituted with a leucine, and, optionally, of which the proline at a position corresponding to position 197 of SEQ ID NO: 10 is substituted with a serine, respectively, and such as the B. juncea ALS-A and -B polypeptides of SEQ ID NO: 6 and 8, of which the tryptophan at a position corresponding to position 574 of SEQ ID NO: 10 is substituted with a leucine, and, optionally, of which the proline at a position corresponding to position 197 of SEQ ID NO: 10 is substituted with a serine, respectively.

The term “ALS” or “AHAS” polypeptide also includes amino acid sequences which comprise an amino acid sequences which is at least 90, 95, 97, 98, 99% or 100% identical to the ALS amino acid sequences as described herein, wherein these at least 90, 95, 97, 98, 99 or 100% identical amino acid sequences comprising at a position corresponding to position 574 of SEQ ID NO: 10 a leucine instead of a tryptophan, or wherein these at least 90, 95, 97, 98, 99 or 100% identical amino acid sequences comprising at a position corresponding to position 197 of SEQ ID NO: 10 a serine instead of a proline and at a position corresponding to position 574 of SEQ ID NO: 10 a leucine instead of a tryptophan. Said X % identical amino acid sequences retain the activity of ALS as described herein, more preferably the ALS polypeptide is tolerant to ALS inhibitor herbicides as described herein. However, such “ALS” or “AHAS” polypeptides still show ALS activity of at least 20%, 30%, 40%, 50%, 60%, 70%, 80%, 90% compared to ALS activity of a protein having the SEQ ID NO: 10.

The term B. napus “ALS” or “AHAS” polypeptide also includes amino acid sequences which comprise an amino acid sequences which is at least 90, 95, 97, 98, 99% or 100% identical to the ALS amino acid sequence of SEQ ID NO: 2 or 4, wherein these at least 90, 95, 97, 98, 99 or 100% identical amino acid sequences comprising at a position corresponding to position 559 of SEQ ID NO: 2 a leucine instead of a tryptophan, and at a position corresponding to position 556 of SEQ ID NO: 4 a leucine instead of a tryptophan, or wherein these at least 90, 95, 97, 98, 99 or 100% identical amino acid sequences comprising at a position corresponding to position 182 of SEQ ID NO: 2 a serine instead of a proline and at a position corresponding to position 559 of SEQ ID NO: 2 a leucine instead of a tryptophan, and at a position corresponding to position 179 of SEQ ID NO: 4 a serine instead of a proline and at a position corresponding to position 556 of SEQ ID NO: 4 a leucine instead of a tryptophan. Said X % identical amino acid sequences retain the activity of ALS as described herein, more preferably the ALS polypeptide is tolerant to ALS inhibitor herbicides as described herein. However, such “ALS” or “AHAS” polypeptides still show ALS activity of at least 20%, 30%, 40%, 50%, 60%, 70%, 80%, 90% compared to ALS activity of an protein having the SEQ ID NO: 2 (when referring to an ALS I protein) or 4 (when referring to an ALS III protein).

The term B. juncea “ALS” or “AHAS” polypeptide also includes amino acid sequences which comprise an amino acid sequences which is at least 90, 95, 97, 98, 99% or 100% identical to the ALS amino acid sequence of SEQ ID NO: 6 or 8, wherein these at least 90, 95, 97, 98, 99 or 100% identical amino acid sequences comprising at a position corresponding to position 556 of SEQ ID NO: 6 a leucine instead of a tryptophan, and at a position corresponding to position 559 of SEQ ID NO: 8 a leucine instead of a tryptophan, or wherein these at least 90, 95, 97, 98, 99 or 100% identical amino acid sequences comprising at a position corresponding to position 179 of SEQ ID NO: 6 a serine instead of a proline and at a position corresponding to position 556 of SEQ ID NO: 6 a leucine instead of a tryptophan, and at a position corresponding to position 182 of SEQ ID NO: 8 a serine instead of a proline and at a position corresponding to position 559 of SEQ ID NO: 8 a leucine instead of a tryptophan. Said X % identical amino acid sequences retain the activity of ALS as described herein, more preferably the ALS polypeptide is tolerant to ALS inhibitor herbicides as described herein. However, such “ALS” or “AHAS” polypeptides still show ALS activity of at least 20%, 30%, 40%, 50%, 60%, 70%, 80%, 90% compared to ALS activity of an protein having the SEQ ID NO: 6 (when referring to an ALS-A protein) or 8 (when referring to an ALS-B protein).

The same techniques, e.g., BLAST, as described above for the alignment of nucleic acid sequences can be used for alignments of protein sequences as well. For Example, a BLAST search can be perdormed from those skilled in the art using ExPASy (see world wide net: http://expasy.org/tools/).

Sequences encoding AHAS polypeptides from other plant species, in particular from polyploid plant species such as wheat, cotton (Gossypium hirsutum), potato, alfalfa, sugar cane, soybeans, leek, tobacco, peanut, kinnow, pelargonium, chrysanthemum, triticale, oat, kiwifruit, strawberry, dahlia, pansies, oca, tulips, lilies, daylilies, apple, banana, citrus, coffee and watermelon, can be identified based on the available sequences as described herein. For example, AHAS sequences from these species can be identified by alignments with sequences from sequence databases, such as by using computer programs such as BLAST (Altschul et al. (1990), Journal of Molecular Biology, 215, 403-410), which stands for Basic Local Alignment Search Tool or ClustalW (Thompson et al. (1994), Nucleic Acid Res., 22, 4673-4680) or any other suitable program which is suitable to generate sequence alignments. Further, the skilled person can identify sequences encoding AHAS polypeptides form other plant species using hybridization using as probes the AHAS nucleotide sequences of parts thereof.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

Other sequences encoding AHAS polypeptides from other plant species may also be obtained by DNA amplification using oligonucleotides specific for genes encoding AHAS as primers, such as but not limited to oligonucleotides comprising or consisting of about 20 to about 50 consecutive nucleotides from the known nucleotide sequences or their complement.

Examples of sequences of AHAS polypeptides and sequences encoding these AHAS polypeptides of polyploid plant species are the Gossypium hirsutum AHAS sequences having GenBank number Z46959 (SEQ ID NO: 11 and 12), GenBank number Z46960 (SEQ ID NO: 13 and 14), the Glycine max AHAS sequences having GenBank number FJ581423 (SEQ ID NO: 15 and 16), NCBI Reference Sequence XM_003545859 (SEQ ID NO: 17), NCBI Reference Sequence: XP_003545907 (SEQ ID NO: 18), NCBI Reference Sequence XM_003528058 (SEQ ID NO: 19), NCBI Reference Sequence XP_003528106 (SEQ ID NO: 20), EnsemblPlants number GLYMA04G37270.1 (SEQ ID NO: 21 and 22), the Nicotiana tabacum AHAS sequences having GenBank number FJ649655 (SEQ ID NO: 23 and 24), ENA number CBV23149 (SEQ ID NO: 25), UniProtKB/Swiss-Prot number P09342.1 (SEQ ID NO: 26), and the Solanum tuberosum sequences having GenBank number HM114275 (SEQ ID NO: 27 and 28), EnsemblPlants number PGSC0003DMT400018236 (SEQ ID NO: 29), Uniprot_TrEMBL number M1AAA1_SOLTU (SEQ ID NO: 30), EnsemblPlants number PGSC0003DMG400013027 (SEQ ID NO: 31 and 32). Partial AHAS sequences of Triticum aestivum are sequences having GenBank number AY210406.1 (SEQ ID NO: 33 and 34) and GenBank number AY210405.1 (SEQ ID NO: 35 and 36). Partial AHAS sequences of Saccharum officinarum are the sequence having GenBank number EU243998.1 (SEQ ID NO: 37 and 38).

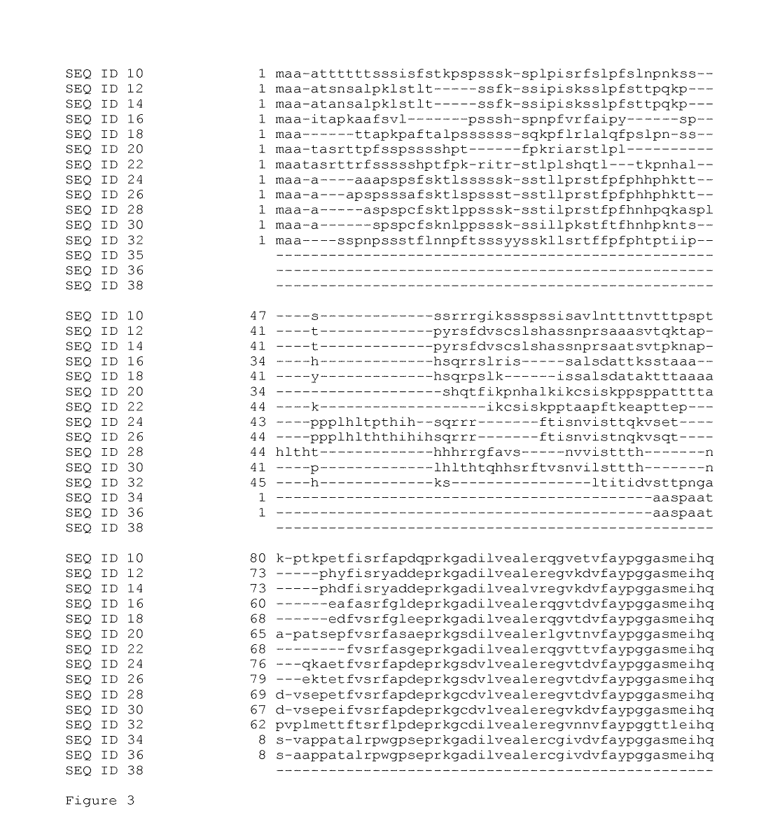

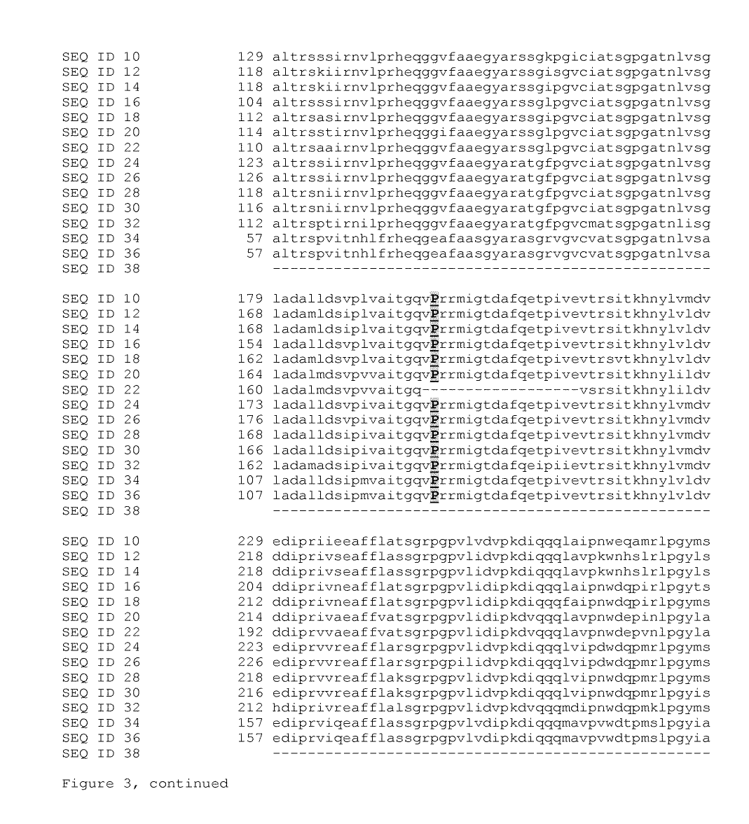

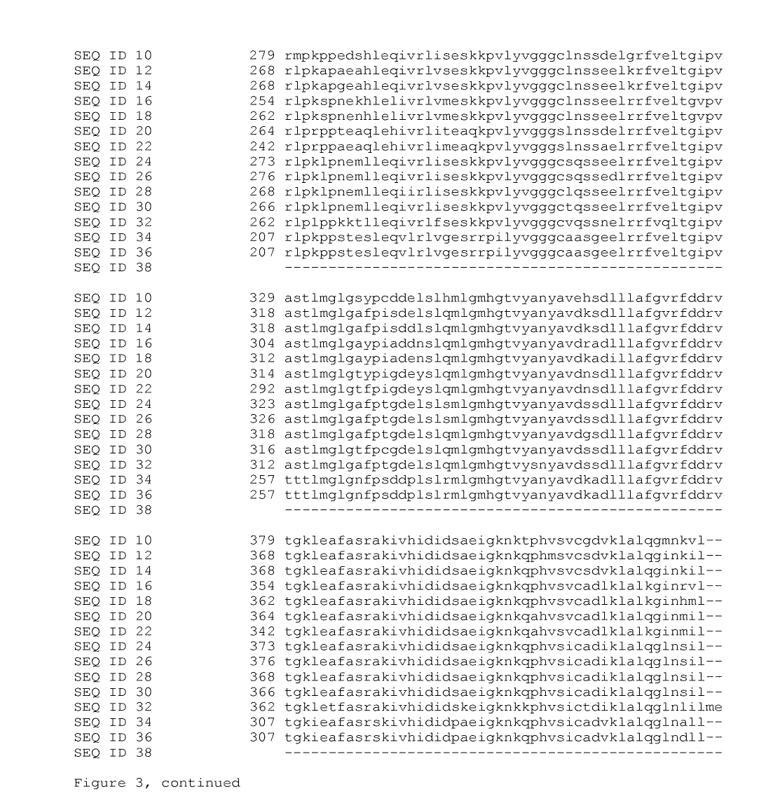

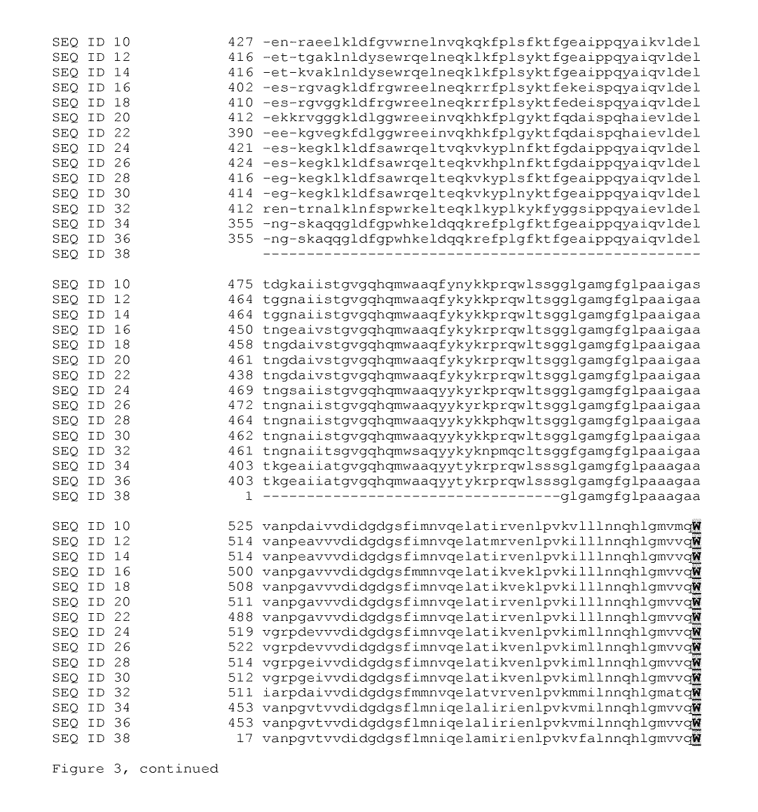

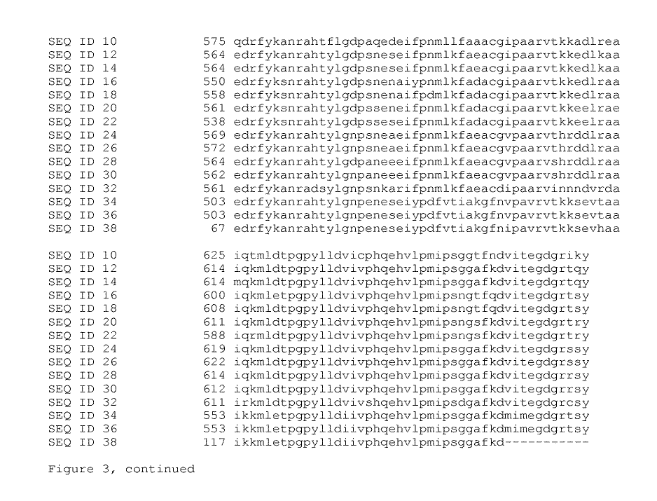

The plants according to the invention comprise at least two AHAS genes encoding an AHAS polypeptide in which the Tryptophan at a position corresponding to position 574 of SEQ ID NO: 10 is substituted with Leucine and and wherein at least one of the ALS genes encodes an ALS polypeptide which further comprises at a position corresponding to position 197 of SEQ ID NO: 10 instead of the naturally encoded amino acid proline the amino acid serine, such as plants comprising at least two AHAS genes encoding an AHAS polypeptide in which the Tryptophan at a position corresponding to position 574 of SEQ ID NO: 10 is substituted with Leucine and wherein one of the ALS genes encodes an ALS polypeptide which further comprises at a position corresponding to position 197 of SEQ ID NO: 10 instead of the naturally encoded amino acid proline the amino acid serine, or such as plants comprising at least two AHAS genes encoding an AHAS polypeptide in which the Tryptophan at a position corresponding to position 574 of SEQ ID NO: 10 is substituted with Leucine and the Proline at a position corresponding to position 197 of SEQ ID NO: 10 is substituted with Serine. In order to determine whether a nucleotide residue or amino acid residue in a given ALS nucleotide/amino acid sequence corresponds to a certain position in the nucleotide sequence of SEQ ID NO: 9, or the corresponding amino acid sequences of SEQ ID 10, respectively, the skilled person can use means and methods well-known in the art, e.g., alignments, either manually or by using computer programs such as BLAST (Altschul et al. (1990), Journal of Molecular Biology, 215, 403-410), which stands for Basic Local Alignment Search Tool or ClustalW (Thompson et al. (1994), Nucleic Acid Res., 22, 4673-4680) or any other suitable program which is suitable to generate sequence alignments. An alignment of the protein sequences of various AHAS polypeptides or partial AHAS polypeptides with reference to the Arabidopsis AHAS polypeptide of SEQ ID NO: 10 is shown in FIG. 3.

### Isolated/Purified

An “isolated” nucleic acid sequence (or DNA) is used herein to refer to a nucleic acid sequence (or DNA) that is no longer in its natural environment, for example in an in vitro or in a recombinant bacterial or plant host cell. In some embodiments, an “isolated” nucleic acid is free of nucleotide sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated nucleic acid molecule encoding an ALS protein can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. An ALS protein that is substantially free of cellular material includes preparations of protein having less than about 30%, 20%, 10%, or 5% (by dry weight) of non-ALS protein (also referred to herein as a “contaminating protein”).

### Amino Acid Substitution

Amino acid substitutions encompass amino acid alterations in which an amino acid is replaced with a different naturally-occurring amino acid residue. Such substitutions may be classified as “conservative”, in which an amino acid residue contained in the wild-type ALS protein is replaced with another naturally-occurring amino acid of similar character, for example Ala↔Val, Trp↔Leu, Gly↔Asp, Gly↔Ala, Val↔Ile↔Leu, Asp↔Glu, Lys↔Arg, Asn↔Gln or Phe↔Trp↔Tyr. Substitutions encompassed by the present invention may also be “non-conservative”, in which an amino acid residue which is present in the wild-type ALS protein is substituted with an amino acid with different properties, such as a naturally-occurring amino acid from a different group. In one embodiment, a plant comprises mutations of its endogenous acetolactate synthase (ALS) genes, wherein an ALS gene encodes an ALS polypeptide comprising at a position corresponding to position 574 of SEQ ID NO: 10 instead of the naturally encoded amino acid tryptophan the amino acid leucine and, optionally, further comprises at a position corresponding to position 197 of SEQ ID NO: 10 instead of the naturally encoded amino acid proline the amino acid serine, such as a Brassica napus plant which comprises mutations of its endogenous ALS genes, wherein an ALS I gene encodes an ALS I polypeptide comprising at a position corresponding to position 559 of SEQ ID NO: 2 instead of the naturally encoded amino acid tryptophan the amino acid leucine and, optionally, further comprises at a position corresponding to position 182 of SEQ ID NO: 2 instead of the naturally encoded amino acid proline the amino acid serine, and wherein an ALS III gene encodes an ALS III polypeptide comprising at a position corresponding to position 556 of SEQ ID NO: 4 instead of the naturally encoded amino acid tryptophan the amino acid leucine and, optionally, further comprises at a position corresponding to position 179 of SEQ ID NO: 4 instead of the naturally encoded amino acid proline the amino acid serine, or such as a Brassica juncea plant which comprises mutations of its endogenous ALS genes, wherein an ALS-A gene encodes an ALS-A polypeptide comprising at a position corresponding to position 556 of SEQ ID NO: 6 instead of the naturally encoded amino acid tryptophan the amino acid leucine and, optionally, further comprises at a position corresponding to position 179 of SEQ ID NO: 6 instead of the naturally encoded amino acid proline the amino acid serine and wherein an ALS-B gene encodes an ALS-B polypeptide comprising at a position corresponding to position 559 of SEQ ID NO: 8 instead of the naturally encoded amino acid tryptophan the amino acid leucine and, optionally, further comprises at a position corresponding to position 182 of SEQ ID NO: 8 instead of the naturally encoded amino acid proline the amino acid serine. In another embodiment, altered ALS gene sequences, such as gene sequences of ALS I gene sequence SEQ ID NO: 1 and/or ALS III gene sequence SEQ ID NO: 3, or such as ALS-A gene sequence SEQ ID NO: 5 and/or ALS-B gene sequence SEQ ID NO: 7 may contain at least one further mutation.

“Similar amino acids”, as used herein, refers to amino acids that have similar amino acid side chains, i.e. amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbours (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine, proline, and tryptophan (all hydrophobic, except for glycine which is neutral).

### Genes/Alleles

Unless indicated otherwise, the terms “wild-type allele,” “wild-type ALS allele”, “wild-type ALS gene” or “wild-type ALS polynucleotide” refer to a nucleotide sequence containing at least 60%, or 70%, or 80%, or 90%, or 95%, or 97%, or 98%, or 99% sequence identity, or is identical to the ALS sequences as described herein, such as a nucleotide sequence containing at least 60%, or 70%, or 80%, or 90%, or 95%, or 97%, or 98%, or 99% sequence identity, or is identical to SEQ ID NO: 1 and or an ALS nucleic acid sequence containing at least 60%, or 70%, or 80%, or 90%, or 95%, or 97%, or 98%, or 99% sequence identity, or is identical to SEQ ID NO: 3, or 70%, or 80%, or 90%, or 95%, or 97%, or 98%, or 99% sequence identity, or is identical to SEQ ID NO: 5, or 70%, or 80%, or 90%, or 95%, or 97%, or 98%, or 99% sequence identity, or is identical to SEQ ID NO: 7, provided that the ALS gene does not carry a mutation in the codon corresponding to the Pro197 codon of SEQ ID NO: 9, such as the ALS I gene, the ALS-III gene, the ALS-A gene and ALS-B gene do not carry a mutation in the Pro197 codon yielding an amino acid different from Pro, wherein the amino acid position referred to is the position in the reference A. thaliana sequence (SEQ ID NO: 10), or that that the ALS gene does not carry a mutation in the codon corresponding to the Trp574 codon of SEQ ID NO: 9, such as the ALS I gene, the ALS-III gene, the ALS-A gene and ALS-B gene do not carry a mutation in the Trp574 codon yielding an amino acid different from Trp, wherein the amino acid position referred to is the position in the reference A. thaliana sequence (SEQ ID NO: 10).

The terms “wild-type ALS I allele,” “wild-type ALS I allele”, “wild-type ALS I gene” or “wild-type ALS I polynucleotide” refer to a nucleotide sequence containing at least 60%, or 70%, or 80%, or 90%, or 95%, or 97%, or 98%, or 99% sequence identity, or is identical to SEQ ID NO: 1, provided that it does not carry a mutation in the Pro197 codon yielding an amino acid different from Pro or a mutation in the Trp574 codon yielding an amino acid different from Trp, wherein the amino acid position referred to is the position in the reference A. thaliana sequence (SEQ ID NO: 10).

The terms “wild-type ALS III allele,” “wild-type ALS III allele”, “wild-type ALS III gene” or “wild-type ALS III polynucleotide” refer to a nucleotide sequence containing at least 60%, or 70%, or 80%, or 90%, or 95%, or 97%, or 98%, or 99% sequence identity, or is identical to SEQ ID NO: 3, provided that it does not carry a mutation in the Pro197 codon yielding an amino acid different from Pro or a mutation in the Trp574 codon yielding an amino acid different from Trp, wherein the amino acid position referred to is the position in the reference A. thaliana sequence (SEQ ID NO: 10).

The terms “wild-type ALS-A allele,” “wild-type ALS-A allele”, “wild-type ALS-A gene” or “wild-type ALS-A polynucleotide” refer to a nucleotide sequence containing at least 60%, or 70%, or 80%, or 90%, or 95%, or 97%, or 98%, or 99% sequence identity, or is identical to SEQ ID NO: 5, provided that it does not carry a mutation in the Pro197 codon yielding an amino acid different from Pro or a mutation in the Trp574 codon yielding an amino acid different from Trp, wherein the amino acid position referred to is the position in the reference A. thaliana sequence (SEQ ID NO: 10).

The terms “wild-type ALS-B allele,” “wild-type ALS-B allele”, “wild-type ALS-B gene” or “wild-type ALS-B polynucleotide” refer to a nucleotide sequence containing at least 60%, or 70%, or 80%, or 90%, or 95%, or 97%, or 98%, or 99% sequence identity, or is identical to SEQ ID NO: 7, provided that it does not carry a mutation in the Pro197 codon yielding an amino acid different from Pro or a mutation in the Trp574 codon yielding an amino acid different from Trp, wherein the amino acid position referred to is the position in the reference A. thaliana sequence (SEQ ID NO: 10).

The term “wild type ALS” protein refers to the protein encoded by the ALS gene, wherein said ALS protein contains at least 90, 95, 97, 98, 99, or 100% sequence identity to the ALS amino acid sequence as described herein, provided that the amino acid at the position corresponding to position 197 of SEQ ID NO: 10 is a Pro and that the amino acid at the position corresponding to position 574 of SEQ ID NO: 10 is a Trp.

The term “wild type ALS I” protein refers to the protein encoded by the ALS I gene, wherein said ALS I protein contains at least 90, 95, 97, 98, 99, or 100% sequence identity to the ALS amino acid sequence of SEQ ID NO: 2, provided that the amino acid at the position corresponding to position 197 of SEQ ID NO: 10 is a Pro and that the amino acid at the position corresponding to position 574 of SEQ ID NO: 10 is a Trp.

The term “wild type ALS III” protein refers to the protein encoded by the ALS III gene, wherein said ALS III protein contains at least 90, 95, 97, 98, 99% or 100% sequence identity to the ALS amino acid sequence of SEQ ID NO: 4, provided that the amino acid at the position corresponding to position 197 of SEQ ID NO: 10 is a Pro and that the amino acid at the position corresponding to position 574 of SEQ ID NO: 10 is a Trp.

The term “wild type ALS-A” protein refers to the protein encoded by the ALS-A gene, wherein said ALS-A protein contains at least 90, 95, 97, 98, 99, or 100% sequence identity to the ALS amino acid sequence of SEQ ID NO: 6, provided that the amino acid at the position corresponding to position 197 of SEQ ID NO: 10 is a Pro and that the amino acid at the position corresponding to position 574 of SEQ ID NO: 10 is a Trp.

The term “wild type ALS-B” protein refers to the protein encoded by the ALS-B gene, wherein said ALS-B protein contains at least 90, 95, 97, 98, 99% or 100% sequence identity to the ALS amino acid sequence of SEQ ID NO: 8, provided that the amino acid at the position corresponding to position 197 of SEQ ID NO: 10 is a Pro and that the amino acid at the position corresponding to position 574 of SEQ ID NO: 10 is a Trp.

Such a “wild-type allele”, “wild-type ALS allele”, “wild-type ALS gene” or “wild-type ALS polynucleotide” may, or may not, comprise mutations, other than the mutation mentioned above. However, SEQ ID NO: 1, SEQ ID NO: 3, SEQ ID NO: 5, SEQ ID NO: 7, and SEQ ID NO: 9 are in any case “wild-type alleles” which can be used as a reference.

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which, when transcribed into mRNA, can be translated into a polypeptide. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

In essence, the difference between a wild-type plant, and a plant of the present invention is that all ALS genes of said plant comprise a codon corresponding to position 1720-1722 of SEQ ID NO: 9 encodes a Leu instead of Trp and that at least one ALS gene of said plant comprises in addition a codon corresponding to position 589-591 of SEQ ID NO: 9 encodes a Ser instead of Pro. Correspondingly, the difference between a wild-type B. napus plant, and a B. napus plant of the present invention is that an ALS I gene comprises a codon—corresponding to position 544-546 of SEQ ID NO: 1—encodes a Ser instead of Pro and a codon—corresponding to position 1675-1677 of SEQ ID NO: 1—encodes a Leu instead of Trp; and that an ALS III gene comprises a codon—corresponding to position 1666-1668 of the SEQ ID NO: 3—encodes Leu instead of Trp, or that an ALS I gene comprises a codon—corresponding to position 1675-1677 of SEQ ID NO: 1—encodes a Leu instead of Trp; and that an ALS III gene comprises a codon—corresponding to position 535-537 of SEQ ID NO: 3—encodes a Ser instead of Pro and a codon—corresponding to position 1666-1668 of the SEQ ID NO: 3—encodes Leu instead of Trp, or that an ALS I gene comprises a codon—corresponding to position 544-546 of SEQ ID NO: 1—encodes a Ser instead of Pro and a codon—corresponding to position 1675-1677 of SEQ ID NO: 1—encodes a Leu instead of Trp; and that an ALS III gene comprises a codon—corresponding to position 535-537 of SEQ ID NO: 3—encodes a Ser instead of Pro and a codon—corresponding to position 1666-1668 of the SEQ ID NO: 3—encodes Leu instead of Trp; the difference between a wild-type B. juncea plant, and a B. juncea plant of the present invention is that an ALS-A gene comprises a codon—corresponding to position 535-537 of SEQ ID NO: 5—encodes a Ser instead of Pro and a codon—corresponding to position 1666-1668 of SEQ ID NO: 5—encodes a Leu instead of Trp; and that an ALS-B gene comprises a codon—corresponding to position 1675-1677 of the SEQ ID NO: 7—encodes Leu instead of Trp, or that an ALS-A gene comprises a codon—corresponding to position 1666-1668 of SEQ ID NO: 5—encodes a Leu instead of Trp; and that an ALS-B gene comprises a codon—corresponding to position 544-546 of SEQ ID NO: 7—encodes a Ser instead of Pro and a codon—corresponding to position 1675-1677 of the SEQ ID NO: 7—encodes Leu instead of Trp, or that an ALS-A gene comprises a codon—corresponding to position 535-537 of SEQ ID NO: 5—encodes a Ser instead of Pro and a codon—corresponding to position 1666-1668 of SEQ ID NO: 5—encodes a Leu instead of Trp; and that an ALS-B gene comprises a codon—corresponding to position 544-546 of SEQ ID NO: 7—encodes a Ser instead of Pro and a codon—corresponding to position 1675-1677 of the SEQ ID NO: 7—encodes Leu instead of Trp.

However, as mentioned above, further differences such as additional mutations may be present between wild-type and the mutant ALS allele as specified herein. Yet, these further differences are not relevant as long as the difference explained before is present.

In one embodiment, a plant according to the present invention comprises at least two ALS genes, wherein all ALS genes encode an ALS protein comprising Leu instead of Trp at a position corresponding to position 574 of SEQ ID NO: 10, and wherein and wherein at least one of the ALS genes encodes an ALS protein further comprising Ser instead of Pro at a position corresponding to position 197 of SEQ ID NO: 10, when comparing said ALS protein with the wild type amino acid sequence of said ALS protein. In a further embodiment, a B. napus plant according to the present invention comprises an ALS I gene which encodes an ALS I protein comprising Ser instead of Pro at a position 182 and Leu instead of Trp at a position 559 when comparing said ALS I protein with the wild type amino acid sequence SEQ ID NO: 2; and comprises an ALS III gene which encodes an ALS III protein comprising Leu instead of Trp at a position 556 when comparing said ALS III protein with the wild type amino acid sequence SEQ ID NO: 4, or a B. napus plant according to the present invention comprises an ALS I gene which encodes an ALS I protein comprising Leu instead of Trp at a position 559 when comparing said ALS I protein with the wild type amino acid sequence SEQ ID NO: 2; and comprises an ALS III gene which encodes an ALS III protein comprising Ser instead of Pro at a position 179 and Leu instead of Trp at a position 556 when comparing said ALS III protein with the wild type amino acid sequence SEQ ID NO: 4, or a B. napus plant according to the present invention comprises an ALS I gene which encodes an ALS I protein comprising Ser instead of Pro at a position 182 and Leu instead of Trp at a position 559 when comparing said ALS I protein with the wild type amino acid sequence SEQ ID NO: 2; and comprises an ALS III gene which encodes an ALS III protein comprising Ser instead of Pro at a position 179 and Leu instead of Trp at a position 556 when comparing said ALS III protein with the wild type amino acid sequence SEQ ID NO: 4. In a further embodiment, a B. juncea plant according to the present invention comprises an ALS-A gene which encodes an ALS-A protein comprising Ser instead of Pro at a position 179 and Leu instead of Trp at a position 556 when comparing said ALS-A protein with the wild type amino acid sequence SEQ ID NO: 6; and comprises an ALS-B gene which encodes an ALS-B protein comprising Leu instead of Trp at a position 559 when comparing said ALS-B protein with the wild type amino acid sequence SEQ ID NO: 8, or a B. juncea plant according to the present invention comprises an ALS-A gene which encodes an ALS-A protein comprising Leu instead of Trp at a position 556 when comparing said ALS-A protein with the wild type amino acid sequence SEQ ID NO: 6; and comprises an ALS-B gene which encodes an ALS-B protein comprising Ser instead of Pro at a position 182 and Leu instead of Trp at a position 559 when comparing said ALS-B protein with the wild type amino acid sequence SEQ ID NO: 8, or a B. juncea plant according to the present invention comprises an ALS-A gene which encodes an ALS-A protein comprising Ser instead of Pro at a position 179 and Leu instead of Trp at a position 556 when comparing said ALS-A protein with the wild type amino acid sequence SEQ ID NO: 6; and comprises an ALS-B gene which encodes an ALS-B protein comprising Ser instead of Pro at a position 182 and Leu instead of Trp at a position 559 when comparing said ALS-B protein with the wild type amino acid sequence SEQ ID NO: 8. The skilled person will understand that such mutated ALS genes, such as ALS I, ALS III, ALS-A and ALS-B genes may comprise further mutations such as one, two or three further mutations.

Consequently, the Pro197Ser substitutions (when the A. thaliana ALS amino acid sequence of SEQ ID NO: 10 is used as reference) are a result of an alteration of codons at a position corresponding to position 589-591 of the nucleotide sequence shown in SEQ ID NO: 9, and the Trp574Leu substitutions (when the A. thaliana ALS amino acid sequence of SEQ ID NO: 10 is used as reference) are a result of an alteration of codons at a position corresponding to position 1720-1722 of the nucleotide sequence shown in SEQ ID NO: 9.

In one embodiment, the substitution at position 197 (when the A. thaliana ALS amino acid sequence of SEQ ID NO: 10 is used as reference) is a P→S substitution, wherein “S” is encoded by any of the codons “TCT”, “TCC”, “TCA”, “TCG”, “AGT”, “AGC”; in a further embodiment said “S” is encoded by the codon “TCT”.

In one embodiment, the substitution at position 574 (when the A. thaliana ALS amino acid sequence of SEQ ID NO: 10 is used as reference) is a W→L substitution, wherein “L” is encoded by any of the codons “CTT”, “CTC”, “CTA”, “CTG”, “TTA”, “TTG”; in a further embodiment said “L” is encoded by the codon “TTG”.

Hence, in one embodiment, the present invention provides a plant comprising at least two ALS genes, wherein the nucleotide sequence of all ALS genes in their endogenous gene loci, at least a codon encoding Leu instead of Trp, at a position corresponding to position 1720-1722 of the A. thaliana ALS nucleic acid sequence of SEQ ID NO: 9, and where the nucleotide sequence of at least one ALS gene in the endogenous gene locus further comprises at least a codon encoding Ser instead of Pro, at a position corresponding to position 589-591 of SEQ ID NO: 9, such as a B. napus plant comprising in the nucleotide sequence of an ALS I gene in its endogenous gene locus, at least a codon encoding Ser instead of Pro, at a position corresponding to position 589-591 and at least a codon encoding Leu instead of Trp, at a position corresponding to position 1720-1722 of the A. thaliana ALS nucleic acid sequence of SEQ ID NO: 9 and comprising in the nucleotide sequence of an ALS III gene in its endogenous gene locus, at least a codon encoding Leu instead of Trp at a position corresponding to position 1720-1722 of the A. thaliana ALS nucleic acid sequence of SEQ ID NO: 9, or such as a B. napus plant comprising in the nucleotide sequence of an ALS I gene in its endogenous gene locus, at least a codon encoding Leu instead of Trp, at a position corresponding to position 1720-1722 of the A. thaliana ALS nucleic acid sequence of SEQ ID NO: 9 and comprising in the nucleotide sequence of an ALS III gene in its endogenous gene locus, at least a codon encoding Ser instead of Pro, at a position corresponding to position 589-591 and at least a codon encoding Leu instead of Trp at a position corresponding to position 1720-1722 of the A. thaliana ALS nucleic acid sequence of SEQ ID NO: 9, or such as a B. napus plant comprising in the nucleotide sequence of an ALS I gene in its endogenous gene locus, at least a codon encoding Ser instead of Pro, at a position corresponding to position 589-591 and at least a codon encoding Leu instead of Trp, at a position corresponding to position 1720-1722 of the A. thaliana ALS nucleic acid sequence of SEQ ID NO: 9 and comprising in the nucleotide sequence of an ALS III gene in its endogenous gene locus, at least a codon encoding Ser instead of Pro, at a position corresponding to position 589-591 and at least a codon encoding Leu instead of Trp at a position corresponding to position 1720-1722 of the A. thaliana ALS nucleic acid sequence of SEQ ID NO: 9, or such as a B. juncea plant comprising in the nucleotide sequence of an ALS-A gene in its endogenous gene locus, at least a codon encoding Ser instead of Pro, at a position corresponding to position 589-591 and at least a codon encoding Leu instead of Trp, at a position corresponding to position 1720-1722 of the A. thaliana ALS nucleic acid sequence of SEQ ID NO: 9 and comprising in the nucleotide sequence of an ALS-B gene in its endogenous gene locus, at least a codon encoding Leu instead of Trp at a position corresponding to position 1720-1722 of the A. thaliana ALS nucleic acid sequence of SEQ ID NO: 9, or such as a B. juncea plant comprising in the nucleotide sequence of an ALS-A gene in its endogenous gene locus, at least a codon encoding Leu instead of Trp, at a position corresponding to position 1720-1722 of the A. thaliana ALS nucleic acid sequence of SEQ ID NO: 9 and comprising in the nucleotide sequence of an ALS-B gene in its endogenous gene locus, at least a codon encoding Ser instead of Pro, at a position corresponding to position 589-591 and at least a codon encoding Leu instead of Trp at a position corresponding to position 1720-1722 of the A. thaliana ALS nucleic acid sequence of SEQ ID NO: 9, or such as a B. juncea plant comprising in the nucleotide sequence of an ALS-A gene in its endogenous gene locus, at least a codon encoding Ser instead of Pro, at a position corresponding to position 589-591 and at least a codon encoding Leu instead of Trp, at a position corresponding to position 1720-1722 of the A. thaliana ALS nucleic acid sequence of SEQ ID NO: 9 and comprising in the nucleotide sequence of an ALS-B gene in its endogenous gene locus, at least a codon encoding Ser instead of Pro, at a position corresponding to position 589-591 and at least a codon encoding Leu instead of Trp at a position corresponding to position 1720-1722 of the A. thaliana ALS nucleic acid sequence of SEQ ID NO: 9.

ALS alleles according to the invention or plants comprising ALS alleles according to the invention can be identified or detected by method known in the art, such as direct sequencing, PCR based assays or hybridization based assays. Alternatively, methods can also be developed using the specific ALS allele specific sequence information provided herein. Such alternative detection methods include linear signal amplification detection methods based on invasive cleavage of particular nucleic acid structures, also known as Invader™ technology, (as described e.g. in U.S. Pat. No. 5,985,557 “Invasive Cleavage of Nucleic Acids”, U.S. Pat. No. 6,001,567 “Detection of Nucleic Acid sequences by Invader Directed Cleavage, incorporated herein by reference), RT-PCR-based detection methods, such as Taqman, or other detection methods, such as SNPlex. Briefly, in the Invader™ technology, the target mutation sequence may e.g. be hybridized with a labeled first nucleic acid oligonucleotide comprising the nucleotide sequence of the mutation sequence or a sequence spanning the joining region between the 5′ flanking region and the mutation region and with a second nucleic acid oligonucleotide comprising the 3′ flanking sequence immediately downstream and adjacent to the mutation sequence, wherein the first and second oligonucleotide overlap by at least one nucleotide. The duplex or triplex structure that is produced by this hybridization allows selective probe cleavage with an enzyme (Cleavase®) leaving the target sequence intact. The cleaved labeled probe is subsequently detected, potentially via an intermediate step resulting in further signal amplification.

The present invention also relates to the combination of ALS alleles according to the invention in one plant, and to the transfer of ALS alleles according to the invention from one plant to another plant.

### ALS Inhibitor Herbicide Tolerance

For the present invention, the terms “herbicide-tolerant” and “herbicide-resistant” are used interchangeably and are intended to have an equivalent meaning and an equivalent scope. Similarly, the terms “herbicide-tolerance” and “herbicide-resistance” are used interchangeably and are intended to have an equivalent meaning and an equivalent scope.

It is preferred that the plants of the present invention are less sensitive to an ALS inhibitor, such as at least 5 times, or 10 times, or 50 times, or 100 times, or 500 times, or 1000 times, or 2000 times less sensitive as compared to wild type plants having not the substitutions of the present invention, such as wild type B. napus plants comprising ALS I polypeptides of SEQ ID NO: 2 and ALS III polypeptides of SEQ ID NO: 4, i.e., wild type plants having not the substitutions of the present invention, or such as wild type B. juncea plants comprising ALS-A polypeptides of SEQ ID NO: 6 and ALS-B polypeptides of SEQ ID NO: 8, i.e., wild type plants having not the substitutions of the present invention. Wild type plants wherein all ALS alleles do not comprise the substitutions of the present invention, such as wild type B. napus plants wherein all ALS I alleles are alleles of SEQ ID NO: 1 and all ALS III alleles are alleles of SEQ ID NO: 3, or such as wild type B. juncea plants wherein all ALS-A alleles are alleles of SEQ ID NO: 5 and all ALS-B alleles are alleles of SEQ ID NO: 7 are preferred references when comparing ALS sensitivity. Less sensitive when used herein may, vice versa, be seen as “more tolerable” or “more resistant”. Similarly, “more tolerable” or “more resistant” may, vice versa, be seen as “less sensitive”.

For example, the B. napus plants of the present invention and in particular the B. napus plant described in the appended Examples are/is at less sensitive to a combination of the ALS inhibitor herbicides foramsulfuron (a member of the ALS inhibitor subclass “sulfonylurea herbicides”) and thiencarbazone-methyl (a member of the ALS inhibitor subclass “sulfonylaminocarbonyltriazolinone herbicides”) compared to the wild type enzyme.

An “herbicide-tolerant” or “herbicide-resistant” plant refers to a plant that is tolerant or resistant to at least one AHAS-inhibiting herbicide at a level that would normally kill, or inhibit the growth of a wild-type plant lacking a mutated AHAS nucleic acid molecule. By “herbicide-resistant AHAS nucleic acid molecule” is intended a nucleic acid molecule comprising one or more mutations that results in one or more amino acid substitutions relative to the non-mutated AHAS protein, where the mutations result in the expression of an herbicide-resistant AHAS protein. By “herbicide-tolerant AHAS protein” or “herbicide-resistant AHAS protein”, it is intended that such an AHAS protein displays higher AHAS activity, relative to the AHAS activity of a wild-type AHAS protein, when in the presence of at least one herbicide that is known to interfere with AHAS activity and at a concentration or level of the herbicide that is to known to inhibit the AHAS activity of the wild-type AHAS protein. Furthermore, the AHAS activity of such an herbicide-tolerant or herbicide-resistant AHAS protein may be referred to herein as “herbicide-tolerant” or “herbicide-resistant” AHAS activity.

Preferably, the plants of the present invention are less sensitive to various members of ALS inhibitor herbicides, like sulfonylurea herbicides, sulfonylamino-carbonyltriazolinone herbicides, and imidazolinone herbicides. Sulfonylurea herbicides and sulfonylaminocarbonyltriazolinone herbicides against which said plants are less sensitive are preferably selected. In a particular preferred embodiment, the plants of the present invention are less sensitive to the ALS inhibitor herbicide foramsulfuron (sulfonylurea herbicide) either alone or in combination with one or more further ALS inhibitor herbicides either from the subclass of the sulfonyurea-herbicides or any other sub-class of the ALS inhibitor herbicides.

Hence, the plants of the present invention which are preferably less sensitive to an ALS inhibitor herbicide can likewise also be characterized to be “more tolerant” to an ALS inhibitor” (i.e. an ALS inhibitor tolerant plant).

Thus, an “ALS inhibitor tolerant” plant refers to a plant, preferably a plant according to the present invention or any of its progenies that is more tolerant to at least one ALS inhibitor herbicide at a level that would normally inhibit the growth of a wild-type plant, preferably the ALS inhibitor herbicide controls a wild-type plant. Said wild-type plant does not comprise in the nucleotide sequence of any allele of any endogenous ALS gene a codon encoding a Ser instead of Pro at a position corresponding to position 589-591 of SEQ ID NO: 9 or a codon encoding a Leu instead of Trp a position corresponding to position 1720-1722 of SEQ ID NO: 9, such as a B. napus plant which does not comprise the nucleotide sequence of any allele of the endogenous ALS I gene, a codon encoding Ser instead of Pro at a position corresponding to position 544-546 of SEQ ID NO: 1 or a codon encoding Leu instead of Trp at a position corresponding to position 1675-1677 of SEQ ID NO: 1 and does not comprise in the nucleotide sequence of any allele of the endogenous ALS III gene, a codon encoding Ser instead of Pro at a position corresponding to position 535-537 of SEQ ID NO: 3 or a codon encoding Leu instead of Trp at a position corresponding to position 1666-1668 of SEQ ID NO: 3, or such as a B. juncea plant which does not comprise the nucleotide sequence of any allele of the endogenous ALS-A gene, a codon encoding Ser instead of Pro at a position corresponding to position 535-537 of SEQ ID NO: 5 or a codon encoding Leu instead of Trp at a position corresponding to position 1666-1668 of SEQ ID NO: 5 and does not comprise in the nucleotide sequence of any allele of the endogenous ALS-B gene, a codon encoding Ser instead of Pro at a position corresponding to position 544-546 of SEQ ID NO: 7 a codon encoding Leu instead of Trp at a position corresponding to position 1675-1677 of SEQ ID NO: 7.

Said nucleotide sequences may generally also be characterized to be “ALS inhibitor herbicide tolerant” nucleotide sequences. By “ALS inhibitor herbicide tolerant nucleotide sequence” is intended a nucleic acid molecule comprising nucleotide sequences encoding for an ALS protein having at least a Leu instead of Trp a position corresponding to position 574 of SEQ ID NO: 10 and, optionally, further comprising a Ser instead of Pro at a position corresponding to position 197 of SEQ ID NO: 10, such as a nucleic acid molecule comprising nucleotide sequences encoding for an ALS I protein having at least a Leu instead of Trp a position corresponding to position 559 of SEQ ID NO: 2 and/or nucleotide sequences encoding for an ALS I protein having at least a Ser instead of Pro a position corresponding to position 182 of SEQ ID NO: 2 and a Leu instead of Trp a position corresponding to position 559 of SEQ ID NO: 2, and/or nucleotide sequences encoding for a ALS III protein having at least a Leu instead of Trp at a position corresponding to position 556 of SEQ ID NO: 4, and/or nucleotide sequences encoding for a ALS III protein having at least a Ser instead of Pro a position corresponding to position 179 of SEQ ID NO: 4 and a Leu instead of Trp at a position corresponding to position 556 of SEQ ID NO: 4, or such as a nucleic acid molecule comprising nucleotide sequences encoding for an ALS-A protein having at least a Leu instead of Trp a position corresponding to position 556 of SEQ ID NO: 6 and/or nucleotide sequences encoding for an ALS-A protein having at least a Ser instead of Pro a position corresponding to position 179 of SEQ ID NO: 6 and a Leu instead of Trp a position corresponding to position 556 of SEQ ID NO: 6 and/or nucleotide sequences encoding for a ALS-B protein having at least a Leu instead of Trp at a position corresponding to position 559 of SEQ ID NO: 8 and/or nucleotide sequences encoding for a ALS-B protein having at least a Ser instead of Pro at a position corresponding to position 182 of SEQ ID NO: 8 a Leu instead of Trp at a position corresponding to position 559 of SEQ ID NO: 8, wherein said at least one mutation or said at least two mutations result in the expression of a less sensitive to an ALS inhibitor herbicide ALS protein.

By “herbicide-tolerant ALS protein”, it is intended that such an ALS protein displays higher ALS activity, relative to the ALS activity of a wild-type ALS protein, in the presence of at least one ALS inhibitor herbicide that is known to interfere with ALS activity and at a concentration or level of said herbicide that is known to inhibit the ALS activity of the wild-type ALS protein.

Similarly, the terms “ALS-inhibitor herbicide(s)” or simply “ALS-inhibitor(s)” are used interchangeably. As used herein, an “ALS-inhibitor herbicide” or an “ALS inhibitor” is not meant to be limited to single herbicide that interferes with the activity of the ALS enzyme. Thus, unless otherwise stated or evident from the context, an “ALS-inhibitor herbicide” or an “ALS inhibitor” can be a one herbicide or a mixture of two, three, four, or more herbicides known in the art, preferably as specified herein, each of which interferes with the activity of the ALS enzyme.

“Herbicide resistance” or “herbicide tolerance” can be measured as described in the present application or, e.g., it can be measured by comparison of AHAS activity obtained from cell extracts from plants containing the mutagenized AHAS sequence and from plants lacking the mutagenized AHAS sequence in the presence of an AHAS inhibitor, such as foramsulfuron or imazamox, using the methods disclosed in Singh, et al. Anal. Biochem., (1988), 171: 173-179. In one embodiment, resistant or tolerant plants demonstrate greater than 25% uninhibition using the methods disclosed in Singh et al (1988) when assayed, e.g., using 10 μM foramsulfuron or 10 μM imazamox.

The activity of specific ALS proteins such as ALS I or ALS III proteins can be measured according to the following method: The coding sequences of wild-type, P197S-W574L-mutant, and W574L-mutant ALS, such as Brassica wild-type, P197S-W574L-mutant, and W574L-mutant ALS I, or wild type, P197S-W574L-mutant, and W574L-mutant ALS III, or wild type, P197S-W574L-mutant, and W574L-mutant ALS-A, or wild-type, P197S-W574L-mutant, or W574L-mutant ALS-B genes can be cloned into Novagen pET-32a(+) vectors and the vectors transformed into Escherichia coli AD494 according to the instructions of the manufacturer. Bacteria are grown at 37° C. in LB-medium containing 100 mg/l carbenicillin and 25 mg/l canamycin, induced with 1 mM isopropyl-β-D-thiogalactopyranoside at an OD₆₀₀ of 0.6, cultivated for 16 hours at 18° C. and harvested by, e.g., centrifugation. Bacterial pellets are resuspended in 100 mM sodium phosphate buffer pH 7.0 containing 0.1 mM thiamine-pyrophosphate, 1 mM MgCl₂, and 1 μM FAD at a concentration of 1 gram wet weight per 25 ml of buffer and disrupted by, e.g., sonification. The crude protein extract obtained after centrifugation is used for ALS activity measurements.

P197S-W574L-mutant ALS refers to an ALS protein comprising a serine instead of a proline at a position corresponding to position 197 of SEQ ID NO: 10, and a leucine instead of a tryptophan at a position corresponding to position 574 of SEQ ID NO: 10. W574L-mutant ALS refers to an ALS protein comprising a leucine instead of a tryptophan at a position corresponding to position 574 of SEQ ID NO: 10.

ALS protein can be extracted from leaves or tissue cultures, such as B. napus or B. juncea leaves, or B. napus or B. juncea tissue cultures as described by Ray (Plant Physiol, 1984, 75:827-831). An ALS assays can be carried out in 96-well microtiter plates using a modification of the procedure described by Ray (1984): The reaction mixture contains 20 mM potassium phosphate buffer pH 7.0, 20 mM sodium pyruvate, 0.45 mM thiamine-pyrophosphate, 0.45 mM MgCl₂, 9 μM FAD. ALS enzyme and various concentrations of ALS inhibitors can be mixed in a final volume of 90 μl. Assays can be initiated by adding enzyme and the assays can be terminated after 75 min incubation at 30° C. by the addition of 40 μl 0.5 M H₂SO₄. After 60 min at room temperature 80 μl of a solution of 1.4% α-naphtol and 0.14% creatine in 0.7 M NaOH can be added and after an additional 45 min incubation at room temperature the absorbance can be determined at 540 nm. pI50-values for inhibition of ALS can be determined as described by Ray (1984), using the XLFit Excel add-in version 4.3.1 curve fitting program of ID Business Solutions Limited.

The ALS nucleotide sequences referred to herein encoding ALS polypeptides preferably confer tolerance to one or more ALS inhibitor herbicides (or, vice versa, less sensitivity to an ALS inhibitor herbicide) as described herein. This is because of the point mutation(s) leading to the amino acid substitution(s) as described herein. In one embodiment, the plants of the present invention show tolerance against a compound of formula (I), e.g., plants according to the invention show essentially no injury (injury below 5%, 1% or even 0%) when 15 g a.i./ha are applied whereas injury of wild type is above 90%.

Surprisingly, it was found that the presence of the W574L mutation in ALS I in combination with the P197S-W574L mutation in ALS III increases herbicide tolerance to ALS inhibitor herbicides of Brassica plants.

One embodiment of the present invention refers to plants and parts thereof and progeny thereof which are heterozygous for the mutations described herein. Thus, also covered by the present invention are plants comprising at least in one allele of the ALS genes in their endogenous gene loci a codon encoding Leu instead of Trp, at a position corresponding to position 1720-1722 of SEQ ID NO: 9 and comprising one or more further ALS alleles encoding independently from each other Trp at a position corresponding to position 1720-1722 of SEQ ID NO: 9 wherein said further allele optionally comprise independently from each other at least one, two or three further mutations, said plants further comprising at least one ALS gene in its endogenous locus in addition to the codon encoding Leu instead of Trp, at a position corresponding to position 1720-1722 of SEQ ID NO: 9, the codon encoding Ser instead of Pro, at a position corresponding to position 589-591 of SEQ ID NO: 9, and comprising one or more further ALS alleles encoding independently from each other Pro at a position corresponding to position 589-591 of SEQ ID NO: 9 and Trp at a position corresponding to position 1720-1722 of SEQ ID NO: 9 wherein said further allele optionally comprise independently from each other at least one, two or three further mutations.

Thus, also covered by the present invention are B. napus plants comprising at least in one allele of the ALS I gene in its endogenous gene locus a codon encoding Ser instead of Pro at a position corresponding to position 544-546 of SEQ ID NO: 1, and Leu instead of Trp, at a position corresponding to position 1675-1677 of SEQ ID NO: 1, and comprising one or more further ALS I alleles encoding independently from each other Pro at a position corresponding to position 544-546 of SEQ ID NO: 1 and Trp at a position corresponding to position 1675-1677 of SEQ ID NO: 1 wherein said further allele optionally comprise independently from each other at least one, two or three further mutations; and comprising in at least one allele of the ALS III gene in its endogenous gene locus a codon encoding Leu instead of Trp at a position corresponding to position 1666-1668 of SEQ ID NO: 3, and comprising one or more further ALS III allele(s) having independently from each other a codon at a position corresponding to position 1666-1668 of SEQ ID NO: 3 encoding Trp wherein said further ALS III alleles optionally comprise independently from each other at least one, two or three further mutations. Also covered by the present invention are B. napus plants comprising at least in one allele of the ALS I gene in its endogenous gene locus a codon encoding Leu instead of Trp, at a position corresponding to position 1675-1677 of SEQ ID NO: 1, and comprising one or more further ALS I alleles encoding independently from each other Trp at a position corresponding to position 1675-1677 of SEQ ID NO: 1 wherein said further allele optionally comprise independently from each other at least one, two or three further mutations; and comprising in at least one allele of the ALS III gene in its endogenous gene locus a codon encoding Ser instead of Pro at a position corresponding to position 535-537 of SEQ ID NO: 3 and a codon encoding Leu instead of Trp at a position corresponding to position 1666-1668 of SEQ ID NO: 3, and comprising one or more further ALS III allele(s) having independently from each other a codon at a position corresponding to position 535-537 of SEQ ID NO: 3 encoding Pro and a codon at a position corresponding to position 1666-1668 of SEQ ID NO: 3 encoding Trp wherein said further ALS III alleles optionally comprise independently from each other at least one, two or three further mutations. Also covered by the present invention are B. napus plants comprising at least in one allele of the ALS I gene in its endogenous gene locus a codon encoding Ser instead of Pro at a position corresponding to position 544-546 of SEQ ID NO: 1, and Leu instead of Trp, at a position corresponding to position 1675-1677 of SEQ ID NO: 1, and comprising one or more further ALS I alleles encoding independently from each other Pro at a position corresponding to position 544-546 of SEQ ID NO: 1 and Trp at a position corresponding to position 1675-1677 of SEQ ID NO: 1 wherein said further allele optionally comprise independently from each other at least one, two or three further mutations; and comprising in at least one allele of the ALS III gene in its endogenous gene locus a codon encoding Ser instead of Pro at a position corresponding to position 535-537 of SEQ ID NO: 3 and a codon encoding Leu instead of Trp at a position corresponding to position 1666-1668 of SEQ ID NO: 3, and comprising one or more further ALS III allele(s) having independently from each other a codon at a position corresponding to position 535-537 of SEQ ID NO: 3 encoding Pro and a codon at a position corresponding to position 1666-1668 of SEQ ID NO: 3 encoding Trp wherein said further ALS III alleles optionally comprise independently from each other at least one, two or three further mutations.

Also covered by the invention are therefore B. juncea plants comprising at least in one allele of the ALS-A gene in its endogenous gene locus a codon encoding Ser instead of Pro at a position corresponding to position 535-537 of SEQ ID NO: 5 and a codon encoding Leu instead of Trp, at a position corresponding to position 1666-1668 of SEQ ID NO: 5, and comprising one or more further ALS-A alleles encoding independently from each other Pro at a position corresponding to position 535-537 of SEQ ID NO: 5 and Trp at a position corresponding to position 1666-1668 of SEQ ID NO: 5 wherein said further allele optionally comprise independently from each other at least one, two or three further mutations; and comprising in at least one allele of the ALS-B gene in its endogenous gene locus a codon encoding Leu instead of Trp at a position corresponding to position 1675-1677 of SEQ ID NO: 7, and comprising one or more further ALS-B allele(s) having independently from each other a codon at a position corresponding to position 1675-1677 of SEQ ID NO: 7 encoding Trp wherein said further ALS-B alleles optionally comprise independently from each other at least one, two or three further mutations. Also covered by the invention are therefore B. juncea plants comprising at least in one allele of the ALS-A gene in its endogenous gene locus a codon encoding Leu instead of Trp, at a position corresponding to position 1666-1668 of SEQ ID NO: 5, and comprising one or more further ALS-A alleles encoding independently from each other Trp at a position corresponding to position 1666-1668 of SEQ ID NO: 5 wherein said further allele optionally comprise independently from each other at least one, two or three further mutations; and comprising in at least one allele of the ALS-B gene in its endogenous gene locus a codon encoding Ser instead of Pro at a position corresponding to position 544-546 of SEQ ID NO: 7 and a codon encoding Leu instead of Trp at a position corresponding to position 1675-1677 of SEQ ID NO: 7, and comprising one or more further ALS-B allele(s) having independently from each other a codon at a position corresponding to position 535-537 of SEQ ID NO: 5 encoding Pro and a codon at a position corresponding to position 1675-1677 of SEQ ID NO: 7 encoding Trp wherein said further ALS-B alleles optionally comprise independently from each other at least one, two or three further mutations. Also covered by the invention are therefore B. juncea plants comprising at least in one allele of the ALS-A gene in its endogenous gene locus a codon encoding Ser instead of Pro at a position corresponding to position 535-537 of SEQ ID NO: 5 and a codon encoding Leu instead of Trp, at a position corresponding to position 1666-1668 of SEQ ID NO: 5, and comprising one or more further ALS-A alleles encoding independently from each other Pro at a position corresponding to position 535-537 of SEQ ID NO: 5 and Trp at a position corresponding to position 1666-1668 of SEQ ID NO: 5 wherein said further allele optionally comprise independently from each other at least one, two or three further mutations; and comprising in at least one allele of the ALS-B gene in its endogenous gene locus a codon encoding Ser instead of Pro at a position corresponding to position 544-546 of SEQ ID NO: 7 and a codon encoding Leu instead of Trp at a position corresponding to position 1675-1677 of SEQ ID NO: 7, and comprising one or more further ALS-B allele(s) having independently from each other a codon at a position corresponding to position 535-537 of SEQ ID NO: 5 encoding Pro and a codon at a position corresponding to position 1675-1677 of SEQ ID NO: 7 encoding Trp wherein said further ALS-B alleles optionally comprise independently from each other at least one, two or three further mutations.

However, one embodiment of the invention refers to polyploid plants and parts thereof which are homozygous regarding the mutation of the ALS genes resulting in a codon encoding Leu instead of Trp at a position corresponding to position 1720-1722 of SEQ ID NO: 9 and which are homozygous regarding the mutation of the ALS genes resulting in a codon encoding Ser instead of Pro at a position corresponding to position 589-591 of SEQ ID NO: 9 and a codon encoding Leu instead of Trp at a position corresponding to position 1720-1722 of SEQ ID NO: 9, such as B. napus plants and parts thereof which are homozygous regarding the mutations of ALS-I genes resulting in a codon encoding Ser instead of Pro at a position corresponding to position 544-546 of SEQ ID NO: 1 and a codon encoding Leu instead of Trp at a position corresponding to position 1675-1677 of SEQ ID NO: 1; and the mutation of ALS III genes resulting in a codon encoding Leu instead of Trp at a position corresponding to position 1666-1668 of SEQ ID NO: 3; or such as B. napus plants and parts thereof which are homozygous regarding the mutation of ALS-I genes resulting in a codon encoding Leu instead of Trp at a position corresponding to position 1675-1677 of SEQ ID NO: 1 and the mutations of ALS III genes resulting in a codon encoding Ser instead of Pro at a position corresponding to position 535-537 of SEQ ID NO: 3 and a codon encoding Leu instead of Trp at a position corresponding to position 1666-1668 of SEQ ID NO: 3; or such as B. napus plants and parts thereof which are homozygous regarding the mutations of ALS-I genes resulting in a codon encoding Ser instead of Pro at a position corresponding to position 544-546 of SEQ ID NO: 1 and a codon encoding Leu instead of Trp at a position corresponding to position 1675-1677 of SEQ ID NO: 1 and the mutations of ALS III genes resulting in a codon encoding Ser instead of Pro at a position corresponding to position 535-537 of SEQ ID NO: 3 and a codon encoding Leu instead of Trp at a position corresponding to position 1666-1668 of SEQ ID NO: 3; or such as B. juncea plants and parts thereof which are homozygous regarding the mutations of ALS-A genes resulting in a codon encoding Ser instead of Pro at a position corresponding to position 535-537 of SEQ ID NO: 5 and a codon encoding Leu instead of Trp at a position corresponding to position 1666-1668 of SEQ ID NO: 5; and the point mutation of ALS-B genes resulting in a codon encoding Leu instead of Trp at a position corresponding to position 1675-1677 of SEQ ID NO: 7, or such as B. juncea plants and parts thereof which are homozygous regarding the mutation of ALS-A genes resulting in a codon encoding Leu instead of Trp at a position corresponding to position 1666-1668 of SEQ ID NO: 5; and the point mutations of ALS-B genes resulting in a codon encoding Ser instead of Pro at a position corresponding to position 544-546 of SEQ ID NO: 7 and a codon encoding Leu instead of Trp at a position corresponding to position 1675-1677 of SEQ ID NO: 7, or such as B. juncea plants and parts thereof which are homozygous regarding the mutations of ALS-A genes resulting in a codon encoding Ser instead of Pro at a position corresponding to position 535-537 of SEQ ID NO: 5 and a codon encoding Leu instead of Trp at a position corresponding to position 1666-1668 of SEQ ID NO: 5 and the mutations of ALS-B genes resulting in a codon encoding Ser instead of Pro at a position corresponding to position 544-546 of SEQ ID NO: 7 and a codon encoding Leu instead of Trp at a position corresponding to position 1675-1677 of SEQ ID NO: 7.

As used herein, the term “heterozygous” means a genetic condition existing when (at least) two different alleles reside at a specific locus, but are positioned individually on corresponding pairs of homologous chromosomes in the cell. In other words, (at least) two different ALS alleles, reside at specific loci but are positioned individually on corresponding pairs of homologous chromosomes in the cell.

Conversely, as used herein, the term “homozygous” means a genetic condition existing when two (all) identical alleles reside at a specific locus, but are positioned individually on corresponding pairs of homologous chromosomes in the cell.

As used herein, the term “locus” (loci plural) means a specific place or places or a site on a chromosome where, e.g., a gene or genetic marker is found.

As mentioned herein, the plant of the present invention comprises in the nucleotide sequence of at least one ALS allele of all endogenous ALS gene loci a codon encoding Leu instead of Trp at a position as specified herein, and at least one ALS allele of an endogenous ALS gene locus a codon encoding Ser instead of Trp at a position specified herein, and a codon encoding Leu instead of Trp at a position as specified herein. By ALS genes in its “endogenous locus” it is meant that the ALS genes comprised by the plant of the present invention is—when compared to a wild-type plant—located in the same locus, i.e., the ALS genes are positioned (located) on the same chromosome in the same chromosomal context (organization) as they are positioned in a wild-type plant (i.e., without there being any human intervention so as to transfer or re-locate the ALS genes comprised by the plant of the present invention to another location such as to another chromosome or genomic locus (position) different from that where the ALS genes are naturally located). Accordingly, the identical genome-specific satellite markers which surround a wild-type ALS gene also surround an ALS gene comprised by the plant of the present invention.

“Positioned in the same chromosomal context (organization)” means that an ALS gene of the plant of the present invention is located on the same chromosome as it is in a wild-type plant. Accordingly, the same genes as in a wild-type plant are adjacent to the 5′- and 3′-end of an ALS gene comprised by the plant of the present invention. Hence, the same nucleotide sequences which are adjacent to the 5′- and 3′-end of the wild-type ALS gene are adjacent to the 5′- and 3′-end of an ALS gene comprised by the plant of the present invention. The similarity of the chromosomal context between an ALS gene comprised by the plant of the present invention and that of an ALS gene of a wild-type plant can, for example, be tested as follows:

Genome-specific satellite markers which surround a wild-type ALS gene and an ALS gene of the present invention can be used together with sequences from the ALS gene (preferably except for the codon at the position as specified herein which is different between the wild-type ALS gene and an ALS gene comprised by the plant of the present invention) for primer design and subsequent nucleic acid amplification, whereby the amplification product will be identical between a wild-type plant and the plant of the present invention. These genome-specific satellite markers can also be used for a fluorescent in situ hybridization (FISH) in order to check the location of the ALS gene (see Schmidt and Heslop-Harrison (1996), Proc. Natl. Acad. Sci. 93:8761-8765 for a FISH protocol of B. napus).

In view of the fact that mutated endogenous ALS genes of the present invention are located at the same chromosome at the same specific location, respectively, the “staining pattern” in FISH of the chromosome on which the wild-type ALS genes are located will be identical to the staining pattern in FISH of the chromosome on which the ALS genes of the present invention are located.

Of course, foreign genes can be transferred to the plant either by genetic engineering or by conventional methods such as crossing. Said genes can be genes conferring herbicide tolerances, preferably conferring herbicide tolerances different from ALS inhibitor herbicide tolerances, genes improving yield, genes improving resistances to biological organisms, and/or genes concerning content modifications.

The plants according to the invention form the basis for the development of commercial varieties including F1 hybrids following procedures known in the breeding community supported by molecular breeding techniques (like marker assisted breeding or marker assisted selection) for speeding up the processes and to secure the correct selection of plants to either obtain the mutation in its homozygous form or in case of comprising one or more mutations at various locations of the ALS encoding endogenous gene to perform the correct selection of heterozygous plants wherein at least at one of the alleles of each ALS gene comprises the Trp574Leu mutation (when referring to SEQ ID NO: 10) according to present invention and at least one of the alleles of one ALS gene comprises the Pro197Ser and the Trp574Leu mutation mutation (when referring to SEQ ID NO: 10) according to present invention.

Calli are obtained by means and methods commonly known in the art, e.g., Alexander Dovzhenko, PhD Thesis, Title: “Towards plastid transformation in rapeseed (Brassica napus L.) and sugarbeet (Beta vulgaris L.)”, Ludwig-Maximilians-Universitit München, Germany, 2001):

B. napus seeds can be immersed for 60 seconds in 70% ethanol, then rinsed twice in sterile water with 0.01% detergent and then incubated for 1-4 hours in 1% NaOCl bleach. After washing with sterile H₂O at 4° C., the embryos can be isolated using, e.g., forceps and scalpel.

The freshly prepared embryos can be immersed in 0.5% NaOCl for 30 min and then washed in sterile H₂O. After the last washing step they can be placed on hormone free MS agar medium (Murashige and Skoog (1962), Physiol. Plantarum, 15, 473-497). Those embryos which developed into sterile seedlings can be used for the initiation of regenerable B. napus cell cultures.

Cotyledons as well as hypocotyls can be cut into 2-5 mm long segments and then cultivated on agar (0.8%) solidified MS agar medium containing either 1 mg/1 Benzylaminopurin (BAP) or 0.25 mg/l Thidiazuron (TDZ). 4 weeks later the developing shoot cultures can be transferred onto fresh MS agar medium of the same composition and then sub-cultured in monthly intervals. The cultures can be kept at 25° C. under dim light at a 12 h/12 h light/dark cycle.

After 7-10 days, subcultures the shoot cultures which were grown on the thidiazuron containing medium formed a distinct callus type, which was fast growing, soft and friable. The colour of this callus type is typically yellowish to light green. Some of these friable calli consistently produced chlorophyll containing shoot primordia from embryo-like structures. These fast growing regenerable calli can be used for the selection of ALS inhibitor herbicide tolerant B. napus mutants.

A particular embodiment of the invention relates to a method to increase the tolerance to ALS inhibitor herbicide(s) of polyploid plants, said method comprising introducing at least two ALS genes, wherein said at least two ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine.

At least two genes which encode an ALS polypeptide comprising at a position corresponding to position 574 of SEQ ID NO: 10 instead of the naturally encoded amino acid tryptophan the amino acid leucine, and at least one gene which encode an ALS polypeptide comprising at a position corresponding to position 197 of SEQ ID NO: 10 instead of the naturally encoded amino acid proline the amino acid serine and at a position corresponding to position 574 of SEQ ID NO: 10 instead of the naturally encoded amino acid tryptophan the amino acid leucine can be introduced by selection methods, such as selection methods described herein in the examples. Upon selection, plants can be identified in which all ALS genes encode an ALS polypeptide comprising at a position corresponding to position 574 of SEQ ID NO: 10 instead of the naturally encoded amino acid tryptophan the amino acid leucine and one ALS gene encodes an ALS polypeptide which further comprises at a position corresponding to position 197 of SEQ ID NO: 10 instead of the naturally encoded amino acid proline the amino acid serine.

Said at genes least two genes which encode an ALS polypeptide comprising at a position corresponding to position 574 of SEQ ID NO: 10 instead of the naturally encoded amino acid tryptophan the amino acid leucine and said at least one gene which encodes an ALS polypeptide comprising at a position corresponding to position 197 of SEQ ID NO: 10 instead of the naturally encoded amino acid proline the amino acid serine and at a position corresponding to position 574 of SEQ ID NO: 10 instead of the naturally encoded amino acid tryptophan the amino acid leucine can also be introduced by crossing a plant comprising at least a first ALS gene encoding an ALS polypeptide comprising at a position corresponding to position 574 of SEQ ID NO: 10 instead of the naturally encoded amino acid tryptophan the amino acid leucine with another plant comprising at least a second ALS gene encoding an ALS polypeptide comprising at a position corresponding to position 197 of SEQ ID NO: 10 instead of the naturally encoded amino acid proline the amino acid serine and at a position corresponding to position 574 of SEQ ID NO: 10 instead of the naturally encoded amino acid tryptophan the amino acid leucine. Said first ALS gene may, optionally, further comprise at a position corresponding to position 197 of SEQ ID NO: 10 instead of the naturally encoded amino acid proline the amino acid serine. Optionally, the progeny plants can be identified using molecular methods as described herein. Alternatively, said at least two genes which encode an ALS polypeptide comprising at a position corresponding to position 574 of SEQ ID NO: 10 instead of the naturally encoded amino acid tryptophan the amino acid leucine and said at least one gene which encodes an ALS polypeptide comprising at a position corresponding to position 197 of SEQ ID NO: 10 instead of the naturally encoded amino acid proline the amino acid serine and at a position corresponding to position 574 of SEQ ID NO: 10 instead of the naturally encoded amino acid tryptophan the amino acid leucine can also be introduced by crossing a plant comprising at least two ALS gene encoding an ALS polypeptide comprising at a position corresponding to position 574 of SEQ ID NO: 10 instead of the naturally encoded amino acid tryptophan the amino acid leucine and at least one gene which encodes an ALS polypeptide comprising at a position corresponding to position 197 of SEQ ID NO: 10 instead of the naturally encoded amino acid proline the amino acid serine and at a position corresponding to position 574 of SEQ ID NO: 10 instead of the naturally encoded amino acid tryptophan the amino acid leucine, with another plant not comprising said at least two ALS genes encoding an ALS polypeptide comprising at a position corresponding to position 574 of SEQ ID NO: 10 instead of the naturally encoded amino acid tryptophan the amino acid leucine, and not comprising said at least one gene which encodes an ALS polypeptide comprising at a position corresponding to position 197 of SEQ ID NO: 10 instead of the naturally encoded amino acid proline the amino acid serine and at a position corresponding to position 574 of SEQ ID NO: 10 instead of the naturally encoded amino acid tryptophan the amino acid leucine. Optionally, the progeny plants can be identified using molecular methods as described herein. It will be clear that the progeny plants contain at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine.

Described herein are methods to increase the tolerance to ALS inhibitor herbicide(s) of Brassica napus plants, said method comprising introducing an ALS I gene encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and introducing an ALS III gene encoding an ALS III polypeptide comprising at a position corresponding to position 556 of SEQ ID NO: 4 instead of the naturally encoded amino acid tryptophan the amino acid leucine, or said method comprising introducing an ALS I gene encoding an ALS I polypeptide comprising at a position corresponding to position 559 of SEQ ID NO: 2 instead of the naturally encoded amino acid tryptophan the amino acid leucine, and introducing an ALS III gene encoding an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or said method comprising introducing an ALS I gene encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and introducing an ALS III gene encoding an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, and methods to increase the tolerance to ALS inhibitor herbicide(s) of Brassica juncea plants, said method comprising introducing an ALS-A gene encoding an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine, and introducing an ALS-B gene encoding an ALS-B polypeptide comprising at a position corresponding to position 559 of SEQ ID NO: 8 instead of the naturally encoded amino acid tryptophan the amino acid leucine, or said method comprising introducing an ALS-A gene encoding an ALS-A polypeptide comprising at a position corresponding to position 556 of SEQ ID NO: 6 instead of the naturally encoded amino acid tryptophan the amino acid leucine, and introducing an ALS-B gene encoding an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine, or said method comprising introducing an ALS-A gene encoding an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine, and introducing an ALS-B gene encoding an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine.

### Use

The present invention further relates to the use of one or more ALS inhibitor herbicide(s) in mutant plants according to the invention comprising mutations of all endogenous acetolactate synthase (ALS) genes, wherein all ALS genes encode an ALS polypeptide containing leucine instead of tryptophan at a position corresponding to 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as B. napus mutants wherein an ALS I gene encodes an ALS I polypeptide containing serine instead of proline at a position 182 and leucine instead of tryptophan at a position 559 of said ALS I polypeptide and wherein an ALS III gene encodes an ALS III polypeptide containing leucine instead of tryptophan at a position 556 of said ALS III polypeptide, such as B. napus mutants wherein an ALS I gene encodes an ALS I polypeptide containing leucine instead of tryptophan at a position 559 of said ALS I polypeptide and wherein an ALS III gene encodes an ALS III polypeptide containing serine instead of proline at a position 179 and leucine instead of tryptophan at a position 556 of said ALS III polypeptide, or such as B. napus mutants wherein an ALS I gene encodes an ALS I polypeptide containing serine instead of proline at a position 182 and leucine instead of tryptophan at a position 559 of said ALS I polypeptide and wherein an ALS III gene encodes an ALS III polypeptide polypeptide containing serine instead of proline at a position 179 and leucine instead of tryptophan at a position 556 of said ALS III polypeptide, or such as B. juncea mutants wherein an ALS-A gene encodes an ALS-A polypeptide containing serine instead of proline at a position 179 and leucine instead of tryptophan at a position 556 of said ALS-A polypeptide and wherein an ALS-B gene encodes an ALS-B polypeptide containing leucine instead of tryptophan at a position 559 of said ALS-B polypeptide, or such as B. juncea mutants wherein an ALS-A gene encodes an ALS-A polypeptide containing leucine instead of tryptophan at a position 556 of said ALS-A polypeptide and wherein an ALS-B gene encodes an ALS-B polypeptide containing serine instead of proline at a position 182 and leucine instead of tryptophan at a position 559 of said ALS-B polypeptide, or such as B. juncea mutants wherein an ALS-A gene encodes an ALS-A polypeptide containing serine instead of proline at a position 179 and leucine instead of tryptophan at a position 556 of said ALS-A polypeptide and wherein an ALS-B gene encodes an ALS-B polypeptide containing serine instead of proline at a position 182 and leucine instead of tryptophan at a position 559 of said ALS-B polypeptide, and wherein the ALS inhibitor herbicide(s) belong to:

the group of the (sulfon)amides (group (A)) consisting of:

the subgroup (A1) of the sulfonylureas, consisting of:
amidosulfuron [CAS RN 120923-37-7] (=A1-1);
azimsulfuron [CAS RN 120162-55-2] (=A1-2);
bensulfuron-methyl [CAS RN 83055-99-6] (=A1-3);
chlorimuron-ethyl [CAS RN 90982-32-4] (=A1-4);
chlorsulfuron [CAS RN 64902-72-3] (=A1-5);
cinosulfuron [CAS RN 94593-91-6] (=A1-6);
cyclosulfamuron [CAS RN 136849-15-5] (=A1-7);
ethametsulfuron-methyl [CAS RN 97780-06-8] (=A1-8);
ethoxysulfuron [CAS RN 126801-58-9] (=A1-9);
flazasulfuron [CAS RN 104040-78-0] (=A1-10);
flucetosulfuron [CAS RN 412928-75-7] (=A1-11);
flupyrsulfuron-methyl-sodium [CAS RN 144740-54-5] (=A1-12);
foramsulfuron [CAS RN 173159-57-4] (=A1-13);
halosulfuron-methyl [CAS RN 100784-20-1] (=A1-14);
imazosulfuron [CAS RN 122548-33-8] (=A1-15);
iodosulfuron-methyl-sodium [CAS RN 144550-36-7] (=A1-16);
mesosulfuron-methyl [CAS RN 208465-21-8] (=A1-17);
metsulfuron-methyl [CAS RN 74223-64-6] (=A1-18);
monosulfuron [CAS RN 155860-63-2] (=A1-19);
nicosulfuron [CAS RN 111991-09-4] (=A1-20);
orthosulfamuron [CAS RN 213464-77-8] (=A1-21);
oxasulfuron [CAS RN 144651-06-9] (=A1-22);
primisulfuron-methyl [CAS RN 86209-51-0] (=A1-23);
prosulfuron [CAS RN 94125-34-5] (=A1-24);
pyrazosulfuron-ethyl [CAS RN 93697-74-6] (=A1-25);
rimsulfuron [CAS RN 122931-48-0] (=A1-26);
sulfometuron-methyl [CAS RN 74222-97-2] (=A1-27);
sulfosulfuron [CAS RN 141776-32-1] (=A1-28);
thifensulfuron-methyl [CAS RN 79277-27-3] (=A1-29);
triasulfuron [CAS RN 82097-50-5] (=A1-30);
tribenuron-methyl [CAS RN 101200-48-0] (=A1-31);
trifloxysulfuron [CAS RN 145099-21-4] (sodium) (=A1-32);
triflusulfuron-methyl [CAS RN 126535-15-7] (=A1-33);
tritosulfuron [CAS RN 142469-14-5] (=A1-34);
NC-330 [CAS RN 104770-29-8] (=A1-35);
NC-620 [CAS RN 868680-84-6] (=A1-36);
TH-547 [CAS RN 570415-88-2] (=A1-37);
monosulfuron-methyl [CAS RN 175076-90-1] (=A1-38);
metazosulfuron [CAS RN 868680-84-6] (=A1-39);
methiopyrsulfuron [CAS RN 441050-97-1] (=A1-40);
iofensulfuron-sodium [CAS RN 1144097-30-2] (=A1-41);
propyrisulfuron [CAS RN 570415-88-2] (=A1-42).

the subgroup of the sulfonylaminocarbonyltriazolinones (subgroup ((A2)), consisting of:
flucarbazone-sodium [CAS RN 181274-17-9] (=A2-1);
propoxycarbazone-sodium [CAS RN 181274-15-7] (=A2-2);
thiencarbazone-methyl [CAS RN 317815-83-1] (=A2-3).

the subgroup of the triazolopyrimidines (subgroup (A3)), consisting of:
cloransulam-methyl [147150-35-4] (=A3-1);
diclosulam [CAS RN 145701-21-9] (=A3-2);
florasulam [CAS RN 145701-23-1] (=A3-3);
flumetsulam [CAS RN 98967-40-9] (=A3-4);
metosulam [CAS RN 139528-85-1] (=A3-5);
penoxsulam [CAS RN 219714-96-2] (=A3-6);
pyroxsulam [CAS RN 422556-08-9] (=A3-7).

the subgroup of the sulfonanilides (subgroup (A4)), consisting of:
compounds or salts thereof, and racemates and enantiomers thereof, from the group described by the general formula (I):

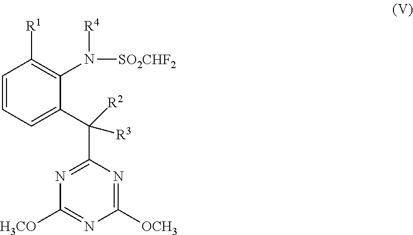

in which
R¹ is halogen, preferably fluorine or chlorine,
R² is hydrogen and R³ is hydroxyl or
R² and R³ together with the carbon atom to which they are attached are a carbonyl group C═O and
R⁴ is hydrogen or methyl;
and more especially compounds of the below given chemical structure (A4-1) to (A4-8)

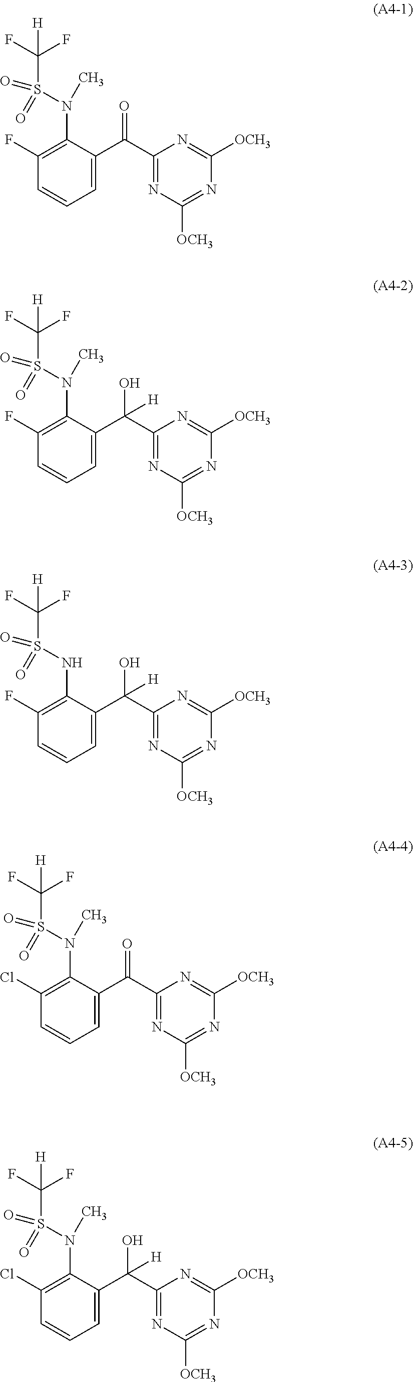 [SMILES: COc1nc(OC)nc(C(=O)c2cccc(F)c2N(C)S(=O)(=O)C(F)F)n1 ; COc1nc(OC)nc(C(O)c2cccc(F)c2N(C)S(=O)(=O)C(F)F)n1 ; COc1nc(OC)nc(C(O)c2cccc(F)c2NS(=O)(=O)C(F)F)n1 ; COc1nc(OC)nc(C(=O)c2cccc(Cl)c2N(C)S(=O)(=O)C(F)F)n1 ; COc1nc(OC)nc(C(O)c2cccc(Cl)c2N(C)S(=O)(=O)C(F)F)n1]

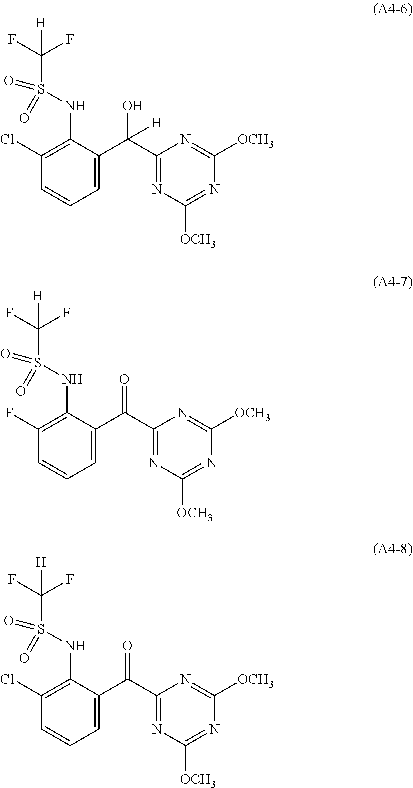 [SMILES: COc1nc(OC)nc(C(O)c2cccc(Cl)c2NS(=O)(=O)C(F)F)n1 ; COc1nc(OC)nc(C(=O)c2cccc(F)c2NS(=O)(=O)C(F)F)n1 ; COc1nc(OC)nc(C(=O)c2cccc(Cl)c2NS(=O)(=O)C(F)F)n1]

the group of the imidazolinones (group (B1)), consisting of:
imazamethabenzmethyl [CAS RN 81405-85-8] (=B1-1);
imazamox [CAS RN 114311-32-9] (=B1-2);
imazapic [CAS RN 104098-48-8] (=B1-3);
imazapyr [CAS RN 81334-34-1] (=B1-4);
imazaquin [CAS RN 81335-37-7] (=B1-5);
imazethapyr [CAS RN 81335-77-5] (=B1-6);
SYP-298 [CAS RN 557064-77-4] (=B1-7);
SYP-300 [CAS RN 374718-10-2] (=B1-8).
   the group of the pyrimidinyl(thio)benzoates (group (C)), consisting of:

the subgroup of the pyrimidinyloxybenzoeacids (subgroup (C1)) consisting of:
bispyribac-sodium [CAS RN 125401-92-5] (=C1-1);
pyribenzoxim [CAS RN 168088-61-7] (=C1-2);
pyriminobac-methyl [CAS RN 136191-64-5] (=C1-3);
pyribambenz-isopropyl [CAS RN 420138-41-6] (=C1-4);
pyribambenz-propyl [CAS RN 420138-40-5] (=C1-5).

the subgroup of the pyrimidinylthiobenzoeacids (subgroup (C2)), consisting of:
pyriftalid [CAS RN 135186-78-6] (=C2-1);
pyrithiobac-sodium [CAS RN 123343-16-8] (=C2-2).

In this context, “tolerance” or “tolerant” means that the application of one or more ALS inhibitor herbicide(s) belonging to any of the above defined groups (A), (B), (C) have reduced apparent effect(s), as compared to effect(s) on wild type plants, concerning the physiological functions/phytotoxicity when applied to the respective plant, such as B. napus plants or B. juncea plants according to the invention, having mutations of its endogenous acetolactate synthase (ALS) genes, wherein a first ALS gene, such as ALS I B. napus, or ALS-A B. juncea, gene encodes a first ALS, such as B. napus or B. juncea, polypeptide containing serine instead of proline at a position corresponding to position 197 of SEQ ID NO: 10 and leucine instead of tryptophan at a position corresponding to position 574 of SEQ ID NO: 10 and wherein a second ALS gene, such as an ALS III B. napus, or ALS-B B. juncea, gene encodes a second ALS, such as B. napus or B. juncea, polypeptide containing leucine instead of tryptophan at a position corresponding to position 574 of SEQ ID NO: 10, or such as B. napus plants or B. juncea plants according to the invention, having mutations of its endogenous acetolactate synthase (ALS) genes, wherein a first ALS gene, such as ALS I B. napus, or ALS-A B. juncea, gene encodes a first ALS, such as B. napus or B. juncea, polypeptide containing leucine instead of tryptophan at a position corresponding to position 574 of SEQ ID NO: 10 and wherein a second ALS gene, such as an ALS III B. napus, or ALS-B B. juncea, gene encodes a second ALS, such as B. napus or B. juncea, polypeptide containing serine instead of proline at a position corresponding to position 197 of SEQ ID NO: 10 and leucine instead of tryptophan at a position corresponding to position 574 of SEQ ID NO: 10, or such as B. napus plants or B. juncea plants according to the invention, having mutations of its endogenous acetolactate synthase (ALS) genes, wherein a first ALS gene, such as ALS I B. napus, or ALS-A B. juncea, gene encodes a first ALS, such as B. napus or B. juncea, polypeptide containing serine instead of proline at a position corresponding to position 197 of SEQ ID NO: 10 and leucine instead of tryptophan at a position corresponding to position 574 of SEQ ID NO: 10 and wherein a second ALS gene, such as an ALS III B. napus, or ALS-B B. juncea, gene encodes a second ALS, such as B. napus or B. juncea, polypeptide containing serine instead of proline at a position corresponding to position 197 of SEQ ID NO: 10 and leucine instead of tryptophan at a position corresponding to position 574 of SEQ ID NO: 10, and whereas the application of the same amount of the respective ALS inhibitor herbicide(s) on non-tolerant plants, such as B. napus or B. juncea, wild type plants leads to significant negative effects concerning plant growth, its physiological functions or shows phytotoxic sypmtoms. Quality and quantity of the observed effects may depend on the chemical composition of the respective ALS inhibitor heribicide(s) applied, dose rate and timing of the application as well growth conditions/stage of the treated plants.

The “CAS RN” stated in square brackets after the names (common names) mentioned under groups A to C corresponds to the “chemical abstract service registry number”, a customary reference number which allows the substances named to be classified unambiguously, since the “CAS RN” distinguishes, inter alia, between isomers including stereoisomers.

ALS inhibitor herbicides which are preferably used for control of unwanted vegetation in plant growing areas, such as B. napus or B. juncea growing areas which plants, such as B. napus plants or B. juncea plants comprise mutations of its endogenous acetolactate synthase (ALS) genes, wherein at least two ALS genes encode an ALS polypeptide containing leucine instead of tryptophan at a position corresponding to position 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing leucine instead of tryptophan at a position 556 of said ALS III polypeptide, or such as B. napus wherein the ALS I gene encodes an ALS I polypeptide containing leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing leucine instead of tryptophan at a position 559 of said ALS-B polypeptide, or such as B. juncea wherein the ALS-A gene encodes an ALS-A polypeptide containing leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, and thereby providing tolerance against the ALS inhibitor herbicide(s) according to this invention belonging to group (A) are:

amidosulfuron [CAS RN 120923-37-7] (=A1-1);
chlorimuron-ethyl [CAS RN 90982-32-4] (=A1-4);
chlorsulfuron [CAS RN 64902-72-3] (=A1-5);
ethametsulfuron-methyl [CAS RN 97780-06-8] (=A1-8);
ethoxysulfuron [CAS RN 126801-58-9] (=A1-9);
flupyrsulfuron-methyl-sodium [CAS RN 144740-54-5] (=A1-12);
foramsulfuron [CAS RN 173159-57-4] (=A1-13);
iodosulfuron-methyl-sodium [CAS RN 144550-36-7] (=A1-16);
mesosulfuron-methyl [CAS RN 208465-21-8] (=A1-17);
metsulfuron-methyl [CAS RN 74223-64-6] (=A1-18);
monosulfuron [CAS RN 155860-63-2] (=A1-19);
nicosulfuron [CAS RN 111991-09-4] (=A1-20);
rimsulfuron [CAS RN 122931-48-0] (=A1-26);
sulfosulfuron [CAS RN 141776-32-1] (=A1-28);
thifensulfuron-methyl [CAS RN 79277-27-3] (=A1-29);
tribenuron-methyl [CAS RN 101200-48-0] (=A1-31);
triflusulfuron-methyl [CAS RN 126535-15-7] (=A1-33);
iofensulfuron-sodium [CAS RN 1144097-30-2] (=A1-41);
flucarbazone-sodium [CAS RN 181274-17-9] (=A2-1);
propoxycarbazone-sodium [CAS RN 181274-15-7] (=A2-2);
thiencarbazone-methyl [CAS RN 317815-83-1] (=A2-3);
florasulam [CAS RN 145701-23-1] (=A3-3);
metosulam [CAS RN 139528-85-1] (=A3-5);
pyroxsulam [CAS RN 422556-08-9] (=A3-7);

### (A4-1); (A4-2) and (A4-3).

ALS inhibitor herbicides which are more preferably used for control of unwanted in plant growing areas, such as B. napus or B. juncea growing areas which plants, such as B. napus plants or B. juncea plants comprise mutations of its endogenous acetolactate synthase (ALS) genes, wherein at least two ALS genes encode an ALS polypeptide containing leucine instead of tryptophan at a position corresponding to position 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing leucine instead of tryptophan at a position 556 of said ALS III polypeptide, or such as B. napus wherein the ALS I gene encodes an ALS I polypeptide containing leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing leucine instead of tryptophan at a position 559 of said ALS-B polypeptide, or such as B. juncea wherein the ALS-A gene encodes an ALS-A polypeptide containing leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, and thereby providing tolerance against the ALS inhibitor herbicide(s) according to this invention belonging to group (A) are:

amidosulfuron [CAS RN 120923-37-7] (=A1-1);
ethoxysulfuron [CAS RN 126801-58-9] (=A1-9);
flupyrsulfuron-methyl-sodium [CAS RN 144740-54-5] (=A1-12);
foramsulfuron [CAS RN 173159-57-4] (=A1-13);
iodosulfuron-methyl-sodium [CAS RN 144550-36-7] (=A1-16);
mesosulfuron-methyl [CAS RN 208465-21-8] (=A1-17);
metsulfuron-methyl [CAS RN 74223-64-6] (=A1-18);
nicosulfuron [CAS RN 111991-09-4] (=A1-20);
rimsulfuron [CAS RN 122931-48-0] (=A1-26);
sulfosulfuron [CAS RN 141776-32-1] (=A1-28);
thifensulfuron-methyl [CAS RN 79277-27-3] (=A1-29);
tribenuron-methyl [CAS RN 101200-48-0] (=A1-31);
iofensulfuron-sodium [CAS RN 1144097-30-2] (=A1-41)
propoxycarbazone-sodium [CAS RN 181274-15-7] (=A2-2);
thiencarbazone-methyl [CAS RN 317815-83-1] (=A2-3);
florasulam [CAS RN 145701-23-1] (=A3-3);
metosulam [CAS RN 139528-85-1] (=A3-5); and
pyroxsulam [CAS RN 422556-08-9] (=A3-7).

ALS inhibitor herbicides which are especially preferably used for control of unwanted vegetation in plant growing areas, such as B. napus or B. juncea growing areas which plants, such as B. napus plants or B. juncea plants comprise mutations of its endogenous acetolactate synthase (ALS) genes, wherein at least two ALS genes encode an ALS polypeptide containing leucine instead of tryptophan at a position corresponding to position 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing leucine instead of tryptophan at a position 556 of said ALS III polypeptide, or such as B. napus wherein the ALS I gene encodes an ALS I polypeptide containing leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing leucine instead of tryptophan at a position 559 of said ALS-B polypeptide, or such as B. juncea wherein the ALS-A gene encodes an ALS-A polypeptide containing leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, and thereby providing tolerance against the ALS inhibitor herbicide(s) according to this invention belonging to group (A) are:

amidosulfuron [CAS RN 120923-37-7] (=A1-1);
foramsulfuron [CAS RN 173159-57-4] (=A1-13);
iofensulfuron-sodium [CAS RN 1144097-30-2] (=A1-41); and
thiencarbazone-methyl [CAS RN 317815-83-1] (=A2-3).

Another ALS inhibitor herbicide which is preferably used for control of unwanted vegetation in plant growing areas, such as B. napus or B. juncea growing areas which plants, such as B. napus plants or B. juncea plants comprise mutations of its endogenous acetolactate synthase (ALS) genes, wherein at least two ALS genes encode an ALS polypeptide containing leucine instead of tryptophan at a position corresponding to position 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing leucine instead of tryptophan at a position 556 of said ALS III polypeptide, or such as B. napus wherein the ALS I gene encodes an ALS I polypeptide containing leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing leucine instead of tryptophan at a position 559 of said ALS-B polypeptide, or such as B. juncea wherein the ALS-A gene encodes an ALS-A polypeptide containing leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, and thereby providing tolerance against the ALS inhibitor herbicide(s) according to this invention belonging to group (B) is imazamox [CAS RN 114311-32-9] (=B1-2).

Another ALS inhibitor herbicide which is preferably used for control of unwanted vegetation in plant growing areas, such as B. napus or B. juncea growing areas which plants, such as B. napus plants or B. juncea plants comprise mutations of its endogenous acetolactate synthase (ALS) genes, wherein at least two ALS genes encode an ALS polypeptide containing leucine instead of tryptophan at a position corresponding to position 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing leucine instead of tryptophan at a position 556 of said ALS III polypeptide, or such as B. napus wherein the ALS I gene encodes an ALS I polypeptide containing leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. napus wherein the ALS I gene encodes an ALS I polypeptide containing serine instead of proline at a position corresponding to position 182 and leucine instead of tryptophan at a position corresponding to position 559 of said first ALS I polypeptide and wherein the ALS III gene encodes an ALS III polypeptide containing serine instead of proline at a position corresponding to position 179 and leucine instead of tryptophan at a position 556 of said ALS III polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing leucine instead of tryptophan at a position 559 of said ALS-B polypeptide, or such as B. juncea wherein the ALS-A gene encodes an ALS-A polypeptide containing leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, or such as B. juncea wherein the ALS-A gene encodes an ALS-A polypeptide containing serine instead of proline at a position corresponding to position 179 and leucine instead of tryptophan at a position corresponding to position 556 of said first ALS-A polypeptide and wherein the ALS-B gene encodes an ALS-B polypeptide containing serine instead of proline at a position corresponding to position 182 and leucine instead of tryptophan at a position 559 of said ALS-B polypeptide, and thereby providing tolerance against the ALS inhibitor herbicide(s) according to this invention belonging to group (C) is bispyribac-sodium [CAS RN 125401-92-5] (=C1-1).

It is to be further understood that concerning all above defined ALS inhibitor herbicides and where not already specified by the respective CAS RN, all use forms, such as acids, and salts can be applied according to the invention.

Additionally, the ALS inhibitor herbicide(s) to be used according to the invention may comprise further components, for example agrochemically active compounds of a different type of mode of action and/or the formulation auxiliaries and/or additives customary in crop protection, or may be used together with these.

In a further embodiment, the herbicide combinations to be used according to the invention comprise effective amounts of the ALS inhibitor herbicide(s) belonging to groups (A), (B) and/or (C) and/or have synergistic actions. The synergistic actions can be observed, for example, when applying one or more ALS inhibitor herbicide(s) belonging to groups (A), (B), and/or (C) together, for example as a coformulation or as a tank mix; however, they can also be observed when the active compounds are applied at different times (splitting). It is also possible to apply the herbicides or the herbicide combinations in a plurality of portions (sequential application), for example pre-emergence applications followed by post-emergence applications or early post-emergence applications followed by medium or late post-emergence applications. Preference is given here to the joint or almost simultaneous application of the ALS-inhibitor herbicides belonging to groups (A), (B) and/or (C) of the combination in question.

The synergistic effects permit a reduction of the application rates of the individual ALS inhibitor herbicides, a higher efficacy at the same application rate, the control of species which were as yet uncontrolled (gaps), control of species which are tolerant or resistant to individual ALS inhibitor herbicides or to a number of ALS inhibitor herbicides, an extension of the period of application and/or a reduction in the number of individual applications required and—as a result for the user—weed control systems which are more advantageous economically and ecologically.

The herbicides to be used according to this invention are all acetolactate synthase (ALS) inhibitor herbicides and thus inhibit protein biosynthesis in plants.

The application rate of the ALS inhibitor herbicides belonging to groups (A), (B) or (C) (as defined above) can vary within a wide range, for example between 0.001 g and 1500 g of ai/ha (ai/ha means here and below “active substance per hectare”=based on 100% pure active compound). Applied at application rates of from 0.001 g to 1500 g of ai/ha, the herbicides belonging to classes A, B and C according to this invention, preferably the compounds A1-1; A1-4; A1-9; A1-12; A1-13; A1-16; A1-17; A1-18; A1-20; A1-26; A1-28; A1-29; A1-31; A1-41; A2-2; A3-3; A3-5; A3-7, control, when used by the pre- and post-emergence method, a relatively wide spectrum of harmful plants, for example of annual and perennial mono- or dicotyledonous weeds, and also of unwanted crop plants (together also defined as “unwanted vegetation).

In many applications according to the invention, the application rates are generally lower, for example in the range of from 0.001 g to 1000 g of ai/ha, preferably from 0.1 g to 500 g of ai/ha, particularly preferably from 0.5 g to 250 g of ai/ha, and even more preferably 1.0 g to 200 g of ai/ha. In cases where the application of several ALS inhibitor herbicides is conducted, the quantity represents the total quantity of all of the applied ALS inhibitor herbicides.

For example, the combinations according to the invention of ALS inhibitor herbicides (belonging to groups (A), (B) and/or (C)) allow the activity to be enhanced synergistically in a manner which, by far and in an unexpected manner, exceeds the activities which can be achieved using the individual ALS inhibitor herbicides (belonging to groups (A), (B) and/or (C)).

For combinations of ALS inhibitor herbicides, the preferred conditions are illustrated below.

Of particular interest according to present invention is the use of herbicidal compositions for control of unwanted vegetation in polyploid plants, such as B. napus or B. juncea plants, preferably in mutated plants as described herein having a content of the following ALS inhibitor herbicides:

(A1-1)+(A1-9); (A1-1)+(A1-12); (A1-1)+(A1-13); (A1-1)+(A1-16); (A1-1)+(A1-17); (A1-1)+(A1-18); (A1-1)+(A1-20); (A1-1)+(A1-26); (A1-1)+(A1-28); (A1-1)+(A1-29); (A1-1)+(A1-31); (A1-1)+(A1-41); (A1-1)+(A2-2); (A1-1)+(A2-3); (A1-1)+(A3-3); (A1-1)+(A3-5); (A1-1)+(A3-7); (A1-1)+(B1-2); (A1-1)+(C1-1);
(A1-9)+(A1-12); (A1-9)+(A1-13); (A1-9)+(A1-16); (A1-9)+(A1-17); (A1-9)+(A1-18); (A1-9)+(A1-20); (A1-9)+(A1-26); (A1-9)+(A1-28); (A1-9)+(A1-29); (A1-9)+(A1-31); (A1-9)+(A1-41); (A1-9)+(A2-2); (A1-9)+(A2-3); (A1-9)+(A3-3); (A1-9)+(A3-5); (A1-9)+(A3-7); (A1-9)+(B1-2); (A1-9)+(C1-1);
(A1-12)+(A1-13); (A1-12)+(A1-16); (A1-12)+(A1-17); (A1-12)+(A1-18); (A1-12)+(A1-20); (A1-12)+(A1-26); (A1-12)+(A1-28); (A1-12)+(A1-29); (A1-12)+(A1-31); (A1-12)+(A1-41); (A1-12)+(A2-2); (A1-12)+(A2-3); (A1-12)+(A3-3); (A1-12)+(A3-5); (A1-12)+(A3-7); (A1-12)+(B1-2); (A1-12)+(C1-1);
(A1-13)+(A1-16); (A1-13)+(A1-17); (A1-13)+(A1-18); (A1-13)+(A1-20); (A1-13)+(A1-26); (A1-13)+(A1-28); (A1-13)+(A1-29); (A1-13)+(A1-31); (A1-13)+(A1-41); (A1-13)+(A2-2); (A1-13)+(A2-3); (A1-13)+(A3-3); (A1-13)+(A3-5); (A1-13)+(A3-7); (A1-13)+(B1-2); (A1-13)+(C1-1);
(A1-16)+(A1-17); (A1-16)+(A1-18); (A1-16)+(A1-20); (A1-16)+(A1-26); (A1-16)+(A1-28); (A1-16)+(A1-29); (A1-16)+(A1-31); (A1-16)+(A1-41); (A1-16)+(A2-2); (A1-16)+(A2-3); (A1-16)+(A3-3); (A1-16)+(A3-5); (A1-16)+(A3-7); (A1-16)+(B1-2); (A1-16)+(C1-1);
(A1-17)+(A1-18); (A1-17)+(A1-20); (A1-17)+(A1-26); (A1-17)+(A1-28); (A1-17)+(A1-29); (A1-17)+(A1-31); (A1-17)+(A1-41); (A1-17)+(A2-2); (A1-17)+(A2-3); (A1-17)+(A3-3); (A1-17)+(A3-5); (A1-17)+(A3-7); (A1-17)+(B1-2); (A1-17)+(C1-1);
(A1-18)+(A1-20); (A1-18)+(A1-26); (A1-18)+(A1-28); (A1-18)+(A1-29); (A1-18)+(A1-31); (A1-18)+(A1-41); (A1-18)+(A2-2); (A1-18)+(A2-3); (A1-18)+(A3-3); (A1-18)+(A3-5); (A1-18)+(A3-7); (A1-18)+(B1-2); (A1-18)+(C1-1);
(A1-20)+(A1-26); (A1-20)+(A1-28); (A1-20)+(A1-29); (A1-20)+(A1-31); (A1-20)+(A1-41); (A1-20)+(A2-2); (A1-20)+(A2-3); (A1-20)+(A3-3); (A1-20)+(A3-5); (A1-20)+(A3-7); (A1-20)+(B1-2); (A1-20)+(C1-1);

### (A1-26)+(A1-28); (A1-26)+(A1-29); (A1-26)+(A1-31); (A1-26)+(A1-41); (A1-26)+(A2-2); (A1-26)+(A2-3); (A1-26)+(A3-3); (A1-26)+(A3-5); (A1-26)+(A3-7); (A1-26)+(B1-2); (A1-26)+(C1-1);

### (A1-28)+(A1-29); (A1-28)+(A1-31); (A1-28)+(A1-41); (A1-28)+(A2-2); (A1-28)+(A2-3); (A1-28)+(A3-3); (A1-28)+(A3-5); (A1-28)+(A3-7); (A1-28)+(B1-2); (A1-28)+(C1-1);

### (A1-29)+(A1-31); (A1-29)+(A1-41); (A1-29)+(A2-2); (A1-29)+(A2-3); (A1-29)+(A3-3); (A1-29)+(A3-5); (A1-29)+(A3-7); (A1-29)+(B1-2); (A1-29)+(C1-1);

### (A1-31)+(A1-41); (A1-31)+(A2-2); (A1-31)+(A2-3); (A1-31)+(A3-3); (A1-31)+(A3-5); (A1-31)+(A3-7); (A1-31)+(B1-2); (A1-31)+(C1-1);

### (A1-41)+(A2-2); (A1-41)+(A2-3); (A1-41)+(A3-3); (A1-41)+(A3-5); (A1-41)+(A3-7); (A1-41)+(B1-2); (A1-41)+(C1-1);

### (A2-2)+(A2-3); (A2-2)+(A3-3); (A2-2)+(A3-5); (A2-2)+(A3-7); (A2-2)+(B1-2); (A2-2)+(C1-1);

### (A2-3)+(A3-3); (A2-3)+(A3-5); (A2-3)+(A3-7); (A2-3)+(B1-2); (A2-3)+(C1-1);

### (A3-3)+(A3-5); (A3-3)+(A3-7); (A3-3)+(B1-2); (A3-3)+(C1-1);

### (A3-5)+(A3-7); (A3-5)+(B1-2); (A3-5)+(C1-1);

### (A3-7)+(B1-2); (A3-7)+(C1-1);

### (B1-2)+(C1-1).

Additionally, the ALS inhibitor herbicides to be used according to the invention may comprise further components, for example agrochemically active compounds of a different type of mode of action and/or the formulation auxiliaries and/or additives customary in crop protection, or may be used together with these.

The ALS inhibitor herbicide(s) to be used according to the invention or combinations of various such ALS inhibitor herbicides may furthermore comprise various agrochemically active compounds, for example from the group of the safeners, fungicides, insecticides, or from the group of the formulation auxiliaries and additives customary in crop protection.

In a further embodiment, the invention relates to the use of effective amounts of ALS inhibitor herbicide(s) (i.e. members of the groups (A), (B) and/or (C)) and non-ALS inhibitor herbicides (i.e. herbicides showing a mode of action that is different to the inhibition of the ALS enzyme [acetohydroxyacid synthase; EC 2.2.1.6] (group B herbicides) in order obtain synergistic effect for the control of unwanted vegetation. Such synergistic actions can be observed, for example, when applying one or more ALS inhibitor herbicides (i.e. members of the groups (A), (B), and/or (C)) and one or more non ALS inhibitor herbicides (group B herbicides) together, for example as a coformulation or as a tank mix; however, they can also be observed when the active compounds are applied at different times (splitting). It is also possible to apply the ALS inhibitor herbicides and non ALS inhibitor herbicides in a plurality of portions (sequential application), for example pre-emergence applications followed by post-emergence applications or early post-emergence applications followed by medium or late post-emergence applications. Preference is given here to the joint or almost simultaneous application of the herbicides ((A), (B) and/or (C)) and (D) of the combination in question.

Suitable partner herbicides to be applied together with ALS inhibitor herbicideds are, for example, the following herbicides which differ structurally from the herbicides belonging to the groups (A), (B), and (C) as defined above, preferably herbicidally active compounds whose action is based on inhibition of, for example, acetyl coenzyme A carboxylase, PS I, PS II, HPPDO, phytoene desaturase, protoporphyrinogen oxidase, glutamine synthetase, cellulose biosynthesis, 5-enolpyruvylshikimate 3-phosphate synthetase, as described, for example, in Weed Research 26, 441-445 (1986), or “The Pesticide Manual”, 14th edition, The British Crop Protection Council, 2007, or 15^{th} edition 2010, or in the corresponding “e-Pesticide Manual”, Version 5 (2010), in each case published by the British Crop Protection Council, (hereinbelow in short also “PM”), and in the literature cited therein. Lists of common names are also available in “The Compendium of Pesticide Common Names” on the internet. Herbicides known from the literature (in brackets behind the common name hereinafter also classified by the indicators D1 to D426), which can be combined with ALS-inhibitor herbicides of groups (A), (B) and/or (C) and to be used according to present invention are, for example, the active compounds listed below: (note: the herbicides are referred to either by the “common name” in accordance with the International Organization for Standardization (ISO) or by the chemical name, together where appropriate with a customary code number, and in each case include all use forms, such as acids, salts, esters and isomers, such as stereoisomers and optical isomers, in particular the commercial form or the commercial forms, unless the context indicates otherwise. The citation given is of one use form and in some cases of two or more use forms):

acetochlor (=D1), acibenzolar (=D2), acibenzolar-S-methyl (=D3), acifluorfen (=D4), acifluorfen-sodium (=D5), aclonifen (=D6), alachlor (=D7), allidochlor (=D8), alloxydim (=D9), alloxydim-sodium (=D10), ametryn (=D11), amicarbazone (=D12), amidochlor (=D13), aminocyclopyrachlor (=D14), aminopyralid (=D15), amitrole (=D16), ammonium sulfamate (=D17), ancymidol (=D18), anilofos (=D19), asulam (=D20), atrazine (=D21), azafenidin (=D22), aziprotryn (=D23), beflubutamid (=D24), benazolin (=D25), benazolin-ethyl (=D26), bencarbazone (=D27), benfluralin (=D28), benfuresate (=D29), bensulide (=D30), bentazone (=D31), benzfendizone (=D32), benzobicyclon (=D33), benzofenap (=D34), benzofluor (=D35), benzoylprop (=D36), bicyclopyrone (=D37), bifenox (=D38), bilanafos (=D39), bilanafos-sodium (=D40), bromacil (=D41), bromobutide (=D42), bromofenoxim (=D43), bromoxynil (=D44), bromuron (=D45), buminafos (=D46), busoxinone (=D47), butachlor (=D48), butafenacil (=D49), butamifos (=D50), butenachlor (=D51), butralin (=D52), butroxydim (=D53), butylate (=D54), cafenstrole (=D55), carbetamide (=D56), carfentrazone (=D57), carfentrazone-ethyl (=D58), chlomethoxyfen (=D59), chloramben (=D60), chlorazifop (=D61), chlorazifop-butyl (=D62), chlorbromuron (=D63), chlorbufam (=D64), chlorfenac (=D65), chlorfenac-sodium (=D66), chlorfenprop (=D67), chlorflurenol (=D68), chlorflurenol-methyl (=D69), chloridazon (=D70), chlormequat-chloride (=D71), chlornitrofen (=D72), chlorophthalim (=D73), chlorthal-dimethyl (=D74), chlorotoluron (=D75), cinidon (=D76), cinidon-ethyl (=D77), cinmethylin (=D78), clethodim (=D79), clodinafop (=D80), clodinafop-propargyl (=D81), clofencet (=D82), clomazone (=D83), clomeprop (=D84), cloprop (=D85), clopyralid (=D86), cloransulam (=D87), cloransulam-methyl (=D88), cumyluron (=D89), cyanamide (=D90), cyanazine (=D91), cyclanilide (=D92), cycloate (=D93), cycloxydim (=D94), cycluron (=D95), cyhalofop (=D96), cyhalofop-butyl (=D97), cyperquat (=D98), cyprazine (=D99), cyprazole (=D100), 2,4-D (=D101), 2,4-DB (=D102), daimuron/dymron (=D103), dalapon (=D104), daminozide (=D105), dazomet (=D106), n-decanol (=D-107), desmedipham (=D108), desmetryn (=D109), detosyl-pyrazolate (=D110), diallate (=D111), dicamba (=D112), dichlobenil (=D113), dichlorprop (=D114), dichlorprop-P (=D115), diclofop (=D116), diclofop-methyl (=D117), diclofop-P-methyl (=D118), diethatyl (=D119), diethatyl-ethyl (=D120), difenoxuron (=D121), difenzoquat (=D122), diflufenican (=D123), diflufenzopyr (=D124), diflufenzopyr-sodium (=D125), dimefuron (=D126), dikegulac-sodium (=D127), dimefuron (=D128), dimepiperate (=D129), dimethachlor (=D130), dimethametryn (=D131), dimethenamid (=D132), dimethenamid-P (=D133), dimethipin (=D134), dimetrasulfuron (=D135), dinitramine (=D136), dinoseb (=D137), dinoterb (=D138), diphenamid (=D139), dipropetryn (=D140), diquat (=D141), diquat-dibromide (=D142), dithiopyr (=D143), diuron (=D144), DNOC (=D145), eglinazine-ethyl (=D146), endothal (=D147), EPTC (=D148), esprocarb (=D149), ethalfluralin (=D150), ethephon (=D151), ethidimuron (=D152), ethiozin (=D153), ethofumesate (=D154), ethoxyfen (=D155), ethoxyfen-ethyl (=D156), etobenzanid (=D157), F-5331 (=2-Chlor-4-fluor-5-[4-(3-fluorpropyl)-4,5-dihydro-5-oxo-1H-tetrazol-1-yl]-phenyl]-ethansulfonamid) (=D158), F-7967 (=3-[7-Chlor-5-fluor-2-(trifluormethyl)-1H-benzimidazol-4-yl]-1-methyl-6-(trifluormethyl)pyrimidin-2,4(1H,3H)-dion) (=D159), fenoprop (=D160), fenoxaprop (=D161), fenoxaprop-P (=D162), fenoxaprop-ethyl (=D163), fenoxaprop-P-ethyl (=D164), fenoxasulfone (=D165), fentrazamide (=D166), fenuron (=D167), flamprop (=D168), flamprop-M-isopropyl (=D169), flamprop-M-methyl (=D170), fluazifop (=D171), fluazifop-P (=D172), fluazifop-butyl (=D173), fluazifop-P-butyl (=D174), fluazolate (=D175), fluchloralin (=D176), flufenacet (thiafluamide) (=D177), flufenpyr (=D178), flufenpyr-ethyl (=D179), flumetralin (=D180), flumiclorac (=D181), flumiclorac-pentyl (=D182), flumioxazin (=D183), flumipropyn (=D184), fluometuron (=D185), fluorodifen (=D186), fluoroglycofen (=D187), fluoroglycofen-ethyl (=D188), flupoxam (=D189), flupropacil (=D190), flupropanate (=D191), flurenol (=D192), flurenol-butyl (=D193), fluridone (=D194), flurochloridone (=D195), fluroxypyr (=D196), fluroxypyr-meptyl (=D197), flurprimidol (=D198), flurtamone (=D199), fluthiacet (=D200), fluthiacet-methyl (=D201), fluthiamide (=D202), fomesafen (=203), forchlorfenuron (=D204), fosamine (=D205), furyloxyfen (=D206), gibberellic acid (=D207), glufosinate (=D208), glufosinate-ammonium (=D209), glufosinate-P (=D210), glufosinate-P-ammonium (=D211), glufosinate-P-sodium (=D212), glyphosate (=D213), glyphosate-isopropylammonium (=D214), H-9201 (═O-(2,4-Dimethyl-6-nitrophenyl)-O-ethyl-isopropylphosphoramidothioat) (=D215), halosafen (=D216), haloxyfop (=D217), haloxyfop-P (=D218), haloxyfop-ethoxyethyl (=D219), haloxyfop-P-ethoxyethyl (=D220), haloxyfop-methyl (=D221), haloxyfop-P-methyl (=D222), hexazinone (=D223), HW-02 (=1-(Dimethoxyphosphoryl)-ethyl(2,4-dichlorphenoxy)acetate) (=D224), inabenfide (=D225), indanofan (=D226), indaziflam (=D227), indol-3-acetic acid (IAA) (=D228), 4-indol-3-ylbutyric acid (IBA) (=D229), ioxynil (=D230), ipfencarbazone (=D231), isocarbamid (=D232), isopropalin (=D233), isoproturon (=D234), isouron (=D235), isoxaben (=D236), isoxachlortole (=D237), isoxaflutole (=D238), isoxapyrifop (=D239), KUH-043 (=3-({[5-(Difluormethyl)-1-methyl-3-(trifluormethyl)-1H-pyrazol-4-yl]methyl}sulfonyl)-5,5-dimethyl-4,5-dihydro-1,2-oxazol) (=D240), karbutilate (=D241), ketospiradox (=D242), lactofen (=D243), lenacil (=D244), linuron (=D245), male ic hydrazide (=D246), MCPA (=D247), MCPB (=D248), MCPB-methyl, -ethyl and -sodium (=D249), mecoprop (=D250), mecoprop-sodium (=D251), mecoprop-butotyl (=D252), mecoprop-P-butotyl (=D253), mecoprop-P-dimethylammonium (=D254), mecoprop-P-2-ethylhexyl (=D255), mecoprop-P-potassium (=D256), mefenacet (=D257), mefluidide (=D258), mepiquat-chloride (=D259), mesotrione (=D260), methabenzthiazuron (=D261), metam (=D262), metamifop (=D263), metamitron (=D264), metazachlor (=D265), metazole (=D266), methiopyrsulfuron (=D267), methiozolin (=D268), methoxyphenone (=D269), methyldymron (=D270), 1-methylcyclopropen (=D271), methylisothiocyanat (=D272), metobenzuron (=D273), metobromuron (=D274), metolachlor (=D275), S-metolachlor (=D-276), metoxuron (=D277), metribuzin (=D278), molinate (=D279), monalide (=D280), monocarbamide (=D281), monocarbamide-dihydrogensulfate (=D282), monolinuron (=D283), monosulfuron-ester (=D284), monuron (=D285), MT-128 (=6-Chlor-N-[(2E)-3-chlorprop-2-en-1-yl]-5-methyl-N-phenylpyridazin-3-amine) (=D286), MT-5950 (═N-[3-Chlor-4-(1-methylethyl)-phenyl]-2-methylpentanamide) (=D287), NGGC-011 (=D288), naproanilide (=D289), napropamide (=D290), naptalam (=D291), NC-310 (=4-(2,4-Dichlorobenzoyl)-1-methyl-5-benzyloxypyrazole) (=D292), neburon (=D293), nipyraclofen (=D294), nitralin (=D295), nitrofen (=D296), nitrophenolat-sodium (isomer mixture) (=D297), nitrofluorfen (=D298), nonanoic acid (=D299), norflurazon (=D300), orbencarb (=D301), oryzalin (=D302), oxadiargyl (=D303), oxadiazon (=D304), oxaziclomefone (=D305), oxyfluorfen (=D306), paclobutrazol (=D307), paraquat (=D308), paraquat-dichloride (=D309), pelargonic acid (nonanoic acid) (=D310), pendimethalin (=D311), pendralin (=D312), pentanochlor (=D313), pentoxazone (=D314), perfluidone (=D315), pethoxamid (=D317), phenisopham (=D318), phenmedipham (=D319), phenmedipham-ethyl (=D320), picloram (=D321), picolinafen (=D322), pinoxaden (=D323), piperophos (=D324), pirifenop (=D325), pirifenop-butyl (=D326), pretilachlor (=D327), probenazole (=D328), profluazol (=D329), procyazine (=D330), prodiamine (=D331), prifluraline (=D332), profoxydim (=D333), prohexadione (=D334), prohexadione-calcium (=D335), prohydrojasmone (=D336), prometon (=D337), prometryn (=D338), propachlor (=D339), propanil (=D340), propaquizafop (=D341), propazine (=D342), propham (=D343), propisochlor (=D344), propyzamide (=D345), prosulfalin (=D346), prosulfocarb (=D347), prynachlor (=D348), pyraclonil (=D349), pyraflufen (=D350), pyraflufen-ethyl (=D351), pyrasulfotole (=D352), pyrazolynate (pyrazolate) (=D353), pyrazoxyfen (=D354), pyribambenz (=D355), pyributicarb (=D356), pyridafol (=D357), pyridate (=D358), pyriminobac (=D359), pyrimisulfan (=D360), pyroxasulfone (=D361), quinclorac (=D362), quinmerac (=D363), quinoclamine (=D364), quizalofop (=D365), quizalofop-ethyl (=D366), quizalofop-P (=D367), quizalofop-P-ethyl (=D368), quizalofop-P-tefuryl (=D369), saflufenacil (=D370), secbumeton (=D371), sethoxydim (=D372), siduron (=D373), simazine (=D374), simetryn (=D375), SN-106279 (=Methyl-(2R)-2-({7-[2-chlor-4-(trifluormethyl)phenoxy]-2-naphthyl}oxy)-propanoate) (=D376), sulcotrione (=D377), sulfallate (CDEC) (=D378), sulfentrazone (=D379), sulfosate (glyphosate-trimesium) (=D380), SYN-523 (=D381), SYP-249 (=1-Ethoxy-3-methyl-1-oxobut-3-en-2-yl-5-[2-chlor-4-(trifluormethyl)phenoxy]-2-nitrobenzoate) (=D382), tebutam (=D383), tebuthiuron (=D384), tecnazene (=D385), tefuryltrione (=D386), tembotrione (=D387), tepraloxydim (=D388), terbacil (=D389), terbucarb (=D390), terbuchlor (=D391), terbumeton (=D392), terbuthylazine (=D393), terbutryn (=D394), thenylchlor (=D395), thiafluamide (=D396), thiazafluron (=D397), thiazopyr (=D398), thidiazimin (=D399), thidiazuron (=D400), thiobencarb (=D401), tiocarbazil (=D402), topramezone (=D403), tralkoxydim (=D404), triallate (=D405), triaziflam (=D406), triazofenamide (=D407), trichloracetic acid (TCA) (=D408), triclopyr (=D409), tridiphane (=D410), trietazine (=D411), trifluralin (=D412), trimeturon (=D413), trinexapac (=D414), trinexapac-ethyl (=D415), tsitodef (=D416), uniconazole (=D417), uniconazole-P (=D418), vernolate (=D419), ZJ-0862 (=3,4-Dichlor-N-{2-[(4,6-dimethoxypyrimidin-2-yl)oxy]benzyl}aniline) (=D420), the below compounds defined by their chemical structure, respectively:

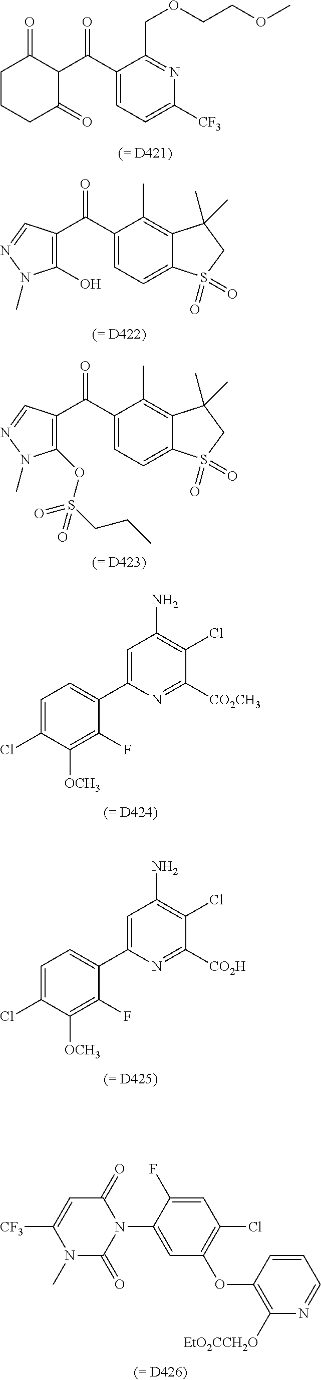

and propachlor (D 427).

Preferably, further herbicides which differ structurally and via their mode of action from the ALS inhibitor herbicides belonging to the groups (A), (B), and (C) as defined above and to be applied according to the present invention for control of unwanted vegetation in ALS inhibitor herbicide tolerant B. napus plants, preferably in mutated B. napus plants as described herein. In connection with ALS inhibitor herbicides belonging to the groups (A), (B), and (C) are those selected from the group consisting of acetochlor (=D1), carbetamide (=D56), fenoxaprop-P-ethyl (=D164), fluazifop-P-butyl (=D174), haloxyfop-P-methyl (=D222), metolachlor (=D275), dimethenamid (=D132), napropamide (=D290), pethoxamid (=D317), propaquizafop (=D341), propisochlor (=D344), propyzamide (=D345), quinmerac (=D363), propachlor (D 427), clomazone (=D83), clopyralid (=D86), dimethachlor (=D130), metazachlor (=D265), picloram (=D321), and quizalofop-P-ethyl (=D368).

Even more preferably, further herbicides which differ from the ALS inhibitor herbicides belonging to the groups (A), (B), and (C) as defined above and to be applied according to the invention in connection with ALS inhibitor herbicides belonging to the groups (A), (B), and (C) are those selected from the group consisting of clomazone (=D83), clopyralid (=D86), dimethachlor (=D130), metazachlor (=D265), picloram (=D321), and quizalofop-P-ethyl (=D368).

Mixtures containing ALS inhibitor herbicides and non ALS inhibitor herbicides, compositions comprising mixtures of one or more ALS inhibitor herbicide(s) (compounds belonging to one or more of groups (A), (B) and (C)) and non ALS inhibitor heribicide(s) (group (D) members; as defined above) that are of very particular interest in order to be used according to present invention for control of unwanted vegetation are:

### (A1-1)+(D83); (A1-1)+(D86); (A1-1)+(D130); (A1-1)+(D265); (A1-1)+(D321); (A1-1)+(D368);

### (A1-9)+(D83); (A1-9)+(D86); (A1-9)+(D130); (A1-9)+(D265); (A1-9)+(D321); (A1-9)+(D368);

### (A1-12)+(D83); (A1-12)+(D86); (A1-12)+(D130); (A1-12)+(D265); (A1-12)+(D321); (A1-12)+(D368);

### (A1-13)+(D83); (A1-13)+(D86); (A1-13)+(D130); (A1-13)+(D265); (A1-13)+(D321); (A1-13)+(D368);

### (A1-16)+(D83); (A1-16)+(D86); (A1-16)+(D130); (A1-16)+(D265); (A1-16)+(D321); (A1-16)+(D368);

### (A1-17)+(D83); (A1-17)+(D86); (A1-17)+(D130); (A1-17)+(D265); (A1-17)+(D321); (A1-17)+(D368);

### (A1-18)+(D83); (A1-18)+(D86); (A1-18)+(D130); (A1-18)+(D265); (A1-18)+(D321); (A1-18)+(D368);

### (A1-20)+(D83); (A1-20)+(D86); (A1-20)+(D130); (A1-20)+(D265); (A1-20)+(D321); (A1-20)+(D368);

### (A1-26)+(D83); (A1-26)+(D86); (A1-26)+(D130); (A1-26)+(D265); (A1-26)+(D321); (A1-26)+(D368);

### (A1-28)+(D83); (A1-28)+(D86); (A1-28)+(D130); (A1-28)+(D265); (A1-28)+(D321); (A1-28)+(D368);

### (A1-29)+(D83); (A1-29)+(D86); (A1-29)+(D130); (A1-29)+(D265); (A1-29)+(D321); (A1-29)+(D368);

### (A1-31)+(D83); (A1-31)+(D86); (A1-31)+(D130); (A1-31)+(D265); (A1-31)+(D321); (A1-31)+(D368);

### (A1-41)+(D83); (A1-41)+(D86); (A1-41)+(D130); (A1-41)+(D265); (A1-41)+(D321); (A1-41)+(D368);

### (A2-2)+(D83); (A2-2)+(D86); (A2-2)+(D130); (A2-2)+(D265); (A2-2)+(D321); (A2-2)+(D368);

### (A2-3)+(D83); (A2-3)+(D86); (A2-3)+(D130); (A2-3)+(D265); (A2-3)+(D321); (A2-3)+(D368);

### (A3-3)+(D83); (A3-3)+(D86); (A3-3)+(D130); (A3-3)+(D265); (A3-3)+(D321); (A3-3)+(D368);

### (A3-5)+(D83); (A3-5)+(D86); (A3-5)+(D130); (A3-5)+(D265); (A3-5)+(D321); (A3-5)+(D368);

### (A3-7)+(D83); (A3-7)+(D86); (A3-7)+(D130); (A3-7)+(D265); (A3-7)+(D321); (A3-7)+(D368);

### (A4-1)+(D83); (A4-1)+(D86); (A4-1)+(D130); (A4-1)+(D265); (A4-1)+(D321); (A4-1)+(D368);

### (A4-2)+(D83); (A4-2)+(D86); (A4-2)+(D130); (A4-2)+(D265); (A4-2)+(D321); (A4-2)+(D368);

### (A4-3)+(D83); (A4-3)+(D86); (A4-3)+(D130); (A4-3)+(D265); (A4-3)+(D321); (A4-3)+(D368);

### (A4-2)+(D83); (A4-2)+(D86); (A4-2)+(D130); (A4-2)+(D265); (A4-2)+(D321); (A4-2)+(D368);

### (B1-2)+(D83); (B1-2)+(D86); (B1-2)+(D130); (B1-2)+(D265); (B1-2)+(D321); (B1-2)+(D368);

### (C1-1)+(D83); (C1-1)+(D86); (C1-1)+(D130); (C1-1)+(D265); (C1-1)+(D321); (C1-1)+(D368).

The application of ALS inhibitor herbicides also act efficiently on perennial weeds which produce shoots from rhizomes, root stocks and other perennial organs and which are difficult to control. Here, the substances can be applied, for example, by the pre-sowing method, the pre-emergence method or the post-emergence method, for example jointly or separately. Preference is given, for example, to application by the post-emergence method, in particular to the emerged harmful plants.

Specific examples may be mentioned of some representatives of the monocotyledonous and dicotyledonous weed flora which can be controlled by the ALS inhibitor herbicides, without the enumeration being restricted to certain species.

Examples of weed species on which the application according to present invention act efficiently are, from amongst the monocotyledonous weed species, Avena spp., Alopecurus spp., Apera spp., Brachiaria spp., Bromus spp., Digitaria spp., Lolium spp., Echinochloa spp., Panicum spp., Phalaris spp., Poa spp., Setaria spp., volunteer cereals (Triticum sp., Hordeum sp.) and also Cyperus species from the annual group, and, among the perennial species, Agropyron, Cynodon, Imperata and Sorghum and also perennial Cyperus species.

In the case of the dicotyledonous weed species, the spectrum of action extends to genera such as, for example, Aethusa spp., Amaranthus spp., Capsella spp, Centaurea spp., Chenopodium spp., Chrysanthemum spp., Galium spp., Geranium spp., Lamium spp., Matricaria spp., Myosotis spp., Papaver spp., Polygonum spp., Sinapis spp., Solanum spp., Stellaria spp., Thlaspi spp., Urtica spp., Veronica spp. and Viola spp., Xanthium spp., among the annuals, and Convolvulus, Cirsium, Rumex and Artemisia in the case of the perennial weeds.

Another embodiment provides a polyploid plant, such as a polyploid Brassica plant, such as B. napus or B. juncea, plant as described herein to which one or more ALS inhibitor herbicide(s) alone or in combination with one or more herbicide(s) that do(es) not belong to the class of ALS inhibitor herbicides are applied for control of unwanted vegetation in polyploid plant, such as Brassica plant, such as B. napus or B. juncea, plant comprising two ALS polypeptides containing leucine instead of tryptophan at a position of said ALS polypeptide corresponding to position 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as an ALS I polypeptide containing serine instead of proline at a position corresponding to position 197 and leucine instead of tryptophan at a position corresponding to position 574 of SEQ ID NO: 10 of said ALS I Brassica, such as B. napus, polypeptide, and an ALS III polypeptide containing leucine instead of tryptophan at a position corresponding to position 574 of SEQ ID NO: 10 of said ALS III Brassica, such as B. napus polypeptide, or such as an ALS I polypeptide containing leucine instead of tryptophan at a position corresponding to position 574 of SEQ ID NO: 10 of said ALS I Brassica, such as B. napus, polypeptide, and an ALS III polypeptide containing serine instead of proline at a position corresponding to position 197 and leucine instead of tryptophan at a position corresponding to position 574 of SEQ ID NO: 10 of said ALS III Brassica, such as B. napus polypeptide, or such as an ALS I polypeptide containing serine instead of proline at a position corresponding to position 197 and leucine instead of tryptophan at a position corresponding to position 574 of SEQ ID NO: 10 of said ALS I Brassica, such as B. napus, polypeptide, and an ALS III polypeptide containing serine instead of proline at a position corresponding to position 197 and leucine instead of tryptophan at a position corresponding to position 574 of SEQ ID NO: 10 of said ALS III Brassica, such as B. napus polypeptide, or such as an ALS-A polypeptide containing serine instead of proline at a position corresponding to position 197 and leucine instead of tryptophan at a position corresponding to position 574 of SEQ ID NO: 10 of said ALS-A Brassica, such as B. juncea, polypeptide, and an ALS-B polypeptide containing leucine instead of tryptophan at a position corresponding to position 574 of SEQ ID NO: 10 of said ALS-B Brassica, such as B. juncea polypeptide, or such as an ALS-A polypeptide containing leucine instead of tryptophan at a position corresponding to position 574 of SEQ ID NO: 10 of said ALS-A Brassica, such as B. juncea, polypeptide, and an ALS-B polypeptide containing serine instead of proline at a position corresponding to position 197 and leucine instead of tryptophan at a position corresponding to position 574 of SEQ ID NO: 10 of said ALS-B Brassica, such as B. juncea polypeptide, or such as an ALS-A polypeptide containing serine instead of proline at a position corresponding to position 197 and leucine instead of tryptophan at a position corresponding to position 574 of SEQ ID NO: 10 of said ALS-A Brassica, such as B. juncea, polypeptide, and an ALS-B polypeptide containing serine instead of proline at a position corresponding to position 197 and leucine instead of tryptophan at a position corresponding to position 574 of SEQ ID NO: 10 of said ALS-B Brassica, such as B. juncea polypeptide.

In another embodiment, a polyploid plant, such as Brassica, such as B. napus or B. juncea, plant is provided as described herein to which one or more ALS inhibitor herbicide(s) alone or in combination with one or more herbicide(s) that do(es) not belong to the class of ALS inhibitor herbicides are applied for control of unwanted vegetation in polyploid plant, such as Brassica, such as B. napus or B. juncea, plant comprising mutations of at least two endogenous acetolactate synthase (ALS) genes, wherein said gene encodes an ALS polypeptide containing leucine instead of tryptophan at a position corresponding to position 574 of SEQ ID NO: 10, and wherein at least one of the ALS genes encodes an ALS polypeptide which further comprises at a position corresponding to position 197 of SEQ ID NO: 10 instead of the naturally encoded amino acid proline the amino acid serine, such as Brassica, such as B. napus, genes, wherein the ALS I Brassica, such as B. napus, gene encodes an ALS I Brassica, such as B. napus, polypeptide containing serine instead of proline at a position corresponding to position 197 of SEQ ID NO: 10 and leucine instead of tryptophan at a position corresponding to position 574 of SEQ ID NO: 10 and wherein the ALS III Brassica, such as B. napus, gene encodes an ALS III Brassica, such as B. napus, polypeptide containing leucine instead of tryptophan at a position corresponding to position 574 of SEQ ID NO: 10, or such as B. napus, gene encodes an ALS I Brassica, such as B. napus, polypeptide containing leucine instead of tryptophan at a position corresponding to position 574 of SEQ ID NO: 10 and wherein the ALS III Brassica, such as B. napus, gene encodes an ALS III Brassica, such as B. napus, polypeptide containing serine instead of proline at a position corresponding to position 197 of SEQ ID NO: 10 and leucine instead of tryptophan at a position corresponding to position 574 of SEQ ID NO: 10, or such as Brassica, such as B. napus, genes, wherein the ALS I Brassica, such as B. napus, gene encodes an ALS I Brassica, such as B. napus, polypeptide containing serine instead of proline at a position corresponding to position 197 of SEQ ID NO: 10 and leucine instead of tryptophan at a position corresponding to position 574 of SEQ ID NO: 10 and wherein the ALS III Brassica, such as B. napus, gene encodes an ALS III Brassica, such as B. napus, polypeptide containing serine instead of proline at a position corresponding to position 197 of SEQ ID NO: 10 and leucine instead of tryptophan at a position corresponding to position 574 of SEQ ID NO: 10, or such as B. juncea, genes, wherein the ALS-A Brassica, such as B. juncea, gene encodes an ALS-A Brassica, such as B. juncea, polypeptide containing serine instead of proline at a position corresponding to position 197 of SEQ ID NO: 10 and leucine instead of tryptophan at a position corresponding to position 574 of SEQ ID NO: 10 and wherein the ALS-B Brassica, such as B. juncea, gene encodes an ALS-B Brassica, such as B. juncea, polypeptide containing leucine instead of tryptophan at a position corresponding to position 574 of SEQ ID NO: 10, or such as B. juncea, genes, wherein the ALS-A Brassica, such as B. juncea, gene encodes an ALS-A Brassica, such as B. juncea, polypeptide containing leucine instead of tryptophan at a position corresponding to position 574 of SEQ ID NO: 10 and wherein the ALS-B Brassica, such as B. juncea, gene encodes an ALS-B Brassica, such as B. juncea, polypeptide containing serine instead of proline at a position corresponding to position 197 of SEQ ID NO: 10 and leucine instead of tryptophan at a position corresponding to position 574 of SEQ ID NO: 10, or such as B. juncea, genes, wherein the ALS-A Brassica, such as B. juncea, gene encodes an ALS-A Brassica, such as B. juncea, polypeptide containing serine instead of proline at a position corresponding to position 197 of SEQ ID NO: 10 and leucine instead of tryptophan at a position corresponding to position 574 of SEQ ID NO: 10 and wherein the ALS-B Brassica, such as B. juncea, gene encodes an ALS-B Brassica, such as B. juncea, polypeptide containing serine instead of proline at a position corresponding to position 197 of SEQ ID NO: 10 and leucine instead of tryptophan at a position corresponding to position 574 of SEQ ID NO: 10.

In yet another embodiment, a plant, such as Brassica, such as B. napus or B. juncea, plant as described herein is homozygous regarding the mutation of the ALS genes as described herein.

In one embodiment, the present invention relates to the use of one or more ALS inhibitor herbicide(s) alone or in combination with one or more non ALS inhibitor herbicide(s) for weed control in plant growing areas, such as in B. napus or in B. juncea growing areas which plants comprise at least two endogenous ALS genes, wherein said ALS genes comprise a codon encoding Leu instead of Trp at a position corresponding to position 1720-1722 of the nucleotide sequence of SEQ ID NO: 9, and wherein at least one of said ALS genes further comprises a codon encoding Ser instead of Pro at a position corresponding to position 589-591 of the nucleotide sequence of SEQ ID NO: 9, which plants are heterozygous or homozygous, preferably homozygous concerning the mutations in codon of the endogenous ALS I gene corresponding to the codon at position 589-591 of SEQ ID NO: 9 and in codon of the endogenous ALS I gene corresponding to the codon at position 1720-1722 of SEQ ID NO: 9, such as B. napus plants which comprise an endogenous ALS I gene, wherein the ALS I gene comprises a codon encoding Ser instead of Pro at a position corresponding to position 544-546 and a codon encoding Leu instead of Trp at a position corresponding to position 1675-1677 of the nucleotide sequence of the B. napus ALS I gene shown in SEQ ID NO: 1, and an endogenous ALS III gene, wherein the ALS III gene comprises Leu instead of Trp at a position corresponding to position 1666-1668 of the nucleotide sequence of the B. napus ALS III gene shown in SEQ ID NO: 3, which plants are heterozygous or homozygous, preferably homozygous concerning the mutation in codon 544-546 and 1675-1677 of the endogenous ALS I gene and the mutation in codon 1666-1668 of the endogenous ALS III gene, or such as B. napus plants which comprise an endogenous ALS I gene, wherein the ALS I gene comprises a codon encoding Leu instead of Trp at a position corresponding to position 1675-1677 of the nucleotide sequence of the B. napus ALS I gene shown in SEQ ID NO: 1, and an endogenous ALS III gene, wherein the ALS III gene comprises Ser instead of Pro at a position corresponding to position 535-537, and Leu instead of Trp at a position corresponding to position 1666-1668 of the nucleotide sequence of the B. napus ALS III gene shown in SEQ ID NO: 3, which plants are heterozygous or homozygous, preferably homozygous concerning the mutation in codon 1675-1677 of the endogenous ALS I gene and the mutation in codon 535-537 and 1666-1668 of the endogenous ALS III gene, or such as B. napus plants which comprise an endogenous ALS I gene, wherein the ALS I gene comprises a codon encoding Ser instead of Pro at a position corresponding to position 544-546 and a codon encoding Leu instead of Trp at a position corresponding to position 1675-1677 of the nucleotide sequence of the B. napus ALS I gene shown in SEQ ID NO: 1, and an endogenous ALS III gene, wherein the ALS III gene comprises Ser instead of Pro at a position corresponding to position 535-537, and Leu instead of Trp at a position corresponding to position 1666-1668 of the nucleotide sequence of the B. napus ALS III gene shown in SEQ ID NO: 3, which plants are heterozygous or homozygous, preferably homozygous concerning the mutation in codon 544-546 and 1675-1677 of the endogenous ALS I gene and the mutation in codon 535-537 and 1666-1668 of the endogenous ALS III gene, or such as B. juncea plants which comprise an endogenous ALS-A gene, wherein the ALS-A gene comprises a codon encoding Ser instead of Pro at a position corresponding to position 535-537 and a codon encoding Leu instead of Trp at a position corresponding to position 1666-1668 of the nucleotide sequence of the B. juncea ALS-A gene shown in SEQ ID NO: 5, and an endogenous ALS-B gene, wherein the ALS-B gene comprises Leu instead of Trp at a position corresponding to position 1675-1677 of the nucleotide sequence of the B. juncea ALS-B gene shown in SEQ ID NO: 7, which plants are heterozygous or homozygous, preferably homozygous concerning the mutation in codon 535-537 and 1666-1668 of the endogenous ALS-A gene and the mutation in codon 1675-1677 of the endogenous ALS-B gene, or such as B. juncea plants which comprise an endogenous ALS-A gene, wherein the ALS-A gene comprises a codon encoding Leu instead of Trp at a position corresponding to position 1666-1668 of the nucleotide sequence of the B. juncea ALS-A gene shown in SEQ ID NO: 5, and an endogenous ALS-B gene, wherein the ALS-B gene comprises Ser instead of Pro at a position corresponding to position 544-546 and Leu instead of Trp at a position corresponding to position 1675-1677 of the nucleotide sequence of the B. juncea ALS-B gene shown in SEQ ID NO: 7, which plants are heterozygous or homozygous, preferably homozygous concerning the mutation in codon 1666-1668 of the endogenous ALS-A gene and the mutation in codon 544-546 and 1675-1677 of the endogenous ALS-B gene, or such as B. juncea plants which comprise an endogenous ALS-A gene, wherein the ALS-A gene comprises a codon encoding Ser instead of Pro at a position corresponding to position 535-537 and a codon encoding Leu instead of Trp at a position corresponding to position 1666-1668 of the nucleotide sequence of the B. juncea ALS-A gene shown in SEQ ID NO: 5, and an endogenous ALS-B gene, wherein the ALS-B gene comprises Ser instead of Pro at a position corresponding to position 544-546 and Leu instead of Trp at a position corresponding to position 1675-1677 of the nucleotide sequence of the B. juncea ALS-B gene shown in SEQ ID NO: 7, which plants are heterozygous or homozygous, preferably homozygous concerning the mutation in codon 535-537 and 1666-1668 of the endogenous ALS-A gene and the mutation in codon 544-546 and 1675-1677 of the endogenous ALS-B gene.

Owing to their herbicidal and plant growth-regulatory properties, ALS inhibitor herbicides belonging to one or more of the groups (A), (B), and (C) either alone or in combination with non ALS inhibitor heribicides can be employed for controlling harmful plants in known plant, such as Brassica, such as B. napus or B. juncea, plants but also in tolerant or genetically modified crop plants that do already exists or need still to be developed. In general, the transgenic plants are distinguished by specific advantageous properties, in addition to tolerances to the ALS inhibitor herbicides according to the invention, for example, by tolerances to non ALS inhibitor herbicides, resistances to plant diseases or the causative organisms of plant diseases such as certain insects or microorganisms, such as fungi, bacteria or viruses. Other specific characteristics relate, for example, to the harvested material with regard to quantity, quality, storability, composition and specific constituents. Thus, transgenic plants are known whose oil content is increased, or whose oil quality is altered, or those where the harvested material has a different fatty acid composition.

Conventional methods of generating novel plants which have modified properties in comparison to plants occurring to date consist, for example, in traditional breeding methods and the generation of mutants. Alternatively, novel plants with altered properties can be generated with the aid of recombinant methods (see, for example, EP-A-0221044, EP-A-0131624). For example, the following have been described in several cases:

the modification, by recombinant technology, of crop plants with the aim of modifying the starch synthesized in the plants (for example WO 92/11376, WO 92/14827, WO 91/19806),

transgenic crop plants which exhibit tolerance to non ALS inhibitor herbicides,

transgenic crop plants with the capability of producing Bacillus thuringiensis toxins (Bt toxins), which make the plants resistant to certain pests (EP-A-0142924, EP-A-0193259),

transgenic crop plants with a modified fatty acid composition (WO 91/13972).

The plants according to the invention may additionally contain an endogenous or a transgene, which confers herbicide resistance, such as the bar or pat gene, which confer resistance to glufosinate ammonium (Liberty or Basta) [EP 0 242 236 and EP 0 242 246 incorporated by reference]; or any modified EPSPS gene, such as the 2mEPSPS gene from maize [EPO 508 909 and EP 0 507 698 incorporated by reference], or glyphosate acetyltransferase, or glyphosate oxidoreductase, which confer resistance to glyphosate (RoundupReady), or bromoxynitril nitrilase to confer bromoxynitril tolerance. Further, the plants according to the invention may additionally contain an endogenous or a transgene which confers increased oil content or improved oil composition, such as a 12:0 ACP thioesteraseincrease to obtain high laureate; which confers increased digestibility, such as 3-phytase; which confers pollination control, such as such as barnase under control of an anther-specific promoter to obtain male sterility, or barstar under control of an anther-specific promoter to confer restoration of male sterility, or such as the Ogura cytoplasmic male sterility and nuclear restorer of fertility.

A large number of techniques in molecular biology are known in principle with the aid of which novel transgenic plants with modified properties can be generated; see, for example, Sambrook et al., 1989, Molecular Cloning, A Laboratory Manual, 2^{nd} Edition, Cold Spring Harbor Laboratory Press, Cold Spring Harbor, N.Y.; or Winnacker “Gene und Klone”, VCH Weinheim 2^{nd} Edition 1996 or Christou, “Trends in Plant Science” 1 (1996) 423-431).

To carry out such recombinant manipulations, nucleic acid molecules which allow mutagenesis or sequence changes by recombination of DNA sequences can be introduced into plasmids. For example, the abovementioned standard methods allow base exchanges to be carried out, subsequences to be removed, or natural or synthetic sequences to be added. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect or by expressing at least one suitably constructed ribozyme which specifically cleaves transcripts of the abovementioned gene product.

To this end, it is possible to use DNA molecules which encompass the entire coding sequence of a gene product inclusive of any flanking sequences which may be present, and also DNA molecules which only encompass portions of the coding sequence, it being necessary for these portions to be long enough to have an antisense effect in the cells. The use of DNA sequences which have a high degree of homology to the coding sequences of a gene product, but are not completely identical to them, is also possible.

When expressing nucleic acid molecules in plants, the protein synthesized can be localized in any desired compartment of the plant cell. However, to achieve localization in a particular compartment, it is possible, for example, to link the coding region with DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give rise to entire plants. Thus, transgenic plants, such as Brassica, such as B. napus or B. juncea, plants can be obtained whose properties are altered by overexpression, suppression or inhibition of homologous (=natural) genes or gene sequences or the expression of heterologous (=foreign) genes or gene sequences.

The present invention furthermore provides a method for controlling unwanted plants in plant, such as B. napus or B. juncea growing areas of plants, such as B. napus or B. juncea plants according to the invention as described herein which comprises applying one or more ALS inhibitor herbicides belonging to groups (A), (B) and/or (C) to the plants (for example harmful plants, such as monocotyledonous or dicotyledonous weeds or unwanted crop plants), the seed (seeds or vegetative propagation organs, such as tubers or shoot parts) or to the area in which the plants grow (for example the area under cultivation), for example together or separately.

The present invention furthermore provides a method for controlling unwanted plants in growing areas of plants, such as B. napus or B. juncea plants according to the invention as described herein which comprises applying one or more ALS inhibitor herbicide(s) belonging to groups (A), (B) and/or (C) alone or in combination with non ALS inhibitor herbicides belonging to class (D) compound according to the invention to the plants (for example harmful plants, such as monocotyledonous or dicotyledonous weeds or unwanted crop plants), the seed (seeds or vegetative propagation organs, such as tubers or shoot parts) or to the area in which the plants grow (for example the area under cultivation), for example together or separately. One or more non ALS inhibitor herbicides may be applied in combination with one or more ALS inhibitor herbicide(s) before, after or simultaneously with the ALS inhibitor herbicide(s) to the plants, the seed or the area in which the plants grow (for example the area under cultivation).

“Unwanted plants” or “unwanted vegetation” are to be understood as meaning all plants which grow in locations where they are unwanted. This can, for example, be harmful plants (for example monocotyledonous or dicotyledonous species or other unwanted crop plants (volunteers)) such as Geranium dissectum, Centaurea cyanus, Sinapis arvensis and/or Alopecurus myosuroides.

In one embodiment, an unwanted plant is at least one dicotyledonous plant selected from the group consisting of Aethusa cynapium, Agrostemma githago, Amaranthus sp., Ambrosia artemisifolia, Ammi majus, Anagallis arvensis, Anchusa officinalis, Anthemis sp., Aphanes arvensis, Arabidopsis thaliana, Artemisia vulgaris, Atriplex sp., Bidens sp., Bifora radians, Brassica nigra, Calendula arvensis, Capsella bursa pastoris, Cardamine hirsute, Cardaria draba, Centaurea cyanus, Cerastium arvense, Chaenorhinum minus, Chenopodium sp., Chrysanthemum segetum, Cirsium arvense, Convolvulus sp., Coronopus sp., Datura stramonium, Daucus carota, Descurainia sophia, Diplotaxis muralis, Echium vulgare, Erigeron Canadensis, Erodium circutarium, Erysium cheiranthoides, Euphorbia sp., Filaginella uliginosa, Fumaria officinalis, Galeopsis sp., Galeopsis tetraclit, Galinsoga parviflora, Galium aparine, Geranium sp., Juncus bufonius, Kickxia spuria, Lactuca sericola, Lamium sp, Lapsana communis, Lathyrus tuberosus, Legousia speculum-veneris, Linaria vulgaris, Lithospermum arvense, Lycopsis arvensis, Malva sp., Matricaria sp., Menta arvensis, Mercurialis annua, Myagrum perfoliatum, Myosotis arvensis, Papaver sp., Picris echioides, Polygonum sp., Portulaca oleracea, Ranunculus sp., Raphanus raphanistrum, Rumex sp., Scandix pecten-veneris, Senecio vulgaris, Silene sp., Sinapis arvensis, Sisymbrium officinale, Solanum nigrum, Sonchus sp., Spergula arvensis, Stachys arvensis, Stellaria media, Thlaspi arvense, Tussilago farfara, Urtica urens, Verbena officinalis, Veronica sp., Vicia sp., Viola arvensis and Xanthium sp. In another embodiment, an unwanted plant is at least one plant selected from the group consisting of Aethusa cynapium, Galium aparine, Geranium sp., Lamium sp, Matricaria sp., Myosotis arvensis, Papaver sp., Polygonum sp., Sisymbrium officinale, Stellaria media, Thlaspi arvense, Urtica urens and Viola arvensis.

In yet another embodiment, an unwanted plant is at least one monocotyledonous plant selected from the group consisting of Agropyron repens, Alopecurus myosuroides, Apera spica-venti, Avena sp., Bromus sp., Cyperus sp., Digitaria sp., Echinochloa sp., Hordeum murinum, Lolium multiflorum, Panicum dichotomiflorum, Phalaris canariensis, Poa sp., Setaria sp., Sorghum halepense, Leptochloa filiformis. In another embodiment, an unwanted plant is at least one plant selected from the group consisting of Agropyron repens, Alopecurus myosuroides, Apera spica-venti, Avena sp. and Poa sp.

In yet another embodiment, an unwanted plant is at least one monocotyledonous plant selected from the group consisting of Beta vulgaris, Helianthus annuus, Solanum tuberosum, Triticum vulgare, Hordeum vulgare, Secale cereale, Avena sativa. In another embodiment, an unwanted plant is Triticum vulgare and Hordeum vulgare.

The herbicide combinations to be used according to the invention can be prepared by known processes, for example as mixed formulations of the individual components, if appropriate with further active compounds, additives and/or customary formulation auxiliaries, which combinations are then applied in a customary manner diluted with water, or as tank mixes by joint dilution of the components, formulated separately or formulated partially separately, with water. Also possible is the split application of the separately formulated or partially separately formulated individual components.

It is also possible to apply ALS inhibitor herbicides or the combination comprising ALS inhibitor herbicide(s) and non ALS inhibitor herbicide(s) in a plurality of portions (sequential application) using, for example, pre-emergence applications followed by post-emergence applications or using early post-emergence applications followed by medium or late post-emergence applications. Preference is given here to the joint or almost simultaneous application of the active compounds of the combination in question.

The herbicides belonging to any of the above defined groups (A), (B), (C) and (D) and to be applied according to present invention can be converted jointly or separately into customary formulations, such as solutions, emulsions suspensions, powders, foams, pastes, granules, aerosols, natural and synthetic materials impregnated with active compound and microencapsulations in polymeric materials. The formulations may comprise the customary auxiliaries and additives.

These formulations are produced in a known manner, for example by mixing the active compounds with extenders, that is liquid solvents, pressurized liquefied gases and/or solid carriers, if appropriate with the use of surfactants, that is emulsifiers and/or dispersants, and/or foam formers.

If the extender used is water, it is also possible to use, for example, organic solvents as auxiliary solvents. Suitable liquid solvents are essentially: aromatics, such as xylene, toluene, alkylnaphthalenes, chlorinated aromatics or chlorinated aliphatic hydrocarbons, such as chlorobenzenes, chloroethylenes, or methylene chloride, aliphatic hydrocarbons, such as cyclohexane or paraffins, for example mineral oil fractions, mineral and vegetable oils, alcohols, such as butanol or glycol, and ethers and esters thereof, ketones, such as acetone, methyl ethyl ketone, methyl isobutyl ketone or cyclohexanone, strongly polar solvents, such as dimethylformamide or dimethyl sulfoxide, and also water.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as sawdust, coconut shells, corn cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins and synthetic phospholipids, can be used in the formulations. Other possible additives are mineral and vegetable oils.

The herbicidal action of the herbicide combinations to be used according to the invention can be improved, for example, by surfactants, preferably by wetting agents from the group of the fatty alcohol polyglycol ethers. The fatty alcohol polyglycol ethers preferably comprise 10-18 carbon atoms in the fatty alcohol radical and 2-20 ethylene oxide units in the polyglycol ether moiety. The fatty alcohol polyglycol ethers may be present in nonionic form, or ionic form, for example in the form of fatty alcohol polyglycol ether sulfates, which may be used, for example, as alkali metal salts (for example sodium salts and potassium salts) or ammonium salts, or even as alkaline earth metal salts, such as magnesium salts, such as C₁₂/C₁₄-fatty alcohol diglycol ether sulfate sodium (Genapol® LRO, Clariant GmbH); see, for example, EP-A-0476555, EP-A-0048436, EP-A-0336151 or U.S. Pat. No. 4,400,196 and also Proc. EWRS Symp. “Factors Affecting Herbicidal Activity and Selectivity”, 227-232 (1988). Nonionic fatty alcohol polyglycol ethers are, for example, (C₁₀-C₁₈)-, preferably (C₁₀-C₁₄)-fatty alcohol polyglycol ethers (for example isotridecyl alcohol polyglycol ethers) which comprise, for example, 2-20, preferably 3-15, ethylene oxide units, for example those from the Genapol® X-series, such as Genapol® X-030, Genapol® X-060, Genapol® X-080 or Genapol® X-150 (all from Clariant GmbH).

The present invention further comprises the combination of ALS inhibitor herbicides belonging to any of the groups (A), (B), and (C) according to present invention with the wetting agents mentioned above from the group of the fatty alcohol polyglycol ethers which preferably contain 10-18 carbon atoms in the fatty alcohol radical and 2-20 ethylene oxide units in the polyglycol ether moiety and which may be present in nonionic or ionic form (for example as fatty alcohol polyglycol ether sulfates). Preference is given to C₁₂/C₁₄-fatty alcohol diglycol ether sulfate sodium (Genapol® LRO, Clariant GmbH) and isotridecyl alcohol polyglycol ether having 3-15 ethylene oxide units, for example from the Genapol® X-series, such as Genapol® X-030, Genapol® X-060, Genapol® X-080 and Genapol® X-150 (all from Clariant GmbH). Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides (see, for example, EP-A-0502014).

Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides (see, for example, EP-A-0502014).

The herbicidal action of the herbicide combinations according to the invention can also be enhanced by using vegetable oils. The term vegetable oils is to be understood as meaning oils of oleaginous plant species, such as soybean oil, rapeseed oil, corn oil, sunflower oil, cottonseed oil, linseed oil, coconut oil, palm oil, thistle oil or castor oil, in particular rapeseed oil, and also their transesterification products, for example alkyl esters, such as rapeseed oil methyl ester or rapeseed oil ethyl ester.

The vegetable oils are preferably esters of C₁₀-C₂₂-, preferably C₁₂-C₂₀-, fatty acids. The C₁₀-C₂₂-fatty acid esters are, for example, esters of unsaturated or saturated C₁₀-C₂₂-fatty acids, in particular those having an even number of carbon atoms, for example erucic acid, lauric acid, palmitic acid and in particular C₁₈-fatty acids, such as stearic acid, oleic acid, linoleic acid or linolenic acid.

Examples of C₁₀-C₂₂-fatty acid esters are esters obtained by reacting glycerol or glycol with the C₁₀-C₂₂-fatty acids contained, for example, in oils of oleaginous plant species, or C₁-C₂₀-alkyl-C₁₀-C₂₂-fatty acid esters which can be obtained, for example, by transesterification of the aforementioned glycerol- or glycol-C₁₀-C₂₂-fatty acid esters with C₁-C₂₀-alcohols (for example methanol, ethanol, propanol or butanol). The transesterification can be carried out by known methods as described, for example, in Rompp Chemie Lexikon, 9th edition, Volume 2, page 1343, Thieme Verlag Stuttgart.

Preferred C₁-C₂₀-alkyl-C₁₀-C₂₂-fatty acid esters are methyl esters, ethyl esters, propyl esters, butyl esters, 2-ethylhexyl esters and dodecyl esters. Preferred glycol- and glycerol-C₁₀-C₂₂-fatty acid esters are the uniform or mixed glycol esters and glycerol esters of C₁₀-C₂₂-fatty acids, in particular fatty acids having an even number of carbon atoms, for example erucic acid, lauric acid, palmitic acid and, in particular, C₁₈-fatty acids, such as stearic acid, oleic acid, linoleic acid or linolenic acid.

In the herbicidal compositions to be used according to the invention, the vegetable oils can be present, for example, in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob®B (Novance, France, hereinbelow referred to as ActirobB, main ingredient: rapeseed oil methyl ester), Rako-Binol® (Bayer AG, Germany, hereinbelow referred to as Rako-Binol, main ingredient: rapeseed oil), Renol® (Stefes, Germany, hereinbelow referred to as Renol, vegetable oil ingredient: rapeseed oil methyl ester) or Stefes Mero® (Stefes, Germany, hereinbelow referred to as Mero, main ingredient: rapeseed oil methyl ester).

In a further embodiment, herbicidal combinations to be used according to present invention can be formulated with the vegetable oils mentioned above, such as rapeseed oil, preferably in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob®B (Novance, France, hereinbelow referred to as ActirobB, main ingredient: rapeseed oil methyl ester), Rako-Binol® (Bayer AG, Germany, hereinbelow referred to as Rako-Binol, main ingredient: rapeseed oil), Renol® (Stefes, Germany, hereinbelow referred to as Renol, vegetable oil ingredient: rapeseed oil methyl ester) or Stefes Mero® (Stefes, Germany, hereinbelow referred to as Mero, main ingredient: rapeseed oil methyl ester).

It is possible to use colorants, such as inorganic pigments, for example iron oxide, titanium oxide, Prussian Blue, and organic dyes, such as alizarin dyes, azo dyes and metal phthalocyanine dyes, and trace nutrients such as salts of iron, manganese, boron, copper, cobalt, molybdenum and zinc.

The formulations to be used according to present invention generally comprise from 0.1 to 95% by weight of active compounds, preferably from 0.5 to 90% by weight.

As such or in their formulations, the ALS inhibitor herbicides belonging to any of the above defined groups (A), (B), and (C) can also be used as a mixture with other agrochemically active compounds, such as known non ALS inibitor herbicides, for controlling unwanted vegetation, for example for controlling weeds or for controlling unwanted crop plants, finished formulations or tank mixes, for example, being possible.

The use of a mixture of ALS inhibitor herbicides belonging to any of the above defined groups (A), (B), and (C) with other known active compounds, such as fungicides, insecticides, acaricides, nematicides, safeners, bird repellants, plant nutrients and soil structure improvers is likewise possible.

The ALS inhibitor herbicides belonging to any of the above defined groups (A), (B), (C) can be used as such, in the form of their formulations or in the use forms prepared therefrom by further dilution, such as ready-to-use solutions, suspensions, emulsions, powders, pastes and granules. Application is carried out in a customary manner, for example by watering, spraying, atomizing, broadcasting.

According to the invention, one or more of the ALS inhibitor herbicides belonging to any of the above defined groups (A), (B), and (C) can be applied either alone or in combination with one or more non ALS inhibitor herbicides belonging to group (DO) to the plants (for example harmful plants, such as monocotyledonous or dicotyledonous weeds or unwanted crop plants), the seed (for example grains, seeds or vegetative propagation organs, such as tubers or shoot parts with buds) or the area under cultivation (for example the soil), preferably to the green plants and parts of plants and, if appropriate, additionally the soil. One possible use is the joint application of the active compounds in the form of tank mixes, where the optimally formulated concentrated formulations of the individual active compounds are, together, mixed in a tank with water, and the spray liquor obtained is applied.

A further embodiment refers to a method to increase the tolerance to ALS inhibitor herbicide(s) of polyploid plants, such as Brassica napus or Brassica juncea plants, said method comprising introducing a first ALS allele encoding an ALS polypeptide comprising at a position corresponding to position 197 of SEQ ID NO: 10 instead of the naturally encoded amino acid proline the amino acid serine, and at a position corresponding to position 574 of SEQ ID NO: 10 instead of the naturally encoded amino acid tryptophan the amino acid leucine, such as an ALS I allele encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, or such as an ALS-A allele encoding an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine, and a second ALS allele encoding an ALS polypeptide comprising at a position corresponding to position 574 of SEQ ID NO: 10 instead of the naturally encoded amino acid tryptophan the amino acid leucine, such as an ALS III allele encoding an ALS III polypeptide comprising at a position corresponding to position 556 of SEQ ID NO: 4 instead of the naturally encoded amino acid tryptophan the amino acid leucine, or such as an ALS-B allele encoding an ALS-B polypeptide comprising at a position corresponding to position 559 of SEQ ID NO: 8 instead of the naturally encoded amino acid tryptophan the amino acid leucine, in the genome of said plant, or said method comprising introducing a first ALS allele encoding an ALS polypeptide comprising at a position corresponding to position 574 of SEQ ID NO: 10 instead of the naturally encoded amino acid tryptophan the amino acid leucine, such as an ALS I allele encoding an ALS I polypeptide comprising at a position corresponding to position 559 of SEQ ID NO: 2 instead of the naturally encoded amino acid tryptophan the amino acid leucine, or such as an ALS-A allele encoding an ALS-A polypeptide comprising at a position corresponding to position 556 of SEQ ID NO: 6 instead of the naturally encoded amino acid tryptophan the amino acid leucine, and a second ALS allele encoding an ALS polypeptide comprising at a position corresponding to position 197 of SEQ ID NO: 10 instead of the naturally encoded amino acid proline the amino acid serine, and at a position corresponding to position 574 of SEQ ID NO: 10 instead of the naturally encoded amino acid tryptophan the amino acid leucine, such as an ALS III allele encoding an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or such as an ALS-B allele encoding an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine, in the genome of said plant, or said method comprising introducing a first and a second ALS allele encoding an ALS polypeptide comprising at a position corresponding to position 197 of SEQ ID NO: 10 instead of the naturally encoded amino acid proline the amino acid serine, and at a position corresponding to position 574 of SEQ ID NO: 10 instead of the naturally encoded amino acid tryptophan the amino acid leucine, such as a first ALS allele which is an ALS I allele encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine and a second ALS allele which is an ALS III allele encoding an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine, or such as a first ALS allele which is ALS-A allele encoding an ALS-A polypeptide comprising at a position corresponding to position 179 of SEQ ID NO: 6 instead of the naturally encoded amino acid proline the amino acid serine and at a position corresponding to position 556 of SEQ ID NO: 6 instead of the naturally encoded amino acid tryptophan the amino acid leucine, and a second ALS allele which is an ALS-B allele encoding an ALS-B polypeptide comprising at a position corresponding to position 182 of SEQ ID NO: 8 instead of the naturally encoded amino acid proline the amino acid serine and at a position corresponding to position 559 of SEQ ID NO: 8 instead of the naturally encoded amino acid tryptophan the amino acid leucine, in the genome of said plant.

An increase in tolerance to ALS inhibitor herbicide(s) can be an increase in tolerance to one or to more of the ALS inhibitor herbicides as described elsewhere in this application.

Introducing an ALS allele, such as an ALS I allele and an ALS III allele or such as an ALS-A allele and an ALS-B allele according to the invention can be, for example, generation of the ALS I mutation as described in the below examples. Introducing an ALS allele, such as an ALS I or an ALS III allele or such as an ALS-A allele and an ALS-B allele according to the invention can also be by crossing with a plant comprising an ALS allele according to the invention and selection of progeny plants comprising the ALS alleles according to the invention.

Progeny plants can be selected by their tolerance to ALS inhibitor herbicide(s). Progeny plants can also be selected using molecular methods well known in the art, such as, for example, direct sequencing or using molecular markers (e.g. AFLP, PCR, Invader™, TaqMan®, KASP, and the like).

### Agronomically Exploitable

The skilled person will understand that it is generally preferred that the polyploid plants, such as B. napus or B. juncea plants of the present invention and parts thereof are agronomically exploitable.

“Agronomically exploitable” means that the plants, such as B. napus or B. juncea plants and parts thereof are useful for agronomical purposes. For example, the B. napus plants should serve for the purpose of being useful for rape seed oil production for, e.g., bio fuel or bar oil for chainsaws, animal feed or honey production, for oil, meal, grain, starch, flour, protein, fiber, industrial chemical, pharmaceutical or neutraceutical production. The term “agronomically exploitable” when used herein also includes that the plants, such as B. napus or B. juncea plants of the present invention are less sensitive against an ALS-inhibitor herbicide, such as 5 times, or 10 times, or 50 times, or 100 times, or 500 times, or 1000 times, or 2000 times less sensitive as compared to wild type plants. The ALS inhibitor herbicide is one or more described herein, preferably it is foramsulfuron either alone or in combination with one or more further ALS-inhibitor herbicide(s) either from the sub-class of the sulfonyurea herbicides or any other sub-class of the ALS-inhibitor herbicides, most preferably it is foramsulfuron in combination with a further sulfonylurea herbicide and/or an ALS-inhibitor of the sulfonylaminocarbonyltriazolinone herbicide sub-class.

Another aspect of the present invention is the use of the plants, such as B. napus or B. juncea plant described herein and/or the harvestable parts or propagation material described herein for the manufacture/breeding of said plants. Methods for the manufacture/breeding of plants, such as B. napus or B. juncea plants are described herein elsewhere. Such manufacture/breeding methods may be used to generate plants of the present invention further comprising novel plant traits such as stress-resistance, like but not limited to drought, heat, cold, or salt stress and the like.

In a still further aspect, the present invention envisages the use of the herbicide tolerant plant described herein and/or harvestable parts or propagation material derived thereof in a screening method for the selection of ALS inhibitor herbicides.

A better understanding of the present invention and of its many advantages will be had from the following examples, offered for illustrative purposes only, and are not intended to limit the scope of the present invention in any way.

The sequence listing contained in the file named “BCS13-2013_ST25.txt”, which is 257 kilobytes (size as measured in Microsoft Windows®), contains 38 sequences SEQ ID NO: 1 through SEQ ID NO: 38 is filed herewith by electronic submission and is incorporated by reference herein.

### SEQUENCES

A. thaliana sequences SEQ ID NOs: 9 (nucleotide AY042819) and 10 (protein AAK68759), and wild type B. napus sequences SEQ ID NOs: 1 (ALS1 nucleotide Z11524) and 3 (ALS3 nucleotide Z11526) were taken from the ncbi-genebank (see world wide web: http://www.ncbi.nlm.nih.gov/genbank/). SEQ ID NOs: 2 and 4 are the protein sequences encoded by SEQ ID NOs: 1 and 3, respectively.

SEQ ID No. 1: Nucleic acid sequence encoding B. napus wild type ALS I gb Z11524.

SEQ ID No. 2: B. napus ALS I amino acid sequence derived from SEQ ID No. 1.

SEQ ID No. 3: Nucleic acid sequence encoding B. napus wild type ALS III gb Z11526.

SEQ ID No. 4: B. napus ALS III amino acid sequence derived from SEQ ID No. 3.

SEQ ID No. 5: Nucleic acid sequence encoding B. juncea wild type ALS-A.

SEQ ID No. 6: B. juncea ALS-A amino acid sequence derived from SEQ ID No. 5.

SEQ ID No. 7: Nucleic acid sequence encoding B. juncea wild type ALS-B.

SEQ ID No. 8: B. juncea ALS-B amino acid sequence derived from SEQ ID No. 7.

SEQ ID No. 9: Nucleic acid sequence encoding A. thaliana ALS gene.

SEQ ID No. 10: A. thaliana amino acid sequence derived from SEQ ID No. 9.

SEQ ID No. 11: Nucleic acid sequence encoding Gossypium hirsutum ALS gene.

SEQ ID No. 12: Gossypium hirsutum amino acid sequence derived from SEQ ID No. 19.

SEQ ID No. 13: Nucleic acid sequence encoding Gossypium hirsutum ALS gene.

SEQ ID No. 14: Gossypium hirsutum amino acid sequence derived from SEQ ID No. 21.

SEQ ID No. 15: Nucleic acid sequence encoding Glycine max ALS gene.

SEQ ID No. 16: Glycine max amino acid sequence derived from SEQ ID No. 23.

SEQ ID No. 17: Nucleic acid sequence encoding Glycine max ALS gene.

SEQ ID No. 18: Glycine max amino acid sequence derived from SEQ ID No. 25.

SEQ ID No. 19: Nucleic acid sequence encoding Glycine max ALS gene.

SEQ ID No. 20: Glycine max amino acid sequence derived from SEQ ID No. 27.

SEQ ID No. 21: Nucleic acid sequence encoding Glycine max ALS gene.

SEQ ID No. 22: Glycine max amino acid sequence derived from SEQ ID No. 29.

SEQ ID No. 23: Nucleic acid sequence encoding Nicotiana tabacum ALS gene.

SEQ ID No. 24: Nicotiana tabacum amino acid sequence derived from SEQ ID No. 31.

SEQ ID No. 25: Nucleic acid sequence encoding Nicotiana tabacum ALS gene.

SEQ ID No. 26: Nicotiana tabacum amino acid sequence derived from SEQ ID No. 33.

SEQ ID No. 27: Nucleic acid sequence encoding Solanum tuberosum ALS gene.

SEQ ID No. 28: Solanum tuberosum amino acid sequence derived from SEQ ID No. 35.

SEQ ID No. 29: Nucleic acid sequence encoding Solanum tuberosum ALS gene.

SEQ ID No. 30: Solanum tuberosum amino acid sequence derived from SEQ ID No. 37.

SEQ ID No. 31: Nucleic acid sequence encoding Solanum tuberosum ALS gene.

SEQ ID No. 32: Solanum tuberosum amino acid sequence derived from SEQ ID No. 39.

SEQ ID No. 33: Nucleic acid sequence encoding Triticum aestivum ALS gene.

SEQ ID No. 34: Triticum aestivum amino acid sequence derived from SEQ ID No. 41.

SEQ ID No. 35: Nucleic acid sequence encoding Triticum aestivum ALS gene.

SEQ ID No. 36: Triticum aestivum amino acid sequence derived from SEQ ID No. 43.

SEQ ID No. 37: Nucleic acid sequence encoding Saccharum officinarum ALS gene.

SEQ ID No. 38: Saccharum officinarum amino acid sequence derived from SEQ ID No. 45.

### EXAMPLES

### Example 1—Generation and Isolation of Mutant Brassica AHAS Alleles

### Mutant HETO134 and HETO 133

Brassica napus lines with the HETO134 mutation, i.e. comprising a G to T substitution at position 1676 of ALS I, resulting in a Tryptophan to Leucine amino acid substitution at position 559 of the encoded protein, and with the HETO133 mutation, i.e. comprising a G to T substitution at position 1667 of ALS III, resulting in a Tryptophan to Leucine amino acid substitution at position 556 of the encoded protein, were generated as follows.

### Seedling Germination and Callus Induction

Aliquots of seeds were sterilized by rinsing for 1 min in 70% ethanol followed by 15 minutes agitation in bleach (6% active chlorine dilution). After 3 washes in sterile water the seeds were sown on 5 cm Petri plates containing 5 ml of M-205 germination medium (25 seeds per plate). M-205 medium is half strength MS macro and micro salts (Murashige and Skoog, 1962), half strength B5 vitamins (Gamborg et al. 1968) containing 10 g/l sucrose and solidified with 8 g/l plant agar (pH 5.6). Plates of seeds were transferred to 2 l sterile glass containers and incubated for 5 days in the light at 24° C.

Five day old seedlings were used for the preparation of hypocotyl segments 7-10 mm in length. Hypocotyl segments were transferred to 14 cm Petri plates containing 75 ml of M-338 [H76] callus induction medium (25 explants/plate). M-338 [H76] medium is MS salts and vitamins (Murashige and Skoog, 1962), 20 g/l sucrose, 0.5 g/l MES, 0.5 g/l adenine sulphate, 5 mg/l silver nitrate, 0.5 mg/l 2,4-D, 0.2 mg/l kinetin and solidified with 5.5 g/l agarose (pH 5.7). Dishes were sealed and cultured at 24° C. with standard light conditions (16 h/day). After 3 weeks, calli developing at the ends of the explants were transferred to fresh M-338 [H76] plates (25 calli/plate). Calli were subcultured to fresh medium every 3 weeks (larger calli cut into two pieces).

### Selection of Mutant HETO134 and of Mutant HETO133

Hypocotyl derived callus (9 weeks old) was used for the selection of herbicide resistant mutants. Small pieces of callus were plated on M-338 [H76] medium containing 25 nM of the ALS inhibitor Foramsulfuron (CAS RN 173159-57-4). The starting concentration of 25 nM Foramsulfuron was chosen to be partially inhibitory to callus growth but not lethal. Dishes were cultured at 24° C. with standard light conditions (16 h/day). After 3 weeks, the calli were divided into 2 pieces each and replated on M-338 [H76] medium containing 50 nM Foramsulfuron. Further increases in Foramsulfuron concentration (e.g. 75 nM, 100 nM, 150 nM, or higher) were made each subculture until rapidly growing green (resistant) calli could clearly be identified from the brown non-resistant tissues. Individual mutant clones were transferred to 9 cm dishes containing M-338 [H76] medium and 250 nM Foramsulfuron (a level clearly inhibitory to WT tissues).

The presence in HETO134 of a single point mutation in the tryptophan 559 codon (corresponding to the tryptophan 574 codon in A. thaliana), i.e. a G to T substitution at position 1676 of the coding sequence of ALS I gene, resulting in a Tryptophan to Leucine amino acid substitution at position 559 of the encoded protein, was confirmed by sequence analysis.

The presence in HETO133 of a single point mutation in the tryptophan 556 codon (corresponding to the tryptophan 574 codon in A. thaliana), i.e. a G to T substitution at position 1667 of the coding sequence of ALS III gene, resulting in a Tryptophan to Leucine amino acid substitution at position 556 of the encoded protein, was confirmed by sequence analysis.

### Mutant HETO132

Brassica napus lines with the HETO132 mutation, i.e. comprising a C to T substitution at position 535 and a G to T substitution at position 1667 in ALS III, resulting in a Proline to Serine amino acid substitution at position 179 and a tryptophan to leucine substitution at position 556 of the ALS III protein were generated as follows.

First, Brassica napus lines with the HETO111 mutation, i.e. comprising a C to T substitution at position 535 in ALS III, resulting in a Proline to Serine amino acid substitution at position 179 of the ALS III protein, were generated and identified as described in WO 2011/076345. Based on lines with the HETO111 mutation, lines with the HETO132 mutation were obtained using methods essentially similar to those for obtaining HETO134 (see above), with the modification that the starting material for selection was seed from lines with the HETO111 mutation, and that selection of the mutants took place using higher levels of Foramsulfuron. Following callus induction on M-338 [H76] medium without selection, calli were plated on M-338 [H76] medium containing 0.5 μM Foramsulfuron. After 3 weeks the calli were divided into smaller pieces and replated to M-338 [H76] medium containing 1.0 μM Foramsulfuron. Further increases in Foramsulfuron concentration (e.g. 1.5 μM, 2.0 μM, 2.5 μM 3.0 μM or higher) were made at each subculture until rapidly growing green (resistant) calli could clearly be identified from the brown non-resistant tissues.

The presence in HETO132 of a point mutation in the proline 179 codon and the tryptophan 556 codon (corresponding to the proline 197 and the tryptophan 574 codon in A. thaliana, respectively), i.e. a C to T substitution at position 535 and a G to T substitution at position 1667 of the coding sequence of ALS III gene, resulting in a Proline to Serine amino acid substitution at position 179, and of a Tryptophan to Leucine amino acid substitution at position 556 of the encoded protein, respectively was confirmed by sequence analysis.

### Mutant HETO136

Brassica napus lines with the HETO136 mutation, i.e. comprising a C to T substitution at position 544 and a G to T substitution at position 1676 in ALS I, resulting in a Proline to Serine amino acid substitution at position 182 and a tryptophan to leucine substitution at position 559 of the ALS I protein were generated as follows.

First, Brassica napus lines with the HETO108 mutation, i.e. comprising a C to T substitution at position 544 in ALS I, resulting in a Proline to Serine amino acid substitution at position 182 of the ALS I protein, were generated and identified as described in WO 2011/076345. Based on lines with the HETO108 mutation, lines with the HETO136 mutation were obtained using methods essentially similar to those for obtaining HETO132 (see above), with the modification that the starting material for selection was seed from lines with the HETO108 mutation.

The presence in HETO136 of a point mutation in the proline 182 codon and the tryptophan 559 codon (corresponding to the proline 197 and the tryptophan 574 codon in A. thaliana, respectively), i.e. a C to T substitution at position 544 and a G to T substitution at position 1676 of the coding sequence of ALS I gene, resulting in a Proline to Serine amino acid substitution at position 182, and of a Tryptophan to Leucine amino acid substitution at position 559 of the encoded protein, respectively was confirmed by sequence analysis.

Shoots were recovered from mutant HETO132, HETO133, HETO134 and HETO136 calli following transfer to M338 [H67] regeneration medium without herbicide selection. M-338 [H67] medium is identical to M-338 [H76] except it contains 3 mg/l zeatin, 0.1 mg/l NAA instead in place of 2,4-D and kinetin. Small shoots were excised from the calli and transferred to Magenta boxes containing 50 ml of M-338 [H13] medium without Foramsulfuron selection for further development. M-338 [H13] medium is identical to M-338 [H67] except it contains 2.5 μg/l zeatin and no NAA. Shoots with normal looking leaves were transferred to 2 l sterile glass containers with 100 ml of M-400 rooting medium. M-400 medium is half strength MS salts and vitamins (Murashige and Skoog, 1962) containing 15 g/l sucrose and solidified with 6 g/l plant agar (pH 6.0). After 4 weeks of culture rooted plants were transferred to the glasshouse.

Seeds comprising HETO132 have been deposited at the NCIMB Limited (Ferguson Building, Craibstone Estate, Bucksburn, Aberdeen, Scotland, AB21 9YA, UK) on Jul. 5, 2013, under accession number NCIMB 42153. Of the deposited seeds, 25% is homozygous for the HET0132 mutation and 50% is heterozygous for the HET0132 mutation, which can be identified using methods as described elsewhere in this application. Seeds homozygous for HETO132 have been deposited at the NCIMB Limited (Ferguson Building, Craibstone Estate, Bucksburn, Aberdeen, Scotland, AB21 9YA, UK) on Jul. 1, 2014, under accession number NCIMB 42259.

Seeds comprising HETO136 have been deposited at the NCIMB Limited (Ferguson Building, Craibstone Estate, Bucksburn, Aberdeen, Scotland, AB21 9YA, UK) on Jul. 1, 2014, under accession number NCIMB 42260. Of the deposited seeds, 25% is homozygous for the HET0136 mutation and 50% is heterozygous for the HET0136 mutation, which can be identified using methods as described elsewhere in this application.

Seeds comprising HETO134 and HETO133 were obtained by crossing plants comprising HETO134 with plants comprising HETO133. Seeds comprising and HETO134 and HETO133 have been deposited at the NCIMB Limited (Ferguson Building, Craibstone Estate, Bucksburn, Aberdeen, Scotland, AB21 9YA, UK) on May 20, 2013, under accession number NCIMB 42145. Of the deposited seeds, 25% is homozygous for the HETO134 mutation and 50% is heterozygous for the HETO134, and of the deposited seeds 25% is homozygous for the HET0133 mutation and 50% is heterozygous for the HET0133 mutation, which can be identified using methods as described elsewhere in this application. Seeds homozygous for HETO133 and HETO134 have been deposited at the NCIMB Limited (Ferguson Building, Craibstone Estate, Bucksburn, Aberdeen, Scotland, AB21 9YA, UK) on May 8, 2014, under accession number NCIMB 42235.

### Example 2—Combination of HETO132 and HETO134 Alleles

Heterozygous Brassica plants comprising HETO132 are crossed with heterozygous Brassica plants comprising HETO134. The F1 plants have been selfed to obtain the following genotypes:

| ALS I | ALS III |
|---|---|
| —/— | —/— |
| —/— | HETO132/— |
| —/— | HETO132/HETO132 |
| HETO134/— | —/— |
| HETO134/— | HETO132/— |
| HETO134/— | HETO132/HETO132 |
| HETO134/HETO134 | —/— |
| HETO134/HETO134 | HETO132/— |
| HETO134/HETO134 | HETO132/HETO132 |

wherein — indicates the wild-type allele for ALS I and ALS III.

### Example 3—Measurement of Herbicide Tolerance of Brassica Plants Comprising Mutant AHAS Alleles

The correlation between the presence of mutant AHAS alleles in a Brassica plant grown in the greenhouse and tolerance to thiencarbazone-methyl and foramsulfuron was determined as follows. Treatment post-emergence at the 1-2 leaf stage was carried out in a spray cabinet with a dose of 5 g a.i./ha of thiencarbazone-methyl and 8.75 g a.i./ha of foramsulfuron. The plants were evaluated for phenotype (height, side branching and leave morphology) on scale of 5 to 1, where; type 5=normal (corresponding to wildtype unsprayed phenotype); type 4=normal height, some side branching, normal leaves; type 3=intermediate height, intermediate side branching, normal leaves; type 2=short, severe side branching (“bushy”), some leave malformations; type 1=short, severe side branching (“bushy”), severe leave malformations. Phytotoxicity (PPTOX) was determined and evaluated on a scale of 1 to 9, where 1=completely yellowing, 5=50% of plant is yellow and 9=no yellowing. For assessment of vigor scores, plants were evaluated on a scale of 1 to 9, where 1=very poor (+/−dead), 5=average, 9=vigorous.

Plants comprising HETO132 (P197S-W574L on ALS III) and HETO134 (W574L on ALS I) were compared to plants comprising W574L on ALS III and P197S on ALS I, to plants comprising W574L on both ALS III and ALS I but lacking the P197S mutation. The HETO132 allele (P197S-W574L on ALS III) was also combined with ALS I having the P197S mutation, and with ALS I comprising on one allele P197S and on the other allele W574L (P197S/W574L).

It can be seen from Table 1 that the plants comprising HETO132 and HETO 134 in homozygous form have the highest herbicide tolerance of all genotypes tested. Moreover, it can be seen that, for plants comprising W574L on all alleles of all ALS genes, the addition of P197S on ALS III further increases herbicide tolerance (compare P197S-W574L/P197S-W574L, W574L/W574L with W574L/W574L, W574L/W574L).

TABLE 1
Vigor scores (before treatment and 7 and 14 days after treatment), phytotoxicity (PPTOX)
(7 days after treatment) and phenotype (pheno) (21 days after treatment) scores upon spay
testing post-emergence of plants comprising P197S, W574L and P197S-W574L alleles.

| | | Vigor (1-9) | | | PPTOX (1-9 | pheno (1-5) |
|---|---|---|---|---|---|---|
| | | before | 7 days after | 14 days after | 7 days after | 21 days after |
| Mutant Genotype (ALSIII, ALSI) | HT | treatment | treatment | treatment | treatment | treatment |
| W574L/W574L, P197S/P197S | + | 5 | 5 | 5+ | 6 | 3 |
| W574L/W574L, P197S/P197S | 0 | 5 | 9− | 9 | 9 | nn |
| W574L/W574L, W574L/W574L | + | 5 | 5 | 7− | 7 | 5 |
| W574L/W574L, W574L/W574L | 0 | 5 | 8+ | 9 | 9 | nn |
| P197S-W574L/P197S-W574L, W574L/W574L | + | 5 | 6+ | 7+ | 7 | 5 |
| P197S-W574L/P197S-W574L, P197S/W574L | + | 5 | 6− | 6 | 8 | 4 |
| P197S-W574L/P197S-W574L, P197S/P197S | + | 5 | 4 | 5+ | 5 | 3 |
| P197S-W574L/—, W574L/W574L | + | 5 | 5 | 6 | 6 | 4 |
| P197S-W574L/—, P197S/W574L | + | 5 | 4+ | 5+ | 6 | 3 |
| P197S-W574L/—, P197S/P197S | + | 5 | 3 | 3 | 4 | 1 |
| —/—, W574L/W574L | + | 5 | 4 | 5− | 5 | 3 |
| —/—, P197S/W574L | + | 5 | 3 | 3 | 4 | 1 |
| —/—, P197S/P197S | + | 5 | 1 | dead | 2 | — |
| mix | 0 | 5 | 9− | 9 | 9 | nn |

HT = Herbicide treatment; 0 is untreated, + is treated.

Further combinations of HETO132, HETO133, HETO134 and HETO136 were made by different crossing plants comprising said mutations. The ALS alleles having the P197S mutation only were derived from HETO111 and HETO108. Plants were sprayed at the 1-2 leaf stage with a dose of 60 g a.i./ha of thiencarbazone-methyl and 100 g a.i./ha of foramsulfuron. Vigor was determined before treatment, and 12 days after treatment; PPTOX was determined 12 days after treatment, and Phenotype was determined 14 days after treatment. The results of this analysis are shown in Table 2.

TABLE 2
Vigor scores (before treatment and 12 days after treatment), phytotoxicity (PPTOX) (12 days after treatment) and phenotype (pheno)
(14 days after treatment) scores upon spay testing post-emergence of plants comprising P197S, W574L and P197S-W574L alleles.

| | | Vigor (1-9) | | | | | |
|---|---|---|---|---|---|---|---|
| | | before treatment | | | | PPTOX (1-9) | Pheno (1-5) |
| | | | Leaf | Leaf | 12 days after | 12 days after | 14 days after |
| Mutant Genotype (ALSIII, ALSI) | HT | Vigor | Blistering | Chlorosis | treatment | treatment | treatment |
| P197S-W574L/P197S-W574L, P197S/P197S | + | 5 | 8 | 9 | 4 | 5 | 3 |
| P197S-W574L/P197S-W574L, P197S/P197S | 0 | | | | 9 | 9 | 5 |
| W574L/W574L, W574L/W574L | + | 4+ | 8 | 9 | 5 | 7 | 4 |
| W574L/W574L, W574L/W574L | 0 | | | | 9− | 9 | 5 |
| P197S-W574L/P197S-W574L, W574L/W574L | + | 5 | 7 | 8 | 8 | 8 | 5 |
| P197S-W574L/P197S-W574L, W574L/W574L | 0 | | | | 9− | 9 | 5 |
| P197S-W574L/P197S-W574L, W574L/W574L | + | 4 | 7 | 7− | 8 | 8− | 5 |
| P197S-W574L/P197S-W574L, W574L/W574L | 0 | | | | 9 | 9 | 5 |
| P197S-W574L/P197S-W574L, P197S-W574L/P197S-W574L | + | 3-5 | 8 | 7 | 8 | 8 | 5 |
| P197S-W574L/P197S-W574L, P197S-W574L/P197S | + | | | | 7 | 7 | 5 |
| P197S-W574L/P197S-W574L, P197S/P197S | + | | | | 5 | 5 | 3 |
| P197S-W574L/P197S, P197S-W574L/P197S-W574L | + | | | | 6 | 6 | 4 |
| P197S/P197S, P197S-W574L/P197S-W574L | + | | | | 4 | 5 | 3 |
| P197S-W574L/P197S, P197S-W574L/P197S | + | | | | 4+ | 5 | 3 |
| P197S-W574L/P197S, P197S/P197S | + | | | | 2+ | 3 | 1 |
| P197S/P197S, P197S-W574L/P197S | + | | | | 2+ | 3 | 1 |
| P197S/P197S, P197S/P197S | + | | | | 1 | 1 | — |
| Mix P197S-W574L, P197S-W574L | 0 | | | | 9− | 9 | 5 |

HT = Herbicide treatment; 0 is untreated, + is treated.

Table 2 shows that, for plants comprising W574L on all alleles of all ALS genes, the addition of P197S on ALS III, or on both ALS I and ALS III, further increases herbicide tolerance (compare P197S-W574L/P197S-W574L, W574L/W574L with W574L/W574L, W574L/W574L). It can further be seen that addition of W574L to an allele already comprising P197S further increases herbicide tolerance.

### Example 4—Measurement of Herbicide Tolerance of Brassica Plants Comprising Mutant AHAS Alleles in the Field

Seeds of spring oilseed rape homozygous for HETO132 and HETO134, and seeds of spring oilseed rape homozygous for HETO133 and HETO 134 were sown in a field according to typical practical agricultural methods. Several ALS inhibitor herbicides were applied to the oilseed rape plants by using specific spray equipment for small plot applications. At 9, 16 and 24 days after application, the visible phytotoxicity on the oilseed rape plant was assessed according to a scale from 0% to 100%: 0%=no phytotoxic effects, comparable to untreated 100%=complete control, all plants killed. The results are shown in Table 3. The use of all herbicides in the test lead to a clearly better tolerance of the spring oilseed rape lines homozygous for HETO132 and HETO134 as compared to a standard variety not comprising ALS alleles according to the invention. Moreover, in particular 24 days after application, plants comprising HETO132 and HETO134 showed a better tolerance than plants comprising HETO133 and HETO134, showing that the addition of P197S on ALSIII in plants comprising W574L on all ALS genes, further increases herbicide tolerance to the majority of tested herbicides, including higher concentrations foramsulfuron and thiencarbazone-methyl.

### Example 5—In Vitro ALS Inhibitor Sensitivity and Kinetic Parameters of Proteins Encoded by Different AHAS Mutants According to the Invention

The ALS inhibitor sensitivity and kinetic parameters of AHAS proteins comprising the P197S mutation, the W574L mutation, and both the P197S and W574L mutation in one ALS protein, were determined essentially as described in WO 2013/127766. ALS inhibitor sensitivity of a mixture of two ALS proteins, one of which comprising the P197S and the other comprising the W574L mutations were calculated as described in WO 2013/127766. The results of this analysis are shown in Table 4. It is shown that AHAS protein comprising both the P197S and W574 mutation on the same protein have increased tolerance to the herbicides that were tested as compared to proteins comprising the P197S mutation, as compared to proteins comprising the W574L mutation, and as compared to the mixture of W574L proteins and P197S proteins.

TABLE 3
Percentage phytotoxicity in wild-type (WT) oilseed rape, oilseed
rape homozygous for HETO133 and HETO134, and oilseed rape homozygous
for HETO132 and HETO134 upon herbicide spraying in the field.

| | | | 9 DAA | | | 16 DAA |
|---|---|---|---|---|---|---|
| | | | wild- | HETO133- | HETO132- | wild- |
| Treatment | Dose rate AI (g ai.ha) | | type | HETO134 | HETO134 | type |
| BIOMASS UNTREATED | | | 90 | 80 | 80 | 95 |
| FORAMSULFURON | 50 | GA/HA | 78.3 | 7.7 | 7.7 | 98.3 |
| THIENCARBAZONE-METHYL | 30 | GA/HA | | | | |
| RAPESEED OIL METHYLATED | 366.5 | GA/HA | | | | |
| FORAMSULFURON | 100 | GA/HA | 78.3 | 21.7 | 21.7 | 98 |
| THIENCARBAZONE-METHYL | 60 | GA/HA | | | | |
| RAPESEED OIL METHYLATED | 366.5 | GA/HA | | | | |
| FORAMSULFURON | 50 | GA/HA | 76.7 | 14 | 14 | 98 |
| THIENCARBAZONE-METHYL | 30 | GA/HA | | | | |
| RAPESEED OIL METHYLATED | 733 | GA/HA | | | | |
| FORAMSULFURON | 100 | GA/HA | 76.7 | 20 | 20 | 98 |
| THIENCARBAZONE-METHYL | 60 | GA/HA | | | | |
| RAPESEED OIL METHYLATED | 733 | GA/HA | | | | |
| IMAZAMOX | 40 | GA/HA | 73.3 | 6.7 | 6.7 | 98 |
| RAPESEED OIL METHYLATED | 366.5 | GA/HA | | | | |
| BISPYRIBAC-SODIUM | 50 | GA/HA | 76.7 | 56.7 | 46.7 | 98.3 |
| RAPESEED OIL METHYLATED | 366.5 | GA/HA | | | | |
| PROPOXYCARBAZONE-SODIUM | 70 | GA/HA | 76.7 | 53.3 | 40 | 97 |
| RAPESEED OIL METHYLATED | 366.5 | GA/HA | | | | |
| MESOSULFURON-METHYL | 60 | GA/HA | 76.7 | 11.7 | 11.7 | 98 |
| RAPESEED OIL METHYLATED | 366.5 | GA/HA | | | | |
| PYROXSULAM | 12.5 | GA/HA | 76.7 | 13.3 | 13.3 | 98 |
| CLOQUINTOCET-MEXYL | 37.5 | GA/HA | | | | |
| RAPESEED OIL METHYLATED | 366.5 | GA/HA | | | | |

| | 16 DAA | | 24 DAA | | |
|---|---|---|---|---|---|
| | HETO133- | HETO132- | wild- | HETO133- | HETO132- |
| Treatment | HETO134 | HETO134 | type | HETO134 | HETO134 |
| BIOMASS UNTREATED | 95 | 95 | 95 | 95 | 95 |
| FORAMSULFURON | 6.7 | 3.7 | 100 | 2.7 | 3.3 |
| THIENCARBAZONE-METHYL | | | | | |
| RAPESEED OIL METHYLATED | | | | | |
| FORAMSULFURON | 20.7 | 18.3 | 100 | 7.3 | 3.3 |
| THIENCARBAZONE-METHYL | | | | | |
| RAPESEED OIL METHYLATED | | | | | |
| FORAMSULFURON | 11 | 12.7 | 100 | 10 | 10.7 |
| THIENCARBAZONE-METHYL | | | | | |
| RAPESEED OIL METHYLATED | | | | | |
| FORAMSULFURON | 32.3 | 23 | 100 | 18.3 | 11.7 |
| THIENCARBAZONE-METHYL | | | | | |
| RAPESEED OIL METHYLATED | | | | | |
| IMAZAMOX | 10 | 8.7 | 99 | 0 | 0 |
| RAPESEED OIL METHYLATED | | | | | |
| BISPYRIBAC-SODIUM | 88.3 | 71.7 | 100 | 92.7 | 43.3 |
| RAPESEED OIL METHYLATED | | | | | |
| PROPOXYCARBAZONE-SODIUM | 82.7 | 36.7 | 97.7 | 76.7 | 11.7 |
| RAPESEED OIL METHYLATED | | | | | |
| MESOSULFURON-METHYL | 13.3 | 11.7 | 96 | 3.3 | 20.3 |
| RAPESEED OIL METHYLATED | | | | | |
| PYROXSULAM | 17 | 16 | 98 | 15 | 5 |
| CLOQUINTOCET-MEXYL | | | | | |
| RAPESEED OIL METHYLATED | | | | | |

AI = active ingredient; gai/ha = gram active ingredient/hectare.
Biomass untreated refers to the biomass (%) as compared to a control variety.

TABLE 4
ALS inhibitor sensitivity - pI50 values and resistance factor, and kinetic
parameters of enzymatic activity for different AHAS mutants.

| | pI50 | | | | | Resistance Factor | | | |
|---|---|---|---|---|---|---|---|---|---|
| | | | | P197S- | | | | P197S- | |
| | | | | W574L | P197S & | | | W574L | P197S & |
| | | | | (in one | W574L | | | (in one | W574L |
| Herbicide | WT | P197S | W574L | protein) | (mixture) | P197S | W574L | protein) | (mixture) |
| Amidosulfuron¹ | 6.7 | <4 | <4 | <4 | <4 | >488 | >488 | >488 | >501 |
| Ethoxysulfuron¹ | 7.9 | <4 | <4 | <4 | <4 | >7717 | >7717 | >7717 | >6309 |
| Flazasulfuron¹ | 9.1 | 7.0 | 5.5 | <4 | 6.2 | 120 | 3802 | >120226 | 794 |
| Flupyrsulfuron-methyl¹ | 8.7 | 7.0 | 5.4 | <4 | 6.2 | 48 | 1905 | >47863 | 316 |
| Foramsulfuron¹ | 8.1 | 7.0 | 4.3 | <4 | 5.6 | 12 | 5888 | >11748 | 398 |
| Iodosulfuron-methy-sodium¹ | 8.5 | 6.4 | 5.8 | <4 | 6.1 | 139 | 555 | >35030 | 316 |
| Mesosulfuron-methyl¹ | 8.8 | 7.0 | 4.4 | <4 | 6.0 | 65 | 25770 | >64730 | 794 |
| Metsulfuron-methyl¹ | 7.9 | 6.3 | 5.0 | <4 | 5.6 | 38 | 755 | >7547 | 251 |
| Nicosulfuron¹ | 6.8 | 6.0 | <4 | <4 | 4.5 | 6 | >607 | >607 | 251 |
| Rimsulfuron¹ | 7.7 | 6.4 | 5.0 | <4 | 5.7 | 18 | 447 | >4466 | 63 |
| Sulfosulfuron¹ | 7.7 | 4.4 | <4 | <4 | 3.6 | 1848 | >4641 | >4641 | 10000 |
| Thifensulfuron-Methyl¹ | 7.4 | 5.1 | 4.2 | <4 | 4.6 | 200 | 1585 | >2511 | 631 |
| Bispyribac-sodium² | 7.8 | 7.1 | 5.1 | <4 | 6.1 | 4 | 447 | >5623 | 40 |
| Imazamox⁴ | 5.4 | 5.3 | <4 | <4 | 4.1 | 1 | >22 | >22 | 20 |
| Florasulam³ | 7.8 | 6.4 | 4.6 | <4 | 5.5 | 28 | 1738 | >6918 | 200 |
| Metosulam³ | 8.5 | 5.8 | 4.7 | <4 | 5.2 | 457 | 5754 | >28840 | 2512 |
| Pyroxsulam³ | 8.6 | 6.2 | <4 | <4 | 4.8 | 261 | >41368 | >41368 | 6310 |
| Propoxycarbazone⁵ | 7.7 | 5.5 | 5.2 | <4 | 5.4 | 147 | 293 | >4641 | 200 |
| Thiencarbazone-methyl⁵ | 7.9 | 4.8 | 4.8 | <4 | 4.8 | 1173 | 1173 | >7399 | 1259 |
| Kₘ (mM) | 1.9 | 5.5 | 7.0 | 31.5 | | | | | |
| Vₘₐₓ (deltaOD₅₅₀/min mg protein) | 38.3 | 50.0 | 54.4 | 87.7 | | | | | |

¹Sulfonylureas,
²Pyrimidinylbenzoates,
³Triazolopyrimidines,
⁴Sulfonylaminocarbonyltriazolinones,
⁵Imidozolinones.

### Example 6—Detection and/or Transfer of Mutant AHAS Alleles into (Elite) Brassica Lines

The mutant AHAS genes are transferred into (elite) Brassica breeding lines by the following method: A plant containing a mutant AHAS gene (donor plant), is crossed with an (elite) Brassica line (elite parent/recurrent parent) or variety lacking the mutant AHAS gene. The following introgression scheme is used (the mutant AHAS allele is abbreviated to ahas while the wild type is depicted as AHAS):

Initial cross: ahas/ahas (donor plant) X AHAS/AHAS (elite parent)
F1 plant: AHAS/ahas
BC1 cross: AHAS/ahas X AHAS/AHAS (recurrent parent)
BC1 plants: 50% AHAS/ahas and 50% AHAS/AHAS
The 50% ahas/AHAS are selected by direct sequencing or using molecular markers (e.g. AFLP, PCR, Invader™, TaqMan® and the like) for the mutant AHAS allele (ahas).
BC2 cross: AHAS/AHAS (BC1 plant) X AHAS/AHAS (recurrent parent)
BC2 plants: 50% AHAS/ahas and 50% AHAS/AHAS
The 50% AHAS/AHAS are selected by direct sequencing or using molecular markers for the mutant AHAS allele (ahas).
Backcrossing is repeated until BC3 to BC6
BC3-6 plants: 50% AHAS/ahas and 50% AHAS/ahas
The 50% AHAS/ahas are selected using molecular markers for the mutant AHAS allele (ahas). To reduce the number of backcrossings (e.g. until BC3 in stead of BC6), molecular markers can be used specific for the genetic background of the elite parent.
BC3-6 S1 cross: AHAS/ahas X AHAS/ahas
BC3-6 S1 plants: 25% AHAS/AHAS and 50% AHAS/ahas and 25% ahas/ahas
Plants containing ahas are selected using molecular markers for the mutant AHAS allele (AHAS). Individual BC3-6 S1 or BC3-6 S2 plants that are homozygous for the mutant AHAS allele (ahas/ahas) are selected using molecular markers for the mutant and the wild-type AHAS alleles. These plants are then used for seed production.

To select for plants comprising a point mutation in an AHAS allele, direct sequencing by standard sequencing techniques known in the art, such as those described in Example 1, can be used.

1. A polyploid ALS inhibitor herbicide tolerant plant or parts thereof comprising at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine.

2. The plant or parts thereof according to claim 1, wherein all ALS genes encode an ALS polypeptide comprising at a position corresponding to position 574 of SEQ ID NO: 10 instead of the naturally encoded amino acid tryptophan the amino acid leucine, and comprising at a position corresponding to position 197 of SEQ ID NO: 10 instead of the naturally encoded amino acid proline the amino acid serine.

3. The plant or parts thereof according to claim 1, which is selected from the group consisting of:
a. Brassica napus comprising an ALS I gene encoding an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 556 of SEQ ID NO: 4 instead of the naturally encoded amino acid tryptophan the amino acid leucine;
b. Brassica napus comprising an ALS I gene encoding an ALS I polypeptide comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine;
c. Brassica napus comprising an ALS I gene encoding an ALS I polypeptide comprising at a position corresponding to position 182 of SEQ ID NO: 2 instead of the naturally encoded amino acid proline the amino acid serine and comprising at a position corresponding to position 559 of SEQ ID NO: 2 instead of the naturally encoded amino acid tryptophan the amino acid leucine, and an ALS III gene encoding an ALS III polypeptide comprising at a position corresponding to position 179 of SEQ ID NO: 4 instead of the naturally encoded amino acid proline the amino acid serine and comprising at a position corresponding to position 556 of SEQ ID NO: 4 instead of the naturally encoded amino acid tryptophan the amino acid leucine;
d. Brassica juncea comprising an ALS-A gene encoding an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 559 of SEQ ID NO: 8 instead of the naturally encoded amino acid tryptophan the amino acid leucine;
e. Brassica juncea comprising an ALS-A gene encoding an ALS-A polypeptide comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine; and
f. Brassica juncea comprising an ALS-A gene encoding an ALS-A polypeptide comprising at a position corresponding to position 179 of SEQ ID NO: 6 instead of the naturally encoded amino acid proline the amino acid serine and comprising at a position corresponding to position 556 of SEQ ID NO: 6 instead of the naturally encoded amino acid tryptophan the amino acid leucine, and an ALS-B gene encoding an ALS-B polypeptide comprising at a position corresponding to position 182 of SEQ ID NO: 8 instead of the naturally encoded amino acid proline the amino acid serine and comprising at a position corresponding to position 559 of SEQ ID NO: 8 instead of the naturally encoded amino acid tryptophan the amino acid leucine.

4. The Brassica plant or parts thereof according to claim 3, which is selected from the group consisting of:
a. B. napus comprising an ALS I gene encoding an ALS I polypeptide which is at least 90% identical to SEQ ID NO: 2 of which the proline at position 182 is substituted with a serine, and of which the tryptophan at position 559 is substituted with a leucine, and an ALS III gene encoding an ALS III polypeptide which is at least 90% identical to SEQ ID NO: 4 of which the tryptophan at position 556 is substituted with a leucine;
b. B. napus comprising an ALS I gene encoding an ALS I polypeptide which is at least 90% identical to SEQ ID NO: 2 of which the tryptophan at position 559 is substituted with a leucine, and an ALS III gene encoding an ALS III polypeptide which is at least 90% identical to SEQ ID NO: 4 of which the proline at position 179 is substituted with a serine and of which the tryptophan at position 556 is substituted with a leucine;
c. B. napus comprising an ALS I gene encoding an ALS I polypeptide which is at least 90% identical to SEQ ID NO: 2 of which the proline at position 182 is substituted with a serine, and of which the tryptophan at position 559 is substituted with a leucine, and an ALS III gene encoding an ALS III polypeptide which is at least 90% identical to SEQ ID NO: 4 of which the proline at position 179 is substituted with a serine and of which the tryptophan at position 556 is substituted with a leucine;
d. B. juncea comprising an ALS-A gene encoding an ALS-A polypeptide which is at least 90% identical to SEQ ID NO: 6 of which the proline at position 179 is substituted with a serine and of which the tryptophan at position 556 is substituted with a leucine, and an ALS-B gene encoding an ALS-B polypeptide which is at least 90% identical to SEQ ID NO: 8 of which the tryptophan at position 559 is substituted with a leucine;
e. B. juncea comprising an ALS-A gene encoding an ALS-A polypeptide which is at least 90% identical to SEQ ID NO: 6 of which the tryptophan at position 556 is substituted with a leucine, and an ALS-B gene encoding an ALS-B polypeptide which is at least 90% identical to SEQ ID NO: 8 of which the proline at position 182 is substituted with a serine, and of which the tryptophan at position 559 is substituted with a leucine; and
f. B. juncea comprising an ALS-A gene encoding an ALS-A polypeptide which is at least 90% identical to SEQ ID NO: 6 of which the proline at position 179 is substituted with a serine and of which the tryptophan at position 556 is substituted with a leucine, and an ALS-B gene encoding an ALS-B polypeptide which is at least 90% identical to SEQ ID NO: 8 of which the proline at position 182 is substituted with a serine, and of which the tryptophan at position 559 is substituted with a leucine.

5. The Brassica plant or parts thereof according to claim 4, which is B. napus, which is selected from the group consisting of:
a. a B. napus plant or parts thereof wherein said ALS I gene comprises the nucleotide sequence corresponding to SEQ ID NO: 1 of which the C at position 544 is substituted with T and of which the G at position 1676 is substituted with T, and said ALS III gene comprises the nucleotide sequence corresponding to SEQ ID NO: 3 of which the G at position 1667 is substituted with T, and which is obtainable from seeds deposited at NCIMB under accession numbers NCIMB 42145, NCIMB 42235, and/or NCIMB 42260;
b. a B. napus plant or parts thereof wherein said ALS I gene comprises the nucleotide sequence corresponding to SEQ ID NO: 1 of which the G at position 1676 is substituted with T, and said ALS III gene comprises the nucleotide sequence corresponding to SEQ ID NO: 3 of which the C at position 535 is substituted with T and of which the G at position 1667 is substituted with T, and which is obtainable from seeds deposited at NCIMB under accession numbers NCIMB 42145, NCIMB 42235, NCIMB 42153, and/or NCIMB 42259; and
c. a B. napus plant or parts thereof wherein said ALS I gene comprises the nucleotide sequence corresponding to SEQ ID NO: 1 of which the C at position 544 is substituted with T and of which the G at position 1676 is substituted with T, and said ALS III gene comprises the nucleotide sequence corresponding to SEQ ID NO: 3 of which the C at position 535 is substituted with T and of which the G at position 1667 is substituted with T, and which is obtainable from seeds deposited at NCIMB under accession numbers NCIMB 42153, NCIMB 42259, and/or NCIMB 42260.

6. The Brassica plant or parts thereof according to claim 4, which is B. napus, which is selected from the group consisting of:
a. a B. napus plant or parts thereof wherein said ALS I gene comprises the nucleotide sequence corresponding to SEQ ID NO: 1 of which the C at position 544 is substituted with T and of which the G at position 1676 is substituted with T, and said ALS III gene comprises the nucleotide sequence corresponding to SEQ ID NO: 3 of which the G at position 1667 is substituted with T, reference seed of said plant having been deposited at NCIMB under accession numbers NCIMB 42145, 42235 and NCIMB 42260,
b. a B. napus plant or parts thereof wherein said ALS I gene comprises the nucleotide sequence corresponding to SEQ ID NO: 1 of which the G at position 1676 is substituted with T, and said ALS III gene comprises the nucleotide sequence corresponding to SEQ ID NO: 3 of which the C at position 535 is substituted with T and of which the G at position 1667 is substituted with T, reference seed of said plant having been deposited at NCIMB under accession numbers NCIMB 42145, NCIMB 42235, NCIMB 42153, and NCIMB 42259, and
c. a B. napus plant or parts thereof wherein said ALS I gene comprises the nucleotide sequence corresponding to SEQ ID NO: 1 of which the C at position 544 is substituted with T and of which the G at position 1676 is substituted with T, and said ALS III gene comprises the nucleotide sequence corresponding to SEQ ID NO: 3 of which the C at position 535 is substituted with T and of which the G at position 1667 is substituted with T, reference seed of said plant having been deposited at NCIMB under accession numbers NCIMB 42153, NCIMB 42259, and NCIMB 42260.

7. The plant or parts thereof according to claim 1 which are tolerant to one or more ALS-inhibitor herbicides belonging to the group consisting of sulfonylurea herbicides, sulfonylaminocarbonyltriazolinone herbicides, imidazolinone herbicides, triazolopyrimidine herbicides, and pyrimidinyl(thio)benzoate herbicides.

8. The plant or parts thereof according to claim 1, characterized in that said plant or parts thereof is homozygous for said ALS genes encoding an ALS polypeptide comprising at a position corresponding to position 574 of SEQ ID NO: 10 instead of the naturally encoded amino acid tryptophan the amino acid leucine, and is homozygous for said ALS genes encoding an ALS polypeptide comprising at a position corresponding to position 197 of SEQ ID NO: 10 instead of the naturally encoded amino acid proline the amino acid serine and comprising at a position corresponding to position 574 of SEQ ID NO: 10 instead of the naturally encoded amino acid tryptophan the amino acid leucine.

9. Food, feed, or an industrial product obtainable from a plant according to claim 1.

10. The food, feed or industrial product according to claim 9, wherein
a) the food or feed is oil, meal, grain, starch, flour or protein; or
b) the industrial product is biofuel, fiber, industrial chemicals, a pharmaceutical or a nutraceutical.

11. Progeny of a plant according to claim 1 obtained by further breeding with said plant, wherein all ALS genes of said progeny encode an ALS polypeptide comprising at a position corresponding to position 574 of SEQ ID NO: 10 instead of the naturally encoded amino acid tryptophan the amino acid leucine and wherein at least one of the ALS genes encodes an ALS polypeptide which further comprises at a position corresponding to position 197 of SEQ ID NO: 10 instead of the naturally encoded amino acid proline the amino acid serine.

12. A method of producing a hybrid seed, comprising crossing a parent plant according to claim 1 with a second parent plant and harvesting a resultant hybrid seed.

13. A hybrid plant produced from crossing a parent plant according to claim 1 with a second parent plant and harvesting a resultant hybrid seed and growing said seed, wherein all ALS genes of said hybrid plant encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine.

14. A method of producing food, feed, or an industrial product comprising
a) obtaining the plant or a part thereof, of claim 1; and
b) preparing the food, feed or industrial product from the plant or part thereof.

15. The method of claim 14 wherein
a) the food or feed is oil, meal, grain, starch, flour or protein; or
b) the industrial product is biofuel, fiber, industrial chemicals, a pharmaceutical or a nutraceutical.

16. A method to increase the tolerance to ALS inhibitor herbicide(s) of polyploid plants, said method comprising introducing at least two ALS genes, wherein all ALS genes encodes an ALS polypeptide comprising at a position corresponding to position 574 of SEQ ID NO: 10 instead of the naturally encoded amino acid tryptophan the amino acid leucine, and wherein one ALS gene encodes an ALS polypeptide which further comprises at a position corresponding to position 197 of SEQ ID NO: 10 instead of the naturally encoded amino acid proline the amino acid serine.

17. A method for controlling unwanted vegetation in a growing area, such as a Brassica growing area, or such as a B. napus growing area, which plants, such as Brassica plants, or such as B. napus plants, comprise at least two ALS genes, wherein all ALS genes encode an ALS polypeptide comprising at a position corresponding to position 574 of SEQ ID NO: 10 instead of the naturally encoded amino acid tryptophan the amino acid leucine, and wherein at least one of the ALS genes encodes an ALS polypeptide which further comprises at a position corresponding to position 197 of SEQ ID NO: 10 instead of the naturally encoded amino acid proline the amino acid serine.

18. The method according to claim 17, wherein the ALS inhibitor herbicide(s) belong(s) to:
the group of the (sulfon)amides (group (A)) consisting of:
the subgroup (A1) of the sulfonylureas, consisting of:
amidosulfuron [CAS RN 120923-37-7] (=A1-1);
azimsulfuron [CAS RN 120162-55-2] (=A1-2);
bensulfuron-methyl [CAS RN 83055-99-6] (=A1-3);
chlorimuron-ethyl [CAS RN 90982-32-4] (=A1-4);
chlorsulfuron [CAS RN 64902-72-3] (=A1-5);
cinosulfuron [CAS RN 94593-91-6] (=A1-6);
cyclosulfamuron [CAS RN 136849-15-5] (=A1-7);
ethametsulfuron-methyl [CAS RN 97780-06-8] (=A1-8);
ethoxysulfuron [CAS RN 126801-58-9] (=A1-9);
flazasulfuron [CAS RN 104040-78-0] (=A1-10);
flucetosulfuron [CAS RN 412928-75-7] (=A1-11);
flupyrsulfuron-methyl-sodium [CAS RN 144740-54-5] (=A1-12);
foramsulfuron [CAS RN 173159-57-4] (=A1-13);
halosulfuron-methyl [CAS RN 100784-20-1] (=A1-14);
imazosulfuron [CAS RN 122548-33-8] (=A1-15);
iodosulfuron-methyl-sodium [CAS RN 144550-36-7] (=A1-16);
mesosulfuron-methyl [CAS RN 208465-21-8] (=A1-17);
metsulfuron-methyl [CAS RN 74223-64-6] (=A1-18);
monosulfuron [CAS RN 155860-63-2] (=A1-19);
nicosulfuron [CAS RN 111991-09-4] (=A1-20);
orthosulfamuron [CAS RN 213464-77-8] (=A1-21);
oxasulfuron [CAS RN 144651-06-9] (=A1-22);
primisulfuron-methyl [CAS RN 86209-51-0] (=A1-23);
prosulfuron [CAS RN 94125-34-5] (=A1-24);
pyrazosulfuron-ethyl [CAS RN 93697-74-6] (=A1-25);
rimsulfuron [CAS RN 122931-48-0] (=A1-26);
sulfometuron-methyl [CAS RN 74222-97-2] (=A1-27);
sulfosulfuron [CAS RN 141776-32-1] (=A1-28);
thifensulfuron-methyl [CAS RN 79277-27-3] (=A1-29);
triasulfuron [CAS RN 82097-50-5] (=A1-30);
tribenuron-methyl [CAS RN 101200-48-0] (=A1-31);
trifloxysulfuron [CAS RN 145099-21-4] (sodium) (=A1-32);
triflusulfuron-methyl [CAS RN 126535-15-7] (=A1-33);
tritosulfuron [CAS RN 142469-14-5] (=A1-34);
NC-330 [CAS RN 104770-29-8] (=A1-35);
NC-620 [CAS RN 868680-84-6] (=A1-36);
TH-547 [CAS RN 570415-88-2] (=A1-37);
monosulfuron-methyl [CAS RN 175076-90-1] (=A1-38);
metazosulfuron [CAS RN 868680-84-6] (=A1-309);
methiopyrsulfuron [CAS RN 441050-97-1] (=A1-40);
iofensulfuron-sodium [CAS RN 1144097-30-2] (=A1-41);
propyrisulfuron [CAS RN 570415-88-2] (=A1-42);
the subgroup of the sulfonylaminocarbonyltriazolinones (subgroup ((A2)), consisting of:
flucarbazone-sodium [CAS RN 181274-17-9] (=A2-1);
propoxycarbazone-sodium [CAS RN 181274-15-7] (=A2-2);
thiencarbazone-methyl [CAS RN 317815-83-1] (=A2-3);
the subgroup of the triazolopyrimidines (subgroup (A3)), consisting of:
cloransulam-methyl [147150-35-4] (=A3-1);
diclosulam [CAS RN 145701-21-9] (=A3-2);
florasulam [CAS RN 145701-23-1] (=A3-3);
flumetsulam [CAS RN 98967-40-9] (=A3-4);
metosulam [CAS RN 139528-85-1] (=A3-5);
penoxsulam [CAS RN 219714-96-2] (=A3-6);
pyroxsulam [CAS RN 422556-08-9] (=A3-7);
the subgroup of the sulfonanilides (subgroup (A4)), consisting of:
compounds or salts thereof, and racemates and enantiomers thereof, from the group described by the general formula (I): 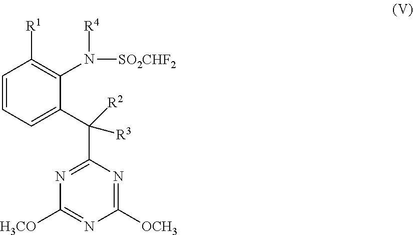
in which
R¹ is halogen, preferably fluorine or chlorine,
R² is hydrogen and R³ is hydroxyl or
R² and R³ together with the carbon atom to which they are attached are a carbonyl group C═O and
R⁴ is hydrogen or methyl;
and more especially compounds of the below given chemical structure (A4-1) to (A4-8) 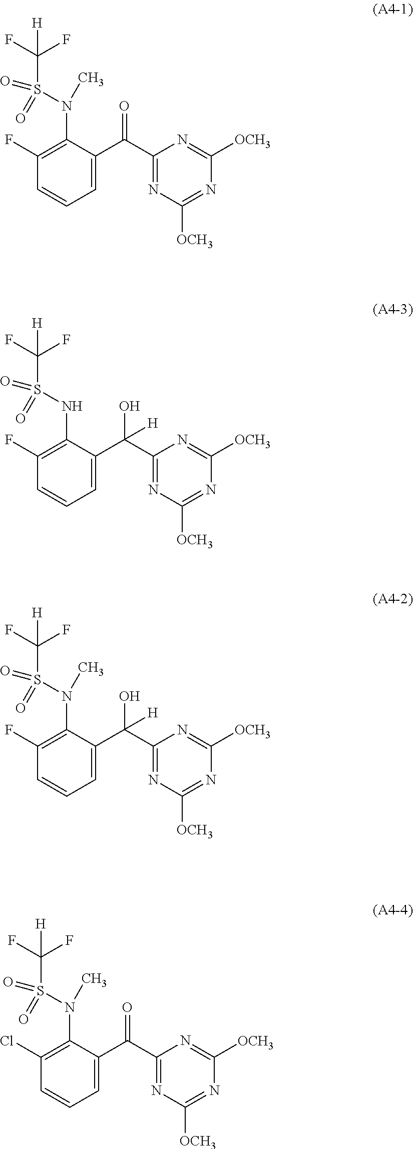 [SMILES: COc1nc(OC)nc(C(=O)c2cccc(F)c2N(C)S(=O)(=O)C(F)F)n1 ; COc1nc(OC)nc(C(O)c2cccc(F)c2NS(=O)(=O)C(F)F)n1 ; COc1nc(OC)nc(C(O)c2cccc(F)c2N(C)S(=O)(=O)C(F)F)n1 ; COc1nc(OC)nc(C(=O)c2cccc(Cl)c2N(C)S(=O)(=O)C(F)F)n1] 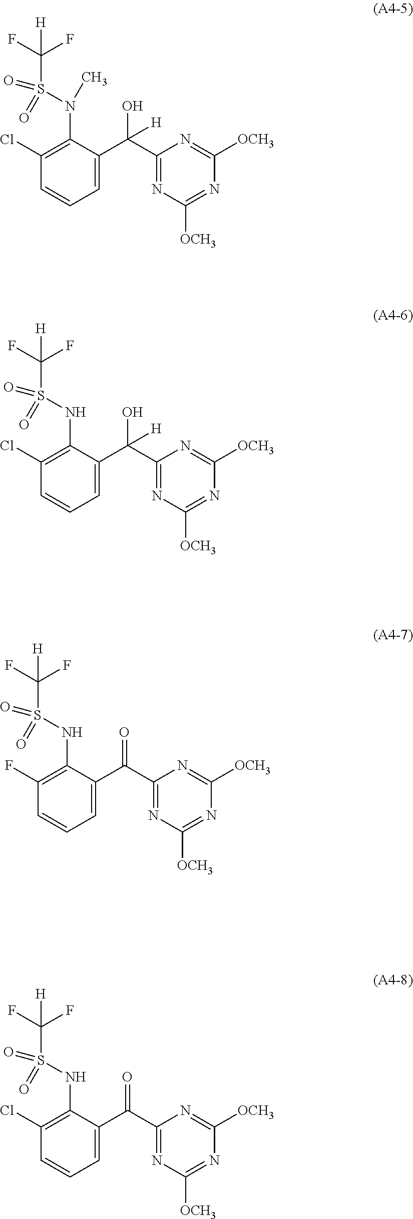 [SMILES: COc1nc(OC)nc(C(O)c2cccc(Cl)c2N(C)S(=O)(=O)C(F)F)n1 ; COc1nc(OC)nc(C(O)c2cccc(Cl)c2NS(=O)(=O)C(F)F)n1 ; COc1nc(OC)nc(C(=O)c2cccc(F)c2NS(=O)(=O)C(F)F)n1 ; COc1nc(OC)nc(C(=O)c2cccc(Cl)c2NS(=O)(=O)C(F)F)n1]
the group of the imidazolinones (group (B)), consisting of:
imazamethabenzmethyl [CAS RN 81405-85-8] (=B1-1);
imazamox [CAS RN 114311-32-9] (=B1-2);
imazapic [CAS RN 104098-48-8] (=B1-3);
imazapyr [CAS RN 81334-34-1] (=B1-4);
imazaquin [CAS RN 81335-37-7] (=B1-5);
imazethapyr [CAS RN 81335-77-5] (=B1-6);
SYP-298 [CAS RN 557064-77-4] (=B1-7); and
SYP-300 [CAS RN 374718-10-2] (=B1-8).
the group of the pyrimidinyl(thio)benzoates (group (C)), consisting of:
the subgroup of the pyrimidinyloxybenzoeacids (subgroup (C1)) consisting of:
bispyribac-sodium [CAS RN 125401-92-5] (=C1-1);
pyribenzoxim [CAS RN 168088-61-7] (=C1-2);
pyriminobac-methyl [CAS RN 136191-64-5] (=C1-3);
pyribambenz-isopropyl [CAS RN 420138-41-6] (=C1-4); and
pyribambenz-propyl [CAS RN 420138-40-5] (=C1-5);
the subgroup of the pyrimidinylthiobenzoeacids (subgroup (C2)), consisting of:
pyriftalid [CAS RN 135186-78-6] (=C2-1); and
pyrithiobac-sodium [CAS RN 123343-16-8] (=C2-2).

19. The method according to claim 17, wherein the ALS inhibitor herbicide(s) belong(s) to the group consisting of:
amidosulfuron [CAS RN 120923-37-7] (=A1-1);
foramsulfuron [CAS RN 173159-57-4] (=A1-13);
iofensulfuron-sodium [CAS RN 1144097-30-2] (=A1-41);
thiencarbazone-methyl [CAS RN 317815-83-1] (=A2-3);
imazamox [CAS RN 114311-32-9] (=B1-2); and
bispyribac-sodium [CAS RN 125401-92-5] (=C1-1).

20. The method according to claim 17, wherein the plants are selected from the group consisting of:
a. B. napus plants comprising an ALS I B. napus gene encoding an ALS I polypeptide comprising at a position corresponding to position 182 of SEQ ID NO: 2 instead of the naturally encoded amino acid proline the amino acid serine and at a position corresponding to position 559 of SEQ ID NO: 2 instead of the naturally encoded amino acid tryptophan the amino acid leucine, and wherein an ALS III B. napus gene encodes an ALS III polypeptide comprising at a position corresponding to position 556 of SEQ ID NO: 4 instead of the naturally encoded amino acid tryptophan the amino acid leucine; and
b. B. napus plants comprising an ALS I B. napus gene encoding an ALS I polypeptide comprising at a position corresponding to position 559 of SEQ ID NO: 2 instead of the naturally encoded amino acid tryptophan the amino acid leucine, and wherein an ALS III B. napus gene encodes an ALS III polypeptide comprising at a position corresponding to position 179 of SEQ ID NO: 4 instead of the naturally encoded amino acid proline the amino acid serine and at a position corresponding to position 556 of SEQ ID NO: 4 instead of the naturally encoded amino acid tryptophan the amino acid leucine.

21. The method according to claim 17, comprising applying the ALS inhibitor herbicide in combination with non-ALS inhibitor herbicides (i.e. herbicides showing a mode of action that is different to the inhibition of the ALS enzyme [acetohydroxyacid synthase; EC 2.2.1.6] (group B herbicides), and wherein the non ALS inhibitor herbicide(s) is/are selected form the group consisting of:
acetochlor (=D1), carbetamide (=D56), fenoxaprop-P-ethyl (=D164), fluazifop-P-butyl (=D174), haloxyfop-P-methyl (=D222), metolachlor (=D275), dimethenamid (=D132), napropamide (=D290), pethoxamid (=D317), propaquizafop (=D341), propisochlor (=D344), propyzamide (=D345), quinmerac (=D363), propachlor (D 427), clomazone (=D83), clopyralid (=D86), dimethachlor (=D130), metazachlor (=D265), picloram (=D321), and quizalofop-P-ethyl (=D368).
